(19) United States
(12) Patent Application Publication
(10) Pub. No.: US 2026/0165026 A1
(43) Pub. Date: Jun. 11, 2026

(54) ORGANIC COMPOUND AND ORGANIC LIGHT-EMITTING DIODE INCLUDING THE SAME

(71) Applicant: Material Science Co., Ltd., Hwaseong-si (KR)

(72) Inventors: Gi Rim LEE, Hwaseong-si (KR); Woo Hyoung CHO, Hwaseong-si (KR); Hee Jun PARK, Hwaseong-si (KR)

(21) Appl. No.: 19/058,600

(22) Filed: Feb. 20, 2025

(30) Foreign Application Priority Data
Feb. 23, 2024 (KR) 10-2024-0026180
Feb. 18, 2025 (KR) 10-2025-0021017

(51) Int. Cl. H10K 85/60; C07D 471/10; H10K 50/12

(52) U.S. Cl. CPC H10K 85/6572; C07D 471/10; H10K 85/615; H10K 85/626; H10K 85/654; H10K 85/6574; H10K 85/6576; H10K 50/12

(57) ABSTRACT

An organic light-emitting diode includes an n-type charge generation layer including a compound represented by a Chemical Formula 1 which facilitates hole and electron migration in a plurality of light emitting stacks. Thus, the organic light-emitting diode including the n-type charge generation layer may secure low operation voltage, high efficiency, and long lifetime characteristics.

### CROSS-REFERENCE TO RELATED APPLICATION

This application claims priority from Korean Patent Application No. 10-2024-0026180 filed on Feb. 23, 2024 and Korean Patent Application No. 10-2025-0021017 filed on Feb. 18, 2025 in the Korean Intellectual Property Office, and all the benefits accruing therefrom under 35 U.S.C. 119, the contents of which in its entirety are herein incorporated by reference.

### BACKGROUND

### Field

The present disclosure relates to an organic compound and an organic light-emitting diode including the same.

### Description of Related Art

An organic light-emitting diode (OLED) has a simpler structure compared to other flat panel display diodes such as a liquid crystal display (LCD), a plasma display panel (PDP), and a field emission display (FED), and has various advantages in terms of a manufacturing process, and has excellent high luminance and wide viewing angle, fast response speed, and low operation voltage, and thus is being actively developed and commercialized as a flat display such as a wall-mounted TV, a backlight for a display, lighting, and billboards.

The organic light-emitting diode has a structure including a positive electrode (a hole injection electrode), a negative electrode (an electron injection electrode), and one or more organic layers disposed between the two electrodes. The organic light-emitting diode has a structure in which a hole injection layer (HIL), a hole transport layer (HTL), a light emitting layer (EML), an electron transport layer (ETL), and an electron injection layer (EIL) are stacked sequentially from the positive electrode. Further, in order to increase the efficiency of the light emitting layer, the organic light-emitting diode may additionally include an electron blocking layer (EBL) or a hole blocking layer (HBL) in contact with the light emitting layer.

### PRIOR ART DOCUMENT

### Patent Document

(Prior Patent Document 1) CN 104119334 A
(Prior Patent Document 2) CN 118791489 A

### SUMMARY

A purpose of the present disclosure is to provide a novel organic compound and an organic light-emitting diode including the same.

The present disclosure is directed to providing a novel organic compound and an organic lighting emitting diode including the same.

In addition to the above objects, embodiments according to the present disclosure may be used to achieve other objects not specifically mentioned.

Purposes of the present disclosure are not limited to the above-mentioned purpose. Other purposes and advantages of the present disclosure that are not mentioned may be understood based on following descriptions, and may be more clearly understood based on embodiments of the present disclosure. Further, it will be easily understood that the purposes and advantages of the present disclosure may be realized using means shown in the claims and combinations thereof.

In order to achieve the above purpose, according to an embodiment of the present disclosure, an organic compound having a novel structure represented by a following Chemical Formula 1 may be provided. For example, an organic compound represented by a Chemical Formula 1-1 may be provided. Definitions of the Chemical Formula 1 and the Chemical Formula 1-1 are the same as those described in the present disclosure.

According to another embodiment of the present disclosure, an organic light emitting display diode includes: an positive electrode; a negative electrode facing the positive electrode; and N light emitting stacks disposed between the positive electrode and the negative electrode, wherein N is an integer of 2 or larger, and the N light emitting stacks include N−1 n-type charge generation layers, each being disposed between different adjacent light emitting stacks, wherein at least one of the n-type charge generation layers may include the compound represented by the Chemical Formula 1, for example, the compound represented by the Chemical Formula 1-1.

Since the n-type charge generation layer including the compound represented by the Chemical Formula 1 according to the present disclosure facilitates hole and electron migration in the plurality of light emitting stacks, the organic light-emitting diode including the n-type charge generation layer may secure low operation voltage, high efficiency, and long lifespan characteristics.

The effects of the present disclosure are not limited to the effects mentioned above, and other effects not mentioned will be clearly understood by those of ordinary skill in the art from the entire description of the present specification. The above effects and additional effects will be described in detail below.

### BRIEF DESCRIPTION OF DRAWINGS

FIG. 1 schematically illustrates an organic light-emitting diode having a tandem structure and including two light emitting stacks and an n-type charge generation layer disposed therebetween according to an embodiment of the present disclosure.

FIG. 2 schematically illustrates an organic light-emitting diode having a tandem structure and including three light emitting stacks and two n-type charge generation layers disposed therebetween according to an embodiment of the present disclosure.

### DETAILED DESCRIPTIONS

Advantages and features of the present disclosure, and a method of achieving the advantages and features will become apparent with reference to embodiments described later in detail together with the accompanying drawings. However, the present disclosure is not limited to the embodiments as disclosed below, but may be implemented in various different forms. Thus, these embodiments are set forth only to make the present disclosure complete, and to completely inform the scope of the present disclosure to those of ordinary skill in the technical field to which the present disclosure belongs, and the present disclosure is only defined by the scope of the claims.

A shape, a size, a ratio, an angle, a number, etc. disclosed in the drawings for describing the embodiments of the present disclosure are illustrative, and the present disclosure is not limited thereto. The same reference numerals refer to the same elements herein. Further, descriptions and details of well-known steps and elements are omitted for simplicity of the description. Furthermore, in the following detailed description of the present disclosure, numerous specific details are set forth in order to provide a thorough understanding of the present disclosure. However, it will be understood that the present disclosure may be practiced without these specific details. In other instances, well-known methods, procedures, components, and circuits have not been described in detail so as not to unnecessarily obscure aspects of the present disclosure.

The terminology used herein is directed to the purpose of describing particular embodiments only and is not intended to be limiting of the present disclosure. As used herein, the singular constitutes “a” and “an” are intended to include the plural constitutes as well, unless the context clearly indicates otherwise. It will be further understood that the terms “comprise”, “comprising”, “include”, and “including” when used in this specification, specify the presence of the stated features, integers, operations, elements, and/or components, but do not preclude the presence or addition of one or more other features, integers, operations, elements, components, and/or portions thereof. As used herein, the term “and/or” includes any and all combinations of one or more of the associated listed items. Expression such as “at least one of” when preceding a list of elements may modify the entire list of elements and may not modify the individual elements of the list. In interpretation of numerical values, an error or tolerance therein may occur even when there is no explicit description thereof.

In addition, it will also be understood that when a first element or layer is referred to as being present “on” a second element or layer, the first element may be disposed directly on the second element or may be disposed indirectly on the second element with a third element or layer being disposed between the first and second elements or layers. It will be understood that when an element or layer is referred to as being “connected to”, or “coupled to” another element or layer, it may be directly on, connected to, or coupled to the other element or layer, or one or more intervening elements or layers may be present. In addition, it will also be understood that when an element or layer is referred to as being “between” two elements or layers, it may be the only element or layer between the two elements or layers, or one or more intervening elements or layers may also be present.

Further, as used herein, when a layer, film, region, plate, or the like is disposed “on” or “on a top” of another layer, film, region, plate, or the like, the former may directly contact the latter or still another layer, film, region, plate, or the like may be disposed between the former and the latter. As used herein, when a layer, film, region, plate, or the like is directly disposed “on” or “on a top” of another layer, film, region, plate, or the like, the former directly contacts the latter and still another layer, film, region, plate, or the like is not disposed between the former and the latter. Further, as used herein, when a layer, film, region, plate, or the like is disposed “below” or “under” another layer, film, region, plate, or the like, the former may directly contact the latter or still another layer, film, region, plate, or the like may be disposed between the former and the latter. As used herein, when a layer, film, region, plate, or the like is directly disposed “below” or “under” another layer, film, region, plate, or the like, the former directly contacts the latter and still another layer, film, region, plate, or the like is not disposed between the former and the latter.

In descriptions of temporal relationships, for example, temporal precedent relationships between two events such as “after”, “subsequent to”, “before”, etc., another event may occur therebetween unless “directly after”, “directly subsequent” or “directly before” is not indicated.

It will be understood that, although the terms “first”, “second”, “third”, and so on may be used herein to describe various elements, components, regions, layers and/or sections, these elements, components, regions, layers and/or sections should not be limited by these terms. These terms are used to distinguish one element, component, region, layer or section from another element, component, region, layer or section. Thus, a first element, component, region, layer or section described below could be termed a second element, component, region, layer or section, without departing from the spirit and scope of the present disclosure.

The features of the various embodiments of the present disclosure may be partially or entirely combined with each other, and may be technically associated with each other or operate with each other. The embodiments may be implemented independently of each other and may be implemented together in an association relationship.

In interpreting a numerical value, the value is interpreted as including an error range unless there is no separate explicit description thereof.

The features of the various embodiments of the present disclosure may be partially or entirely combined with each other, and may be technically associated with each other or operate with each other. The embodiments may be implemented independently of each other and may be implemented together in an association relationship.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The term “metal component” used in the present disclosure refers to a metal element component compound and a derivative compound thereof that may be used in a constituent layer of an organic light-emitting diode. For example, the metal element component compound may include an alkali metal such as lithium (Li), sodium (Na), potassium (K), and cesium (Cs), an alkaline earth metal such as magnesium (Mg), strontium (Sr), barium (Ba), and radium (Ra), a lanthanide metal such as ytterbium (Yb), and a combination thereof. However, the present disclosure is not limited thereto. In one example, the derivative compound of the metal element component may include an alkali metal halide-based material or an alkaline earth metal halide-based material, such as NaF, LiF, CsF, BaF₂, MgF₂, or the like, and/or an organometallic material such as Liq, lithium benzoate, sodium stearate, or the like. However, the present disclosure is not limited thereto.

As used herein, the term “halogen group” includes fluorine, chlorine, bromine and iodine.

As used herein, the term “alkyl group” refers to both straight-chain alkyl radicals and branched-chain alkyl radicals. Unless otherwise specified, an alkyl group contains 1 to 30 carbon atoms. In this case, the alkyl group may include methyl, ethyl, propyl, isopropyl, butyl, secondary butyl, isobutyl, ter-butyl, pentyl, isoamyl, hexyl, etc. However, the present disclosure is not limited thereto. Additionally, the alkyl group may be optionally substituted.

As used herein, the term “cycloalkyl group” refers to a cyclic alkyl radical. Unless otherwise specified, a cycloalkyl group contains 3 to 20 carbon atoms. In this case, the cycloalkyl group may include cyclopropyl, cyclobutyl, cyclopentyl, cyclohexyl, norbornyl, adamantyl, etc. However, the present disclosure is not limited thereto. Additionally, the cycloalkyl group may be optionally substituted.

As used herein, the term “alkenyl group” refers to both straight-chain alkenyl radicals and branched-chain alkenyl radicals having one or more carbon-carbon double bonds. Unless otherwise specified, an alkenyl group contains 2 to 30 carbon atoms. In this case, the alkenyl group may include vinyl, allyl, isopropenyl, 2-butenyl, etc. However, the present disclosure is not limited thereto. Additionally, the alkenyl group may be optionally substituted.

As used herein, the term “cycloalkenyl group” refers to a cyclic alkenyl radical. Unless otherwise specified, a cycloalkenyl group contains 3 to 20 carbon atoms. Additionally, the cycloalkenyl group may be optionally substituted.

As used herein, the term “alkynyl group” refers to both straight-chain and branched-chain alkynyl radicals having one or more carbon-carbon triple bonds. Unless otherwise specified, an alkynyl group contains 2 to 30 carbon atoms. In this case, an alkynyl group may include, but is not limited to, ethynyl, 2-propanyl, etc. Additionally, the alkynyl group may be optionally substituted.

As used herein, the term “cycloalkynyl group” refers to a cyclic alkynyl radical. Unless otherwise specified, a cycloalkynyl group contains 3 to 20 carbon atoms. Additionally, cycloalkynyl groups may be optionally substituted.

The terms “aralkyl group” and “arylalkyl group” as used herein are used interchangeably with each other and refer to an alkyl group having an aromatic group as a substituent. Additionally, the aralkyl group (arylalkyl group) may be optionally substituted.

The terms “aryl group” and “aromatic group” as used herein are used as having the same meaning, and the aryl group includes both a monocyclic group and a polycyclic group. The polycyclic group may include a “fused ring” in which two or more rings are fused with each other such that two carbons are common to two adjacent rings. Moreover, in the polycyclic group, two or more rings may be simply attached or fused to each other. Unless otherwise specified, the aryl group contains 6 to 30 carbon atoms. In this case, the aryl group may include phenyl, naphthyl, anthracenyl, phenanthryl, fluorenyl, dimethylfluorenyl, diphenylfluorenyl, and spirofluorenyl, etc. but is not limited thereto. Additionally, the aryl group may be optionally substituted.

The terms “heteroaryl group” and “heteroaromatic group” as used herein are used as having the same meaning, and the heteroaryl group includes both a monocyclic group and a polycyclic group. The polycyclic group may include a “fused ring” in which two or more rings are fused with each other such that two carbons or heteroatoms are common to two adjacent rings. Moreover, in the polycyclic group, two or more rings may be simply attached or fused to each other. Unless otherwise specified, the heteroaryl group contains 5 to 60 carbon atoms. In this regard, one or more carbons of a ring are replaced with heteroatoms such as oxygen (O), nitrogen (N), sulfur(S), or selenium (Se). In this case, the heteroaryl group may include a 6-membered monocyclic ring such as pyridyl, pyrazinyl, pyrimidinyl, pyridazinyl, and triazinyl, a polycyclic ring such as phenoxathiinyl, indolizinyl, indolyl, purinyl, quinolyl, isoquinolyl, benzooxazolyl, benzothiazolyl, benzoimidazolyl, benzofuranyl, dibenzofuranyl, benzothiophenyl, dibenzothiophenyl, phenylcarbazolyl, 9-phenylcarbazolyl, and carbazolyl, and 2-furanyl, N-imidazolyl, 2-isoxazolyl, 2-pyridinyl, 2-pyrimidinyl, etc, but is not limited thereto. Additionally, the heteroaryl group may be optionally substituted.

The term “heterocyclic group” as used herein means that at least one of the carbon atoms constituting an aryl group, a cycloalkyl group, a cycloalkenyl group, a cycloalkynyl group, an arylalkyl group, an arylamino group, etc. is substituted with a heteroatom such as oxygen (O), nitrogen (N), sulfur(S), etc. Referring to the above definition, the heterocyclic group may include a heteroaryl group, a heterocycloalkyl group, a heterocycloalkenyl group, a heterocycloalkynyl group, a heteroarylalkyl group, a heteroarylamino group, etc. Additionally, the heterocyclic group may be optionally substituted.

Unless otherwise specified, the term “carbon ring” as used herein may be used as including all of a “cycloalkyl group,” “cycloalkenyl group,” “cycloalkynyl group” as an alicyclic group, “aryl group (aromatic group”) as an aromatic ring group, and “heteroaryl group” as a heteroaromatic ring group.

Each of the terms “heteroalkyl group”, “heteroalkenyl group”, “heteroalkynyl group”, and “heteroarylalkyl group” as used herein means that one or more of the carbon atoms constituting the group is substituted with a heteroatom such as oxygen (O), nitrogen (N), sulfur (S). Additionally, each of the heteroalkyl group, heteroalkenyl group, heteroalkynyl group, and heteroarylalkyl group may be optionally substituted.

As used herein, the terms “alkylamino group”, “arylalkylamino group”, “arylamino group”, and “heteroarylamino group” refer to groups in which the alkyl group, arylalkyl group, aryl group, or a heteroaryl group as a hetero-ring is substituted with an amine group. In this regard, the amino group may include all of the primary, secondary, and tertiary amines. Additionally, the alkylamino group, the arylalkylamino group, the arylamino group, and the heteroarylamino group may be optionally substituted

As used herein, the terms “alkylsilyl group”, “arylsilyl group”, “alkoxy group”, “aryloxy group”, “alkylthio group”, or “arylthio group” refer to groups in which each of the alkyl group and the aryl group is substituted with each of a silyl group, an oxy group, and a thio group. Additionally, the alkylsilyl group, the arylsilyl group, the alkoxy group, the aryloxy group, the alkylthio group, and the arylthio group may be optionally substituted.

The terms “arylene group”, “arylalkylene group”, “heteroarylene group”, or “heteroarylalkylene group” as used herein means a group having two-substitutions in which the aryl group, arylalkyl group, heteroaryl group, or heteroarylalkyl group further includes one substitution. Additionally, the arylene group, arylalkylene group, heteroarylene group, and heteroarylalkylene group may be optionally substituted.

As used herein, the term “substituted” means that a substituent other than hydrogen binds to a corresponding carbon atom. When there are a plurality of substituents, the substituents may be identical with or different from each other.

Unless otherwise specified, a position at which the substitution occurs is not particularly limited as long as a hydrogen atom can be substituted with a substituent, that is, the substituent can replace the hydrogen atom at the position. When two or more substituents, that is, the plurality of substituents are present, the substituents may be identical to or different from each other.

Subjects and substituents as defined in the present disclosure may be identical with or different from each other unless otherwise specified.

As used herein, a unit is based on weight (wt), unless specifically stated. For example, when “%” is written, this is interpreted as weight % (wt %).

Hereinafter, an organic compound and an organic light-emitting diode including the same according to the present disclosure will be described in detail.

An organic compound according to the present disclosure may be represented by a following Chemical Formula 1:

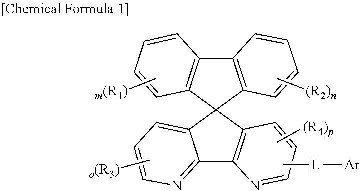

wherein in the Chemical Formula 1,
m and n are identical with or different from each other, and each of m and n is independently an integer of 1 to 4,
and p are identical with or different from each other, and each of o and p is independently an integer of 1 to 3,
L is independently selected from a single bond, a substituted or unsubstituted arylene group having 6 to 30 carbon atoms, a substituted or unsubstituted arylalkylene group having 7 to 30 carbon atoms, a substituted or unsubstituted heteroarylene group having 5 to 60 carbon atoms, and a substituted or unsubstituted heteroarylalkylene group having 6 to 60 carbon atoms,
Ar is independently selected from a substituted or unsubstituted aryl group having 6 to 30 carbon atoms, a substituted or unsubstituted arylalkyl group having 7 to 30 carbon atoms, a substituted or unsubstituted heteroaryl group having 5 to 60 carbon atoms, a substituted or unsubstituted heteroarylalkyl group having 6 to 60 carbon atoms, a substituted or unsubstituted arylamino group having 6 to 30 carbon atoms, a substituted or unsubstituted arylalkylamino group having 6 to 30 carbon atoms, a substituted or unsubstituted heteroarylamino group having 5 to 60 carbon atoms, a substituted or unsubstituted arylsilyl group having 6 to 30 carbon atoms, and a substituted or unsubstituted aryloxy group having 6 to 30 carbon atoms, and binds to an adjacent group thereto to form a substituted or unsubstituted ring,
R₁ to R₄ are identical with or different from each other, and each of R₁ to R₄ is independently selected from hydrogen, a substituted or unsubstituted aryl group having 6 to 30 carbon atoms, a substituted or unsubstituted arylalkyl group having 7 to 30 carbon atoms, a substituted or unsubstituted heteroaryl group having 5 to 60 carbon atoms, a substituted or unsubstituted heteroarylalkyl group having 6 to 60 carbon atoms, a substituted or unsubstituted arylamino group having 6 to 30 carbon atoms, a substituted or unsubstituted arylalkylamino group having 6 to 30 carbon atoms, a substituted or unsubstituted heteroarylamino group having 5 to 60 carbon atoms, a substituted or unsubstituted arylsilyl group having 6 to 30 carbon atoms, and a substituted or unsubstituted aryloxy group having 6 to 30 carbon atoms, and binds to an adjacent group thereto to form a substituted or unsubstituted ring,
wherein when L, Ar, R₁, R₂, R₃, and R₄ are respectively substituted with substituents, the substituents are identical with or different from each other, wherein each of the substituents independently includes at least one selected from deuterium, a cyano group, a trifluoromethyl group, a nitro group, a halogen group, a hydroxy group, a trimethylsilyl group (TMS), an alkyl group having 1 to 30 carbon atoms, a cycloalkyl group having 3 to 20 carbon atoms, an alkenyl group having 2 to 30 carbon atoms, a cycloalkenyl group having 3 to 20 carbon atoms, an alkynyl group having 2 to 30 carbon atoms, a cycloalkynyl group having 3 to 20 carbon atoms, an aryl group having 6 to 30 carbon atoms, an arylalkyl group having 7 to 30 carbon atoms, a heteroaryl group having 5 to 60 carbon atoms, a heteroarylalkyl group having 6 to 60 carbon atoms, an amine group, an alkylamino group having 1 to 30 carbon atoms, an arylalkylamino group having 7 to 30 carbon atoms, an arylamino group having 6 to 30 carbon atoms, a heteroarylamino group having 5 to 60 carbon atoms, a silyl group, an alkylsilyl group having 1 to 30 carbon atoms, an arylsilyl group having 6 to 30 carbon atoms, an alkoxy group having 1 to 30 carbon atoms, an aryloxy group having 6 to 30 carbon atoms, an alkylthio group having 1 to 30 carbon atoms, and an arylthio group having 6 to 30 carbon atoms, when a plurality of substituents are present, each substituent is the same as or different from each other, wherein the substituent binds to an adjacent group thereto to form a substituted or unsubstituted ring. According to an embodiment of the present disclosure, Ar is selected from hydrogen, deuterium, a substituted or unsubstituted aryl group having 6 to 30 carbon atoms, a substituted or unsubstituted arylalkyl group having 7 to 30 carbon atoms, a substituted or unsubstituted heteroaryl group having 5 to 60 carbon atoms, a substituted or unsubstituted heteroarylalkyl group having 6 to 60 carbon atoms, a substituted or unsubstituted alkyl group having 1 to 30 carbon atoms, a substituted or unsubstituted arylamino group having 6 to 30 carbon atoms, a substituted or unsubstituted arylalkylamino group having 6 to 30 carbon atoms, a substituted or unsubstituted heteroarylamino group having 5 to 60 carbon atoms, a substituted or unsubstituted arylsilyl group having 6 to 30 carbon atoms, and a substituted or unsubstituted aryloxy having 6 to 30 carbon atoms,
R₁ to R₄ are identical with or different from each other, and each of R₁ to R₄ is independently hydrogen, deuterium, a substituted or unsubstituted aryl group having 6 to 30 carbon atoms, a substituted or unsubstituted arylalkyl group having 7 to 30 carbon atoms, a substituted or unsubstituted heteroaryl group having 5 to 60 carbon atoms, a substituted or unsubstituted heteroarylalkyl group having 6 to 60 carbon atoms, a substituted or unsubstituted alkyl group having 1 to 30 carbon atoms, a substituted or unsubstituted arylamino group having 6 to 30 carbon atoms, a substituted or unsubstituted arylalkylamino group having 6 to 30 carbon atoms, a substituted or unsubstituted heteroarylamino group having 5 to 60 carbon atoms, a substituted or unsubstituted arylsilyl group having 6 to 30 carbon atoms, and a substituted or unsubstituted aryloxy group having 6 to 30 carbon atoms.

According to an embodiment of the present disclosure, in the compound represented by the Chemical Formula 1, L may be selected from a single bond, a substituted or unsubstituted arylene group having 6 to 30 carbon atoms, and a substituted or unsubstituted heteroarylene group having 5 to 60 carbon atoms. For example, L may be selected from a substituted or unsubstituted phenyl group, a substituted or unsubstituted biphenyl group, a substituted or unsubstituted naphthyl group, a substituted or unsubstituted anthracenyl group, a substituted or unsubstituted phenanthrenyl group, a substituted or unsubstituted dibenzofuranyl group, a substituted or unsubstituted dibenzothiophenyl group, and a combination thereof. However, the present disclosure is not limited thereto.

According to an embodiment of the present disclosure, in the compound represented by the Chemical Formula 1, L may be a mono, bi, or tri-cyclic aryl group or a mono, bi, or tri-cyclic heteroaryl group. For example, L may be a phenyl group, a biphenyl group, a naphthyl group, an anthracenyl group, a phenanthrenyl group, a dibenzofuranyl group, a dibenzothiophenyl group, or a combination thereof. However, the present disclosure is not limited thereto. When L is an aryl group, deviation from a preferred HOMO energy level or LUMO energy level of the compound represented by the Chemical Formula 1 may be minimized. In addition, when L is a naphthyl group or a dibenzofuranyl group, the compound represented by the Chemical Formula 1 may have characteristics suitable for the n-type charge generation layer.

According to an embodiment of the present disclosure, in the compound represented by the Chemical Formula 1, Ar may be deuterium, a substituted or unsubstituted aryl group having 6 to 30 carbon atoms, a substituted or unsubstituted heteroaryl group having 5 to 60 carbon atoms, or a substituted or unsubstituted alkyl group having 1 to 30 carbon atoms.

According to an embodiment of the present disclosure, in the compound represented by the Chemical Formula 1, Ar may be selected from deuterium, a mono, bi, tri, or tetra-cyclic aryl group, a mono, bi, tri, tetra, penta or hexa-cyclic heteroaryl group, and a substituted or unsubstituted alkyl group of 1 to 30 carbon atoms. In addition, Ar may be selected from deuterium, a substituted or unsubstituted aryl group having 6 to 20 carbon atoms, a substituted or unsubstituted heteroaryl group having 5 to 30 carbon atoms, or a substituted or unsubstituted alkyl group having 1 to 20 carbon atoms. For example, Ar may be selected from deuterium, CD₃, a substituted or unsubstituted phenyl group, a substituted or unsubstituted biphenyl group, a substituted or unsubstituted naphthyl group, a substituted or unsubstituted anthracenyl group, a substituted or unsubstituted phenanthrenyl group, a substituted or unsubstituted pyrenyl group, a substituted or unsubstituted carbazolyl group, a substituted or unsubstituted pyridinyl group, a substituted or unsubstituted dibenzofuranyl group, a substituted or unsubstituted dibenzothiophenyl group, a substituted or unsubstituted pyrimidinyl group, a substituted or unsubstituted quinolinyl group, a substituted or unsubstituted isoquinolinyl group, a substituted or unsubstituted benzooxazolyl group, a substituted or unsubstituted benzothiazolyl group, a substituted or unsubstituted benzoimidazolyl group, a substituted or unsubstituted phenanthroline group, a substituted or unsubstituted spiro-dipyridine fluorenyl group, and a combination thereof. However, the present disclosure is not limited thereto.

According to an embodiment of the present disclosure, in the compound represented by the Chemical Formula 1, Ar may be a mono, bi, tri, or tetra-cyclic aryl group, or a mono, bi, tri, or tetra-cyclic heteroaryl group. In this case, since the compound represented by the Chemical Formula 1 may have a high glass transition temperature (Tg) even at a low molecular weight, a deposition temperature thereof may be lowered when the compound is applied to the deposition process. Thus, the thermal stability of the compound may be improved when the organic light-emitting diode operates after the deposition process and the manufacturing of the organic light-emitting diode. For example, Ar may be selected from a phenyl group, a biphenyl group, a naphthyl group, an anthracenyl group, a phenanthrenyl group, a pyrenyl group, a carbazolyl group, a pyridinyl group, a dibenzofuranyl group, a dibenzothiophenyl group, a pyrimidinyl group, a quinolinyl group, an isoquinolinyl group, a benzooxazolyl group, a benzothiazolyl group, a benzoimidazolyl group, a phenanthroline group, and a combination thereof. However, the present disclosure is not limited thereto.

According to an embodiment of the present disclosure, in the compound represented by the Chemical Formula 1, each of R₁, R₂, R₃, and R₄ may be selected from hydrogen, deuterium, a substituted or unsubstituted aryl group having 6 to 30 carbon atoms, a substituted or unsubstituted heteroaryl group having 5 to 60 carbon atoms, or a substituted or unsubstituted alkyl group having 1 to 30 carbon atoms. For example, each of R₁, R₂, R₃, and R₄ may be selected from hydrogen, deuterium, CD₃, a substituted or unsubstituted phenyl group, a substituted or unsubstituted naphthyl group, a substituted or unsubstituted anthracenyl group, a substituted or unsubstituted phenanthrenyl group, a substituted or unsubstituted pyridinyl group, a substituted or unsubstituted dibenzofuranyl group, a substituted or unsubstituted dibenzothiophenyl group, a substituted or unsubstituted pyrimidinyl group, a substituted or unsubstituted phenanthroline group, and a combination thereof. However, the present disclosure is not limited thereto.

In the compound represented by the Chemical Formula 1, R₃ may be selected from deuterium, CD₃, a substituted or unsubstituted phenyl group, a substituted or unsubstituted naphthyl group, a substituted or unsubstituted anthracenyl group, a substituted or unsubstituted phenanthrenyl group, a substituted or unsubstituted pyridinyl group, a substituted or unsubstituted dibenzofuranyl group, a substituted or unsubstituted dibenzothiophenyl group, a substituted or unsubstituted pyrimidinyl group, a substituted or unsubstituted phenanthroline group, and a combination thereof. When R₃ is selected as described above, the structural stability and thermal stability of the compound represented by the Chemical Formula 1 are excellent.

According to an embodiment of the present disclosure, the compound represented by the Chemical Formula 1 of the present disclosure includes spiro-dipyridine fluorene as a core. The compound includes nitrogen (N) of a sp2 hybrid orbital in which electrons are relatively abundant. In this regard, N binds to an alkali metal or an alkaline earth metal that is a dopant of an n-type charge generation layer to form a gap state. The formed gap state may promote the transfer of electrons from the n-type charge generation layer to the electron transport layer and, further, may prevent the alkali metal or the alkaline earth metal from diffusing into the p-type charge generation layer, thereby improving the lifespan of the diode. In addition, according to the present disclosure, a spiro structure which has not been introduced in the conventional n-type charge generation layer is introduced to the present n-type charge generation layer, thereby improving the thermal stability thereof and the characteristics of the organic light-emitting diode including the same.

According to an embodiment of the present disclosure, a linker L of the compound represented by the Chemical Formula 1 of the present disclosure serves as a channel through which the rich electrons of the core reaches Ar via conjugation adjustment, and controls the highest occupied molecular orbital (HOMO) and lowest unoccupied molecular orbital (LUMO). In addition, Ar improves pi (π)-electron density and adjusts the energy level of each of HOMO and LUMO to an appropriate level. Therefore, the electron mobility of the electron transport layer ETL may be improved, such that the transfer of electrons from the electron transport layer ETL to the light emitting stack may be promoted.

According to an embodiment of the present disclosure, in the compound represented by the Chemical Formula 1 of the present disclosure, R₃ and Ar may be identical with or different from each other. For example, when R₃ and Ar are identical with each other such that the compound has a symmetrical structure, the structural stability of the compound may be excellent, and characteristics of the organic light-emitting diode including the same may be improved.

According to an embodiment of the present disclosure, the organic compound of the Chemical Formula 1 may be represented by one of following Chemical Formulas 2 to 9. However, the present disclosure is not limited thereto.

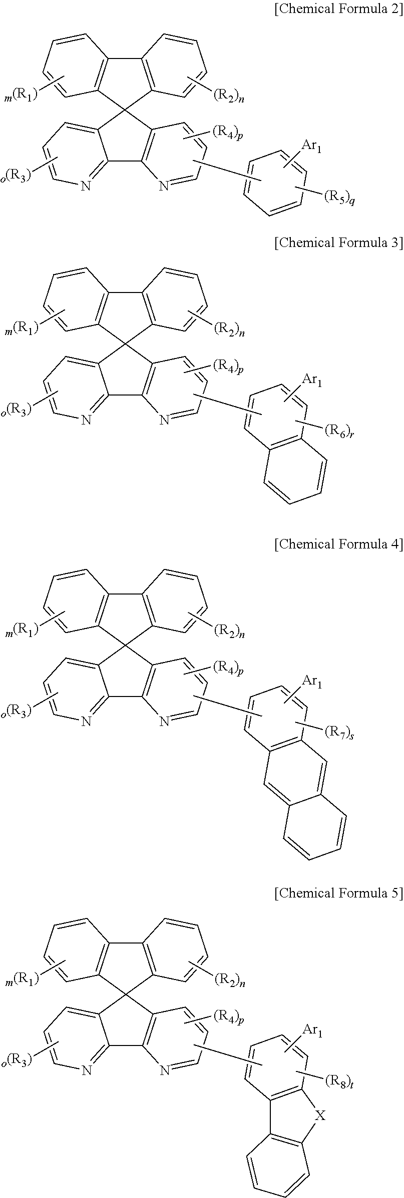

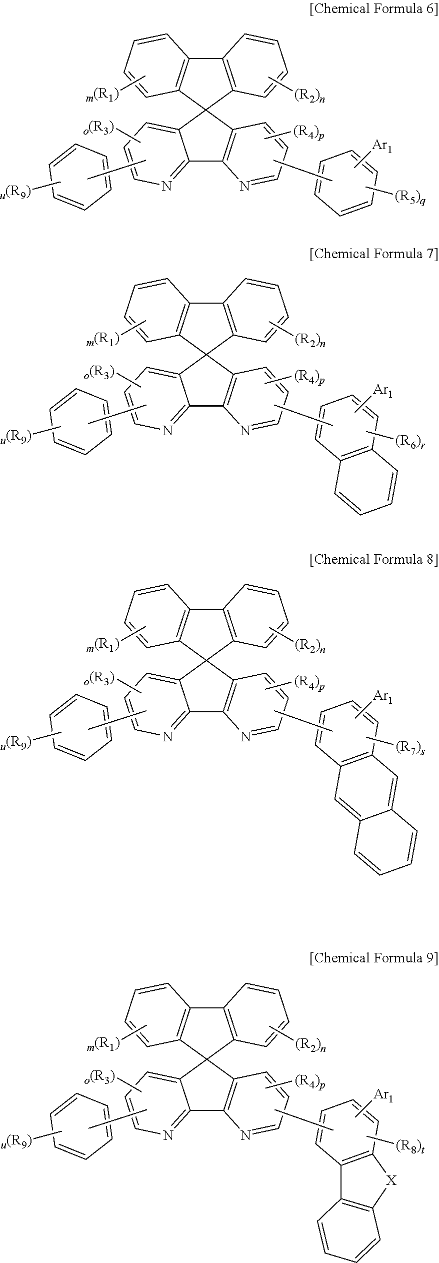

wherein in the Chemical Formulas 2 to 9,
R₁ to R₄, n, m, o, and p are the same as defined in the Chemical Formula 1, q is an integer of 1 to 4, r is an integer of 1 to 6, s is an integer of 1 to 8, t is an integer of 1 to 6, and u is an integer of 1 to 4,
X is oxygen (O) or sulfur(S),
R₅ to R₉ are identical with or different from each other, and each of R₅ to R₉ is independently hydrogen, a substituted or unsubstituted aryl group having 6 to 30 carbon atoms, a substituted or unsubstituted arylalkyl group having 7 to 30 carbon atoms, a substituted or unsubstituted heteroaryl group having 5 to 60 carbon atoms, a substituted or unsubstituted heteroarylalkyl group having 6 to 60 carbon atoms, a substituted or unsubstituted arylamino group having 6 to 30 carbon atoms, a substituted or unsubstituted arylalkylamino group having 6 to 30 carbon atoms, a substituted or unsubstituted heteroarylamino group having 5 to 60 carbon atoms, a substituted or unsubstituted arylsilyl group having 6 to 30 carbon atoms, and a substituted or unsubstituted aryloxy group having 6 to 30 carbon atoms,
Ar₁ is selected from hydrogen, deuterium, a substituted or unsubstituted aryl group having 6 to 30 carbon atoms, a substituted or unsubstituted arylalkyl group having 7 to 30 carbon atoms, a substituted or unsubstituted heteroaryl group having 5 to 60 carbon atoms, a substituted or unsubstituted heteroarylalkyl group having 6 to 60 carbon atoms, a substituted or unsubstituted alkyl group having 1 to 30 carbon atoms, a substituted or unsubstituted arylamino group having 6 to 30 carbon atoms, a substituted or unsubstituted arylalkylamino group having 6 to 30 carbon atoms, a substituted or unsubstituted heteroarylamino group having 5 to 60 carbon atoms, a substituted or unsubstituted arylsilyl group having 6 to 30 carbon atoms, and a substituted or unsubstituted aryloxy group having 6 to 30 carbon atoms,
wherein when R₅ to R₉ and Ar₁ are respectively substituted with substituents, the substituents are identical with or different from each other, wherein each of the substituents independently includes at least one selected from deuterium, a cyano group, a trifluoromethyl group, a nitro group, a halogen group, a hydroxy group, a trimethylsilyl group (TMS), an alkyl group having 1 to 30 carbon atoms, a cycloalkyl group having 3 to 20 carbon atoms, an alkenyl group having 2 to 30 carbon atoms, a cycloalkenyl group having 3 to 20 carbon atoms, an alkynyl group having 2 to 30 carbon atoms, a cycloalkynyl group having 3 to 20 carbon atoms, an aryl group having 6 to 30 carbon atoms, an arylalkyl group having 7 to 30 carbon atoms, a heteroaryl group having 5 to 60 carbon atoms, a heteroarylalkyl group having 6 to 60 carbon atoms, an amine group, an alkylamino group having 1 to 30 carbon atoms, an arylalkylamino group having 7 to 30 carbon atoms, an arylamino group having 6 to 30 carbon atoms, a heteroarylamino group having 5 to 60 carbon atoms, a silyl group, an alkylsilyl group having 1 to 30 carbon atoms, an arylsilyl group having 6 to 30 carbon atoms, an alkoxy group having 1 to 30 carbon atoms, an aryloxy group having 6 to 30 carbon atoms, an alkylthio group having 1 to 30 carbon atoms, and an arylthio group having 6 to 30 carbon atoms, when a plurality of substituents are present, each substituent is the same as or different from each other.

The substituents of R₁ to R₄ are the same as defined in Chemical Formula 1.

According to an embodiment of the present disclosure, t may be an integer of 1 to 2.

According to an embodiment of the present disclosure, in the Chemical Formulas 2 to 9, R₅ to R₉ are identical with or different from each other, and each of R₅ to R₉ may be independently selected from hydrogen, deuterium, a substituted or unsubstituted aryl group having 6 to 30 carbon atoms, a substituted or unsubstituted heteroaryl group having 5 to 60 carbon atoms, or a substituted or unsubstituted alkyl group having 1 to 30 carbon atoms. For example, hydrogen, deuterium, CD3, a substituted or unsubstituted phenyl group, a substituted or unsubstituted naphthyl group, a substituted or unsubstituted anthracenyl group, a substituted or unsubstituted phenanthrenyl group, a substituted or unsubstituted pyridinyl group, a substituted or unsubstituted dibenzofuranyl group, a substituted or unsubstituted dibenzothiophenyl group, a substituted or unsubstituted pyrimidinyl group, a substituted or unsubstituted phenanthroline group, or a combination thereof. However, the present disclosure is not limited thereto.

According to an embodiment of the present disclosure, in the Chemical Formulas 2 to 9, Ar₁ may be selected from be hydrogen, deuterium, a substituted or unsubstituted aryl group having 6 to 30 carbon atoms, a substituted or unsubstituted heteroaryl group having 5 to 60 carbon atoms, or a substituted or unsubstituted alkyl group having 1 to 30 carbon atoms. For example, Ar₁ may be selected from a substituted or unsubstituted phenyl group, a substituted or unsubstituted biphenyl group, a substituted or unsubstituted naphthyl group, a substituted or unsubstituted anthracenyl group, a substituted or unsubstituted phenanthrenyl group, a substituted or unsubstituted pyrenyl group, a substituted or unsubstituted carbazolyl group, a substituted or unsubstituted pyridinyl group, a substituted or unsubstituted dibenzofuranyl group, a substituted or unsubstituted dibenzothiophenyl group, a substituted or unsubstituted pyrimidinyl group, a substituted or unsubstituted quinolinyl group, a substituted or unsubstituted isoquinolinyl group, a substituted or unsubstituted benzooxazolyl group, a substituted or unsubstituted benzothiazolyl group, a substituted or unsubstituted benzoimidazolyl group, a substituted or unsubstituted phenanthroline group, a substituted or unsubstituted spiro-dipyridine fluorenyl group, or a combination thereof. However, the present disclosure is not limited thereto.

According to an embodiment of the present disclosure, in the Chemical Formula 1, L may be selected from a single bond or M1 to M15 as set forth below. However, the present disclosure is not limited thereto.

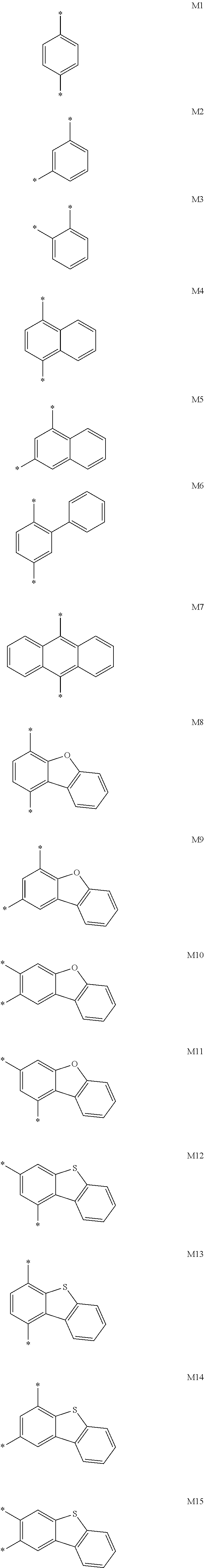

In one embodiment of the present disclosure, in the Chemical Formula 1, Ar may be selected from deuterium, CD₃ or F1 to F59 as set forth below. However, the present disclosure is not limited thereto.

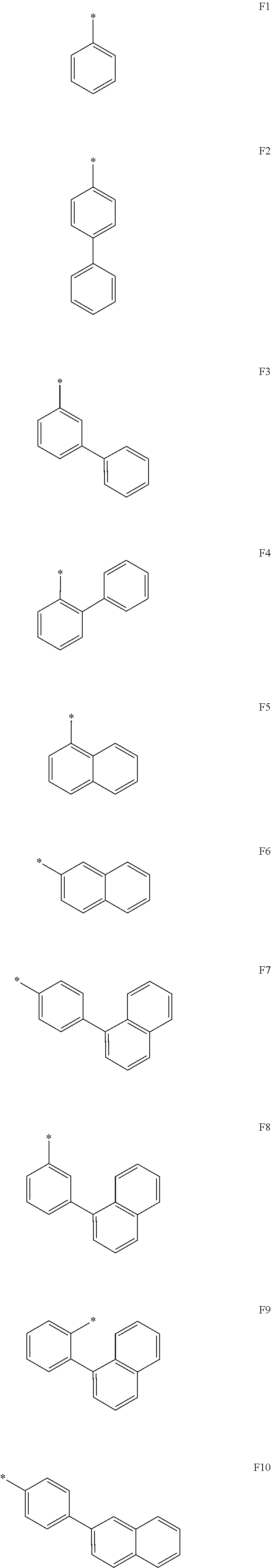

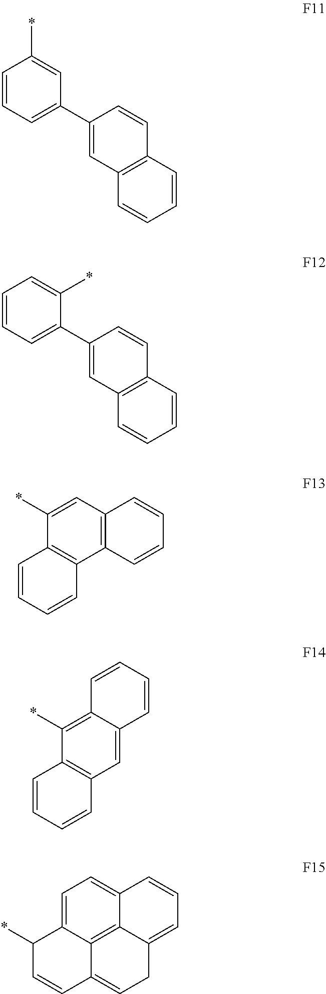 [SMILES: *c1cccc(-c2ccc3ccccc3c2)c1 ; *c1ccccc1-c1ccc2ccccc2c1 ; *c1cc2ccccc2c2ccccc12 ; *c1c2ccccc2cc2ccccc12 ; *C1C=CC2=CCc3cccc4ccc1c2c34]

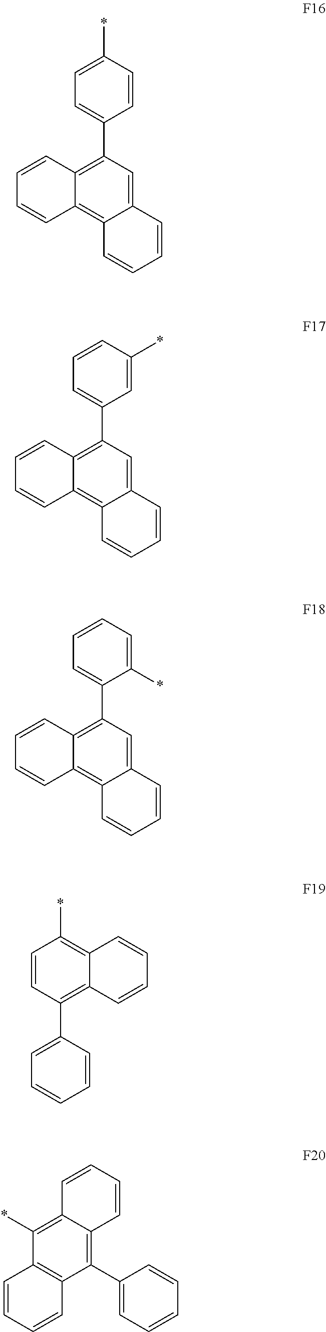 [SMILES: *c1ccc(-c2cc3ccccc3c3ccccc23)cc1 ; *c1cccc(-c2cc3ccccc3c3ccccc23)c1 ; *c1ccccc1-c1cc2ccccc2c2ccccc12 ; *c1ccc(-c2ccccc2)c2ccccc12 ; *c1c2ccccc2c(-c2ccccc2)c2ccccc12]

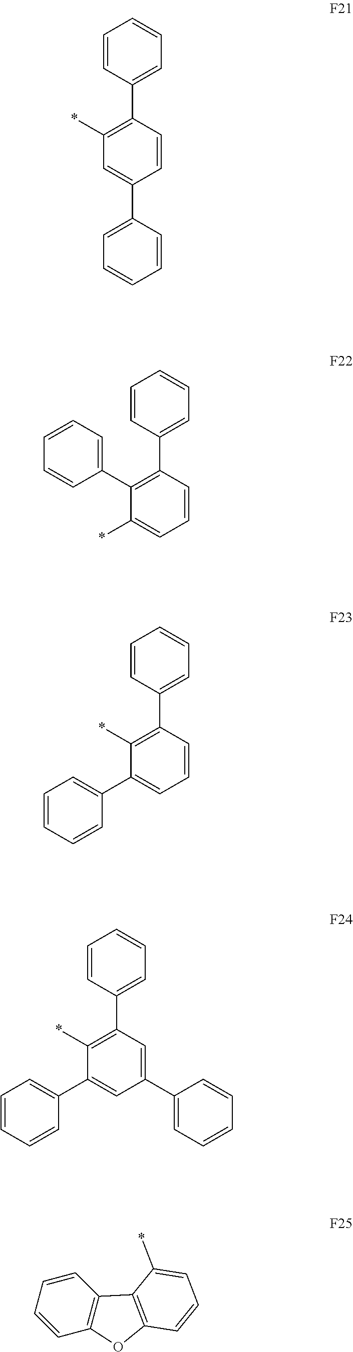 [SMILES: *c1cc(-c2ccccc2)ccc1-c1ccccc1 ; *c1cccc(-c2ccccc2)c1-c1ccccc1 ; *c1c(-c2ccccc2)cccc1-c1ccccc1 ; *c1c(-c2ccccc2)cc(-c2ccccc2)cc1-c1ccccc1 ; *c1cccc2oc3ccccc3c12]

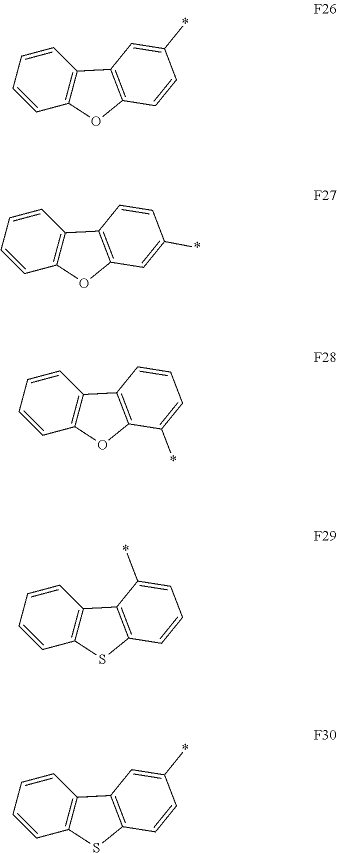 [SMILES: *c1ccc2oc3ccccc3c2c1 ; *c1ccc2c(c1)oc1ccccc12 ; *c1cccc2c1oc1ccccc12 ; *c1cccc2sc3ccccc3c12 ; *c1ccc2sc3ccccc3c2c1]

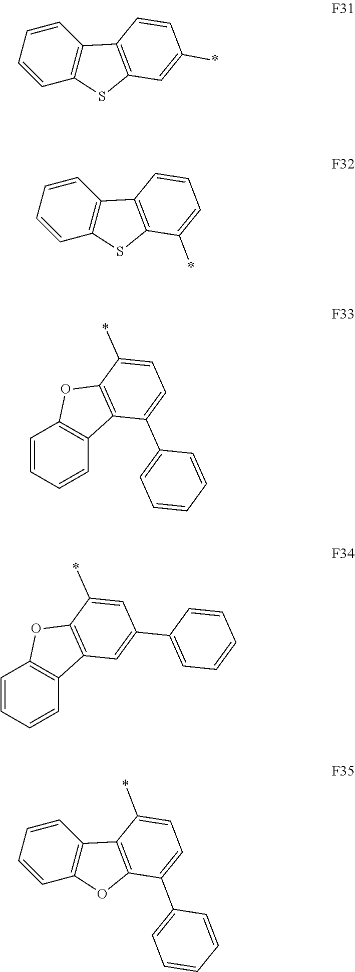 [SMILES: *c1ccc2c(c1)sc1ccccc12 ; *c1cccc2c1sc1ccccc12 ; *c1ccc(-c2ccccc2)c2c1oc1ccccc12 ; *c1cc(-c2ccccc2)cc2c1oc1ccccc12 ; *c1ccc(-c2ccccc2)c2oc3ccccc3c12]

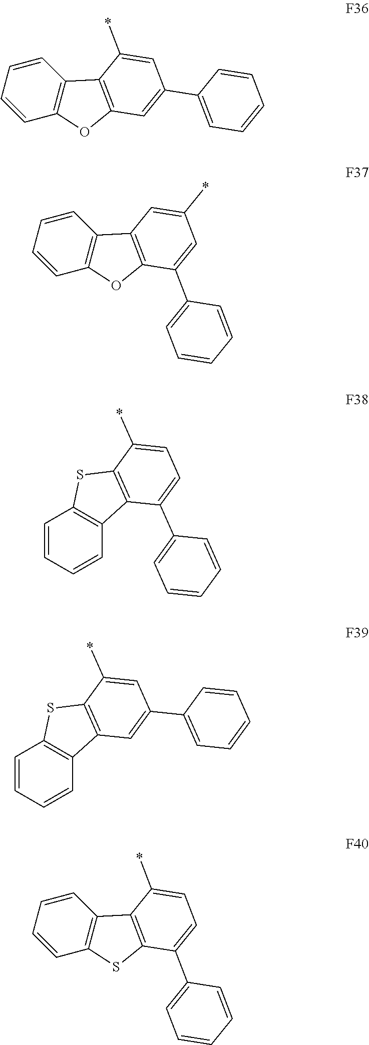 [SMILES: *c1cc(-c2ccccc2)cc2oc3ccccc3c12 ; *c1cc(-c2ccccc2)c2oc3ccccc3c2c1 ; *c1ccc(-c2ccccc2)c2c1sc1ccccc12 ; *c1cc(-c2ccccc2)cc2c1sc1ccccc12 ; *c1ccc(-c2ccccc2)c2sc3ccccc3c12]

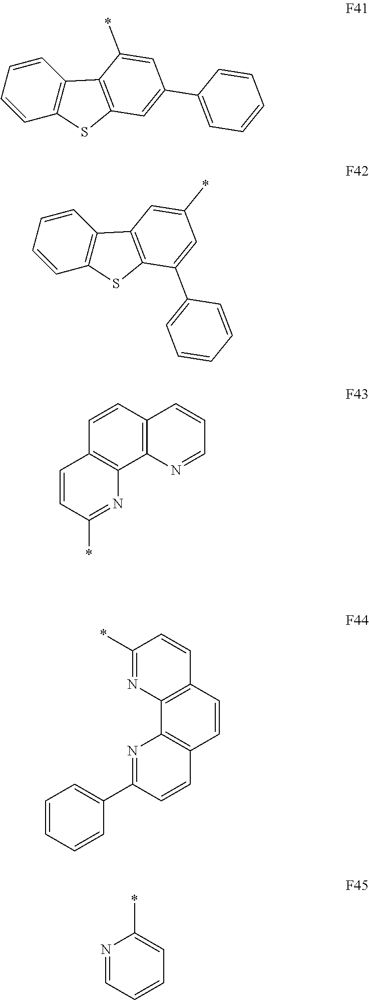 [SMILES: *c1cc(-c2ccccc2)cc2sc3ccccc3c12 ; *c1cc(-c2ccccc2)c2sc3ccccc3c2c1 ; *c1ccc2ccc3cccnc3c2n1 ; *c1ccc2ccc3ccc(-c4ccccc4)nc3c2n1 ; *c1ccccn1]

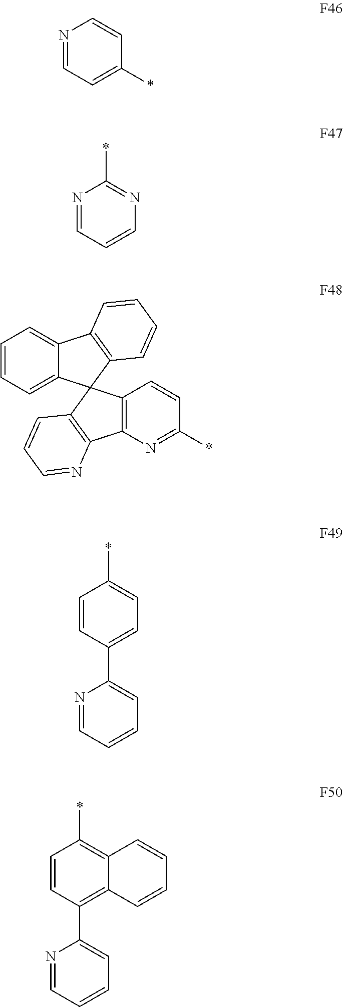 [SMILES: *c1ccncc1 ; *c1ncccn1 ; *c1ccc2c(n1)-c1ncccc1C21c2ccccc2-c2ccccc21 ; *c1ccc(-c2ccccn2)cc1 ; *c1ccc(-c2ccccn2)c2ccccc12]

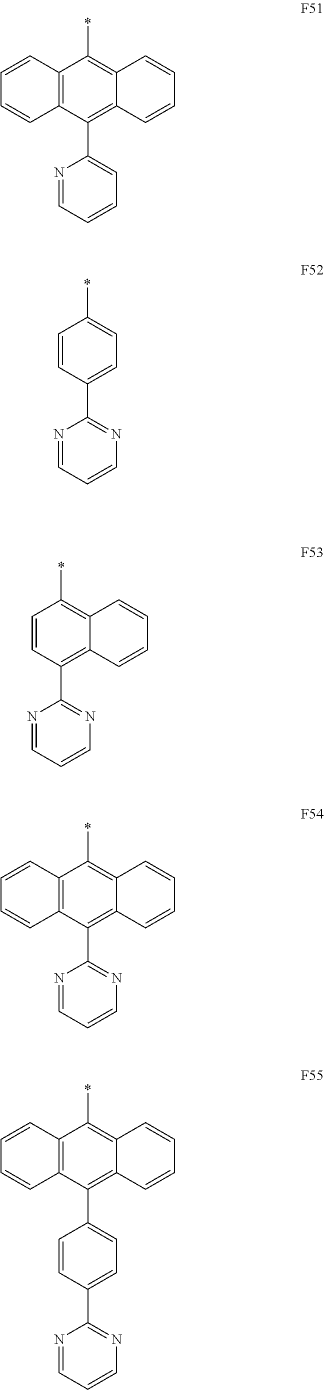 [SMILES: *c1c2ccccc2c(-c2ccccn2)c2ccccc12 ; *c1ccc(-c2ncccn2)cc1 ; *c1ccc(-c2ncccn2)c2ccccc12 ; *c1c2ccccc2c(-c2ncccn2)c2ccccc12 ; *c1c2ccccc2c(-c2ccc(-c3ncccn3)cc2)c2ccccc12]

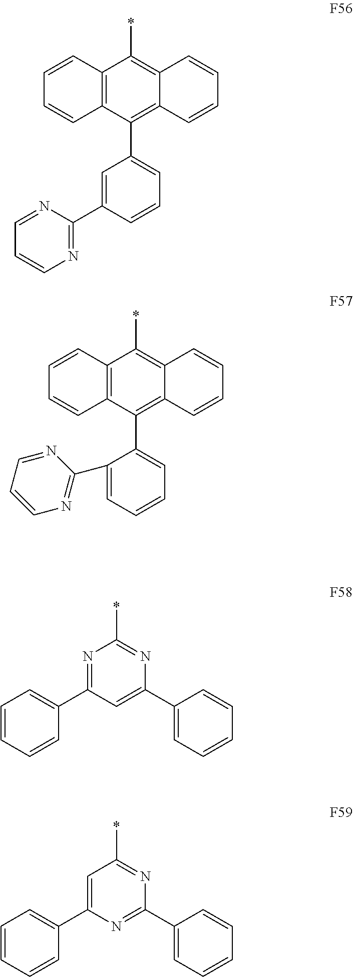 [SMILES: *c1c2ccccc2c(-c2cccc(-c3ncccn3)c2)c2ccccc12 ; *c1c2ccccc2c(-c2ccccc2-c2ncccn2)c2ccccc12 ; *c1nc(-c2ccccc2)cc(-c2ccccc2)n1 ; *c1cc(-c2ccccc2)nc(-c2ccccc2)n1]

In one embodiment of the present disclosure, in the Chemical Formula 1, R₃ may be selected from hydrogen or A1 to A43 as set forth below. However, the present disclosure is not limited thereto.

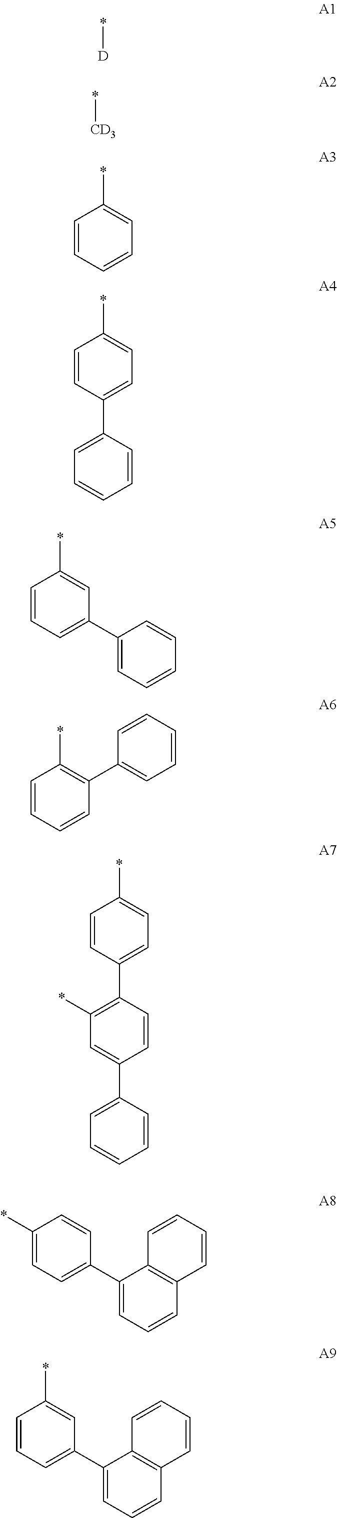

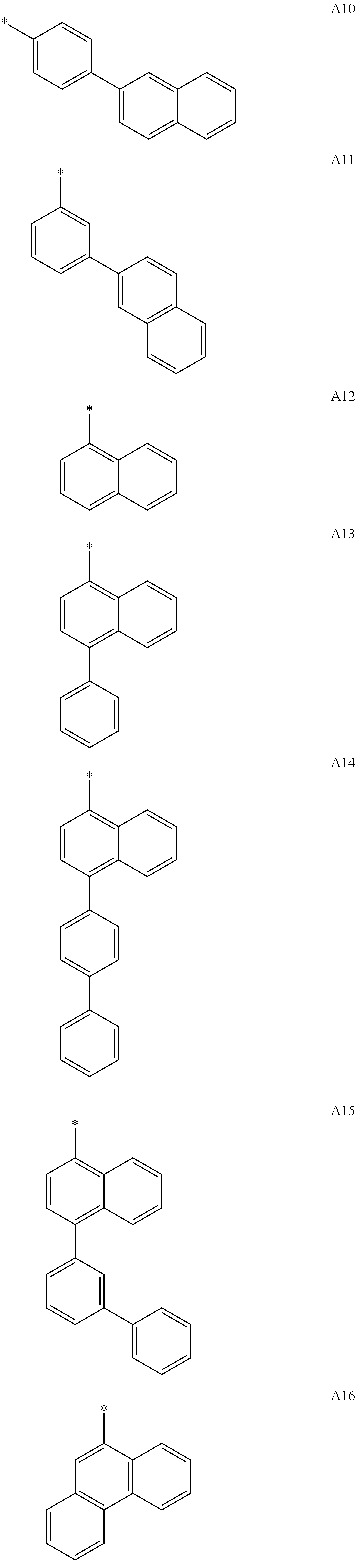

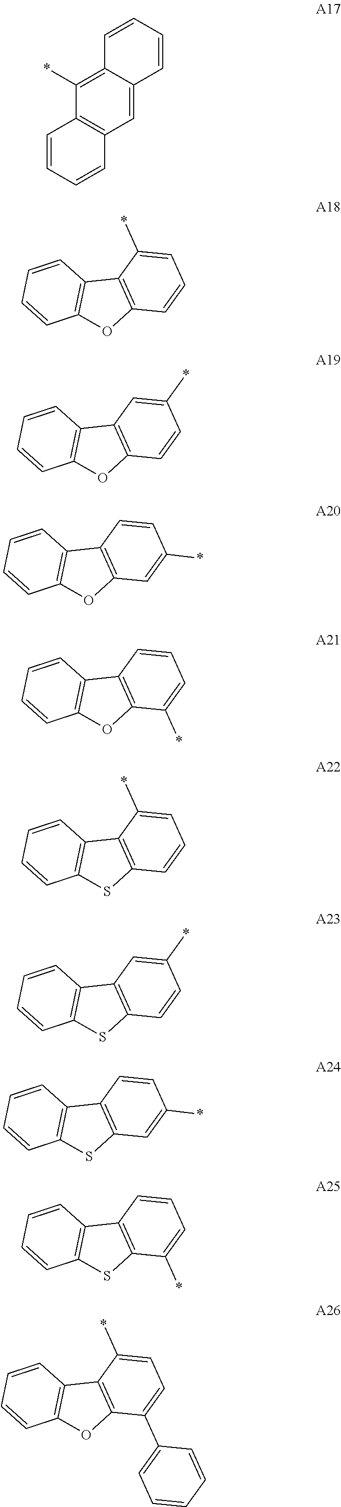

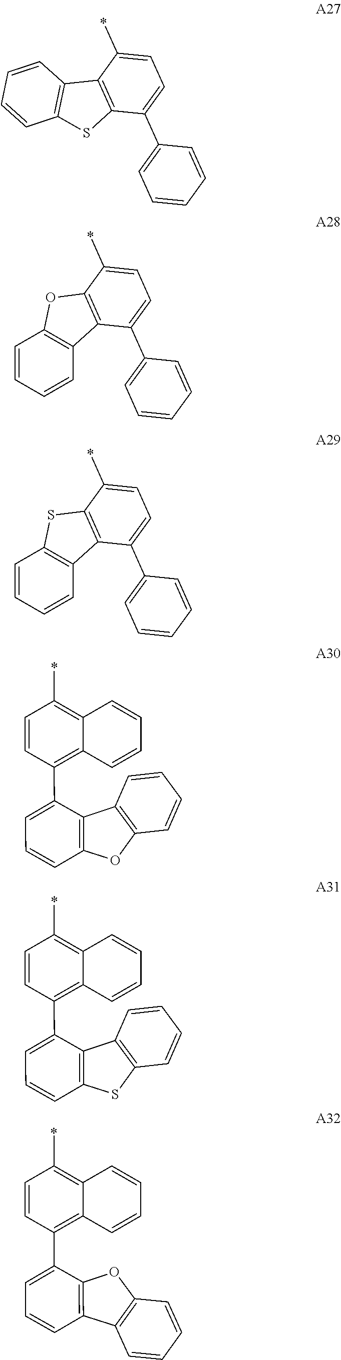

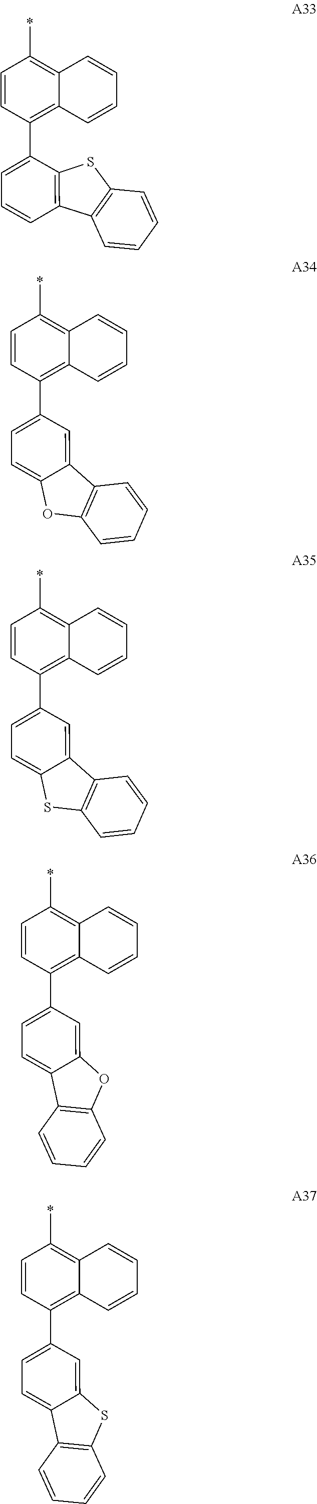 [SMILES: *c1ccc(-c2cccc3c2sc2ccccc23)c2ccccc12 ; *c1ccc(-c2ccc3oc4ccccc4c3c2)c2ccccc12 ; *c1ccc(-c2ccc3sc4ccccc4c3c2)c2ccccc12 ; *c1ccc(-c2ccc3c(c2)oc2ccccc23)c2ccccc12 ; *c1ccc(-c2ccc3c(c2)sc2ccccc23)c2ccccc12]

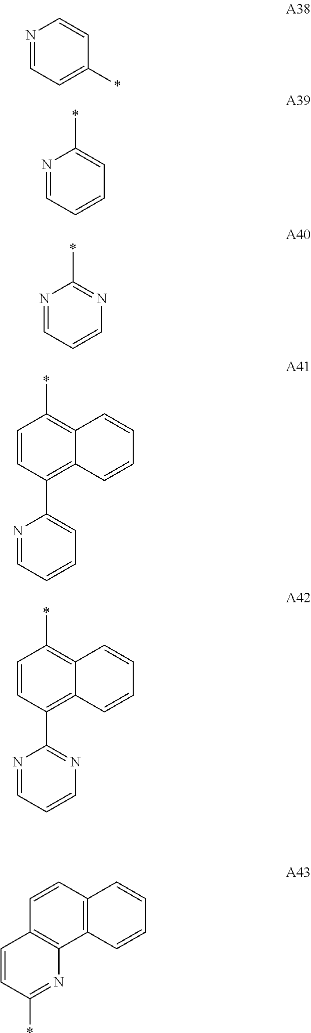

According to one embodiment of the present disclosure, the organic compound of the Chemical Formula 1 may be represented by a following Chemical Formula 1-1.

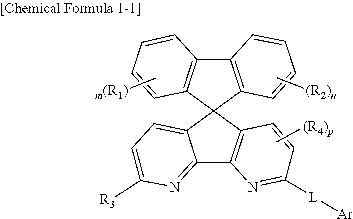

According to one embodiment of the present disclosure, R₁ to R₄, L, Ar, m, n and p are as defined in the Chemical Formula 1.

According to one embodiment of the present disclosure, m and n are identical with or different from each other, and each of m and n is independently an integer of 1 to 4, and p is an integer of 1 to 2.

According to one embodiment of the present disclosure, L is selected from a single bond, a substituted or unsubstituted arylene group having 6 to 30 carbon atoms, and a substituted or unsubstituted heteroarylene group having 5 to 60 carbon atoms.

According to one embodiment of the present disclosure, Ar is selected from hydrogen, deuterium, a substituted or unsubstituted aryl group having 6 to 30 carbon atoms, a substituted or unsubstituted arylalkyl group having 7 to 30 carbon atoms, a substituted or unsubstituted heteroaryl group having 5 to 60 carbon atoms, a substituted or unsubstituted heteroarylalkyl group having 6 to 60 carbon atoms, a substituted or unsubstituted alkyl group having 1 to 30 carbon atoms, a substituted or unsubstituted arylamino group having 6 to 30 carbon atoms, a substituted or unsubstituted arylalkylamino group having 6 to 30 carbon atoms, a substituted or unsubstituted heteroarylamino group having 5 to 60 carbon atoms, a substituted or unsubstituted arylsilyl group having 6 to 30 carbon atoms, and a substituted or unsubstituted aryloxy having 6 to 30 carbon atoms.

According to one embodiment of the present disclosure, R₁ to R₄ are identical with or different from each other, and each of R₁ to R₄ is independently selected from hydrogen, deuterium, a substituted or unsubstituted aryl group having 6 to 30 carbon atoms, a substituted or unsubstituted arylalkyl group having 7 to 30 carbon atoms, a substituted or unsubstituted heteroaryl group having 5 to 60 carbon atoms, a substituted or unsubstituted heteroarylalkyl group having 6 to 60 carbon atoms, a substituted or unsubstituted alkyl group having 1 to 30 carbon atoms, a substituted or unsubstituted arylamino group having 6 to 30 carbon atoms, a substituted or unsubstituted arylalkylamino group having 6 to 30 carbon atoms, a substituted or unsubstituted heteroarylamino group having 5 to 60 carbon atoms, a substituted or unsubstituted arylsilyl group having 6 to 30 carbon atoms, and a substituted or unsubstituted aryloxy group.

According to one embodiment of the present disclosure, when L, Ar, R₁, R₂, R₃, and R₄ are respectively substituted with substituents, the substituents are identical with or different from each other, wherein each of the substituents independently includes at least one selected from deuterium, a cyano group, a trifluoromethyl group, a nitro group, a halogen group, a hydroxy group, a trimethylsilyl group (TMS), an alkyl group having 1 to 30 carbon atoms, a cycloalkyl group having 3 to 20 carbon atoms, an alkenyl group having 2 to 30 carbon atoms, a cycloalkenyl group having 3 to 20 carbon atoms, an alkynyl group having 2 to 30 carbon atoms, a cycloalkynyl group having 3 to 20 carbon atoms, an aryl group having 6 to 30 carbon atoms, an arylalkyl group having 7 to 30 carbon atoms, a heteroaryl group having 5 to 60 carbon atoms, a heteroarylalkyl group having 6 to 60 carbon atoms, an amine group, an alkylamino group having 1 to 30 carbon atoms, an arylalkylamino group having 7 to 30 carbon atoms, an arylamino group having 6 to 30 carbon atoms, a heteroarylamino group having 5 to 60 carbon atoms, a silyl group, an alkylsilyl group having 1 to 30 carbon atoms, an arylsilyl group having 6 to 30 carbon atoms, an alkoxy group having 1 to 30 carbon atoms, an aryloxy group having 6 to 30 carbon atoms, an alkylthio group having 1 to 30 carbon atoms, and an arylthio group having 6 to 30 carbon atoms, when a plurality of substituents are present, each substituent is the same as or different from each other.

According to one embodiment of the present disclosure, the Chemical Formula 1-1 may be represented as one of following Chemical Formulas 10 to 21:

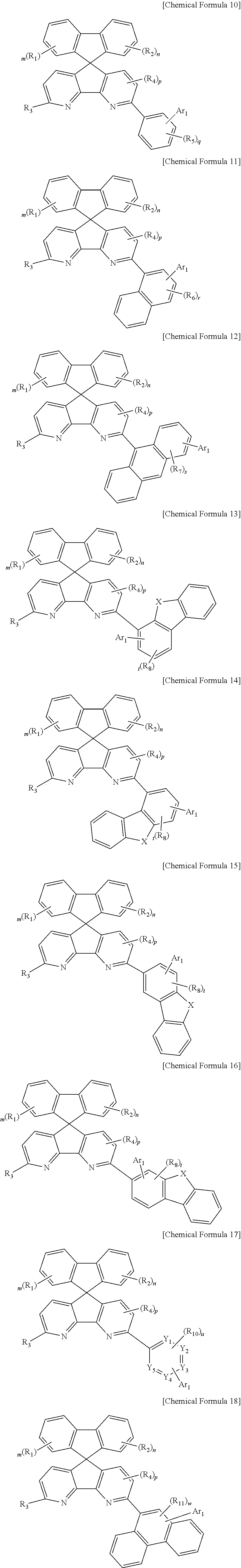

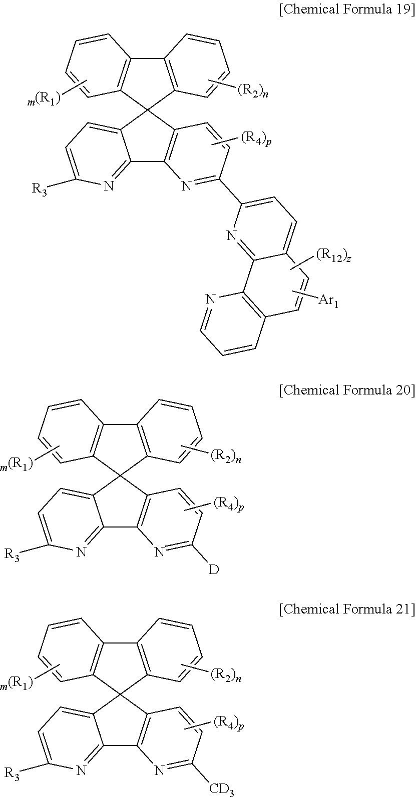

In the above Chemical Formulae 10 to 21, the substituents of X, Ar₁, R₁ to R₈, Ar₁, R₁ to R₈, n, m, p, q, r, s, t are as defined in the above Chemical Formula 1 or the Chemical Formulae 2 to 9.

According to one embodiment of the present disclosure, R₁₀ to R₁₂ are identical with or different from each other, and each of R₁₀ to R₁₂ is independently selected from hydrogen, deuterium, a substituted or unsubstituted aryl group having 6 to 30 carbon atoms, a substituted or unsubstituted arylalkyl group having 7 to 30 carbon atoms, a substituted or unsubstituted heteroaryl group having 5 to 60 carbon atoms, a substituted or unsubstituted heteroarylalkyl group having 6 to 60 carbon atoms, a substituted or unsubstituted arylamino group having 6 to 30 carbon atoms, a substituted or unsubstituted arylalkylamino group having 6 to 30 carbon atoms, a substituted or unsubstituted heteroarylamino group having 5 to 60 carbon atoms, a substituted or unsubstituted arylsilyl group having 6 to 30 carbon atoms, and a substituted or unsubstituted aryloxy group having 6 to 30 carbon atoms.

According to one embodiment of the present disclosure, when R₁₀ to R₁₂ are respectively substituted with substituents, the substituents are identical with or different from each other, wherein each of the substituents independently includes at least one selected from deuterium, a cyano group, a trifluoromethyl group, a nitro group, a halogen group, a hydroxy group, a trimethylsilyl group (TMS), an alkyl group having 1 to 30 carbon atoms, a cycloalkyl group having 3 to 20 carbon atoms, an alkenyl group having 2 to 30 carbon atoms, a cycloalkenyl group having 3 to 20 carbon atoms, an alkynyl group having 2 to 30 carbon atoms, a cycloalkynyl group having 3 to 20 carbon atoms, an aryl group having 6 to 30 carbon atoms, an arylalkyl group having 7 to 30 carbon atoms, a heteroaryl group having 5 to 60 carbon atoms, a heteroarylalkyl group having 6 to 60 carbon atoms, an amine group, an alkylamino group having 1 to 30 carbon atoms, an arylalkylamino group having 7 to 30 carbon atoms, an arylamino group having 6 to 30 carbon atoms, a heteroarylamino group having 5 to 60 carbon atoms, a silyl group, an alkylsilyl group having 1 to 30 carbon atoms, an arylsilyl group having 1 to 30 carbon atoms, an alkoxy group having 1 to 30 carbon atoms, an aryloxy group having 6 to 30 carbon atoms, an alkylthio group having 1 to 30 carbon atoms, and an arylthio group having 6 to 30 carbon atoms, when a plurality of substituents are present, each substituent is the same as or different from each other.

According to one embodiment of the present disclosure, u is an integer of 0 to 4, w is an integer of 0 to 9, and z is an integer of 0 to 7.

According to one embodiment of the present disclosure, Y₁ to Y₅ are C or N, with at least one being N.

According to one embodiment of the present disclosure, the compound represented by the Chemical Formula 1 may be selected from the group consisting of following compounds. However, the present disclosure is not limited thereto.

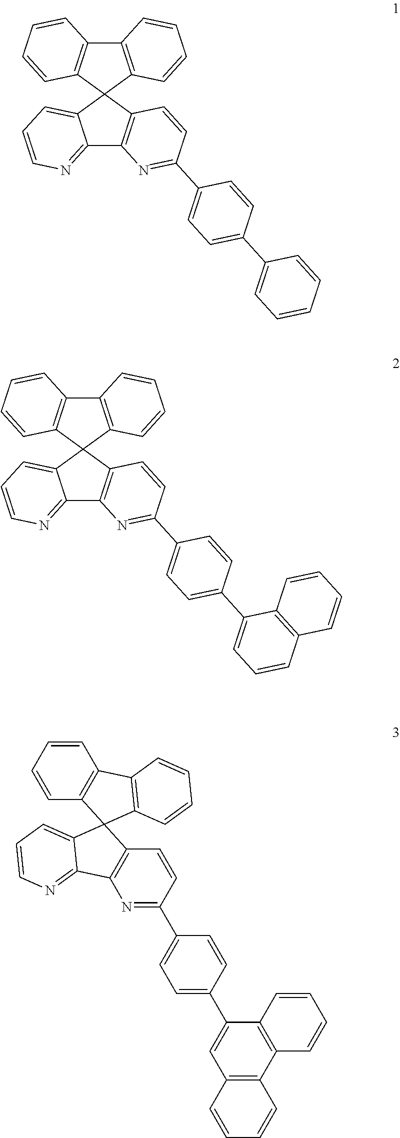 [SMILES: c1ccc(-c2ccc(-c3ccc4c(n3)-c3ncccc3C43c4ccccc4-c4ccccc43)cc2)cc1 ; c1ccc2c(c1)-c1ccccc1C21c2cccnc2-c2nc(-c3ccc(-c4cccc5ccccc45)cc3)ccc21 ; c1ccc2c(c1)-c1ccccc1C21c2cccnc2-c2nc(-c3ccc(-c4cc5ccccc5c5ccccc45)cc3)ccc21]

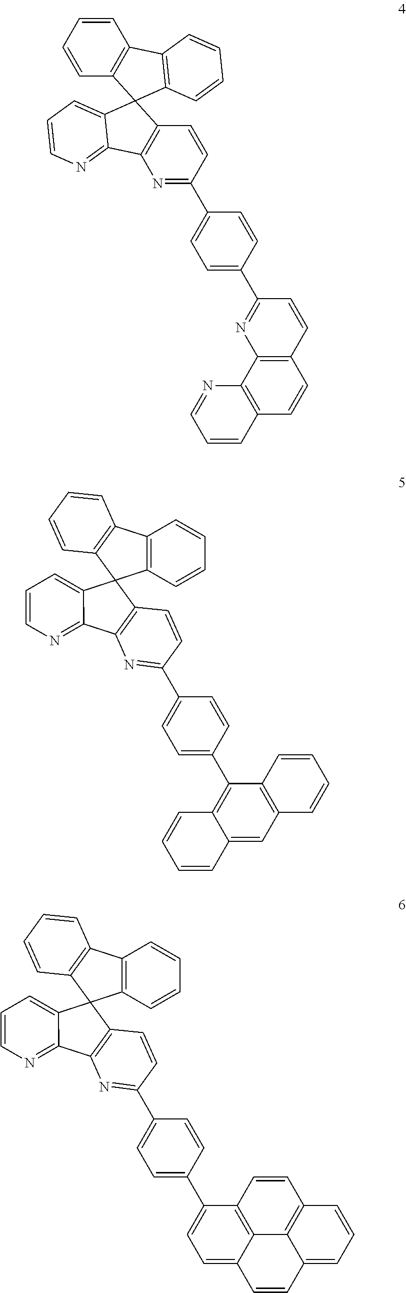 [SMILES: c1ccc2c(c1)-c1ccccc1C21c2cccnc2-c2nc(-c3ccc(-c4ccc5ccc6cccnc6c5n4)cc3)ccc21 ; c1ccc2c(c1)-c1ccccc1C21c2cccnc2-c2nc(-c3ccc(-c4c5ccccc5cc5ccccc45)cc3)ccc21 ; c1ccc2c(c1)-c1ccccc1C21c2cccnc2-c2nc(-c3ccc(-c4ccc5ccc6cccc7ccc4c5c67)cc3)ccc21]

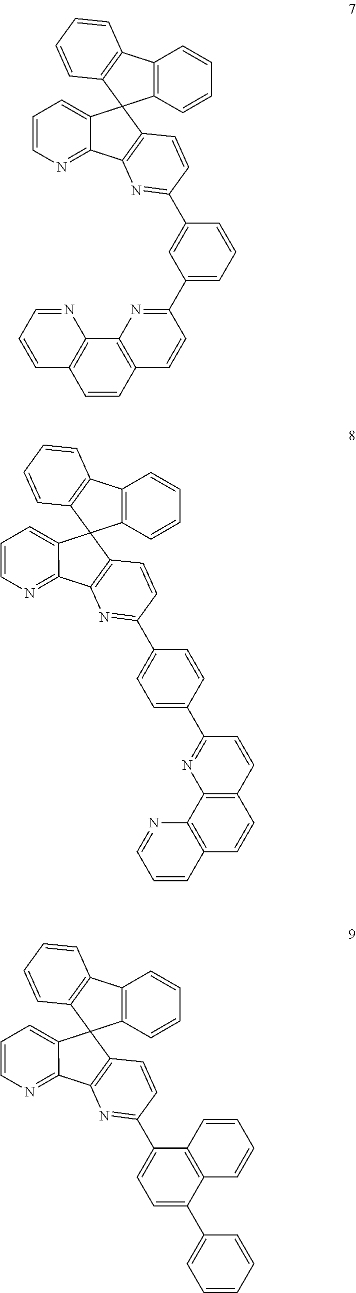 [SMILES: c1cc(-c2ccc3c(n2)-c2ncccc2C32c3ccccc3-c3ccccc32)cc(-c2ccc3ccc4cccnc4c3n2)c1 ; c1ccc2c(c1)-c1ccccc1C21c2cccnc2-c2nc(-c3ccc(-c4ccc5ccc6cccnc6c5n4)cc3)ccc21 ; c1ccc(-c2ccc(-c3ccc4c(n3)-c3ncccc3C43c4ccccc4-c4ccccc43)c3ccccc23)cc1]

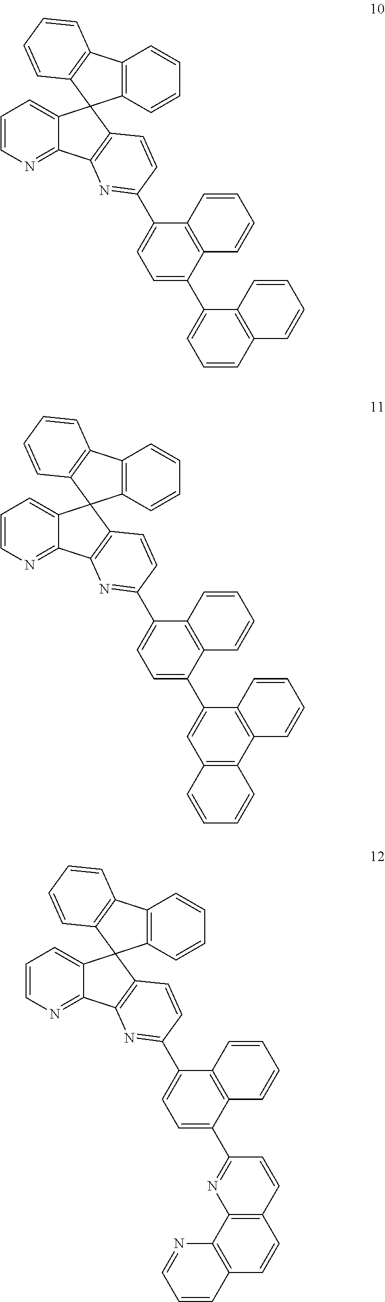 [SMILES: c1ccc2c(c1)-c1ccccc1C21c2cccnc2-c2nc(-c3ccc(-c4cccc5ccccc45)c4ccccc34)ccc21 ; c1ccc2c(c1)-c1ccccc1C21c2cccnc2-c2nc(-c3ccc(-c4cc5ccccc5c5ccccc45)c4ccccc34)ccc21 ; c1ccc2c(c1)-c1ccccc1C21c2cccnc2-c2nc(-c3ccc(-c4ccc5ccc6cccnc6c5n4)c4ccccc34)ccc21]

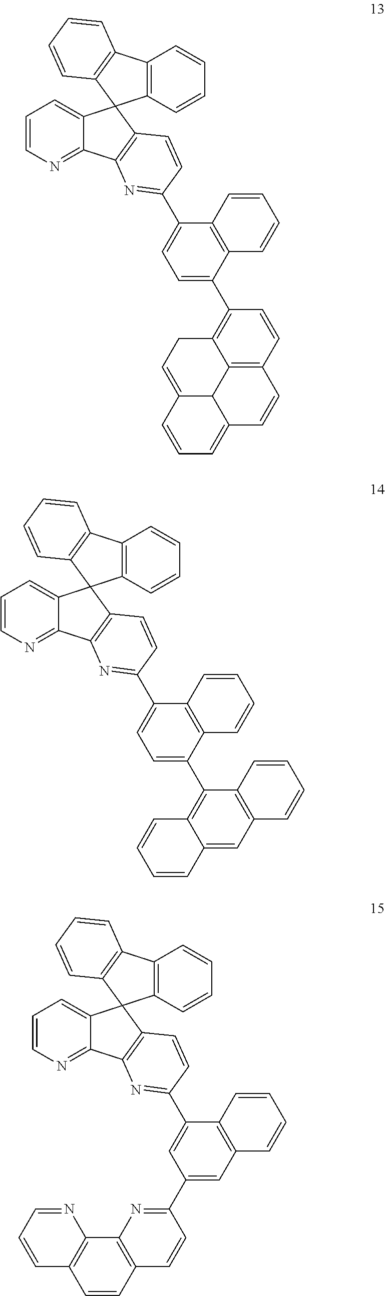 [SMILES: C1=CC2=CCc3c(-c4ccc(-c5ccc6c(n5)-c5ncccc5C65c6ccccc6-c6ccccc65)c5ccccc45)ccc4c3C2C(=C1)C=C4 ; c1ccc2c(c1)-c1ccccc1C21c2cccnc2-c2nc(-c3ccc(-c4c5ccccc5cc5ccccc45)c4ccccc34)ccc21 ; c1ccc2c(c1)-c1ccccc1C21c2cccnc2-c2nc(-c3cc(-c4ccc5ccc6cccnc6c5n4)cc4ccccc34)ccc21]

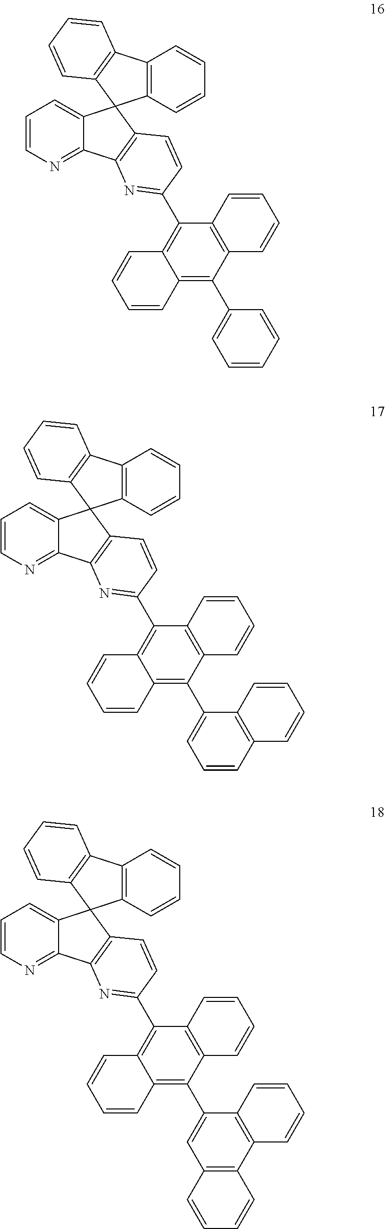 [SMILES: c1ccc(-c2c3ccccc3c(-c3ccc4c(n3)-c3ncccc3C43c4ccccc4-c4ccccc43)c3ccccc23)cc1 ; c1ccc2c(c1)-c1ccccc1C21c2cccnc2-c2nc(-c3c4ccccc4c(-c4cccc5ccccc45)c4ccccc34)ccc21 ; c1ccc2c(c1)-c1ccccc1C21c2cccnc2-c2nc(-c3c4ccccc4c(-c4cc5ccccc5c5ccccc45)c4ccccc34)ccc21]

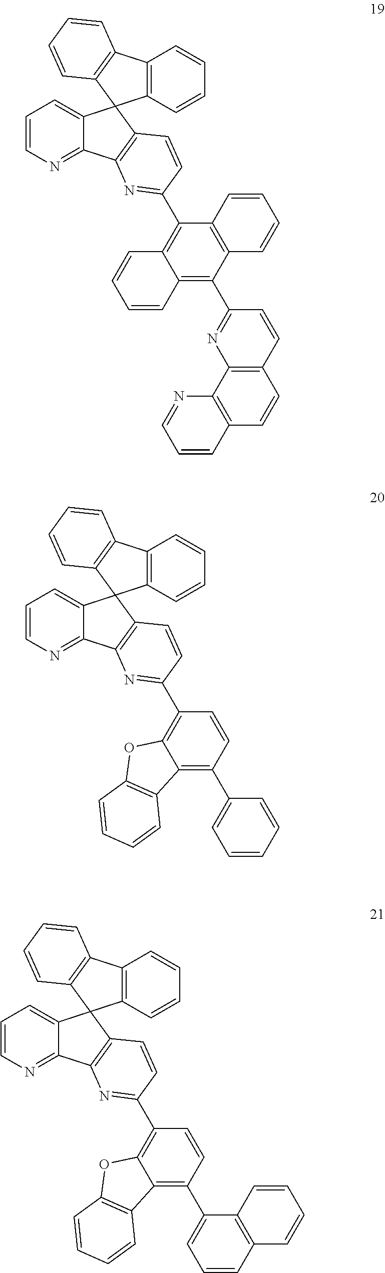 [SMILES: c1ccc2c(c1)-c1ccccc1C21c2cccnc2-c2nc(-c3c4ccccc4c(-c4ccc5ccc6cccnc6c5n4)c4ccccc34)ccc21 ; c1ccc(-c2ccc(-c3ccc4c(n3)-c3ncccc3C43c4ccccc4-c4ccccc43)c3oc4ccccc4c23)cc1 ; c1ccc2c(c1)-c1ccccc1C21c2cccnc2-c2nc(-c3ccc(-c4cccc5ccccc45)c4c3oc3ccccc34)ccc21]

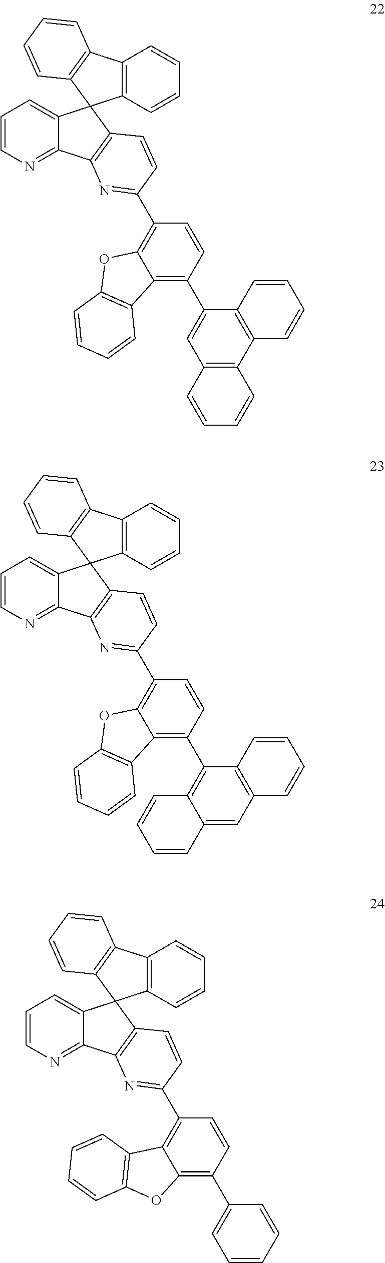 [SMILES: c1ccc2c(c1)-c1ccccc1C21c2cccnc2-c2nc(-c3ccc(-c4cc5ccccc5c5ccccc45)c4c3oc3ccccc34)ccc21 ; c1ccc2c(c1)-c1ccccc1C21c2cccnc2-c2nc(-c3ccc(-c4c5ccccc5cc5ccccc45)c4c3oc3ccccc34)ccc21 ; c1ccc(-c2ccc(-c3ccc4c(n3)-c3ncccc3C43c4ccccc4-c4ccccc43)c3c2oc2ccccc23)cc1]

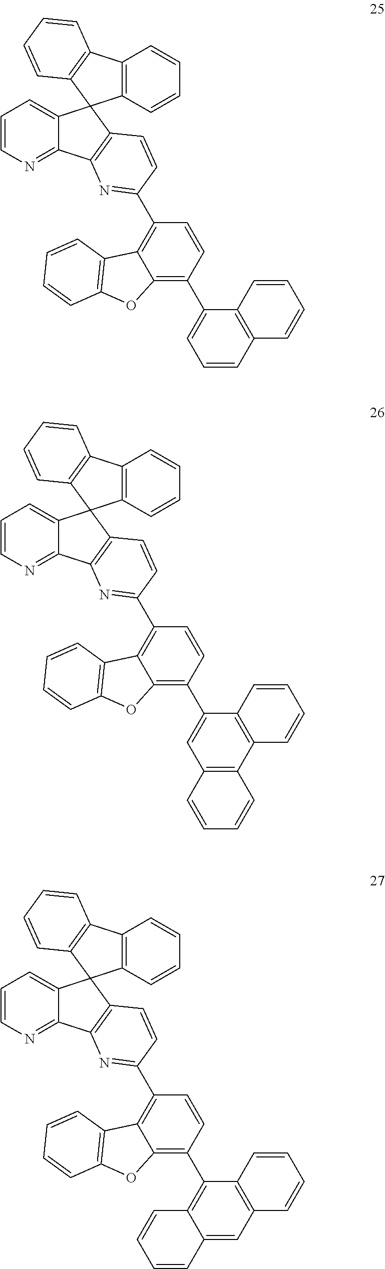 [SMILES: c1ccc2c(c1)-c1ccccc1C21c2cccnc2-c2nc(-c3ccc(-c4cccc5ccccc45)c4oc5ccccc5c34)ccc21 ; c1ccc2c(c1)-c1ccccc1C21c2cccnc2-c2nc(-c3ccc(-c4cc5ccccc5c5ccccc45)c4oc5ccccc5c34)ccc21 ; c1ccc2c(c1)-c1ccccc1C21c2cccnc2-c2nc(-c3ccc(-c4c5ccccc5cc5ccccc45)c4oc5ccccc5c34)ccc21]

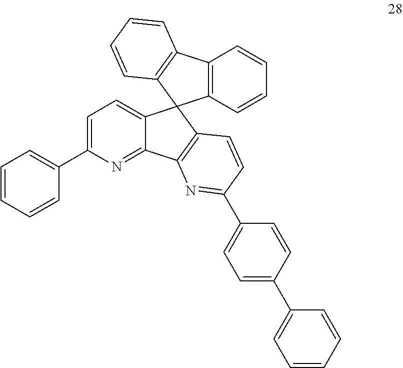 [SMILES: c1ccc(-c2ccc(-c3ccc4c(n3)-c3nc(-c5ccccc5)ccc3C43c4ccccc4-c4ccccc43)cc2)cc1]

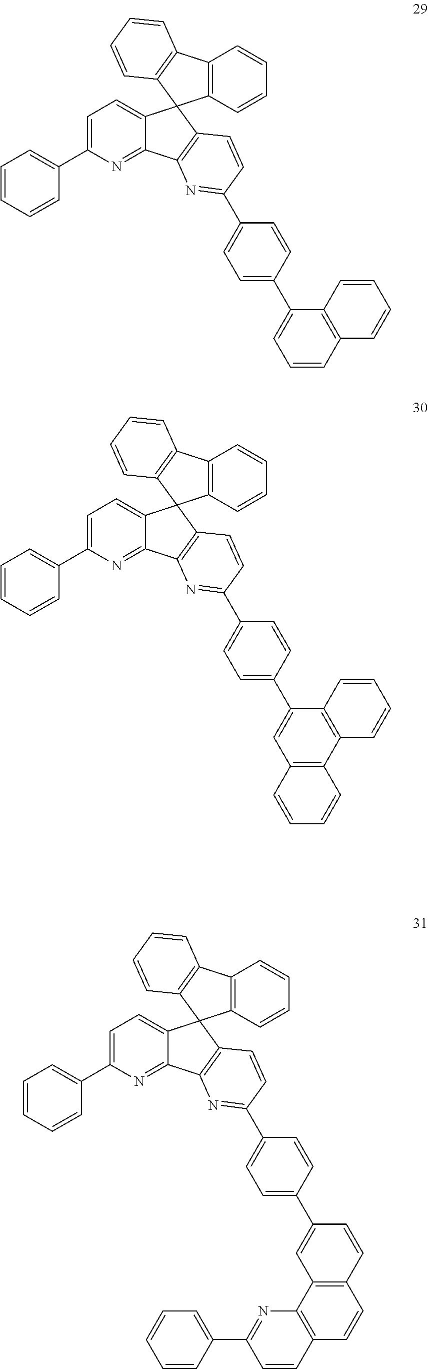 [SMILES: c1ccc(-c2ccc3c(n2)-c2nc(-c4ccc(-c5cccc6ccccc56)cc4)ccc2C32c3ccccc3-c3ccccc32)cc1 ; c1ccc(-c2ccc3c(n2)-c2nc(-c4ccc(-c5cc6ccccc6c6ccccc56)cc4)ccc2C32c3ccccc3-c3ccccc32)cc1 ; c1ccc(-c2ccc3c(n2)-c2nc(-c4ccc(-c5ccc6ccc7ccc(-c8ccccc8)nc7c6c5)cc4)ccc2C32c3ccccc3-c3ccccc32)cc1]

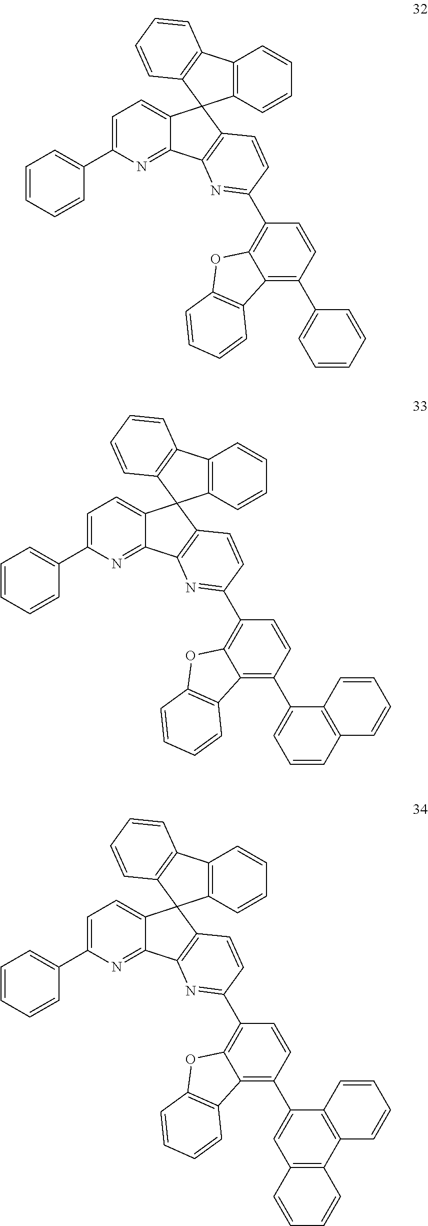 [SMILES: c1ccc(-c2ccc3c(n2)-c2nc(-c4ccc(-c5ccccc5)c5c4oc4ccccc45)ccc2C32c3ccccc3-c3ccccc32)cc1 ; c1ccc(-c2ccc3c(n2)-c2nc(-c4ccc(-c5cccc6ccccc56)c5c4oc4ccccc45)ccc2C32c3ccccc3-c3ccccc32)cc1 ; c1ccc(-c2ccc3c(n2)-c2nc(-c4ccc(-c5cc6ccccc6c6ccccc56)c5c4oc4ccccc45)ccc2C32c3ccccc3-c3ccccc32)cc1]

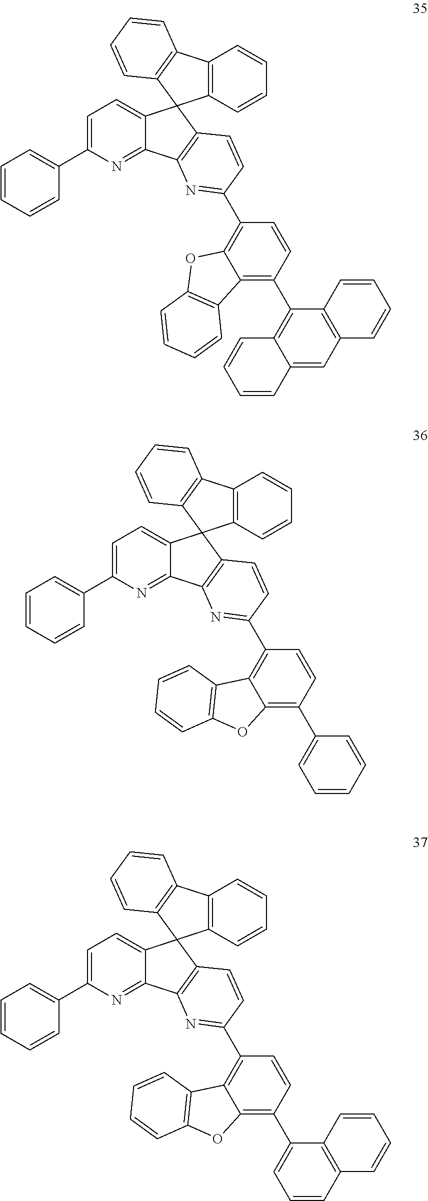 [SMILES: c1ccc(-c2ccc3c(n2)-c2nc(-c4ccc(-c5c6ccccc6cc6ccccc56)c5c4oc4ccccc45)ccc2C32c3ccccc3-c3ccccc32)cc1 ; c1ccc(-c2ccc3c(n2)-c2nc(-c4ccc(-c5ccccc5)c5oc6ccccc6c45)ccc2C32c3ccccc3-c3ccccc32)cc1 ; c1ccc(-c2ccc3c(n2)-c2nc(-c4ccc(-c5cccc6ccccc56)c5oc6ccccc6c45)ccc2C32c3ccccc3-c3ccccc32)cc1]

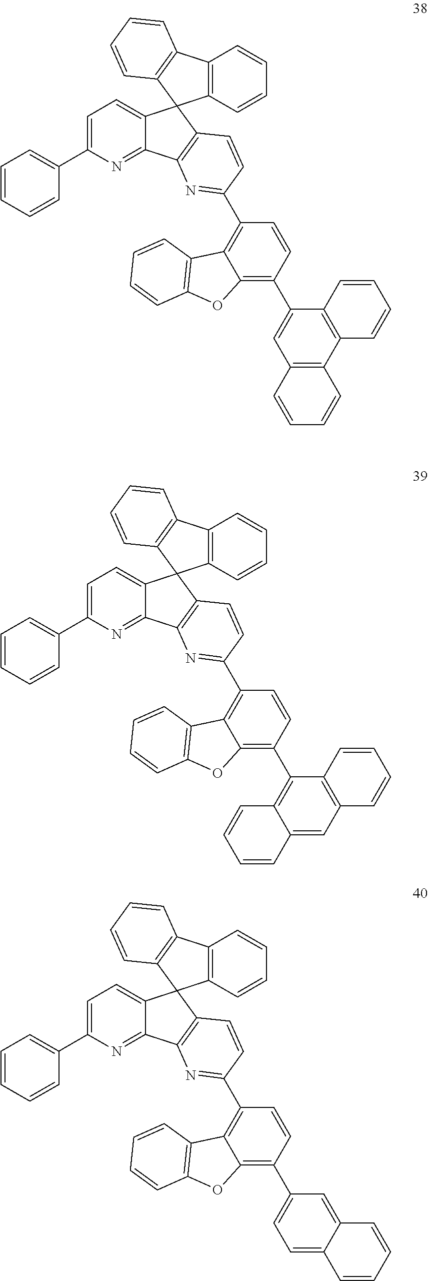 [SMILES: c1ccc(-c2ccc3c(n2)-c2nc(-c4ccc(-c5cc6ccccc6c6ccccc56)c5oc6ccccc6c45)ccc2C32c3ccccc3-c3ccccc32)cc1 ; c1ccc(-c2ccc3c(n2)-c2nc(-c4ccc(-c5c6ccccc6cc6ccccc56)c5oc6ccccc6c45)ccc2C32c3ccccc3-c3ccccc32)cc1 ; c1ccc(-c2ccc3c(n2)-c2nc(-c4ccc(-c5ccc6ccccc6c5)c5oc6ccccc6c45)ccc2C32c3ccccc3-c3ccccc32)cc1]

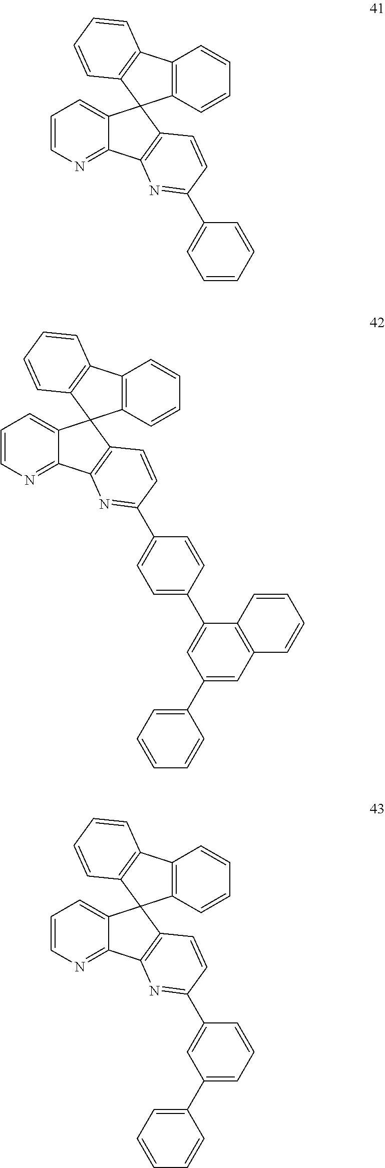 [SMILES: c1ccc(-c2ccc3c(n2)-c2ncccc2C32c3ccccc3-c3ccccc32)cc1 ; c1ccc(-c2cc(-c3ccc(-c4ccc5c(n4)-c4ncccc4C54c5ccccc5-c5ccccc54)cc3)c3ccccc3c2)cc1 ; c1ccc(-c2cccc(-c3ccc4c(n3)-c3ncccc3C43c4ccccc4-c4ccccc43)c2)cc1]

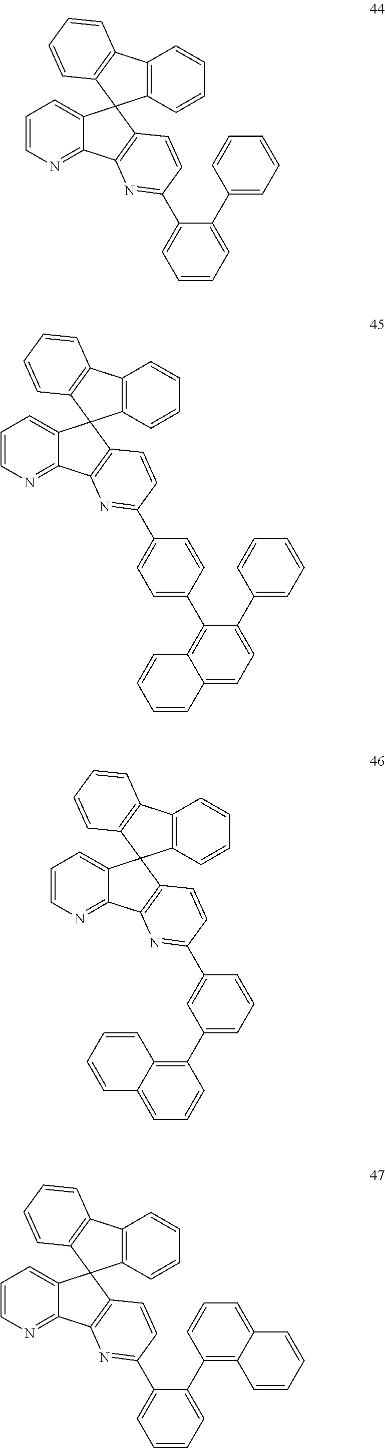 [SMILES: c1ccc(-c2ccccc2-c2ccc3c(n2)-c2ncccc2C32c3ccccc3-c3ccccc32)cc1 ; c1ccc(-c2ccc3ccccc3c2-c2ccc(-c3ccc4c(n3)-c3ncccc3C43c4ccccc4-c4ccccc43)cc2)cc1 ; c1cc(-c2ccc3c(n2)-c2ncccc2C32c3ccccc3-c3ccccc32)cc(-c2cccc3ccccc23)c1 ; c1ccc(-c2cccc3ccccc23)c(-c2ccc3c(n2)-c2ncccc2C32c3ccccc3-c3ccccc32)c1]

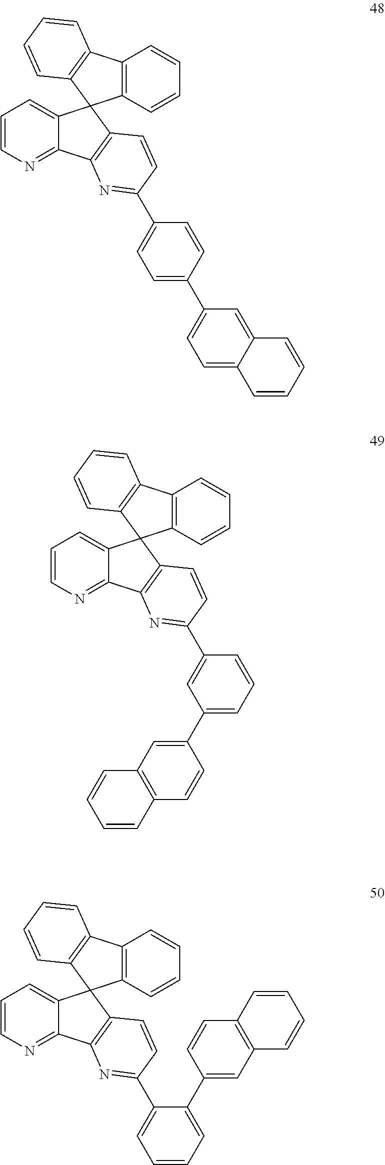 [SMILES: c1ccc2c(c1)-c1ccccc1C21c2cccnc2-c2nc(-c3ccc(-c4ccc5ccccc5c4)cc3)ccc21 ; c1cc(-c2ccc3ccccc3c2)cc(-c2ccc3c(n2)-c2ncccc2C32c3ccccc3-c3ccccc32)c1 ; c1ccc(-c2ccc3c(n2)-c2ncccc2C32c3ccccc3-c3ccccc32)c(-c2ccc3ccccc3c2)c1]

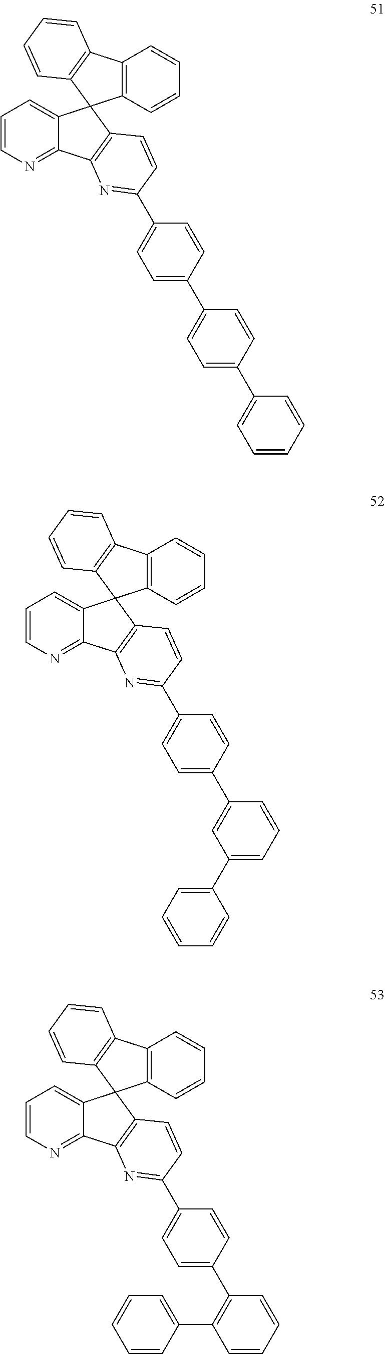 [SMILES: c1ccc(-c2ccc(-c3ccc(-c4ccc5c(n4)-c4ncccc4C54c5ccccc5-c5ccccc54)cc3)cc2)cc1 ; c1ccc(-c2cccc(-c3ccc(-c4ccc5c(n4)-c4ncccc4C54c5ccccc5-c5ccccc54)cc3)c2)cc1 ; c1ccc(-c2ccccc2-c2ccc(-c3ccc4c(n3)-c3ncccc3C43c4ccccc4-c4ccccc43)cc2)cc1]

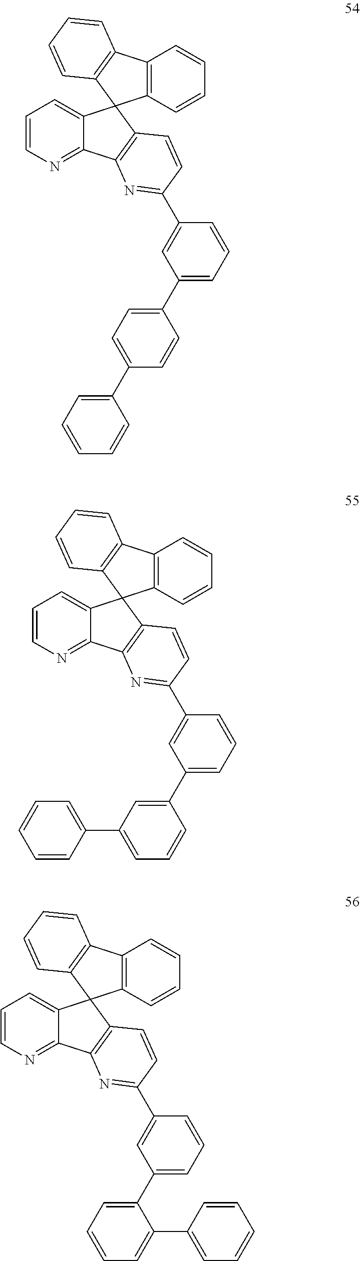 [SMILES: c1ccc(-c2ccc(-c3cccc(-c4ccc5c(n4)-c4ncccc4C54c5ccccc5-c5ccccc54)c3)cc2)cc1 ; c1ccc(-c2cccc(-c3cccc(-c4ccc5c(n4)-c4ncccc4C54c5ccccc5-c5ccccc54)c3)c2)cc1 ; c1ccc(-c2ccccc2-c2cccc(-c3ccc4c(n3)-c3ncccc3C43c4ccccc4-c4ccccc43)c2)cc1]

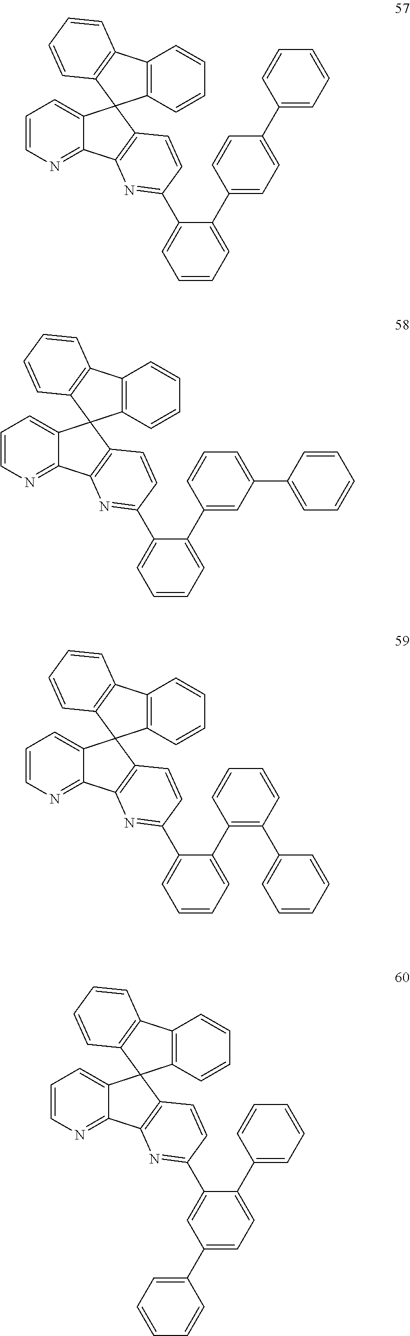 [SMILES: c1ccc(-c2ccc(-c3ccccc3-c3ccc4c(n3)-c3ncccc3C43c4ccccc4-c4ccccc43)cc2)cc1 ; c1ccc(-c2cccc(-c3ccccc3-c3ccc4c(n3)-c3ncccc3C43c4ccccc4-c4ccccc43)c2)cc1 ; c1ccc(-c2ccccc2-c2ccccc2-c2ccc3c(n2)-c2ncccc2C32c3ccccc3-c3ccccc32)cc1 ; c1ccc(-c2ccc(-c3ccccc3)c(-c3ccc4c(n3)-c3ncccc3C43c4ccccc4-c4ccccc43)c2)cc1]

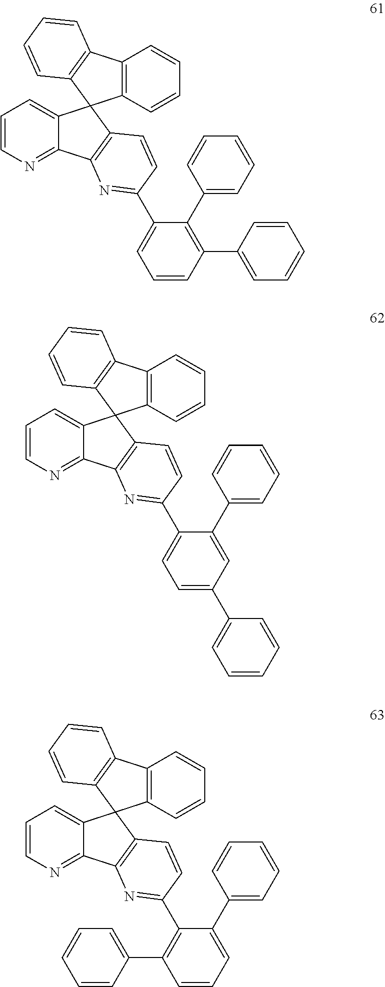 [SMILES: c1ccc(-c2cccc(-c3ccc4c(n3)-c3ncccc3C43c4ccccc4-c4ccccc43)c2-c2ccccc2)cc1 ; c1ccc(-c2ccc(-c3ccc4c(n3)-c3ncccc3C43c4ccccc4-c4ccccc43)c(-c3ccccc3)c2)cc1 ; c1ccc(-c2cccc(-c3ccccc3)c2-c2ccc3c(n2)-c2ncccc2C32c3ccccc3-c3ccccc32)cc1]

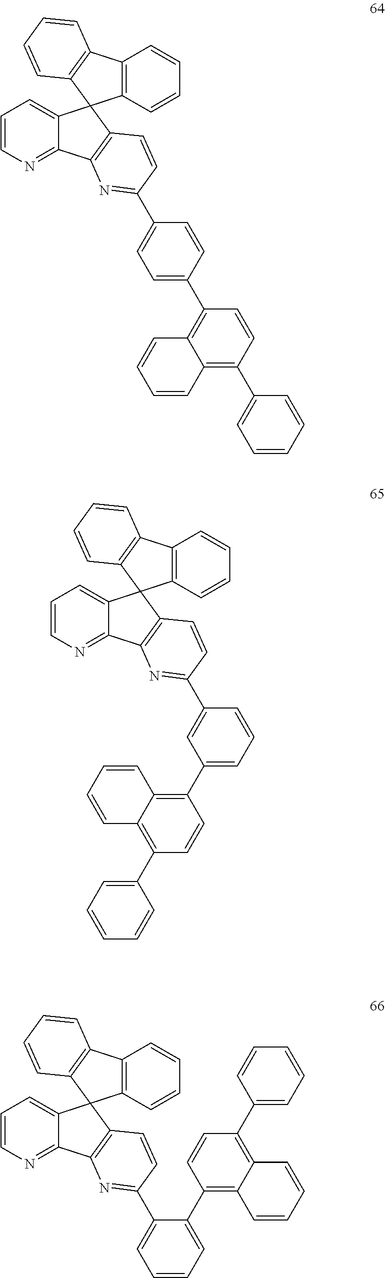 [SMILES: c1ccc(-c2ccc(-c3ccc(-c4ccc5c(n4)-c4ncccc4C54c5ccccc5-c5ccccc54)cc3)c3ccccc23)cc1 ; c1ccc(-c2ccc(-c3cccc(-c4ccc5c(n4)-c4ncccc4C54c5ccccc5-c5ccccc54)c3)c3ccccc23)cc1 ; c1ccc(-c2ccc(-c3ccccc3-c3ccc4c(n3)-c3ncccc3C43c4ccccc4-c4ccccc43)c3ccccc23)cc1]

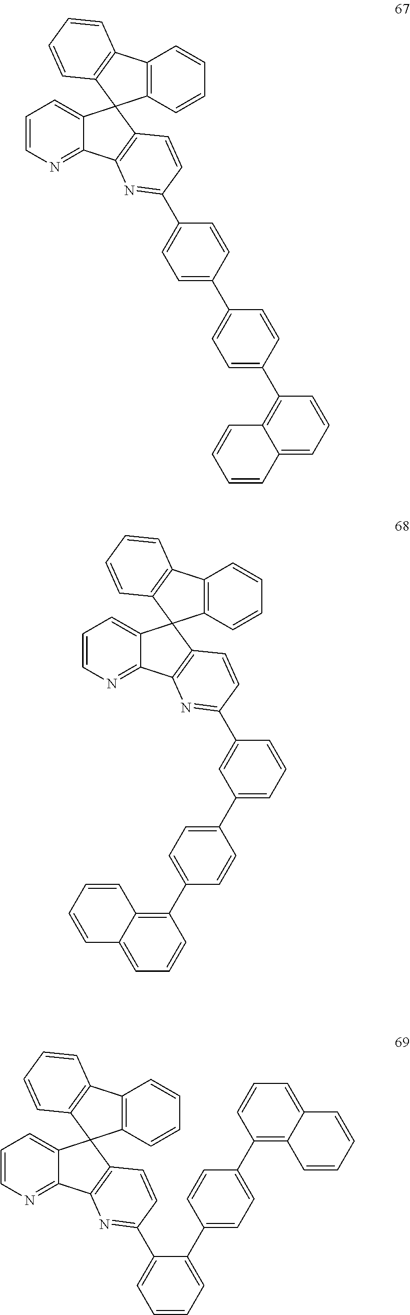 [SMILES: c1ccc2c(c1)-c1ccccc1C21c2cccnc2-c2nc(-c3ccc(-c4ccc(-c5cccc6ccccc56)cc4)cc3)ccc21 ; c1cc(-c2ccc(-c3cccc4ccccc34)cc2)cc(-c2ccc3c(n2)-c2ncccc2C32c3ccccc3-c3ccccc32)c1 ; c1ccc(-c2ccc3c(n2)-c2ncccc2C32c3ccccc3-c3ccccc32)c(-c2ccc(-c3cccc4ccccc34)cc2)c1]

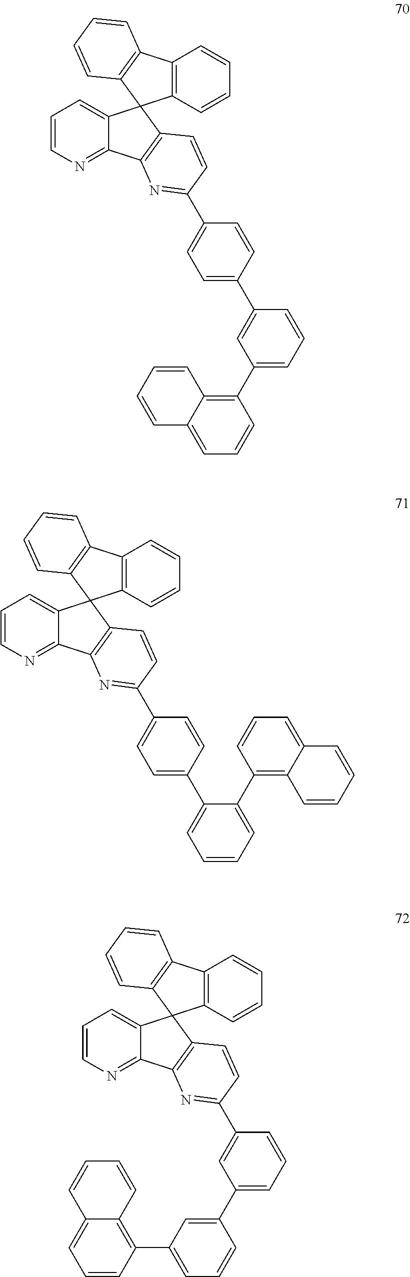 [SMILES: c1cc(-c2ccc(-c3ccc4c(n3)-c3ncccc3C43c4ccccc4-c4ccccc43)cc2)cc(-c2cccc3ccccc23)c1 ; c1ccc(-c2cccc3ccccc23)c(-c2ccc(-c3ccc4c(n3)-c3ncccc3C43c4ccccc4-c4ccccc43)cc2)c1 ; c1cc(-c2cccc(-c3cccc4ccccc34)c2)cc(-c2ccc3c(n2)-c2ncccc2C32c3ccccc3-c3ccccc32)c1]

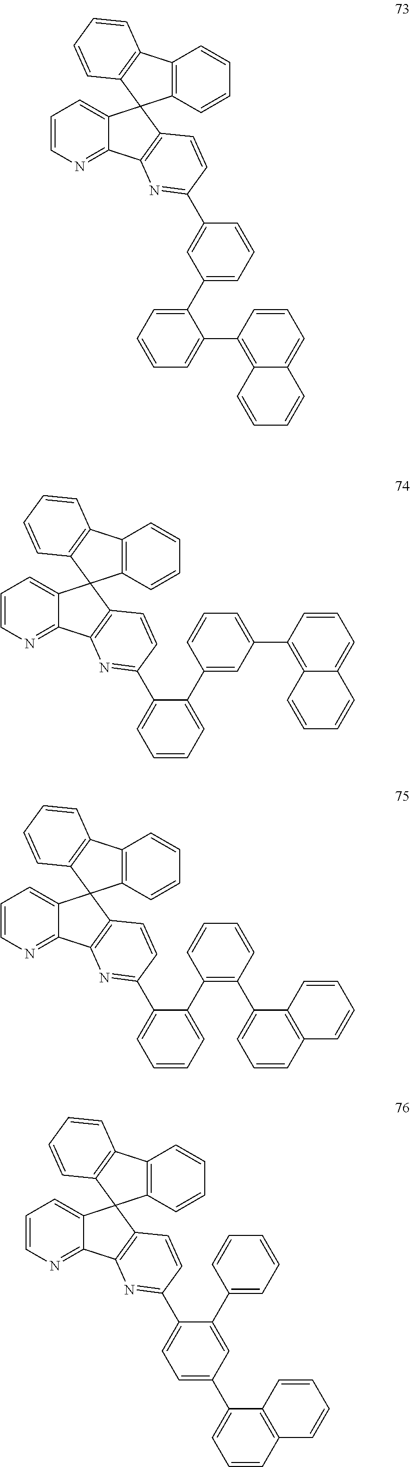 [SMILES: c1cc(-c2ccc3c(n2)-c2ncccc2C32c3ccccc3-c3ccccc32)cc(-c2ccccc2-c2cccc3ccccc23)c1 ; c1cc(-c2ccccc2-c2ccc3c(n2)-c2ncccc2C32c3ccccc3-c3ccccc32)cc(-c2cccc3ccccc23)c1 ; c1ccc(-c2ccccc2-c2cccc3ccccc23)c(-c2ccc3c(n2)-c2ncccc2C32c3ccccc3-c3ccccc32)c1 ; c1ccc(-c2cc(-c3cccc4ccccc34)ccc2-c2ccc3c(n2)-c2ncccc2C32c3ccccc3-c3ccccc32)cc1]

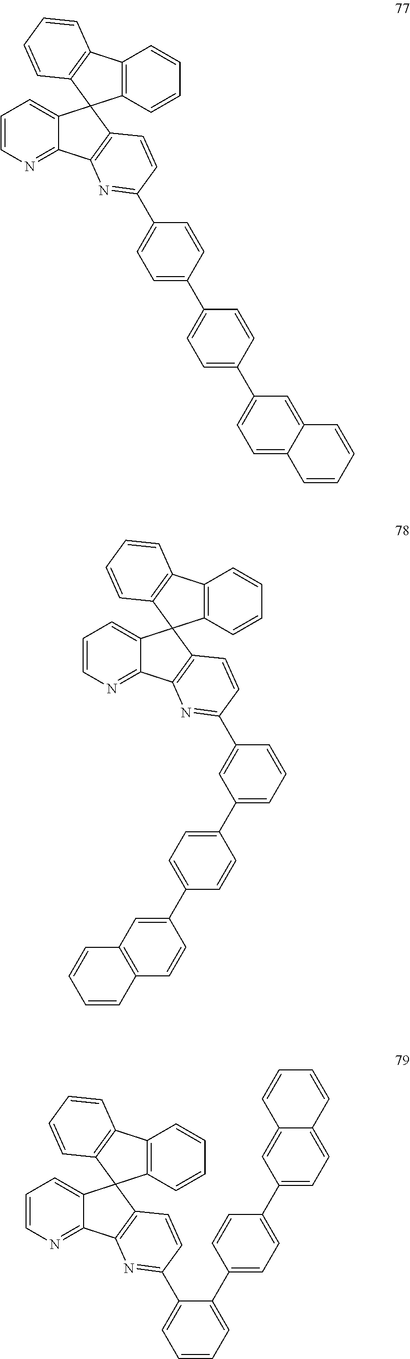 [SMILES: c1ccc2c(c1)-c1ccccc1C21c2cccnc2-c2nc(-c3ccc(-c4ccc(-c5ccc6ccccc6c5)cc4)cc3)ccc21 ; c1cc(-c2ccc(-c3ccc4ccccc4c3)cc2)cc(-c2ccc3c(n2)-c2ncccc2C32c3ccccc3-c3ccccc32)c1 ; c1ccc(-c2ccc3c(n2)-c2ncccc2C32c3ccccc3-c3ccccc32)c(-c2ccc(-c3ccc4ccccc4c3)cc2)c1]

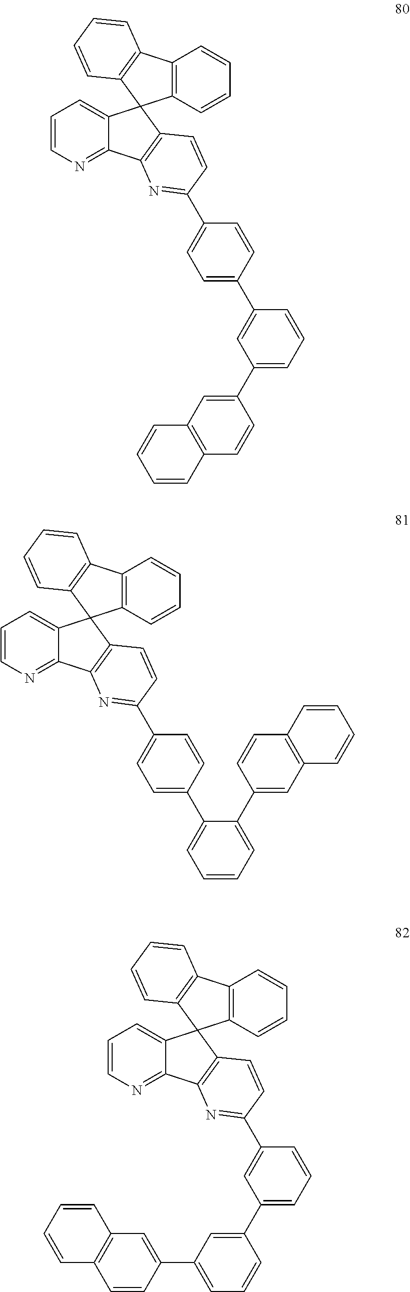 [SMILES: c1cc(-c2ccc(-c3ccc4c(n3)-c3ncccc3C43c4ccccc4-c4ccccc43)cc2)cc(-c2ccc3ccccc3c2)c1 ; c1ccc(-c2ccc3ccccc3c2)c(-c2ccc(-c3ccc4c(n3)-c3ncccc3C43c4ccccc4-c4ccccc43)cc2)c1 ; c1cc(-c2cccc(-c3ccc4c(n3)-c3ncccc3C43c4ccccc4-c4ccccc43)c2)cc(-c2ccc3ccccc3c2)c1]

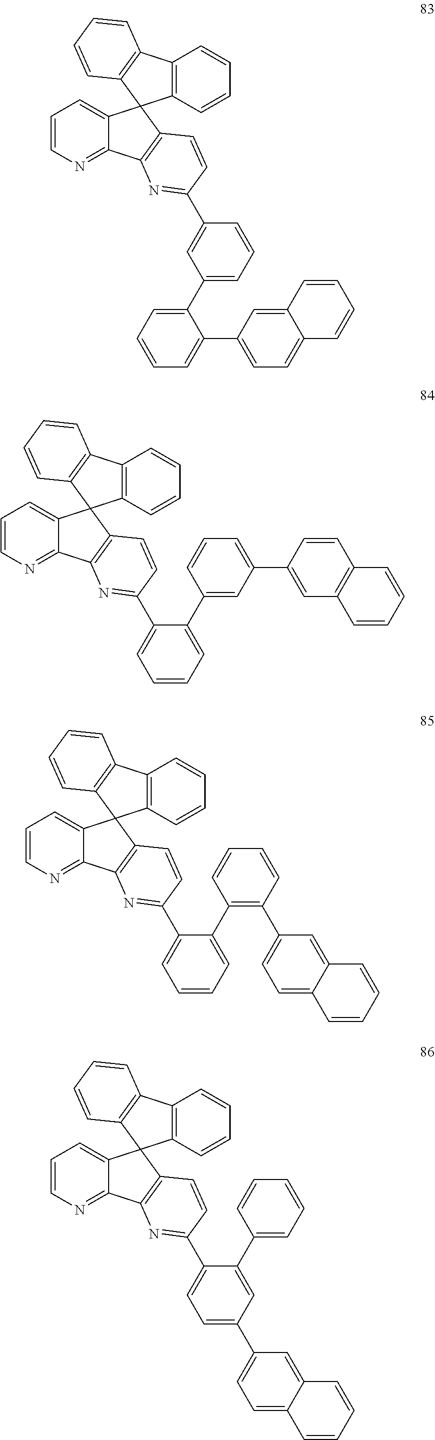 [SMILES: c1cc(-c2ccc3c(n2)-c2ncccc2C32c3ccccc3-c3ccccc32)cc(-c2ccccc2-c2ccc3ccccc3c2)c1 ; c1cc(-c2ccc3ccccc3c2)cc(-c2ccccc2-c2ccc3c(n2)-c2ncccc2C32c3ccccc3-c3ccccc32)c1 ; c1ccc(-c2ccccc2-c2ccc3c(n2)-c2ncccc2C32c3ccccc3-c3ccccc32)c(-c2ccc3ccccc3c2)c1 ; c1ccc(-c2cc(-c3ccc4ccccc4c3)ccc2-c2ccc3c(n2)-c2ncccc2C32c3ccccc3-c3ccccc32)cc1]

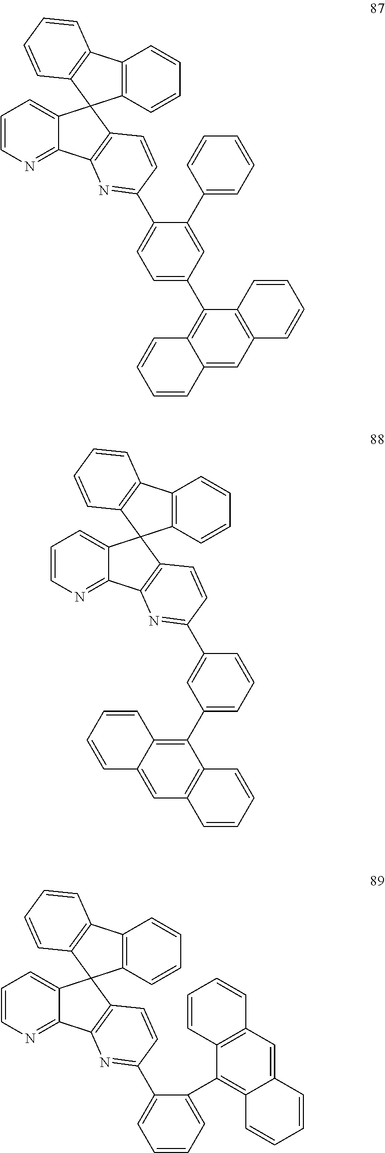 [SMILES: c1ccc(-c2cc(-c3c4ccccc4cc4ccccc34)ccc2-c2ccc3c(n2)-c2ncccc2C32c3ccccc3-c3ccccc32)cc1 ; c1cc(-c2ccc3c(n2)-c2ncccc2C32c3ccccc3-c3ccccc32)cc(-c2c3ccccc3cc3ccccc23)c1 ; c1ccc(-c2c3ccccc3cc3ccccc23)c(-c2ccc3c(n2)-c2ncccc2C32c3ccccc3-c3ccccc32)c1]

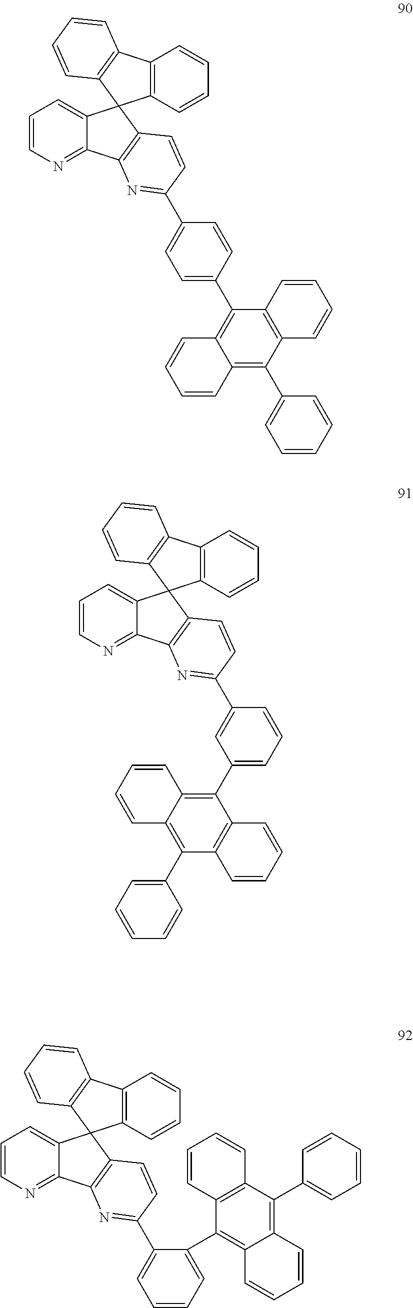 [SMILES: c1ccc(-c2c3ccccc3c(-c3ccc(-c4ccc5c(n4)-c4ncccc4C54c5ccccc5-c5ccccc54)cc3)c3ccccc23)cc1 ; c1ccc(-c2c3ccccc3c(-c3cccc(-c4ccc5c(n4)-c4ncccc4C54c5ccccc5-c5ccccc54)c3)c3ccccc23)cc1 ; c1ccc(-c2c3ccccc3c(-c3ccccc3-c3ccc4c(n3)-c3ncccc3C43c4ccccc4-c4ccccc43)c3ccccc23)cc1]

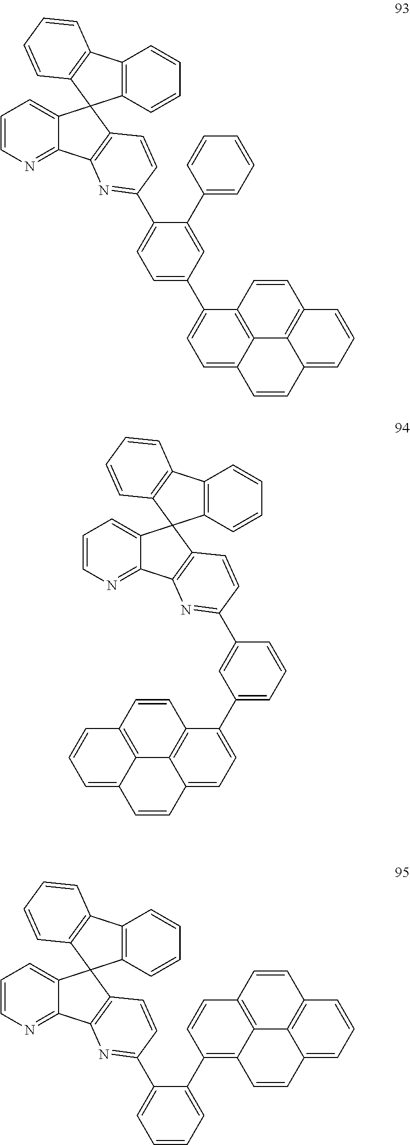 [SMILES: c1ccc(-c2cc(-c3ccc4ccc5cccc6ccc3c4c56)ccc2-c2ccc3c(n2)-c2ncccc2C32c3ccccc3-c3ccccc32)cc1 ; c1cc(-c2ccc3c(n2)-c2ncccc2C32c3ccccc3-c3ccccc32)cc(-c2ccc3ccc4cccc5ccc2c3c45)c1 ; c1ccc(-c2ccc3ccc4cccc5ccc2c3c45)c(-c2ccc3c(n2)-c2ncccc2C32c3ccccc3-c3ccccc32)c1]

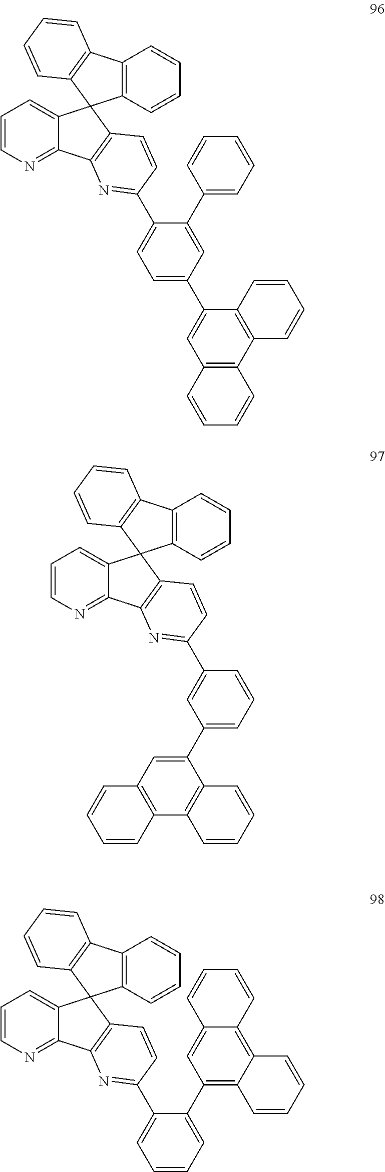 [SMILES: c1ccc(-c2cc(-c3cc4ccccc4c4ccccc34)ccc2-c2ccc3c(n2)-c2ncccc2C32c3ccccc3-c3ccccc32)cc1 ; c1cc(-c2ccc3c(n2)-c2ncccc2C32c3ccccc3-c3ccccc32)cc(-c2cc3ccccc3c3ccccc23)c1 ; c1ccc(-c2cc3ccccc3c3ccccc23)c(-c2ccc3c(n2)-c2ncccc2C32c3ccccc3-c3ccccc32)c1]

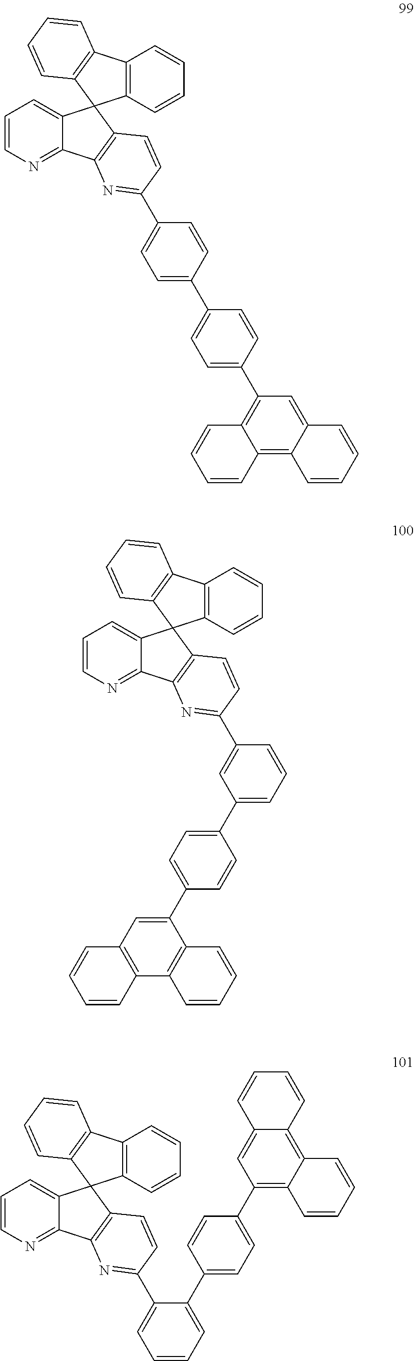 [SMILES: c1ccc2c(c1)-c1ccccc1C21c2cccnc2-c2nc(-c3ccc(-c4ccc(-c5cc6ccccc6c6ccccc56)cc4)cc3)ccc21 ; c1cc(-c2ccc(-c3cc4ccccc4c4ccccc34)cc2)cc(-c2ccc3c(n2)-c2ncccc2C32c3ccccc3-c3ccccc32)c1 ; c1ccc(-c2ccc3c(n2)-c2ncccc2C32c3ccccc3-c3ccccc32)c(-c2ccc(-c3cc4ccccc4c4ccccc34)cc2)c1]

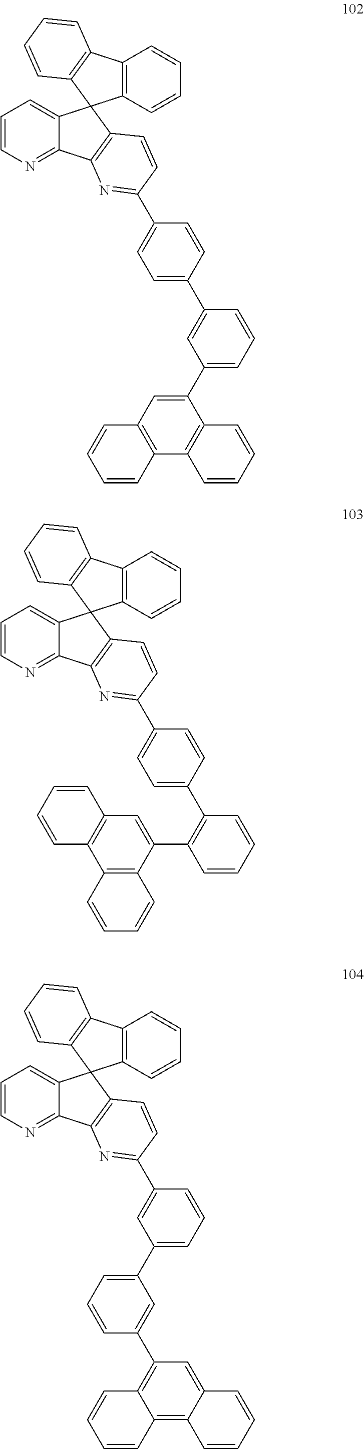 [SMILES: c1cc(-c2ccc(-c3ccc4c(n3)-c3ncccc3C43c4ccccc4-c4ccccc43)cc2)cc(-c2cc3ccccc3c3ccccc23)c1 ; c1ccc(-c2cc3ccccc3c3ccccc23)c(-c2ccc(-c3ccc4c(n3)-c3ncccc3C43c4ccccc4-c4ccccc43)cc2)c1 ; c1cc(-c2cccc(-c3cc4ccccc4c4ccccc34)c2)cc(-c2ccc3c(n2)-c2ncccc2C32c3ccccc3-c3ccccc32)c1]

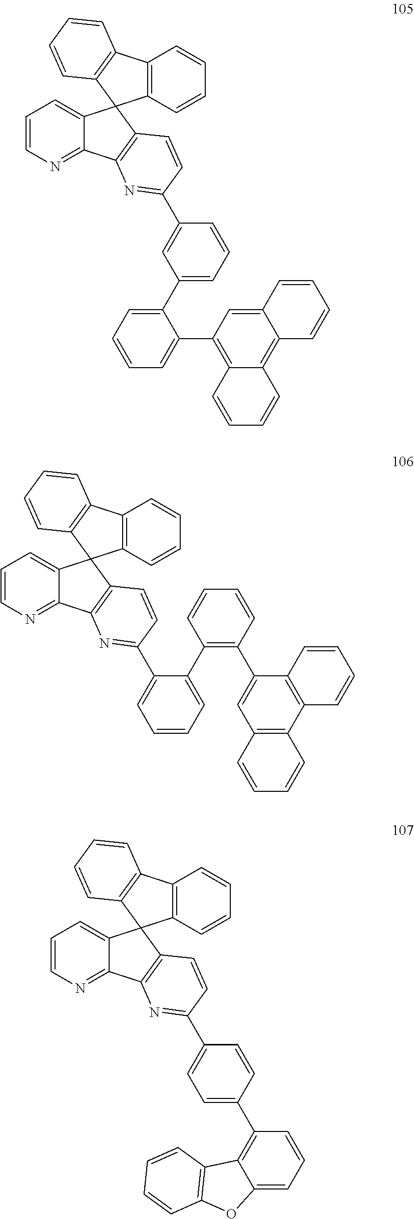 [SMILES: c1cc(-c2ccc3c(n2)-c2ncccc2C32c3ccccc3-c3ccccc32)cc(-c2ccccc2-c2cc3ccccc3c3ccccc23)c1 ; c1ccc(-c2ccccc2-c2cc3ccccc3c3ccccc23)c(-c2ccc3c(n2)-c2ncccc2C32c3ccccc3-c3ccccc32)c1 ; c1ccc2c(c1)-c1ccccc1C21c2cccnc2-c2nc(-c3ccc(-c4cccc5oc6ccccc6c45)cc3)ccc21]

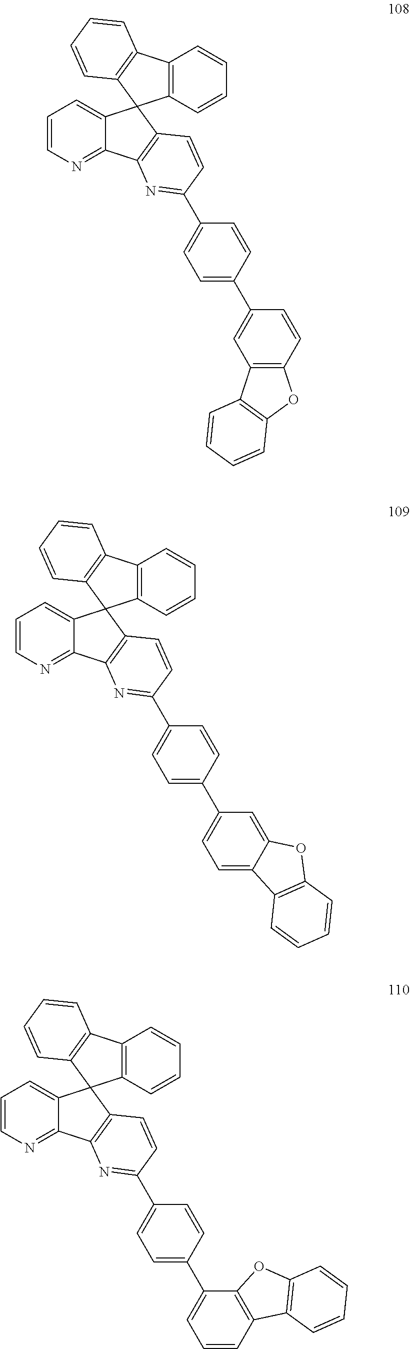 [SMILES: c1ccc2c(c1)-c1ccccc1C21c2cccnc2-c2nc(-c3ccc(-c4ccc5oc6ccccc6c5c4)cc3)ccc21 ; c1ccc2c(c1)-c1ccccc1C21c2cccnc2-c2nc(-c3ccc(-c4ccc5c(c4)oc4ccccc45)cc3)ccc21 ; c1ccc2c(c1)-c1ccccc1C21c2cccnc2-c2nc(-c3ccc(-c4cccc5c4oc4ccccc45)cc3)ccc21]

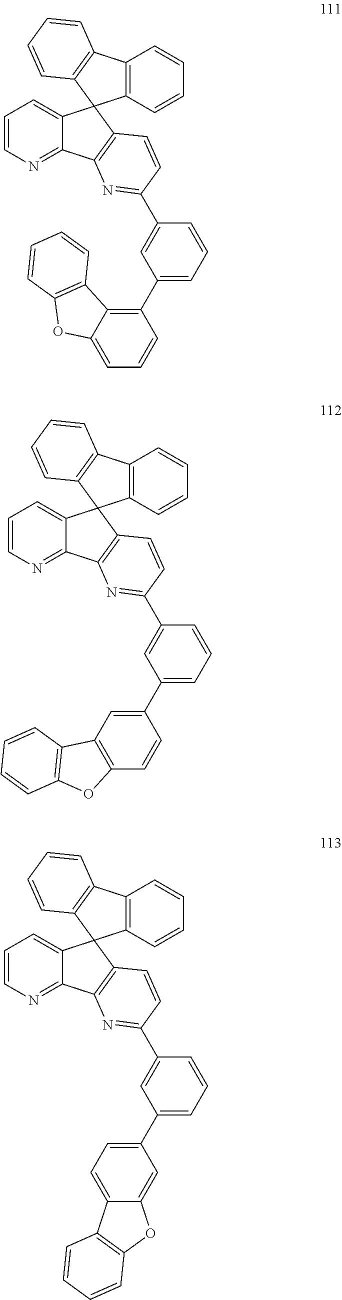 [SMILES: c1cc(-c2ccc3c(n2)-c2ncccc2C32c3ccccc3-c3ccccc32)cc(-c2cccc3oc4ccccc4c23)c1 ; c1cc(-c2ccc3oc4ccccc4c3c2)cc(-c2ccc3c(n2)-c2ncccc2C32c3ccccc3-c3ccccc32)c1 ; c1cc(-c2ccc3c(c2)oc2ccccc23)cc(-c2ccc3c(n2)-c2ncccc2C32c3ccccc3-c3ccccc32)c1]

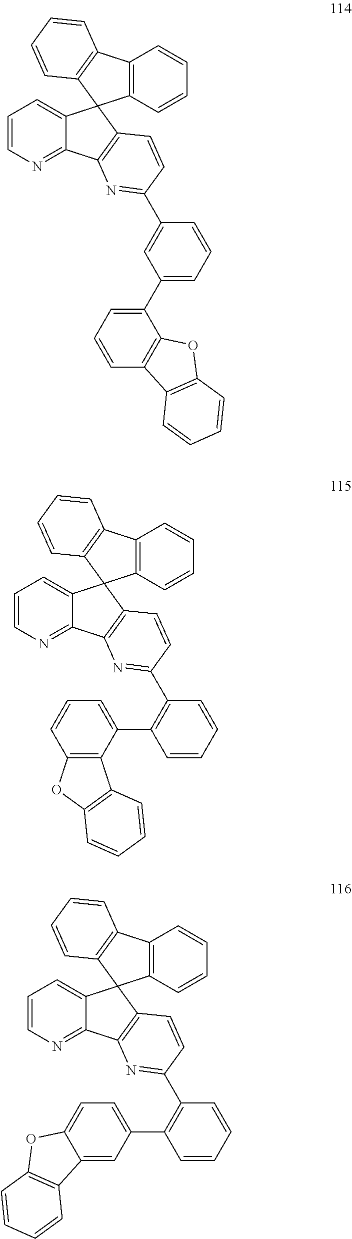 [SMILES: c1cc(-c2ccc3c(n2)-c2ncccc2C32c3ccccc3-c3ccccc32)cc(-c2cccc3c2oc2ccccc23)c1 ; c1ccc(-c2cccc3oc4ccccc4c23)c(-c2ccc3c(n2)-c2ncccc2C32c3ccccc3-c3ccccc32)c1 ; c1ccc(-c2ccc3c(n2)-c2ncccc2C32c3ccccc3-c3ccccc32)c(-c2ccc3oc4ccccc4c3c2)c1]

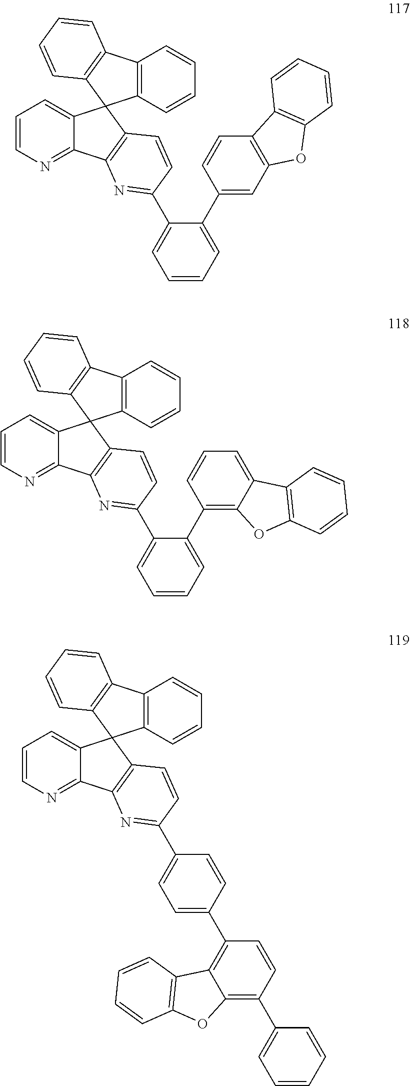 [SMILES: c1ccc(-c2ccc3c(n2)-c2ncccc2C32c3ccccc3-c3ccccc32)c(-c2ccc3c(c2)oc2ccccc23)c1 ; c1ccc(-c2cccc3c2oc2ccccc23)c(-c2ccc3c(n2)-c2ncccc2C32c3ccccc3-c3ccccc32)c1 ; c1ccc(-c2ccc(-c3ccc(-c4ccc5c(n4)-c4ncccc4C54c5ccccc5-c5ccccc54)cc3)c3c2oc2ccccc23)cc1]

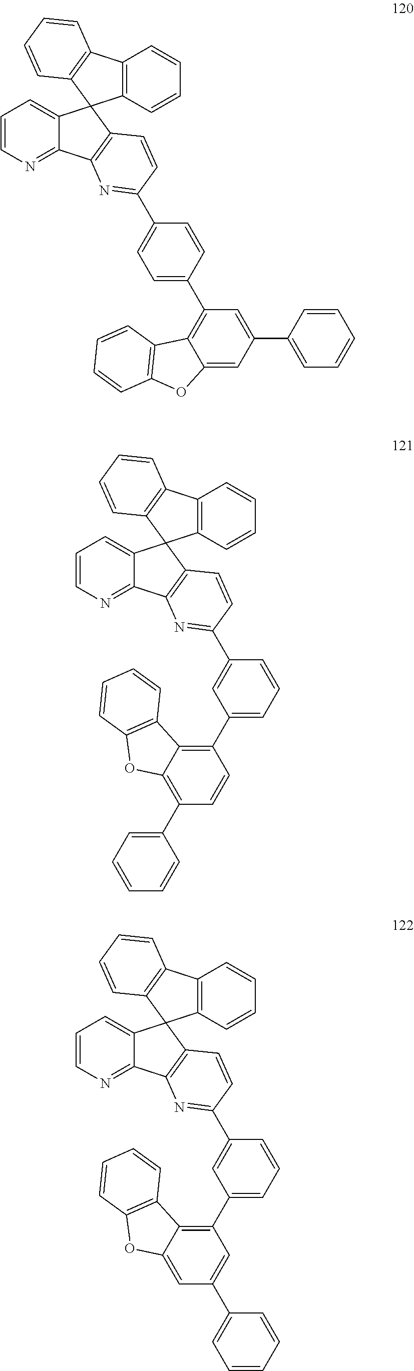

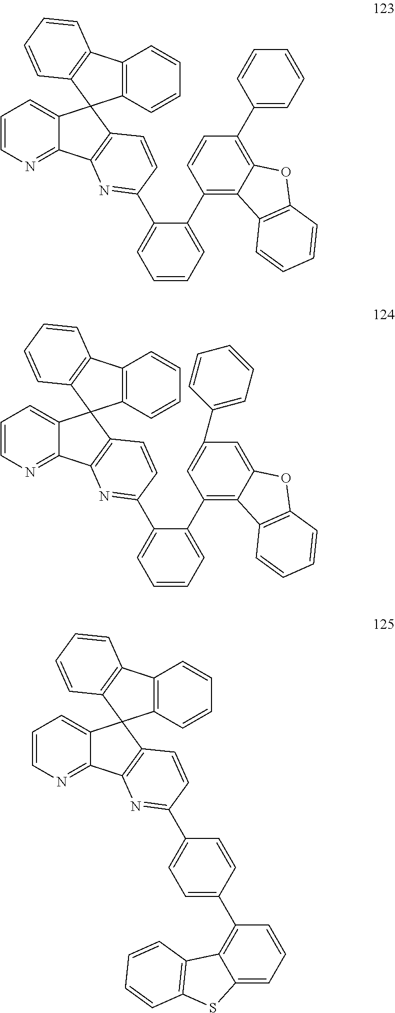 [SMILES: c1ccc(-c2ccc(-c3ccccc3-c3ccc4c(n3)-c3ncccc3C43c4ccccc4-c4ccccc43)c3c2oc2ccccc23)cc1 ; c1ccc(-c2cc(-c3ccccc3-c3ccc4c(n3)-c3ncccc3C43c4ccccc4-c4ccccc43)c3c(c2)oc2ccccc23)cc1 ; c1ccc2c(c1)-c1ccccc1C21c2cccnc2-c2nc(-c3ccc(-c4cccc5sc6ccccc6c45)cc3)ccc21]

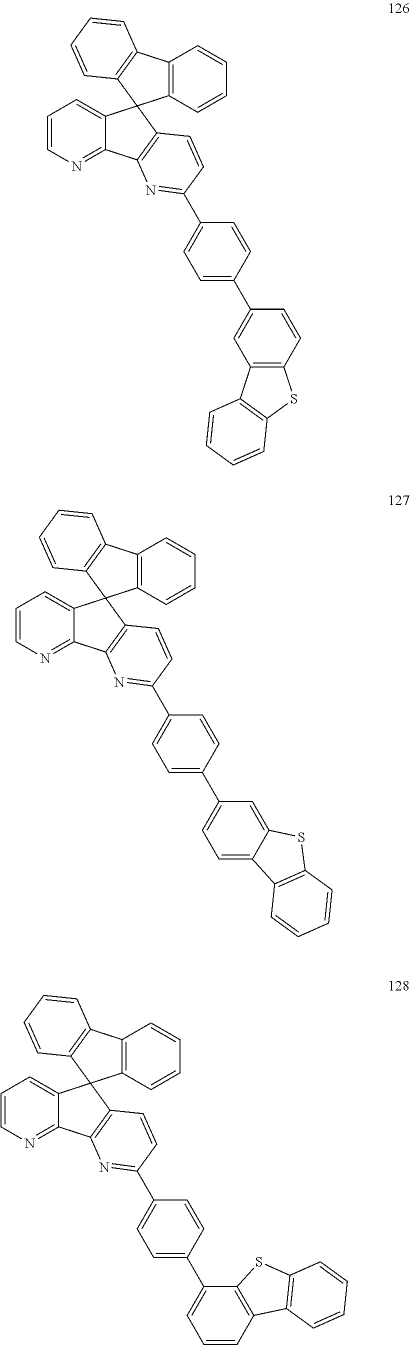 [SMILES: c1ccc2c(c1)-c1ccccc1C21c2cccnc2-c2nc(-c3ccc(-c4ccc5sc6ccccc6c5c4)cc3)ccc21 ; c1ccc2c(c1)-c1ccccc1C21c2cccnc2-c2nc(-c3ccc(-c4ccc5c(c4)sc4ccccc45)cc3)ccc21 ; c1ccc2c(c1)-c1ccccc1C21c2cccnc2-c2nc(-c3ccc(-c4cccc5c4sc4ccccc45)cc3)ccc21]

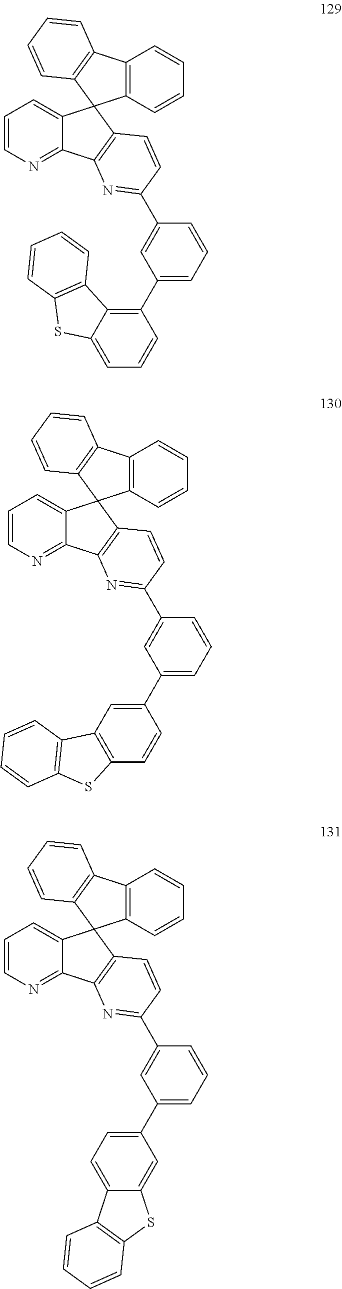 [SMILES: c1cc(-c2ccc3c(n2)-c2ncccc2C32c3ccccc3-c3ccccc32)cc(-c2cccc3sc4ccccc4c23)c1 ; c1cc(-c2ccc3sc4ccccc4c3c2)cc(-c2ccc3c(n2)-c2ncccc2C32c3ccccc3-c3ccccc32)c1 ; c1cc(-c2ccc3c(c2)sc2ccccc23)cc(-c2ccc3c(n2)-c2ncccc2C32c3ccccc3-c3ccccc32)c1]

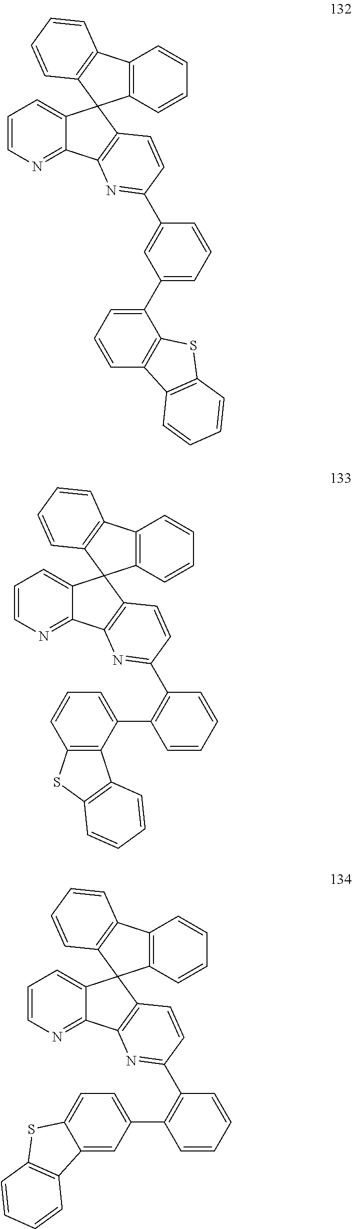

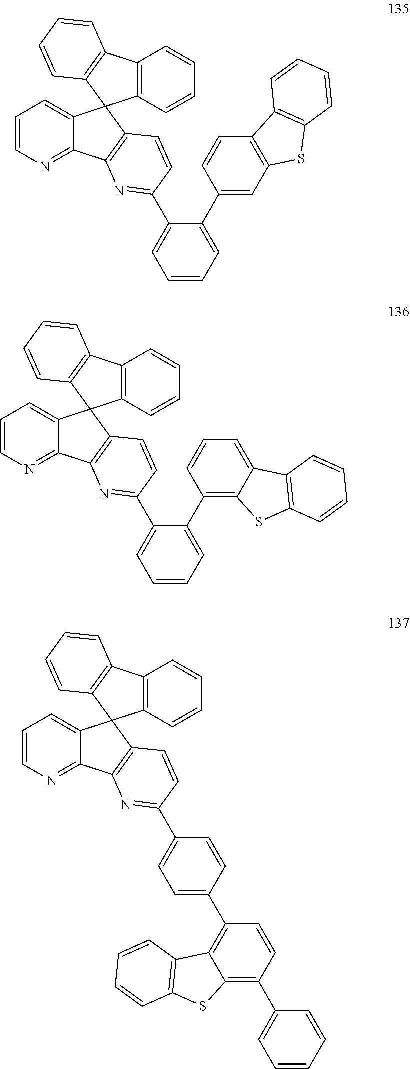 [SMILES: c1ccc(-c2ccc3c(n2)-c2ncccc2C32c3ccccc3-c3ccccc32)c(-c2ccc3c(c2)sc2ccccc23)c1 ; c1ccc(-c2cccc3c2sc2ccccc23)c(-c2ccc3c(n2)-c2ncccc2C32c3ccccc3-c3ccccc32)c1 ; c1ccc(-c2ccc(-c3ccc(-c4ccc5c(n4)-c4ncccc4C54c5ccccc5-c5ccccc54)cc3)c3c2sc2ccccc23)cc1]

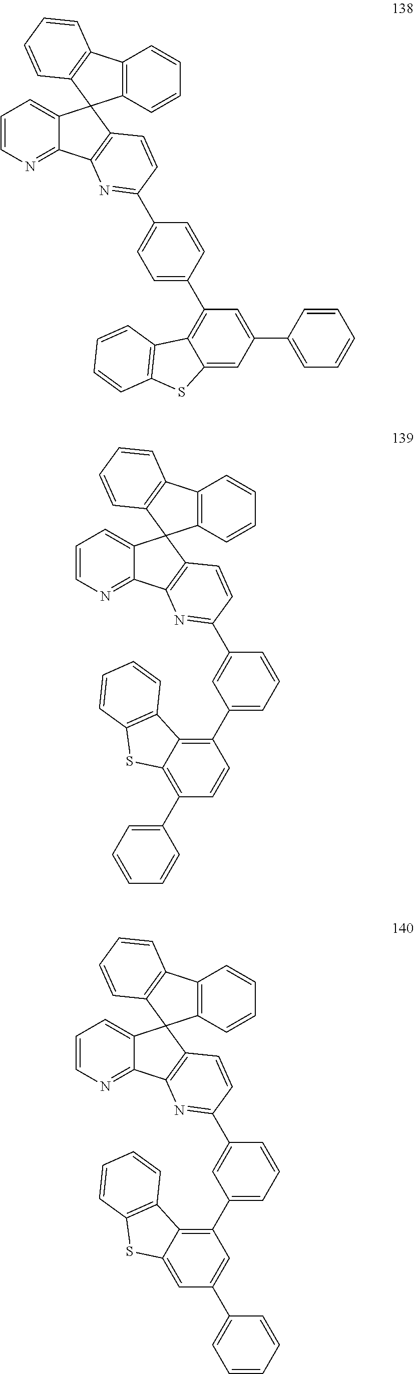 [SMILES: c1ccc(-c2cc(-c3ccc(-c4ccc5c(n4)-c4ncccc4C54c5ccccc5-c5ccccc54)cc3)c3c(c2)sc2ccccc23)cc1 ; c1ccc(-c2ccc(-c3cccc(-c4ccc5c(n4)-c4ncccc4C54c5ccccc5-c5ccccc54)c3)c3c2sc2ccccc23)cc1 ; c1ccc(-c2cc(-c3cccc(-c4ccc5c(n4)-c4ncccc4C54c5ccccc5-c5ccccc54)c3)c3c(c2)sc2ccccc23)cc1]

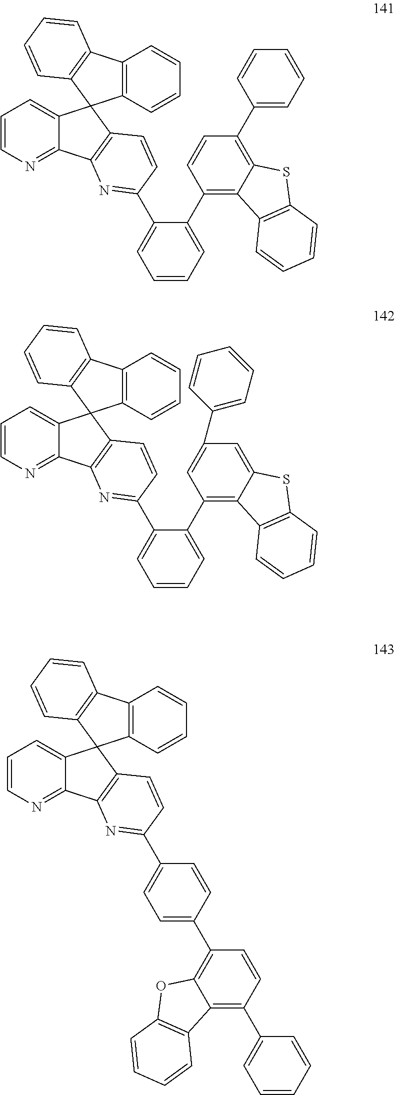 [SMILES: c1ccc(-c2ccc(-c3ccccc3-c3ccc4c(n3)-c3ncccc3C43c4ccccc4-c4ccccc43)c3c2sc2ccccc23)cc1 ; c1ccc(-c2cc(-c3ccccc3-c3ccc4c(n3)-c3ncccc3C43c4ccccc4-c4ccccc43)c3c(c2)sc2ccccc23)cc1 ; c1ccc(-c2ccc(-c3ccc(-c4ccc5c(n4)-c4ncccc4C54c5ccccc5-c5ccccc54)cc3)c3oc4ccccc4c23)cc1]

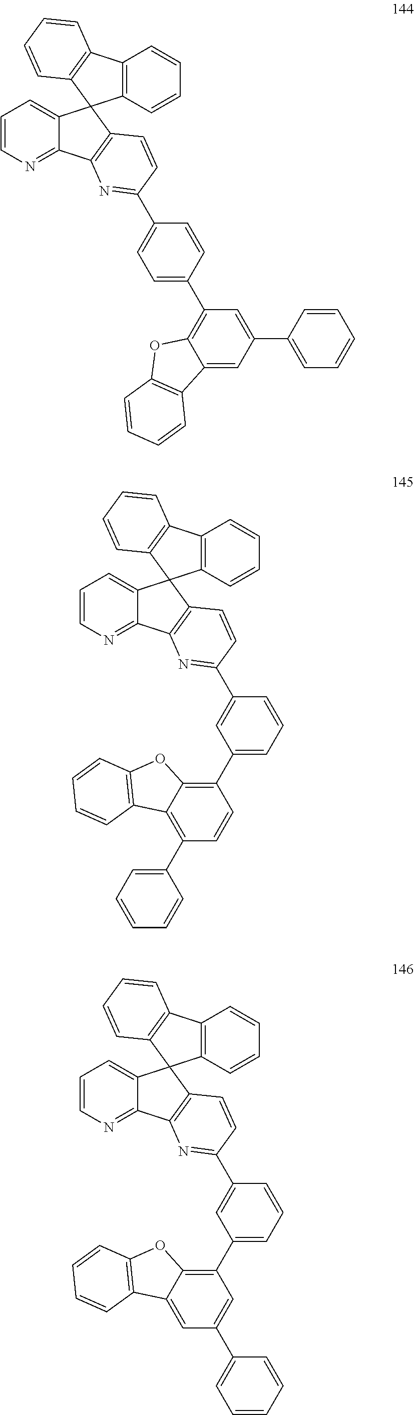 [SMILES: c1ccc(-c2cc(-c3ccc(-c4ccc5c(n4)-c4ncccc4C54c5ccccc5-c5ccccc54)cc3)c3oc4ccccc4c3c2)cc1 ; c1ccc(-c2ccc(-c3cccc(-c4ccc5c(n4)-c4ncccc4C54c5ccccc5-c5ccccc54)c3)c3oc4ccccc4c23)cc1 ; c1ccc(-c2cc(-c3cccc(-c4ccc5c(n4)-c4ncccc4C54c5ccccc5-c5ccccc54)c3)c3oc4ccccc4c3c2)cc1]

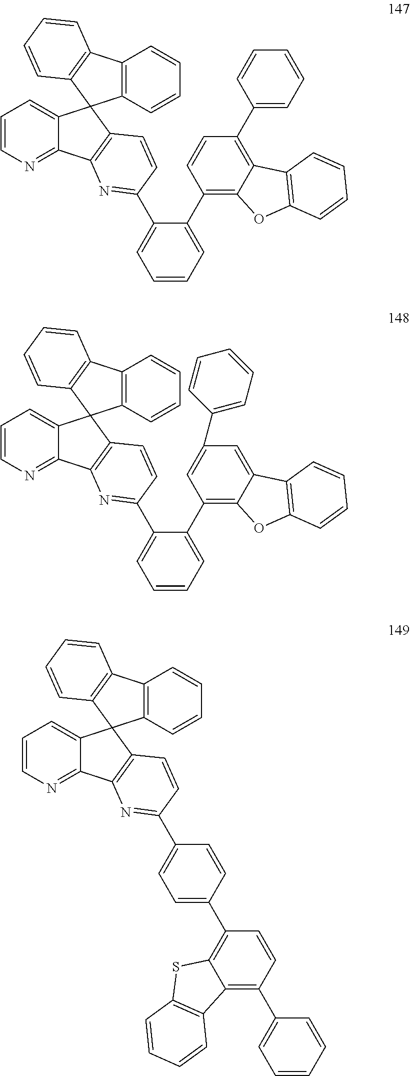 [SMILES: c1ccc(-c2ccc(-c3ccccc3-c3ccc4c(n3)-c3ncccc3C43c4ccccc4-c4ccccc43)c3oc4ccccc4c23)cc1 ; c1ccc(-c2cc(-c3ccccc3-c3ccc4c(n3)-c3ncccc3C43c4ccccc4-c4ccccc43)c3oc4ccccc4c3c2)cc1 ; c1ccc(-c2ccc(-c3ccc(-c4ccc5c(n4)-c4ncccc4C54c5ccccc5-c5ccccc54)cc3)c3sc4ccccc4c23)cc1]

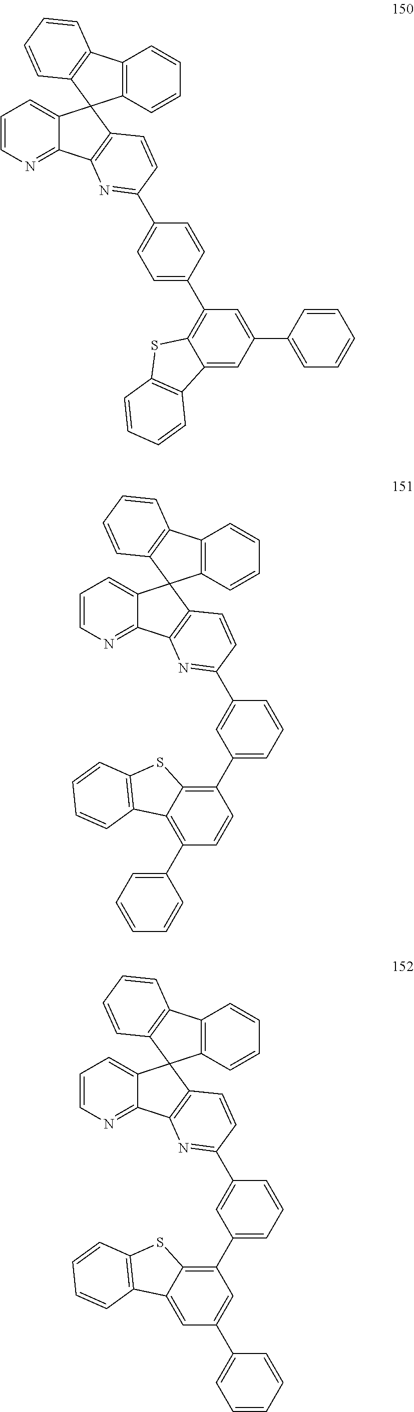

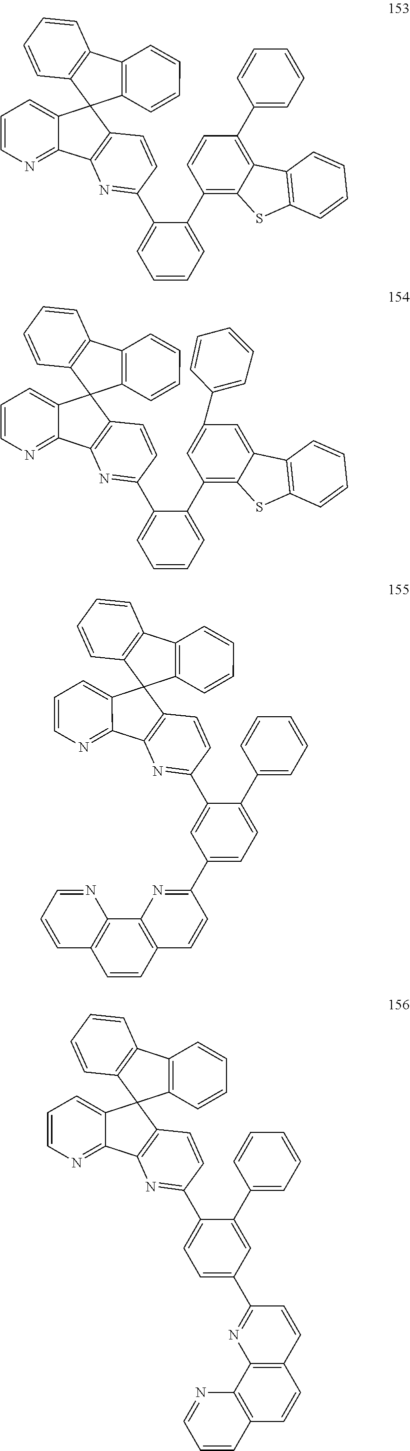

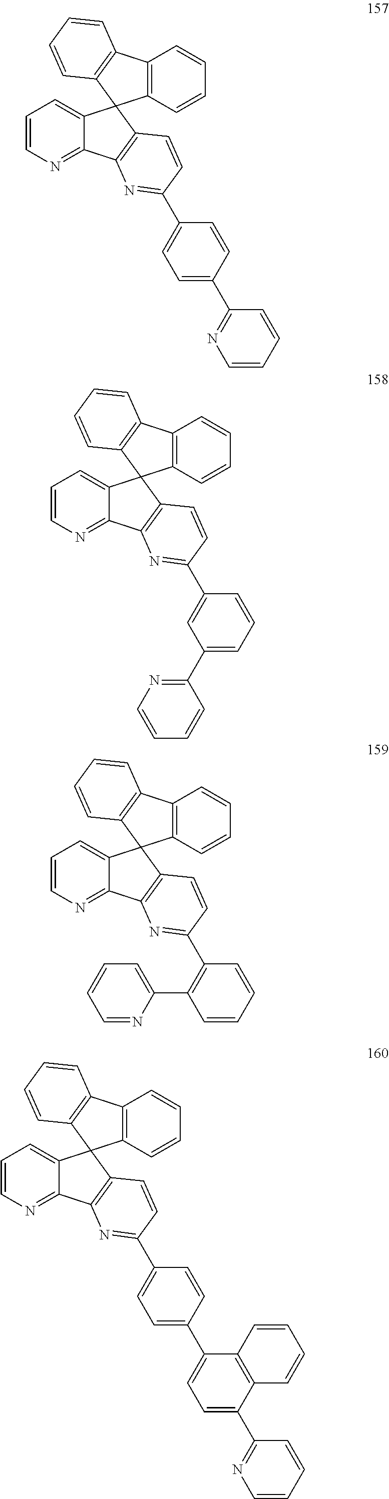

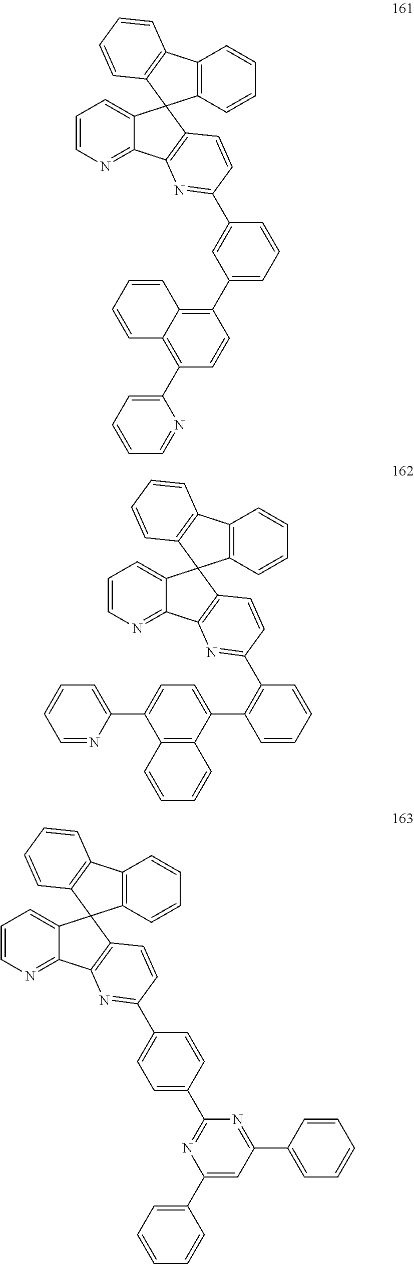

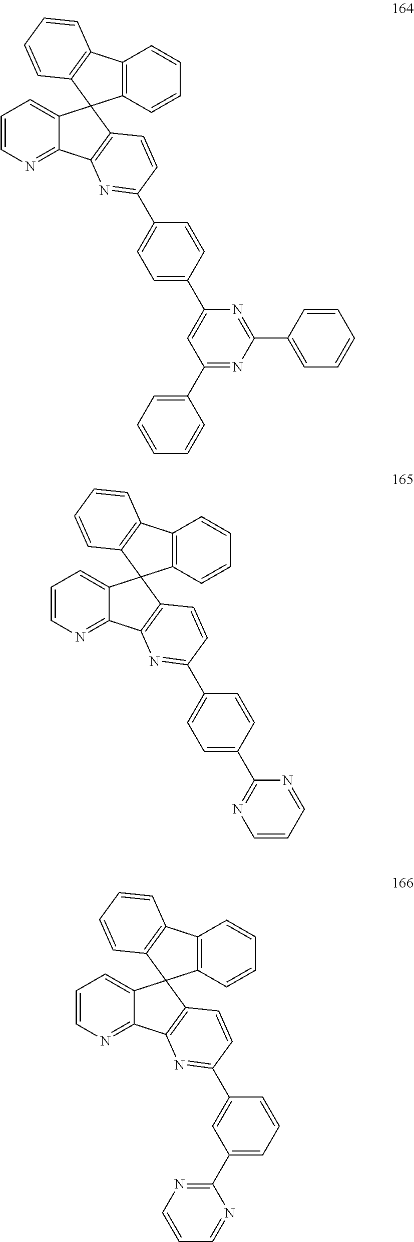 [SMILES: c1ccc(-c2cc(-c3ccc(-c4ccc5c(n4)-c4ncccc4C54c5ccccc5-c5ccccc54)cc3)nc(-c3ccccc3)n2)cc1 ; c1cnc(-c2ccc(-c3ccc4c(n3)-c3ncccc3C43c4ccccc4-c4ccccc43)cc2)nc1 ; c1cnc(-c2cccc(-c3ccc4c(n3)-c3ncccc3C43c4ccccc4-c4ccccc43)c2)nc1]

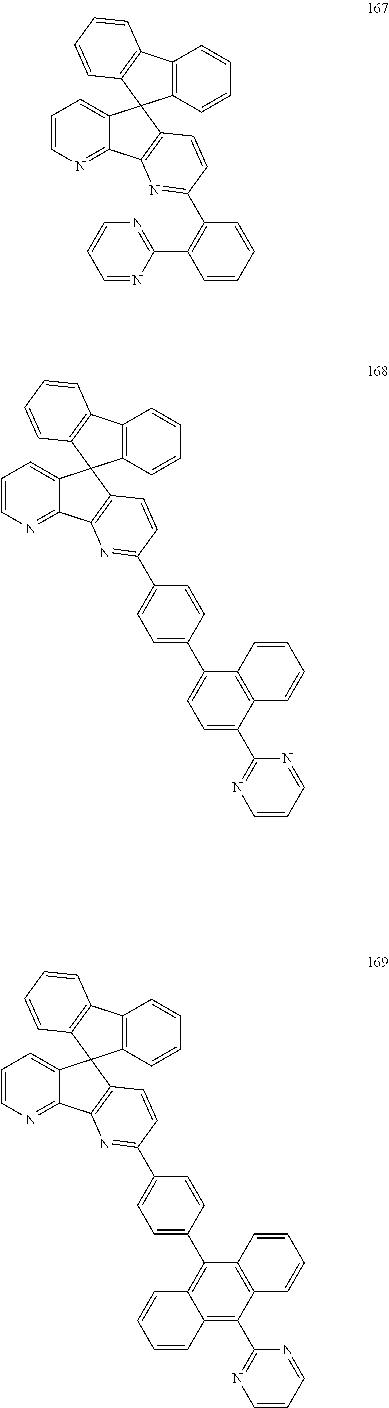 [SMILES: c1cnc(-c2ccccc2-c2ccc3c(n2)-c2ncccc2C32c3ccccc3-c3ccccc32)nc1 ; c1cnc(-c2ccc(-c3ccc(-c4ccc5c(n4)-c4ncccc4C54c5ccccc5-c5ccccc54)cc3)c3ccccc23)nc1 ; c1cnc(-c2c3ccccc3c(-c3ccc(-c4ccc5c(n4)-c4ncccc4C54c5ccccc5-c5ccccc54)cc3)c3ccccc23)nc1]

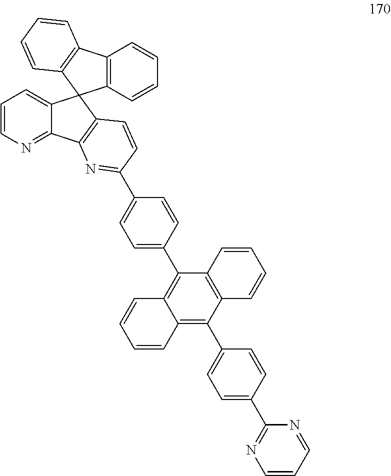 [SMILES: c1cnc(-c2ccc(-c3c4ccccc4c(-c4ccc(-c5ccc6c(n5)-c5ncccc5C65c6ccccc6-c6ccccc65)cc4)c4ccccc34)cc2)nc1]

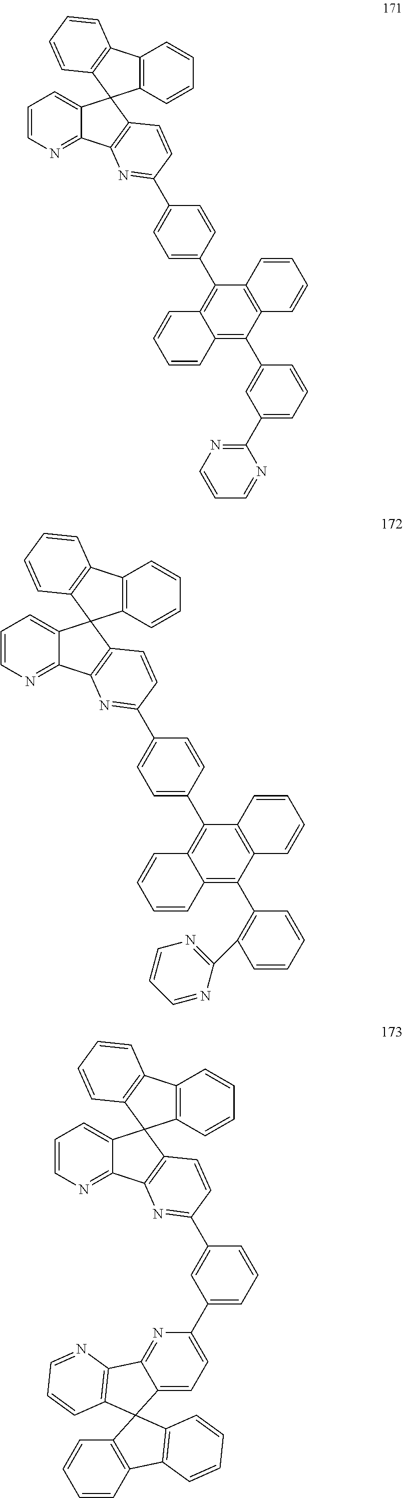 [SMILES: c1cnc(-c2cccc(-c3c4ccccc4c(-c4ccc(-c5ccc6c(n5)-c5ncccc5C65c6ccccc6-c6ccccc65)cc4)c4ccccc34)c2)nc1 ; c1cnc(-c2ccccc2-c2c3ccccc3c(-c3ccc(-c4ccc5c(n4)-c4ncccc4C54c5ccccc5-c5ccccc54)cc3)c3ccccc23)nc1 ; c1cc(-c2ccc3c(n2)-c2ncccc2C32c3ccccc3-c3ccccc32)cc(-c2ccc3c(n2)-c2ncccc2C32c3ccccc3-c3ccccc32)c1]

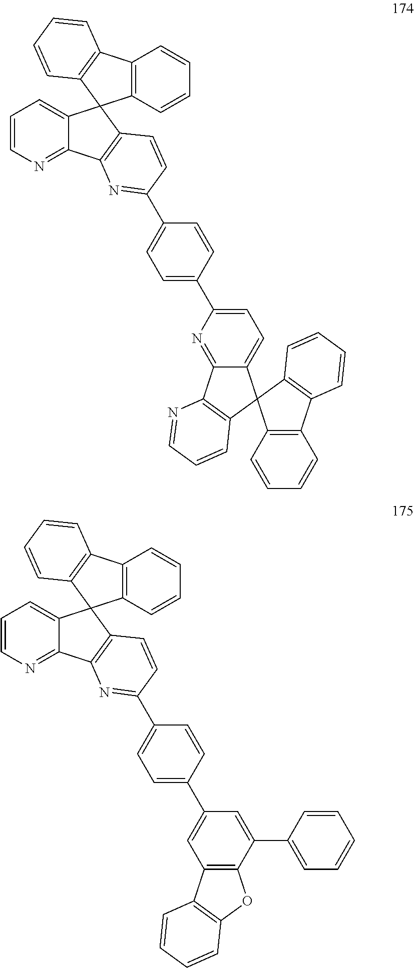 [SMILES: c1ccc2c(c1)-c1ccccc1C21c2cccnc2-c2nc(-c3ccc(-c4ccc5c(n4)-c4ncccc4C54c5ccccc5-c5ccccc54)cc3)ccc21 ; c1ccc(-c2cc(-c3ccc(-c4ccc5c(n4)-c4ncccc4C54c5ccccc5-c5ccccc54)cc3)cc3c2oc2ccccc23)cc1]

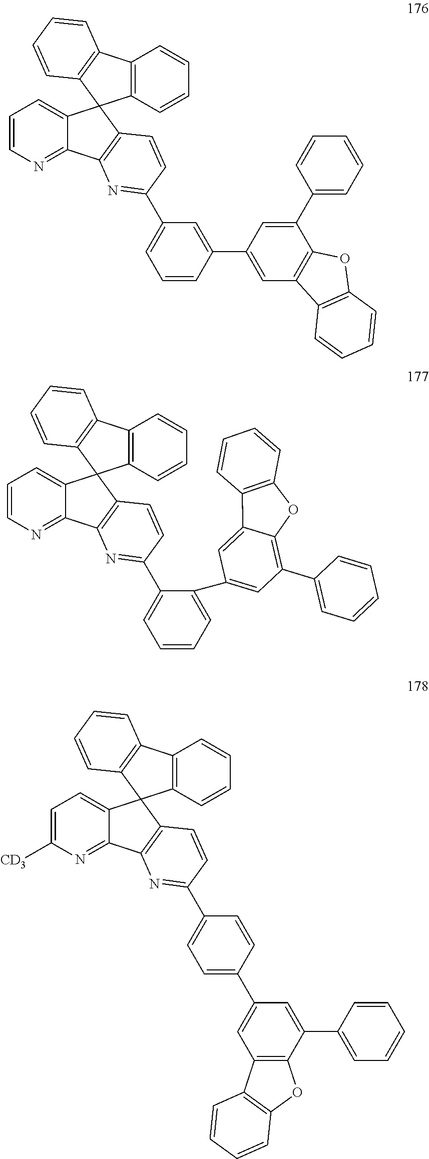 [SMILES: c1ccc(-c2cc(-c3cccc(-c4ccc5c(n4)-c4ncccc4C54c5ccccc5-c5ccccc54)c3)cc3c2oc2ccccc23)cc1 ; c1ccc(-c2cc(-c3ccccc3-c3ccc4c(n3)-c3ncccc3C43c4ccccc4-c4ccccc43)cc3c2oc2ccccc23)cc1 ; [2H]C([2H])([2H])c1ccc2c(n1)-c1nc(-c3ccc(-c4cc(-c5ccccc5)c5oc6ccccc6c5c4)cc3)ccc1C21c2ccccc2-c2ccccc21]

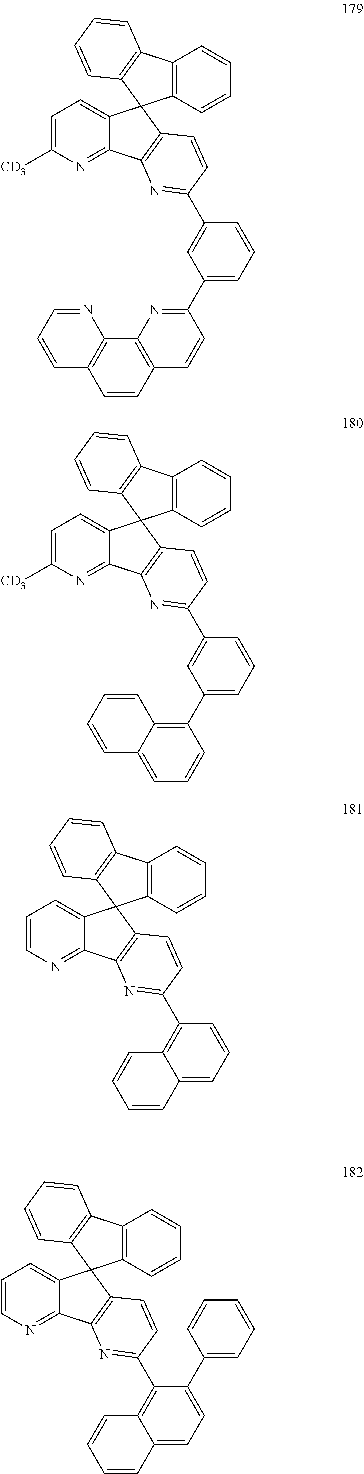

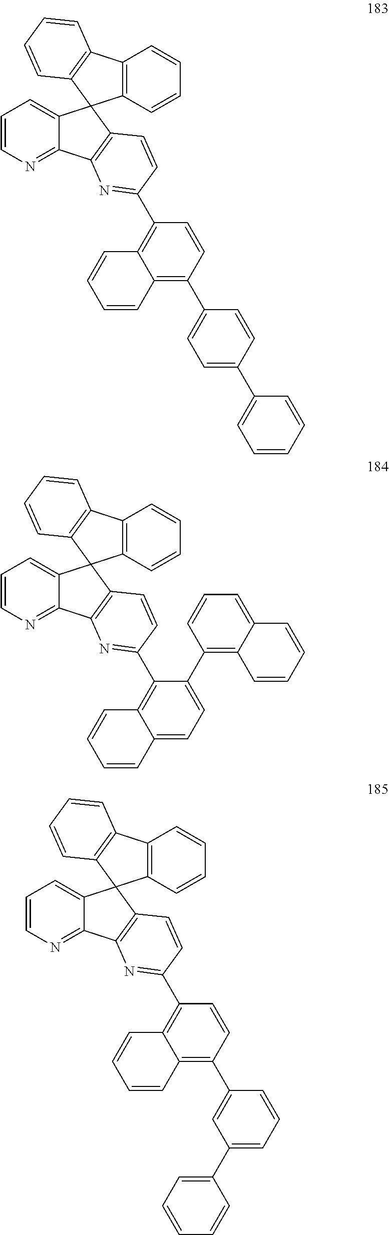

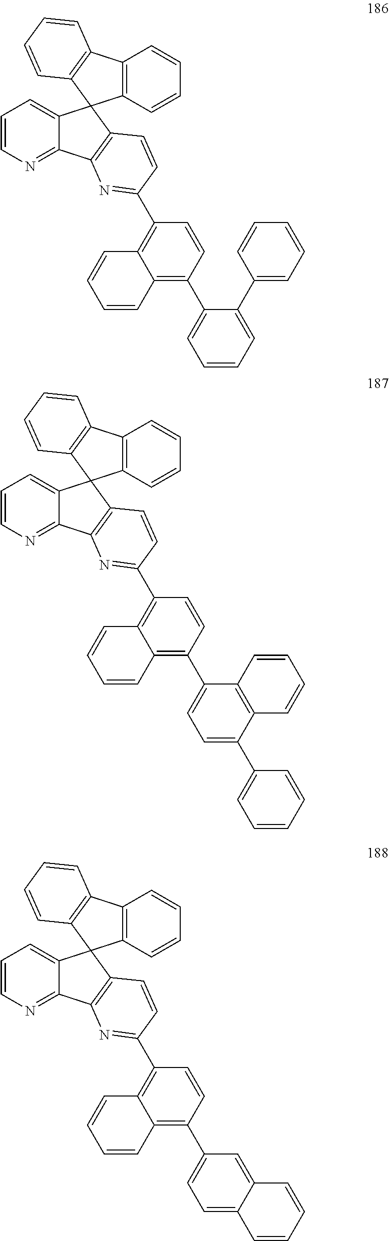 [SMILES: c1ccc(-c2ccccc2-c2ccc(-c3ccc4c(n3)-c3ncccc3C43c4ccccc4-c4ccccc43)c3ccccc23)cc1 ; c1ccc(-c2ccc(-c3ccc(-c4ccc5c(n4)-c4ncccc4C54c5ccccc5-c5ccccc54)c4ccccc34)c3ccccc23)cc1 ; c1ccc2c(c1)-c1ccccc1C21c2cccnc2-c2nc(-c3ccc(-c4ccc5ccccc5c4)c4ccccc34)ccc21]

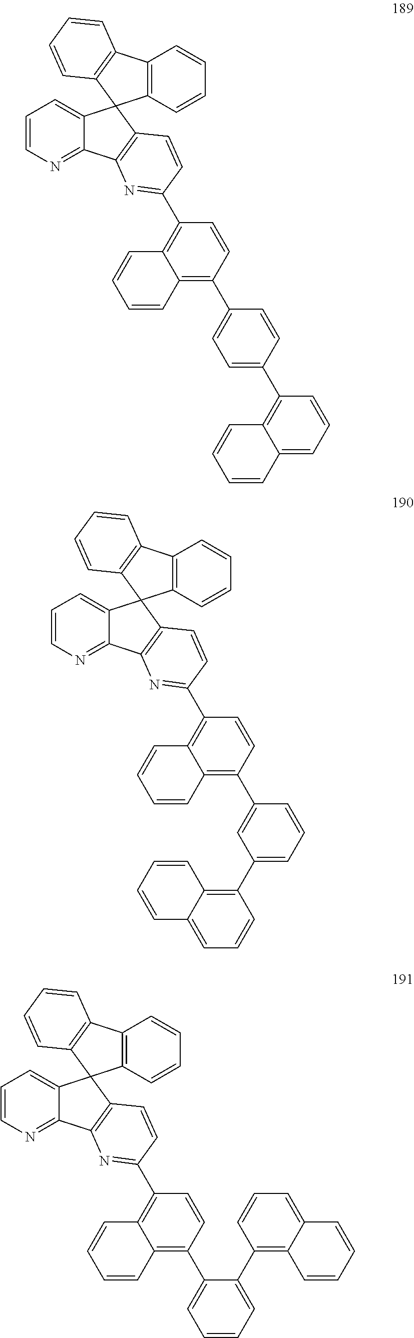 [SMILES: c1ccc2c(c1)-c1ccccc1C21c2cccnc2-c2nc(-c3ccc(-c4ccc(-c5cccc6ccccc56)cc4)c4ccccc34)ccc21 ; c1cc(-c2cccc3ccccc23)cc(-c2ccc(-c3ccc4c(n3)-c3ncccc3C43c4ccccc4-c4ccccc43)c3ccccc23)c1 ; c1ccc(-c2ccc(-c3ccc4c(n3)-c3ncccc3C43c4ccccc4-c4ccccc43)c3ccccc23)c(-c2cccc3ccccc23)c1]

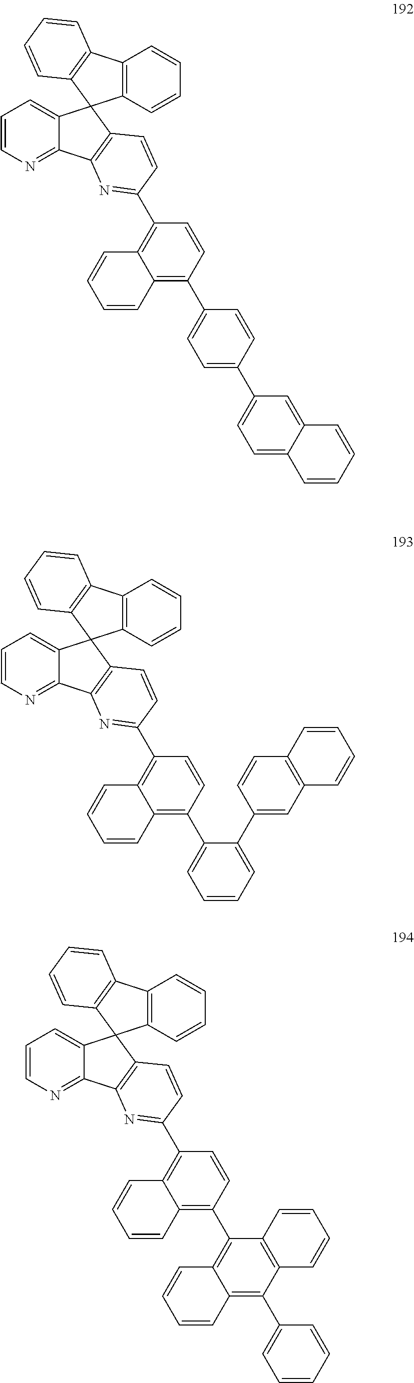 [SMILES: c1ccc2c(c1)-c1ccccc1C21c2cccnc2-c2nc(-c3ccc(-c4ccc(-c5ccc6ccccc6c5)cc4)c4ccccc34)ccc21 ; c1ccc(-c2ccc(-c3ccc4c(n3)-c3ncccc3C43c4ccccc4-c4ccccc43)c3ccccc23)c(-c2ccc3ccccc3c2)c1 ; c1ccc(-c2c3ccccc3c(-c3ccc(-c4ccc5c(n4)-c4ncccc4C54c5ccccc5-c5ccccc54)c4ccccc34)c3ccccc23)cc1]

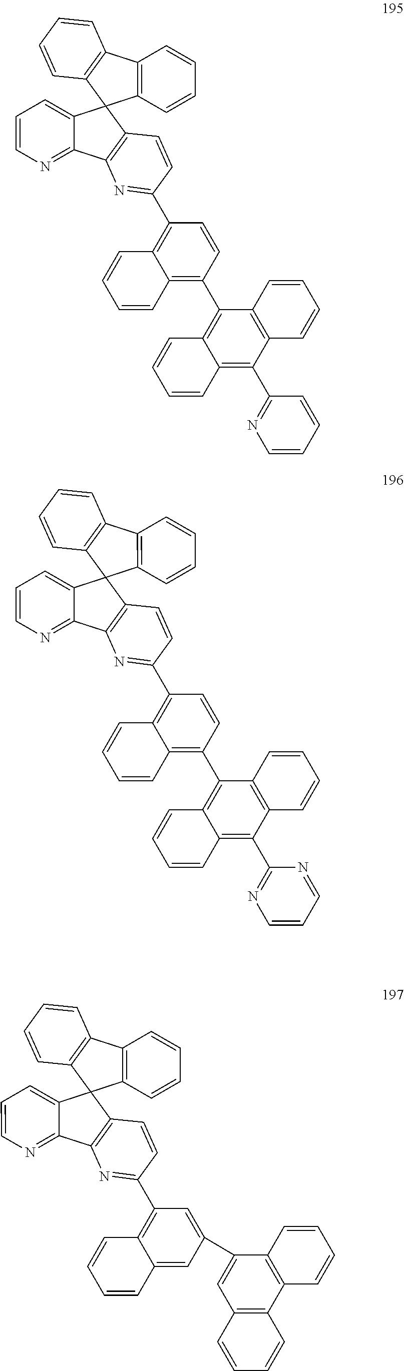 [SMILES: c1ccc(-c2c3ccccc3c(-c3ccc(-c4ccc5c(n4)-c4ncccc4C54c5ccccc5-c5ccccc54)c4ccccc34)c3ccccc23)nc1 ; c1cnc(-c2c3ccccc3c(-c3ccc(-c4ccc5c(n4)-c4ncccc4C54c5ccccc5-c5ccccc54)c4ccccc34)c3ccccc23)nc1 ; c1ccc2c(c1)-c1ccccc1C21c2cccnc2-c2nc(-c3cc(-c4cc5ccccc5c5ccccc45)cc4ccccc34)ccc21]

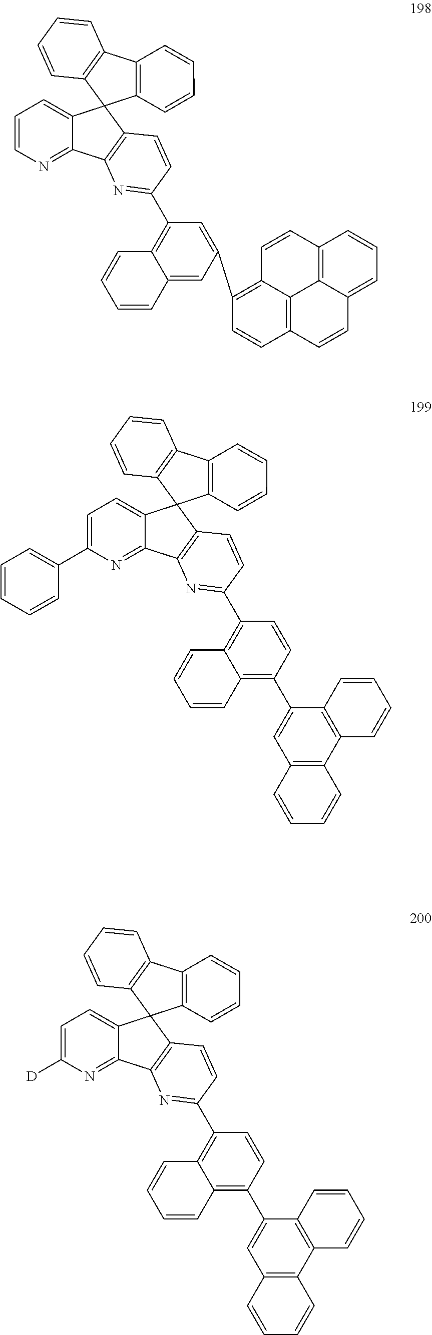 [SMILES: c1ccc2c(c1)-c1ccccc1C21c2cccnc2-c2nc(-c3cc(-c4ccc5ccc6cccc7ccc4c5c67)cc4ccccc34)ccc21 ; c1ccc(-c2ccc3c(n2)-c2nc(-c4ccc(-c5cc6ccccc6c6ccccc56)c5ccccc45)ccc2C32c3ccccc3-c3ccccc32)cc1 ; [2H]c1ccc2c(n1)-c1nc(-c3ccc(-c4cc5ccccc5c5ccccc45)c4ccccc34)ccc1C21c2ccccc2-c2ccccc21]

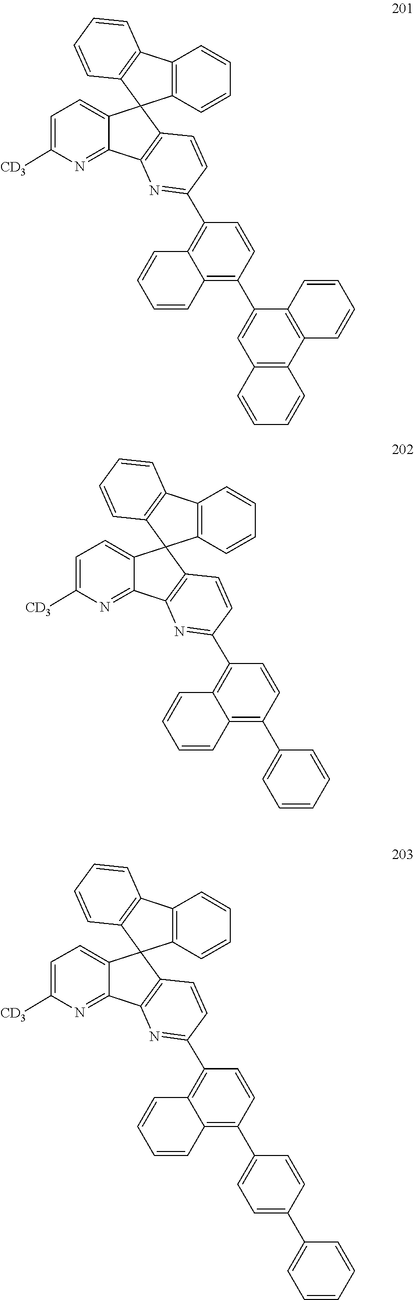 [SMILES: [2H]C([2H])([2H])c1ccc2c(n1)-c1nc(-c3ccc(-c4cc5ccccc5c5ccccc45)c4ccccc34)ccc1C21c2ccccc2-c2ccccc21 ; [2H]C([2H])([2H])c1ccc2c(n1)-c1nc(-c3ccc(-c4ccccc4)c4ccccc34)ccc1C21c2ccccc2-c2ccccc21 ; [2H]C([2H])([2H])c1ccc2c(n1)-c1nc(-c3ccc(-c4ccc(-c5ccccc5)cc4)c4ccccc34)ccc1C21c2ccccc2-c2ccccc21]

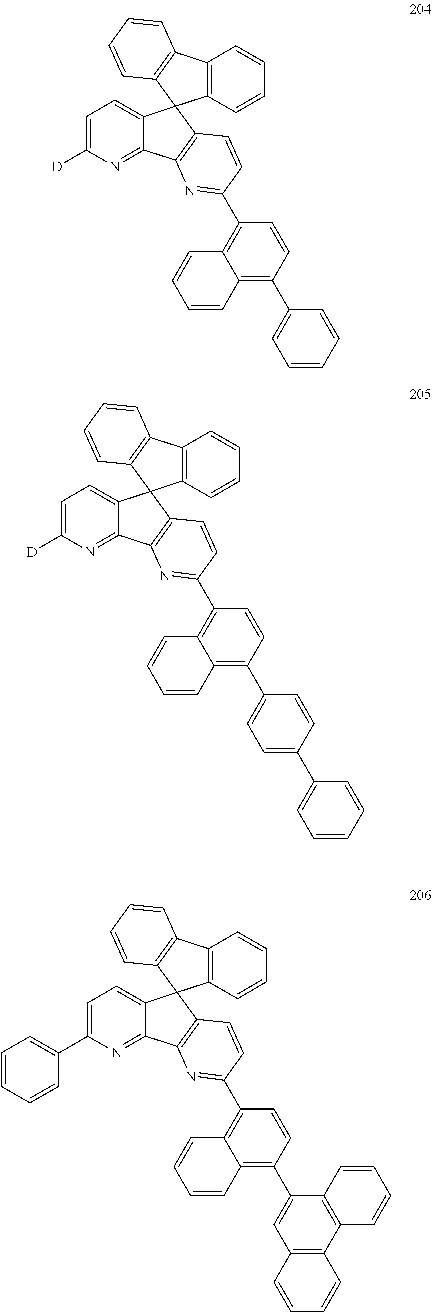 [SMILES: [2H]c1ccc2c(n1)-c1nc(-c3ccc(-c4ccccc4)c4ccccc34)ccc1C21c2ccccc2-c2ccccc21 ; [2H]c1ccc2c(n1)-c1nc(-c3ccc(-c4ccc(-c5ccccc5)cc4)c4ccccc34)ccc1C21c2ccccc2-c2ccccc21 ; c1ccc(-c2ccc3c(n2)-c2nc(-c4ccc(-c5cc6ccccc6c6ccccc56)c5ccccc45)ccc2C32c3ccccc3-c3ccccc32)cc1]

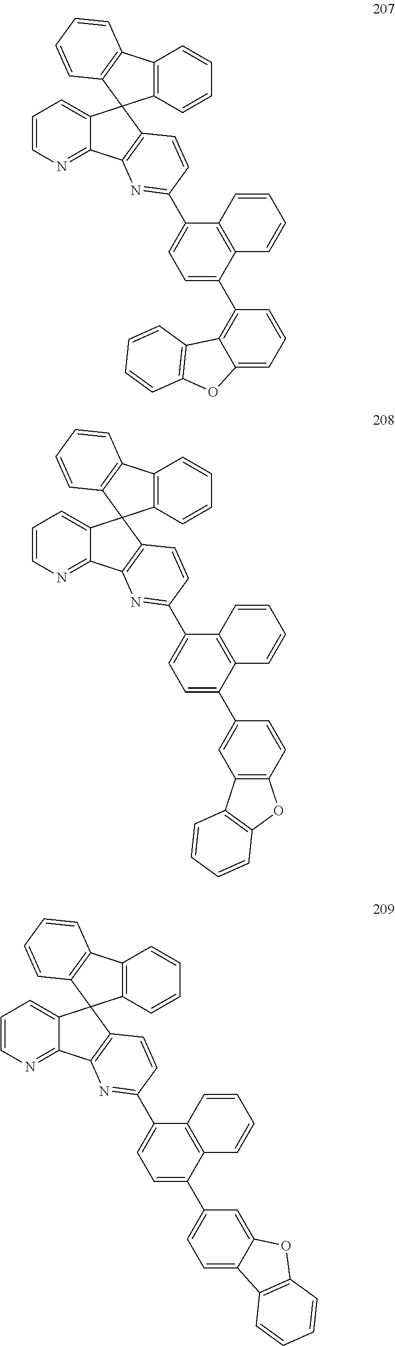 [SMILES: c1ccc2c(c1)-c1ccccc1C21c2cccnc2-c2nc(-c3ccc(-c4cccc5oc6ccccc6c45)c4ccccc34)ccc21 ; c1ccc2c(c1)-c1ccccc1C21c2cccnc2-c2nc(-c3ccc(-c4ccc5oc6ccccc6c5c4)c4ccccc34)ccc21 ; c1ccc2c(c1)-c1ccccc1C21c2cccnc2-c2nc(-c3ccc(-c4ccc5c(c4)oc4ccccc45)c4ccccc34)ccc21]

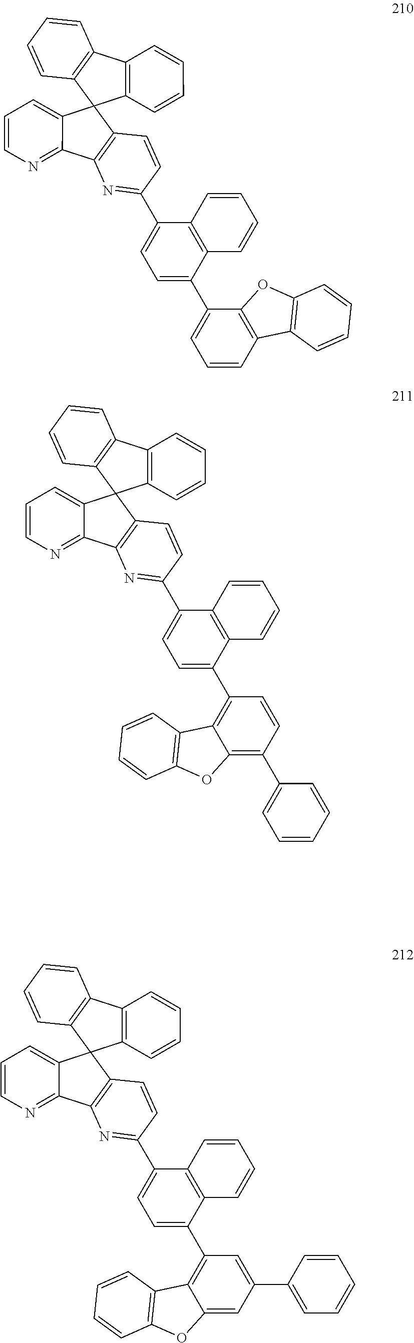 [SMILES: c1ccc2c(c1)-c1ccccc1C21c2cccnc2-c2nc(-c3ccc(-c4cccc5c4oc4ccccc45)c4ccccc34)ccc21 ; c1ccc(-c2ccc(-c3ccc(-c4ccc5c(n4)-c4ncccc4C54c5ccccc5-c5ccccc54)c4ccccc34)c3c2oc2ccccc23)cc1 ; c1ccc(-c2cc(-c3ccc(-c4ccc5c(n4)-c4ncccc4C54c5ccccc5-c5ccccc54)c4ccccc34)c3c(c2)oc2ccccc23)cc1]

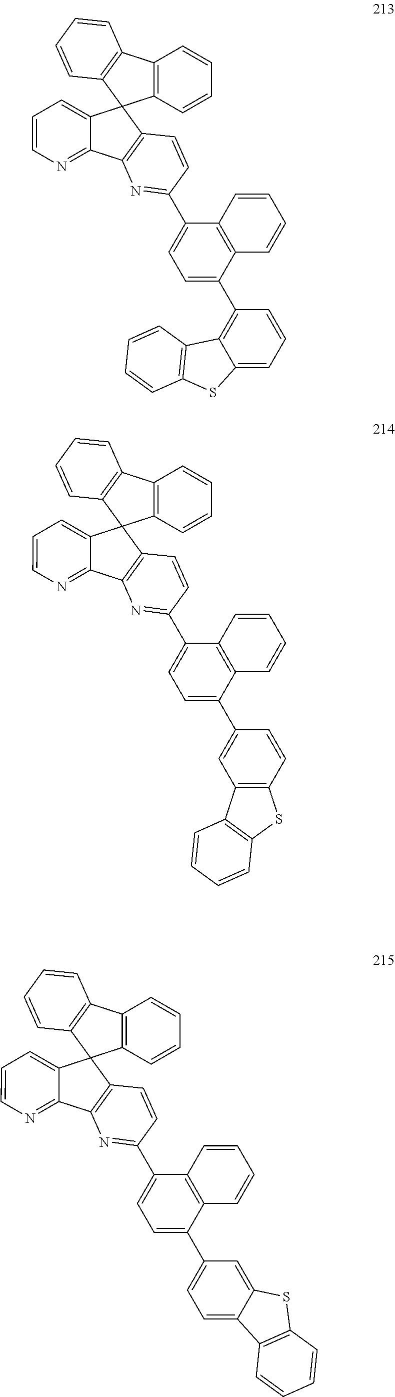 [SMILES: c1ccc2c(c1)-c1ccccc1C21c2cccnc2-c2nc(-c3ccc(-c4cccc5sc6ccccc6c45)c4ccccc34)ccc21 ; c1ccc2c(c1)-c1ccccc1C21c2cccnc2-c2nc(-c3ccc(-c4ccc5sc6ccccc6c5c4)c4ccccc34)ccc21 ; c1ccc2c(c1)-c1ccccc1C21c2cccnc2-c2nc(-c3ccc(-c4ccc5c(c4)sc4ccccc45)c4ccccc34)ccc21]

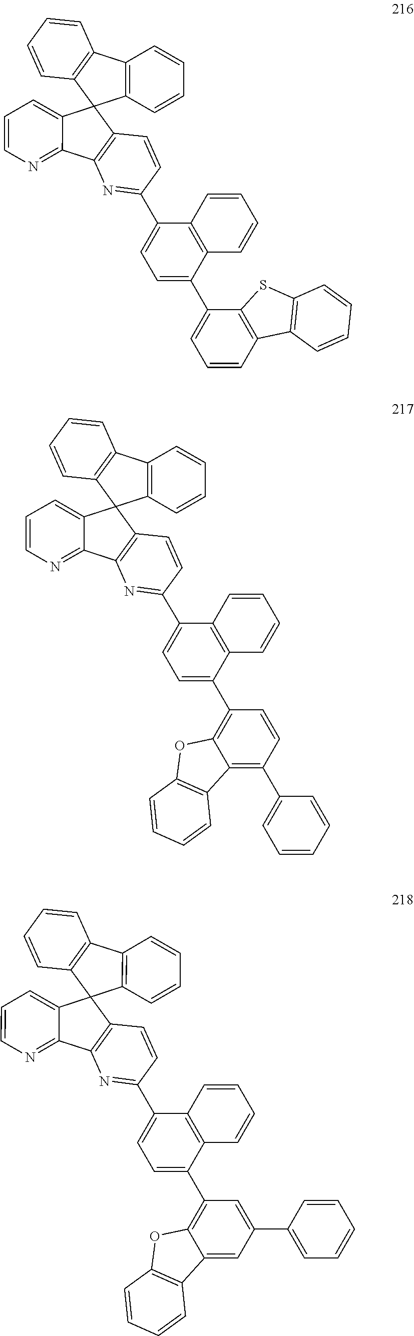 [SMILES: c1ccc2c(c1)-c1ccccc1C21c2cccnc2-c2nc(-c3ccc(-c4cccc5c4sc4ccccc45)c4ccccc34)ccc21 ; c1ccc(-c2ccc(-c3ccc(-c4ccc5c(n4)-c4ncccc4C54c5ccccc5-c5ccccc54)c4ccccc34)c3oc4ccccc4c23)cc1 ; c1ccc(-c2cc(-c3ccc(-c4ccc5c(n4)-c4ncccc4C54c5ccccc5-c5ccccc54)c4ccccc34)c3oc4ccccc4c3c2)cc1]

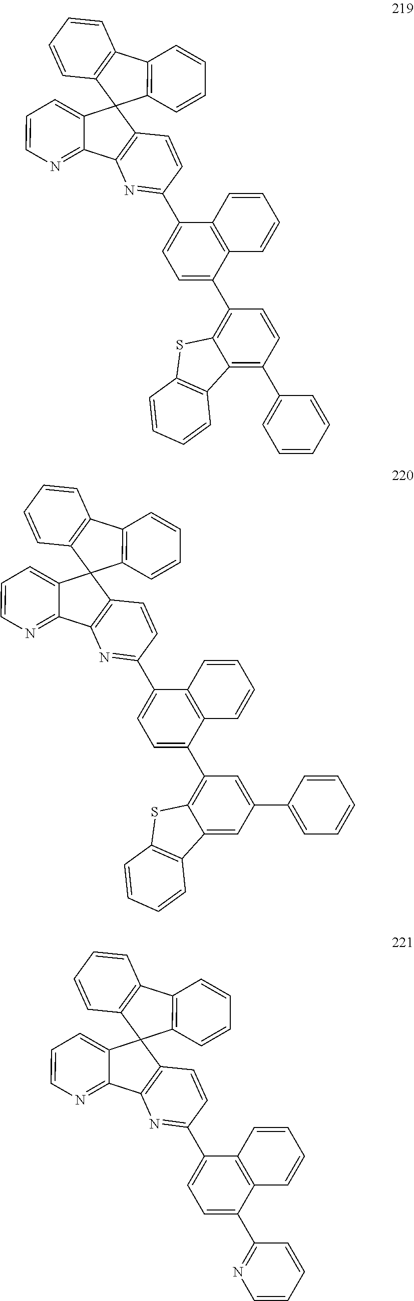 [SMILES: c1ccc(-c2ccc(-c3ccc(-c4ccc5c(n4)-c4ncccc4C54c5ccccc5-c5ccccc54)c4ccccc34)c3sc4ccccc4c23)cc1 ; c1ccc(-c2cc(-c3ccc(-c4ccc5c(n4)-c4ncccc4C54c5ccccc5-c5ccccc54)c4ccccc34)c3sc4ccccc4c3c2)cc1 ; c1ccc(-c2ccc(-c3ccc4c(n3)-c3ncccc3C43c4ccccc4-c4ccccc43)c3ccccc23)nc1]

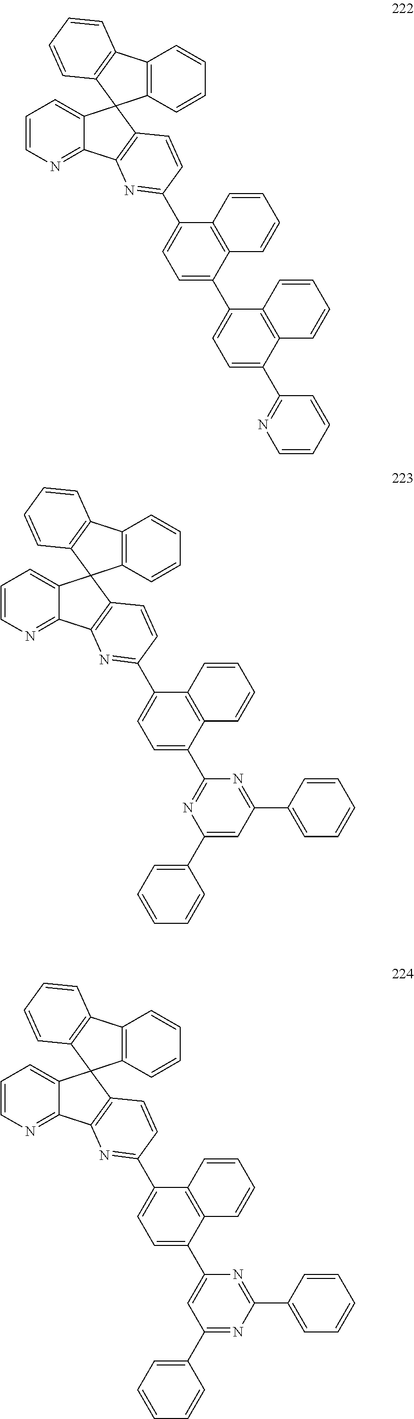 [SMILES: c1ccc(-c2ccc(-c3ccc(-c4ccc5c(n4)-c4ncccc4C54c5ccccc5-c5ccccc54)c4ccccc34)c3ccccc23)nc1 ; c1ccc(-c2cc(-c3ccccc3)nc(-c3ccc(-c4ccc5c(n4)-c4ncccc4C54c5ccccc5-c5ccccc54)c4ccccc34)n2)cc1 ; c1ccc(-c2cc(-c3ccc(-c4ccc5c(n4)-c4ncccc4C54c5ccccc5-c5ccccc54)c4ccccc34)nc(-c3ccccc3)n2)cc1]

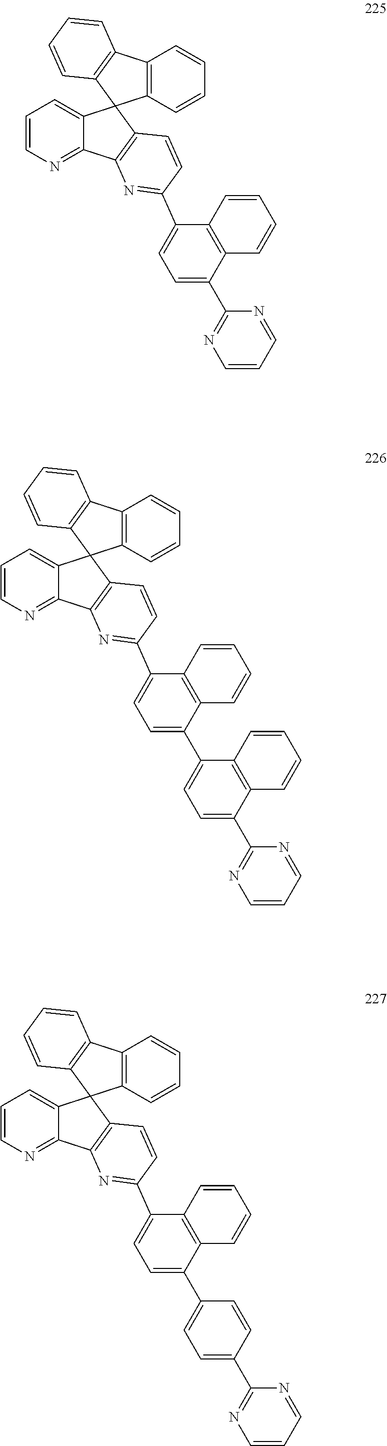 [SMILES: c1cnc(-c2ccc(-c3ccc4c(n3)-c3ncccc3C43c4ccccc4-c4ccccc43)c3ccccc23)nc1 ; c1cnc(-c2ccc(-c3ccc(-c4ccc5c(n4)-c4ncccc4C54c5ccccc5-c5ccccc54)c4ccccc34)c3ccccc23)nc1 ; c1cnc(-c2ccc(-c3ccc(-c4ccc5c(n4)-c4ncccc4C54c5ccccc5-c5ccccc54)c4ccccc34)cc2)nc1]

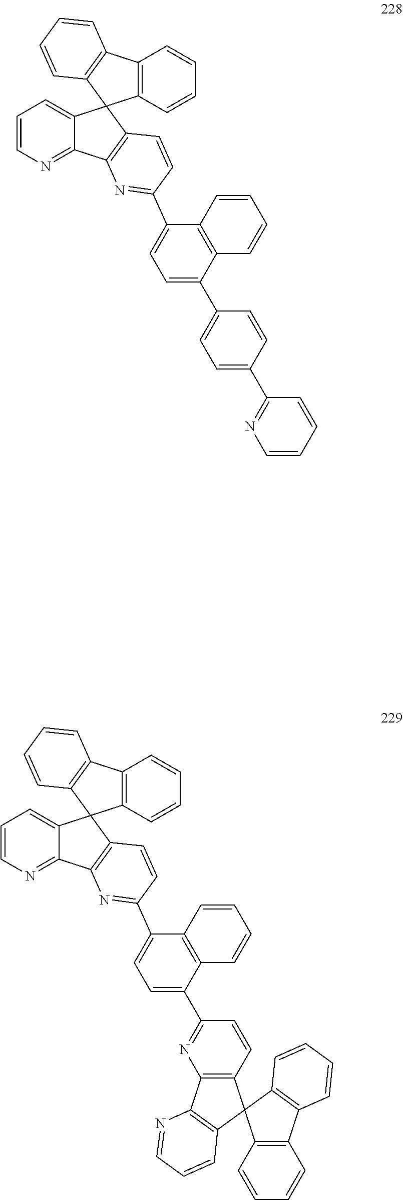 [SMILES: c1ccc(-c2ccc(-c3ccc(-c4ccc5c(n4)-c4ncccc4C54c5ccccc5-c5ccccc54)c4ccccc34)cc2)nc1 ; c1ccc2c(c1)-c1ccccc1C21c2cccnc2-c2nc(-c3ccc(-c4ccc5c(n4)-c4ncccc4C54c5ccccc5-c5ccccc54)c4ccccc34)ccc21]

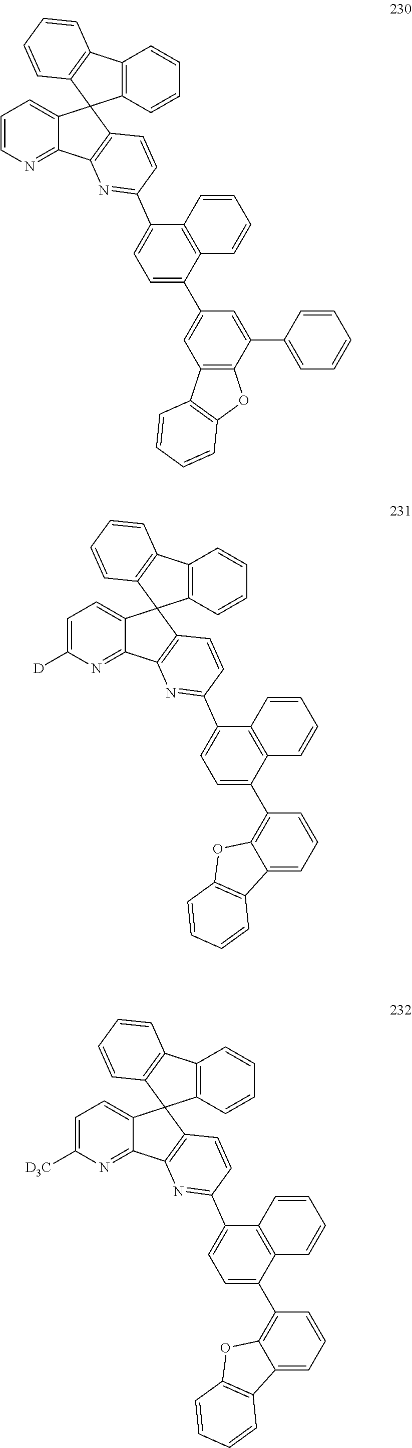 [SMILES: c1ccc(-c2cc(-c3ccc(-c4ccc5c(n4)-c4ncccc4C54c5ccccc5-c5ccccc54)c4ccccc34)cc3c2oc2ccccc23)cc1 ; [2H]c1ccc2c(n1)-c1nc(-c3ccc(-c4cccc5c4oc4ccccc45)c4ccccc34)ccc1C21c2ccccc2-c2ccccc21 ; [2H]C([2H])([2H])c1ccc2c(n1)-c1nc(-c3ccc(-c4cccc5c4oc4ccccc45)c4ccccc34)ccc1C21c2ccccc2-c2ccccc21]

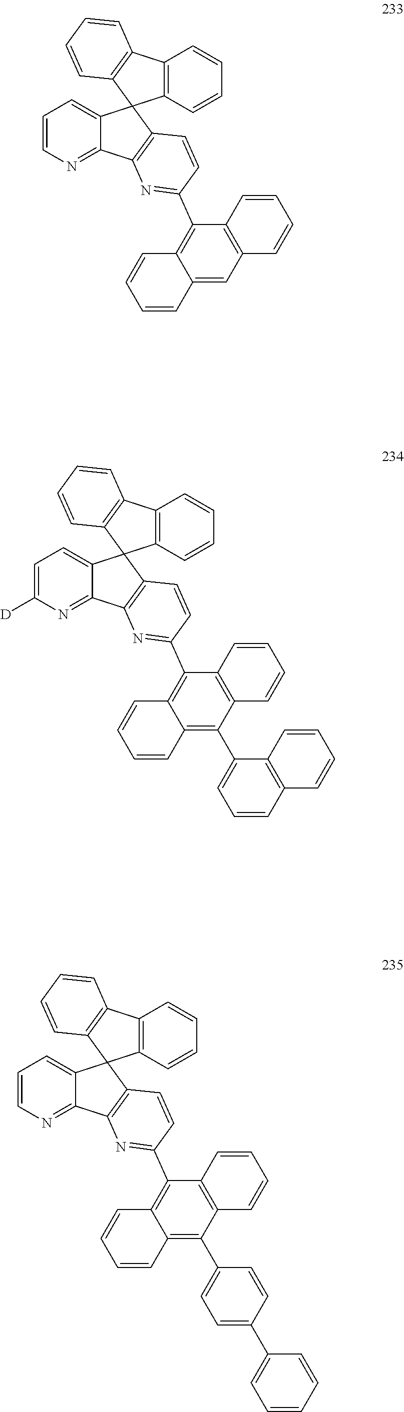 [SMILES: c1ccc2c(c1)-c1ccccc1C21c2cccnc2-c2nc(-c3c4ccccc4cc4ccccc34)ccc21 ; [2H]c1ccc2c(n1)-c1nc(-c3c4ccccc4c(-c4cccc5ccccc45)c4ccccc34)ccc1C21c2ccccc2-c2ccccc21 ; c1ccc(-c2ccc(-c3c4ccccc4c(-c4ccc5c(n4)-c4ncccc4C54c5ccccc5-c5ccccc54)c4ccccc34)cc2)cc1]

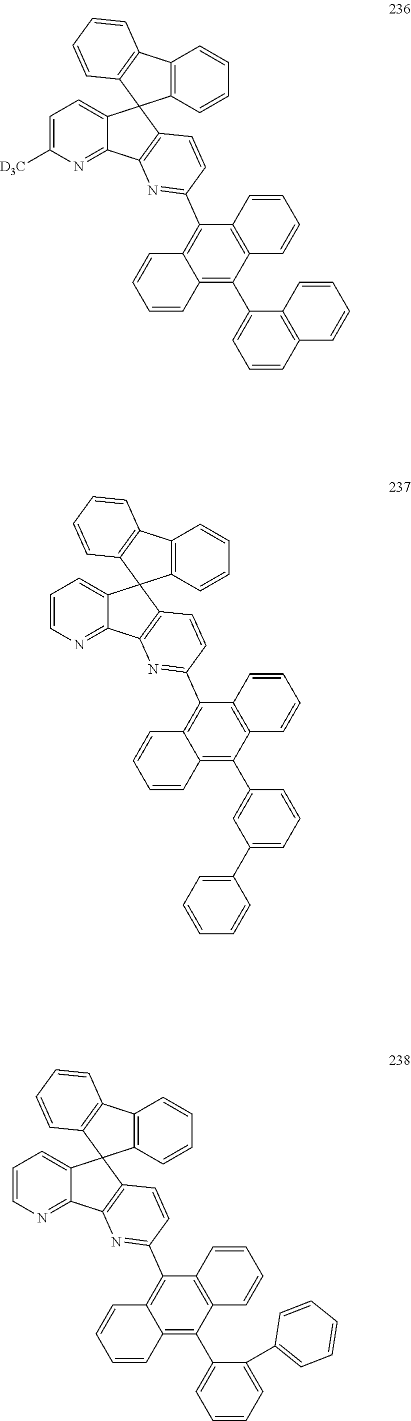 [SMILES: [2H]C([2H])([2H])c1ccc2c(n1)-c1nc(-c3c4ccccc4c(-c4cccc5ccccc45)c4ccccc34)ccc1C21c2ccccc2-c2ccccc21 ; c1ccc(-c2cccc(-c3c4ccccc4c(-c4ccc5c(n4)-c4ncccc4C54c5ccccc5-c5ccccc54)c4ccccc34)c2)cc1 ; c1ccc(-c2ccccc2-c2c3ccccc3c(-c3ccc4c(n3)-c3ncccc3C43c4ccccc4-c4ccccc43)c3ccccc23)cc1]

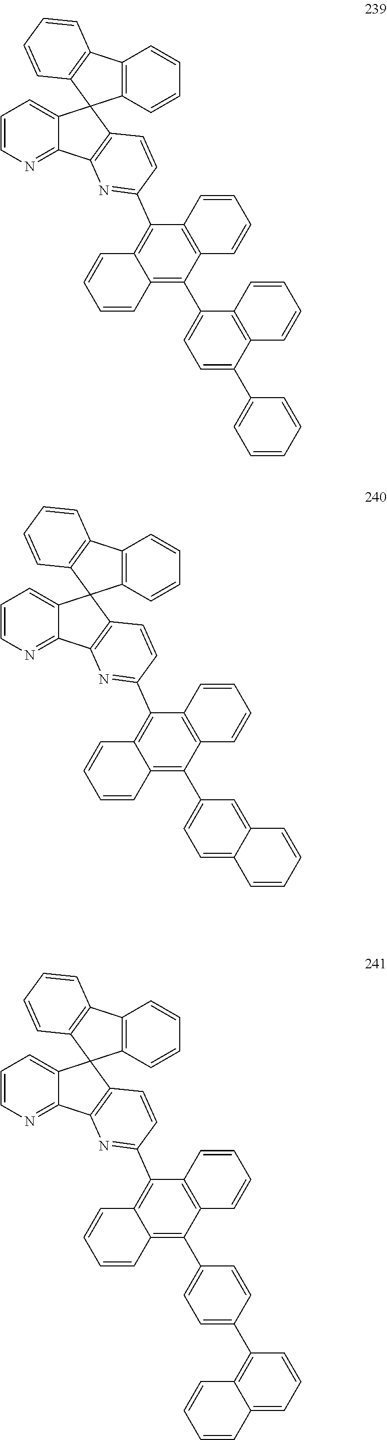 [SMILES: c1ccc(-c2ccc(-c3c4ccccc4c(-c4ccc5c(n4)-c4ncccc4C54c5ccccc5-c5ccccc54)c4ccccc34)c3ccccc23)cc1 ; c1ccc2c(c1)-c1ccccc1C21c2cccnc2-c2nc(-c3c4ccccc4c(-c4ccc5ccccc5c4)c4ccccc34)ccc21 ; c1ccc2c(c1)-c1ccccc1C21c2cccnc2-c2nc(-c3c4ccccc4c(-c4ccc(-c5cccc6ccccc56)cc4)c4ccccc34)ccc21]

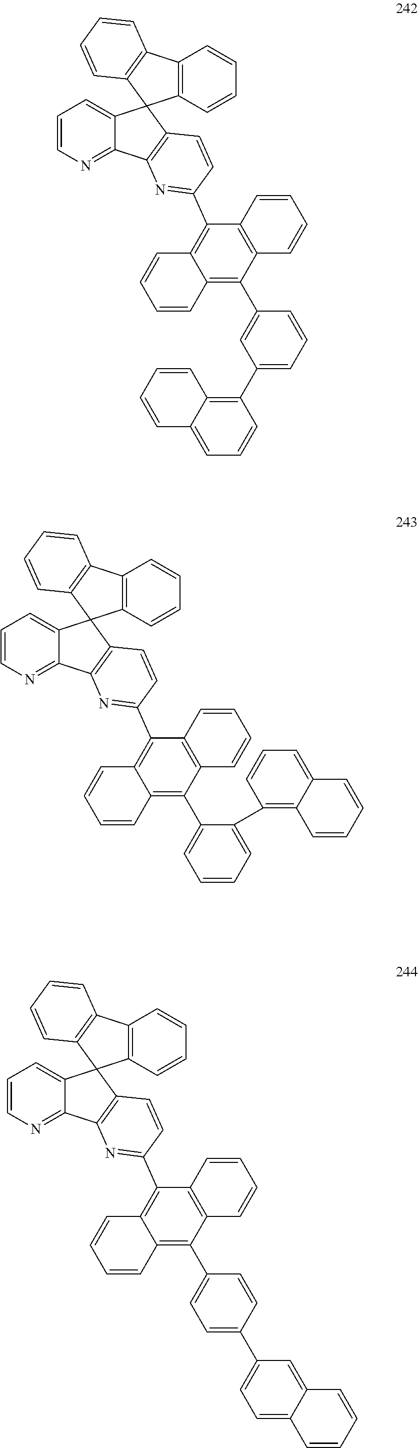 [SMILES: c1cc(-c2cccc3ccccc23)cc(-c2c3ccccc3c(-c3ccc4c(n3)-c3ncccc3C43c4ccccc4-c4ccccc43)c3ccccc23)c1 ; c1ccc(-c2c3ccccc3c(-c3ccc4c(n3)-c3ncccc3C43c4ccccc4-c4ccccc43)c3ccccc23)c(-c2cccc3ccccc23)c1 ; c1ccc2c(c1)-c1ccccc1C21c2cccnc2-c2nc(-c3c4ccccc4c(-c4ccc(-c5ccc6ccccc6c5)cc4)c4ccccc34)ccc21]

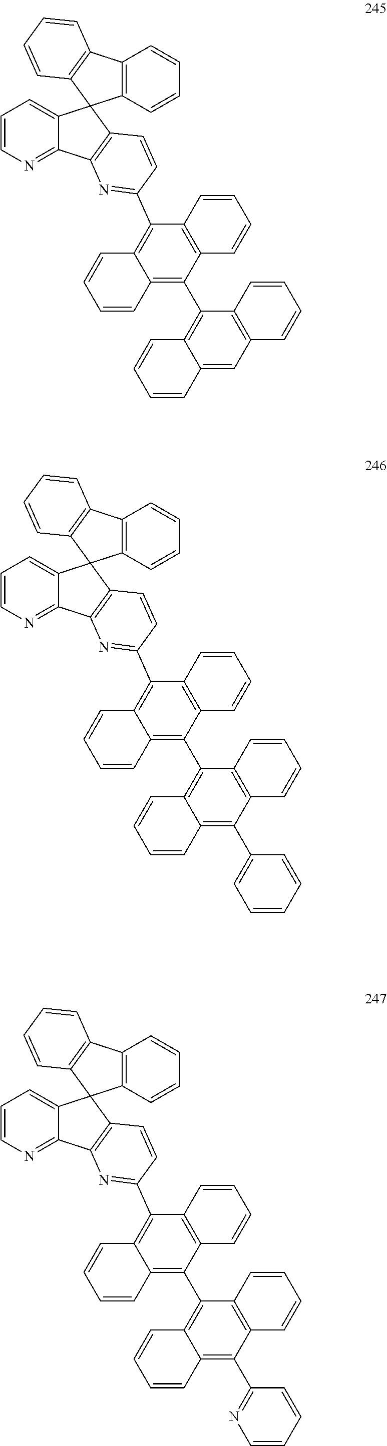 [SMILES: c1ccc2c(c1)-c1ccccc1C21c2cccnc2-c2nc(-c3c4ccccc4c(-c4c5ccccc5cc5ccccc45)c4ccccc34)ccc21 ; c1ccc(-c2c3ccccc3c(-c3c4ccccc4c(-c4ccc5c(n4)-c4ncccc4C54c5ccccc5-c5ccccc54)c4ccccc34)c3ccccc23)cc1 ; c1ccc(-c2c3ccccc3c(-c3c4ccccc4c(-c4ccc5c(n4)-c4ncccc4C54c5ccccc5-c5ccccc54)c4ccccc34)c3ccccc23)nc1]

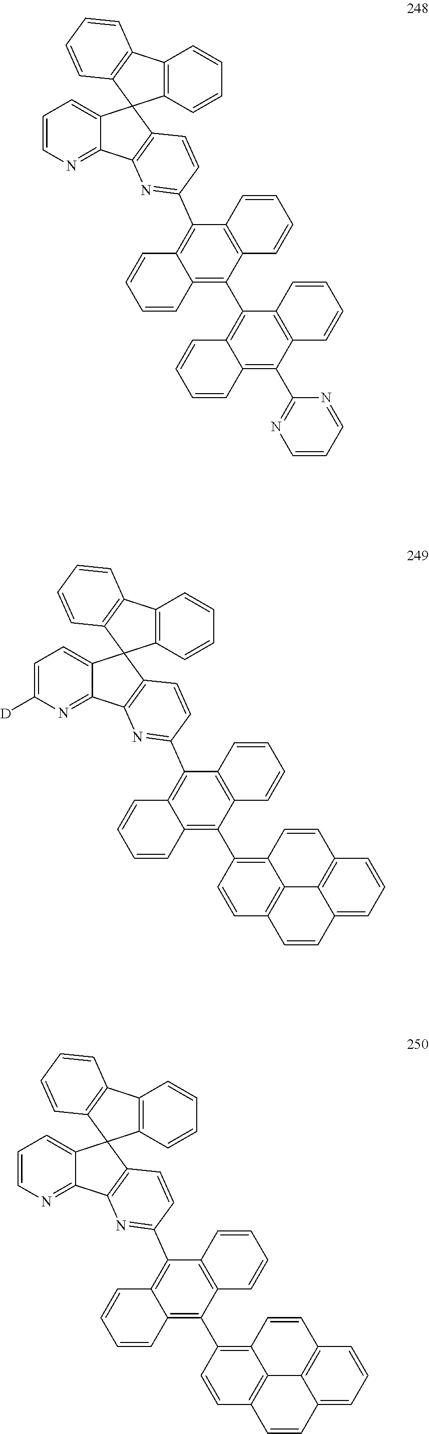 [SMILES: c1cnc(-c2c3ccccc3c(-c3c4ccccc4c(-c4ccc5c(n4)-c4ncccc4C54c5ccccc5-c5ccccc54)c4ccccc34)c3ccccc23)nc1 ; [2H]c1ccc2c(n1)-c1nc(-c3c4ccccc4c(-c4ccc5ccc6cccc7ccc4c5c67)c4ccccc34)ccc1C21c2ccccc2-c2ccccc21 ; c1ccc2c(c1)-c1ccccc1C21c2cccnc2-c2nc(-c3c4ccccc4c(-c4ccc5ccc6cccc7ccc4c5c67)c4ccccc34)ccc21]

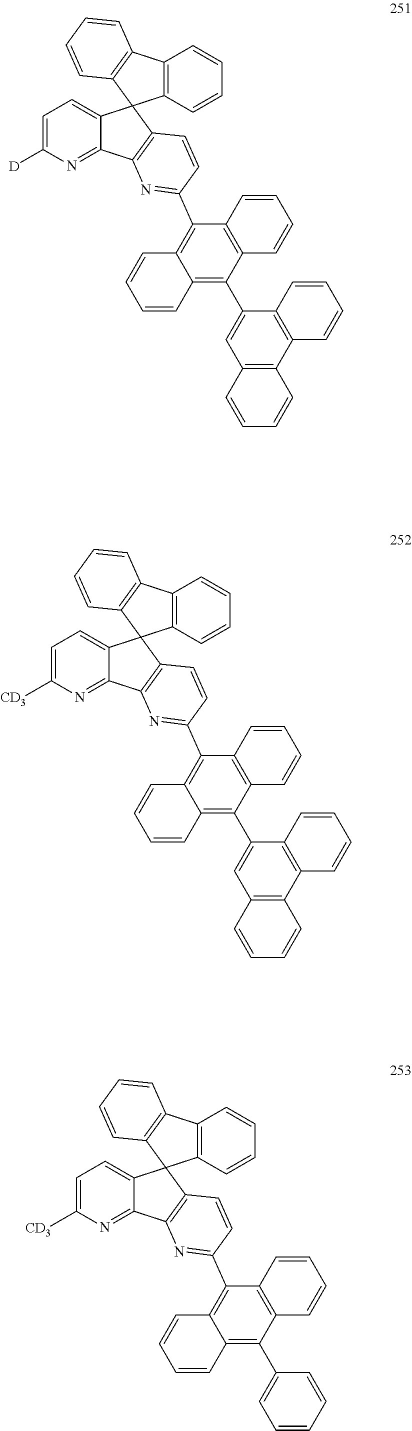 [SMILES: [2H]c1ccc2c(n1)-c1nc(-c3c4ccccc4c(-c4cc5ccccc5c5ccccc45)c4ccccc34)ccc1C21c2ccccc2-c2ccccc21 ; [2H]C([2H])([2H])c1ccc2c(n1)-c1nc(-c3c4ccccc4c(-c4cc5ccccc5c5ccccc45)c4ccccc34)ccc1C21c2ccccc2-c2ccccc21 ; [2H]C([2H])([2H])c1ccc2c(n1)-c1nc(-c3c4ccccc4c(-c4ccccc4)c4ccccc34)ccc1C21c2ccccc2-c2ccccc21]

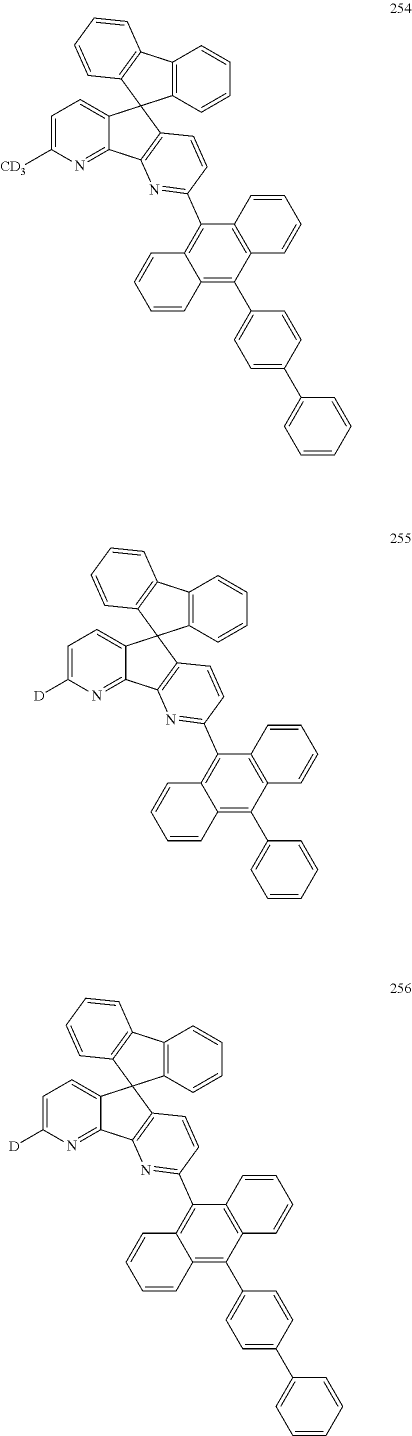 [SMILES: [2H]C([2H])([2H])c1ccc2c(n1)-c1nc(-c3c4ccccc4c(-c4ccc(-c5ccccc5)cc4)c4ccccc34)ccc1C21c2ccccc2-c2ccccc21 ; [2H]c1ccc2c(n1)-c1nc(-c3c4ccccc4c(-c4ccccc4)c4ccccc34)ccc1C21c2ccccc2-c2ccccc21 ; [2H]c1ccc2c(n1)-c1nc(-c3c4ccccc4c(-c4ccc(-c5ccccc5)cc4)c4ccccc34)ccc1C21c2ccccc2-c2ccccc21]

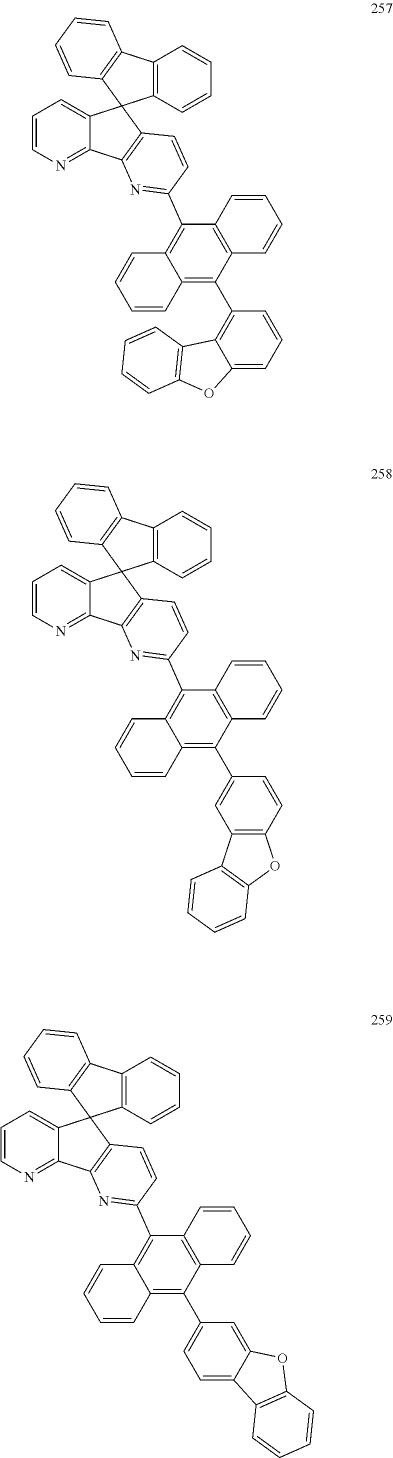 [SMILES: c1ccc2c(c1)-c1ccccc1C21c2cccnc2-c2nc(-c3c4ccccc4c(-c4cccc5oc6ccccc6c45)c4ccccc34)ccc21 ; c1ccc2c(c1)-c1ccccc1C21c2cccnc2-c2nc(-c3c4ccccc4c(-c4ccc5oc6ccccc6c5c4)c4ccccc34)ccc21 ; c1ccc2c(c1)-c1ccccc1C21c2cccnc2-c2nc(-c3c4ccccc4c(-c4ccc5c(c4)oc4ccccc45)c4ccccc34)ccc21]

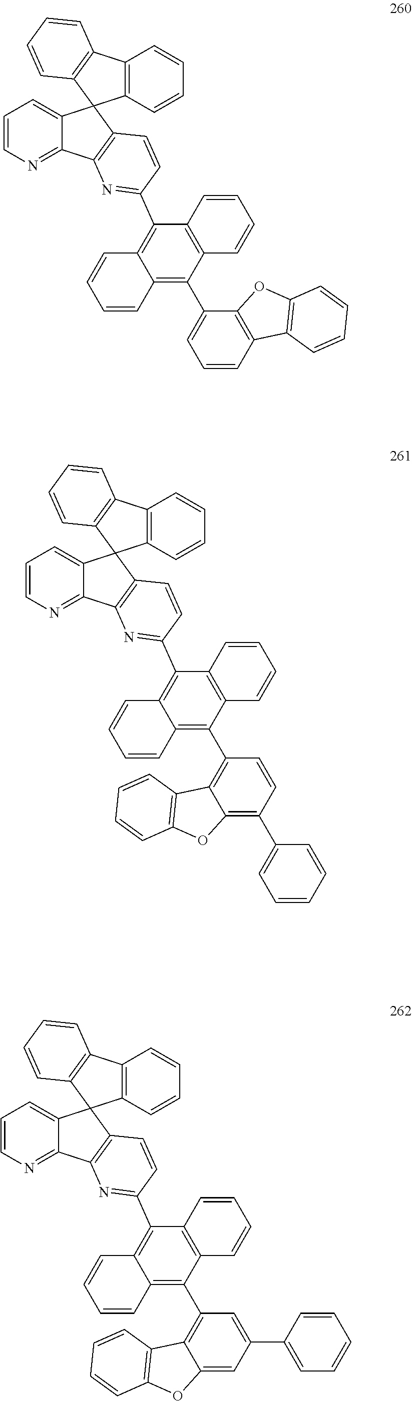 [SMILES: c1ccc2c(c1)-c1ccccc1C21c2cccnc2-c2nc(-c3c4ccccc4c(-c4cccc5c4oc4ccccc45)c4ccccc34)ccc21 ; c1ccc(-c2ccc(-c3c4ccccc4c(-c4ccc5c(n4)-c4ncccc4C54c5ccccc5-c5ccccc54)c4ccccc34)c3c2oc2ccccc23)cc1 ; c1ccc(-c2cc(-c3c4ccccc4c(-c4ccc5c(n4)-c4ncccc4C54c5ccccc5-c5ccccc54)c4ccccc34)c3c(c2)oc2ccccc23)cc1]

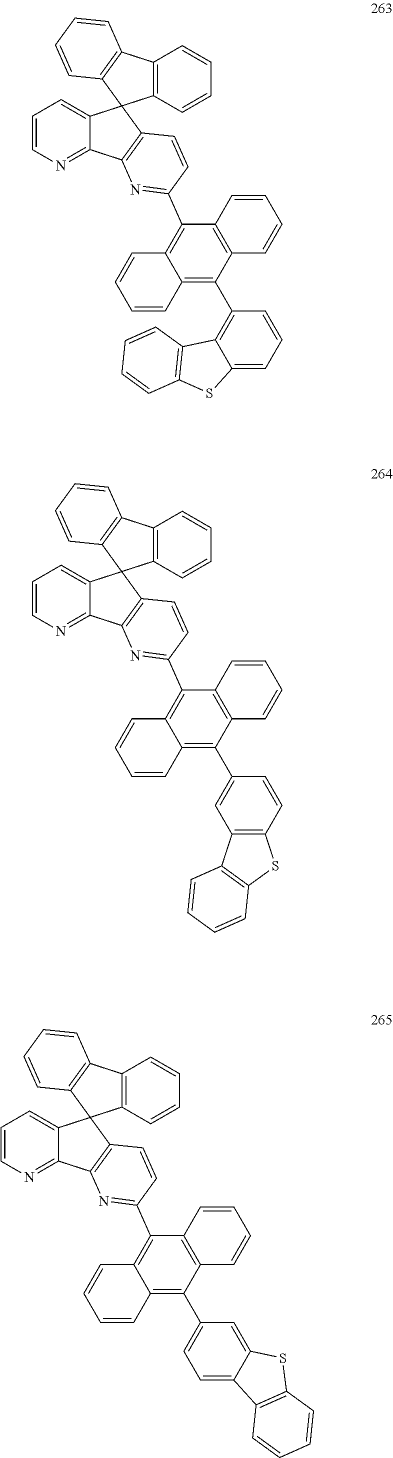 [SMILES: c1ccc2c(c1)-c1ccccc1C21c2cccnc2-c2nc(-c3c4ccccc4c(-c4cccc5sc6ccccc6c45)c4ccccc34)ccc21 ; c1ccc2c(c1)-c1ccccc1C21c2cccnc2-c2nc(-c3c4ccccc4c(-c4ccc5sc6ccccc6c5c4)c4ccccc34)ccc21 ; c1ccc2c(c1)-c1ccccc1C21c2cccnc2-c2nc(-c3c4ccccc4c(-c4ccc5c(c4)sc4ccccc45)c4ccccc34)ccc21]

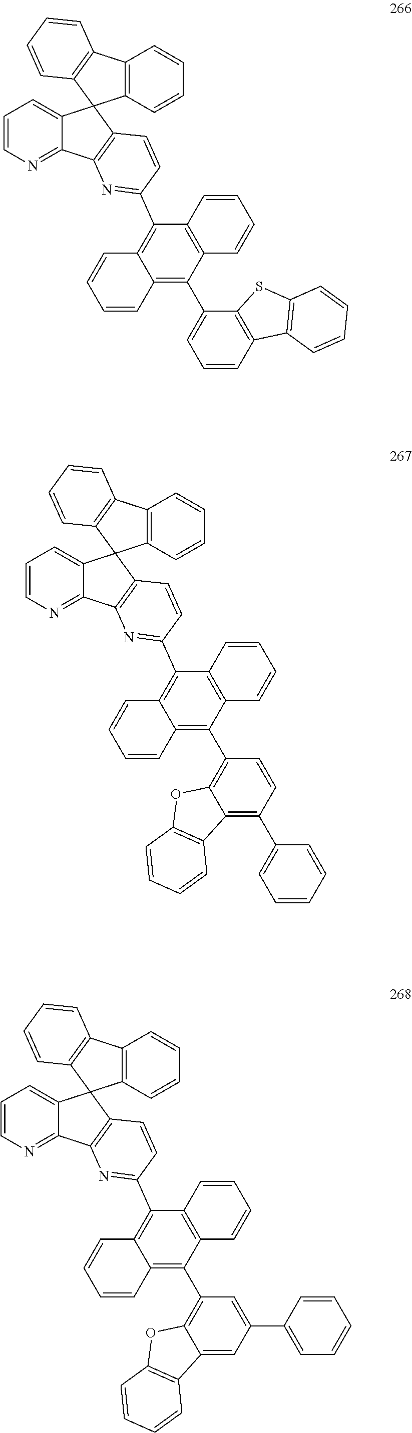 [SMILES: c1ccc2c(c1)-c1ccccc1C21c2cccnc2-c2nc(-c3c4ccccc4c(-c4cccc5c4sc4ccccc45)c4ccccc34)ccc21 ; c1ccc(-c2ccc(-c3c4ccccc4c(-c4ccc5c(n4)-c4ncccc4C54c5ccccc5-c5ccccc54)c4ccccc34)c3oc4ccccc4c23)cc1 ; c1ccc(-c2cc(-c3c4ccccc4c(-c4ccc5c(n4)-c4ncccc4C54c5ccccc5-c5ccccc54)c4ccccc34)c3oc4ccccc4c3c2)cc1]

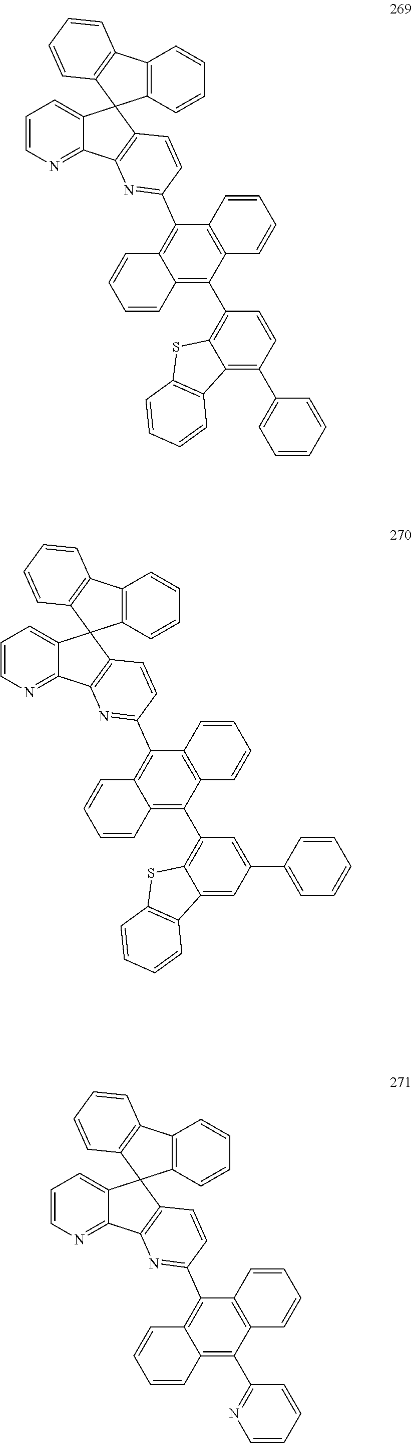 [SMILES: c1ccc(-c2ccc(-c3c4ccccc4c(-c4ccc5c(n4)-c4ncccc4C54c5ccccc5-c5ccccc54)c4ccccc34)c3sc4ccccc4c23)cc1 ; c1ccc(-c2cc(-c3c4ccccc4c(-c4ccc5c(n4)-c4ncccc4C54c5ccccc5-c5ccccc54)c4ccccc34)c3sc4ccccc4c3c2)cc1 ; c1ccc(-c2c3ccccc3c(-c3ccc4c(n3)-c3ncccc3C43c4ccccc4-c4ccccc43)c3ccccc23)nc1]

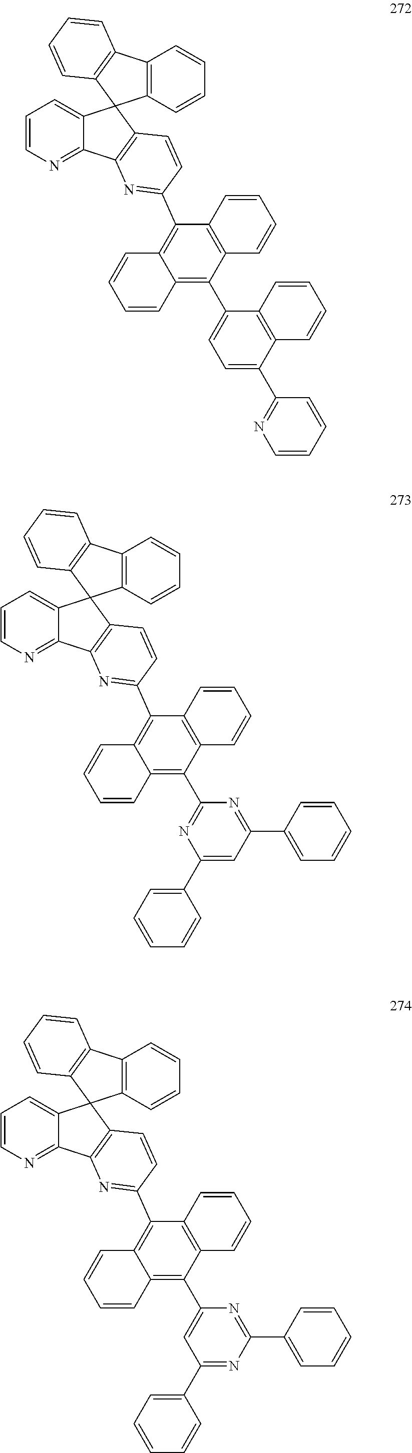 [SMILES: c1ccc(-c2ccc(-c3c4ccccc4c(-c4ccc5c(n4)-c4ncccc4C54c5ccccc5-c5ccccc54)c4ccccc34)c3ccccc23)nc1 ; c1ccc(-c2cc(-c3ccccc3)nc(-c3c4ccccc4c(-c4ccc5c(n4)-c4ncccc4C54c5ccccc5-c5ccccc54)c4ccccc34)n2)cc1 ; c1ccc(-c2cc(-c3c4ccccc4c(-c4ccc5c(n4)-c4ncccc4C54c5ccccc5-c5ccccc54)c4ccccc34)nc(-c3ccccc3)n2)cc1]

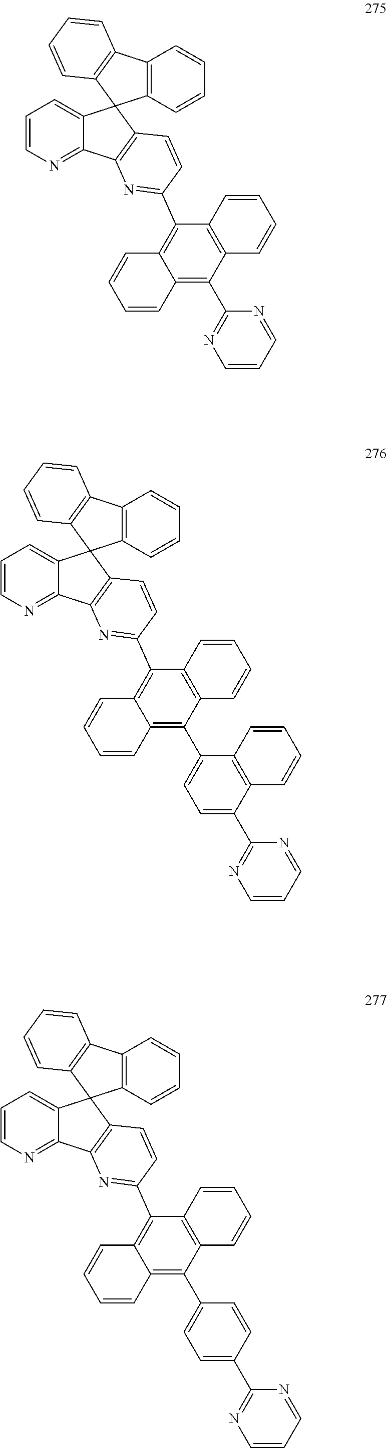 [SMILES: c1cnc(-c2c3ccccc3c(-c3ccc4c(n3)-c3ncccc3C43c4ccccc4-c4ccccc43)c3ccccc23)nc1 ; c1cnc(-c2ccc(-c3c4ccccc4c(-c4ccc5c(n4)-c4ncccc4C54c5ccccc5-c5ccccc54)c4ccccc34)c3ccccc23)nc1 ; c1cnc(-c2ccc(-c3c4ccccc4c(-c4ccc5c(n4)-c4ncccc4C54c5ccccc5-c5ccccc54)c4ccccc34)cc2)nc1]

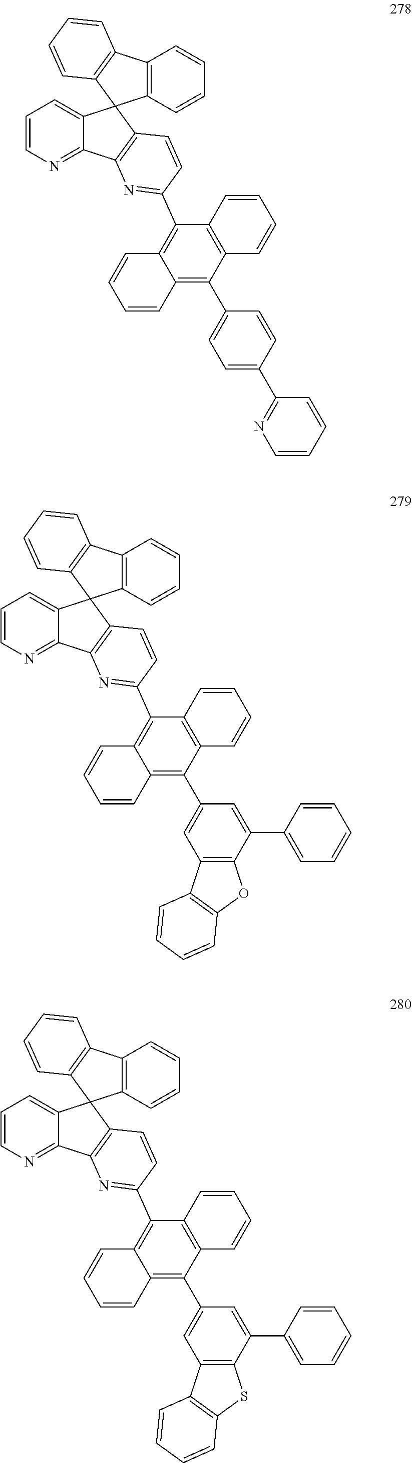 [SMILES: c1ccc(-c2ccc(-c3c4ccccc4c(-c4ccc5c(n4)-c4ncccc4C54c5ccccc5-c5ccccc54)c4ccccc34)cc2)nc1 ; c1ccc(-c2cc(-c3c4ccccc4c(-c4ccc5c(n4)-c4ncccc4C54c5ccccc5-c5ccccc54)c4ccccc34)cc3c2oc2ccccc23)cc1 ; c1ccc(-c2cc(-c3c4ccccc4c(-c4ccc5c(n4)-c4ncccc4C54c5ccccc5-c5ccccc54)c4ccccc34)cc3c2sc2ccccc23)cc1]

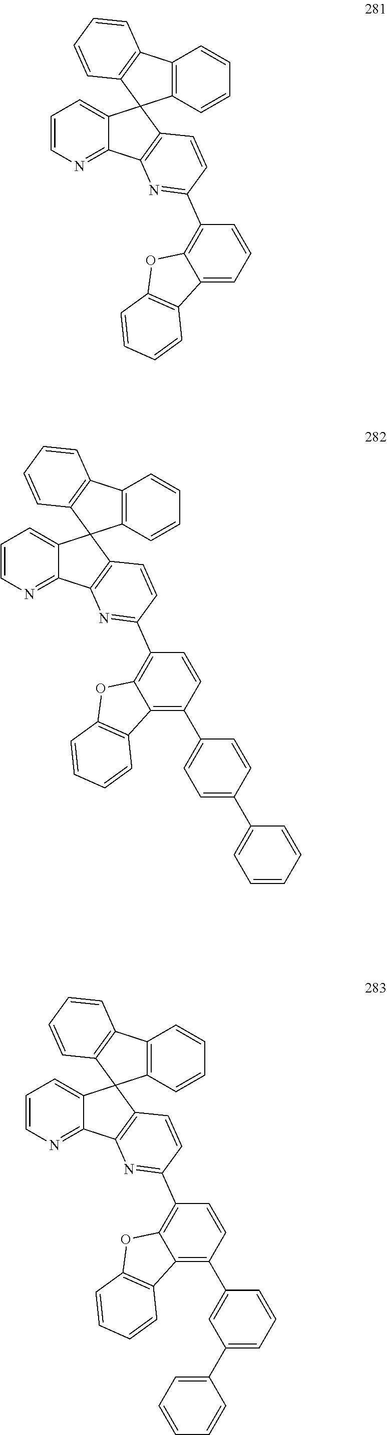 [SMILES: c1ccc2c(c1)-c1ccccc1C21c2cccnc2-c2nc(-c3cccc4c3oc3ccccc34)ccc21 ; c1ccc(-c2ccc(-c3ccc(-c4ccc5c(n4)-c4ncccc4C54c5ccccc5-c5ccccc54)c4oc5ccccc5c34)cc2)cc1 ; c1ccc(-c2cccc(-c3ccc(-c4ccc5c(n4)-c4ncccc4C54c5ccccc5-c5ccccc54)c4oc5ccccc5c34)c2)cc1]

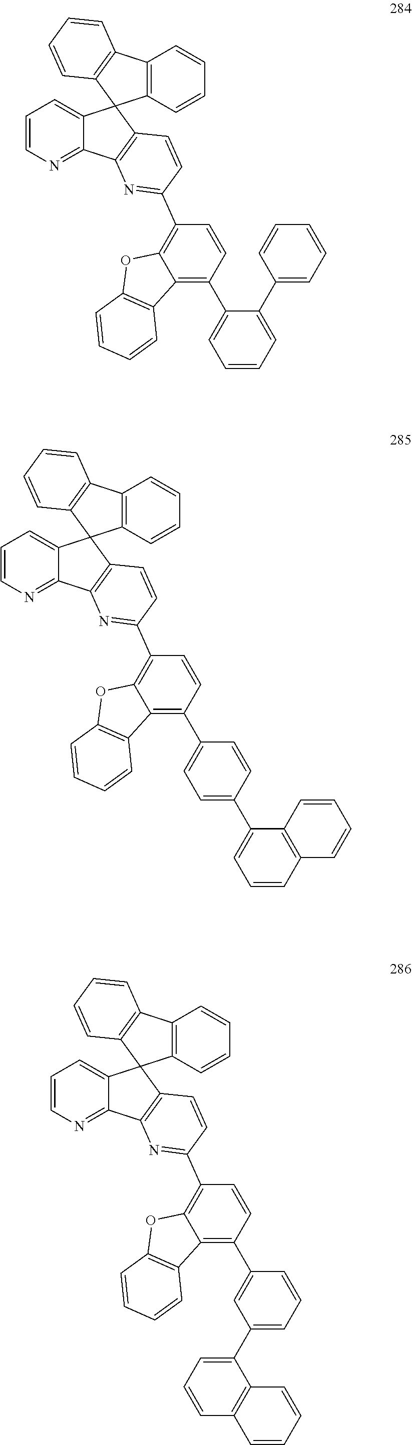 [SMILES: c1ccc(-c2ccccc2-c2ccc(-c3ccc4c(n3)-c3ncccc3C43c4ccccc4-c4ccccc43)c3oc4ccccc4c23)cc1 ; c1ccc2c(c1)-c1ccccc1C21c2cccnc2-c2nc(-c3ccc(-c4ccc(-c5cccc6ccccc56)cc4)c4c3oc3ccccc34)ccc21 ; c1cc(-c2cccc3ccccc23)cc(-c2ccc(-c3ccc4c(n3)-c3ncccc3C43c4ccccc4-c4ccccc43)c3oc4ccccc4c23)c1]

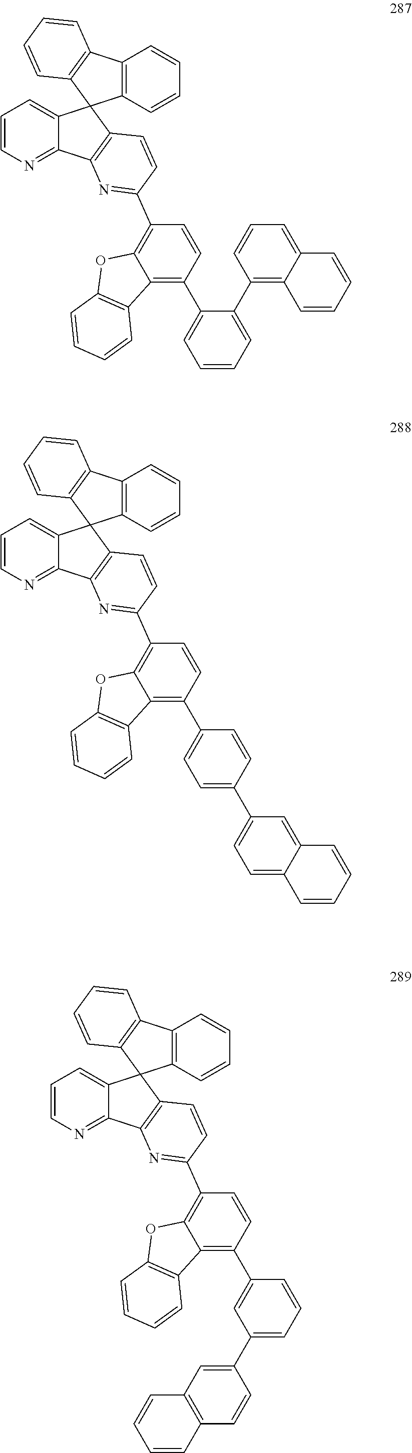 [SMILES: c1ccc(-c2ccc(-c3ccc4c(n3)-c3ncccc3C43c4ccccc4-c4ccccc43)c3oc4ccccc4c23)c(-c2cccc3ccccc23)c1 ; c1ccc2c(c1)-c1ccccc1C21c2cccnc2-c2nc(-c3ccc(-c4ccc(-c5ccc6ccccc6c5)cc4)c4c3oc3ccccc34)ccc21 ; c1cc(-c2ccc3ccccc3c2)cc(-c2ccc(-c3ccc4c(n3)-c3ncccc3C43c4ccccc4-c4ccccc43)c3oc4ccccc4c23)c1]

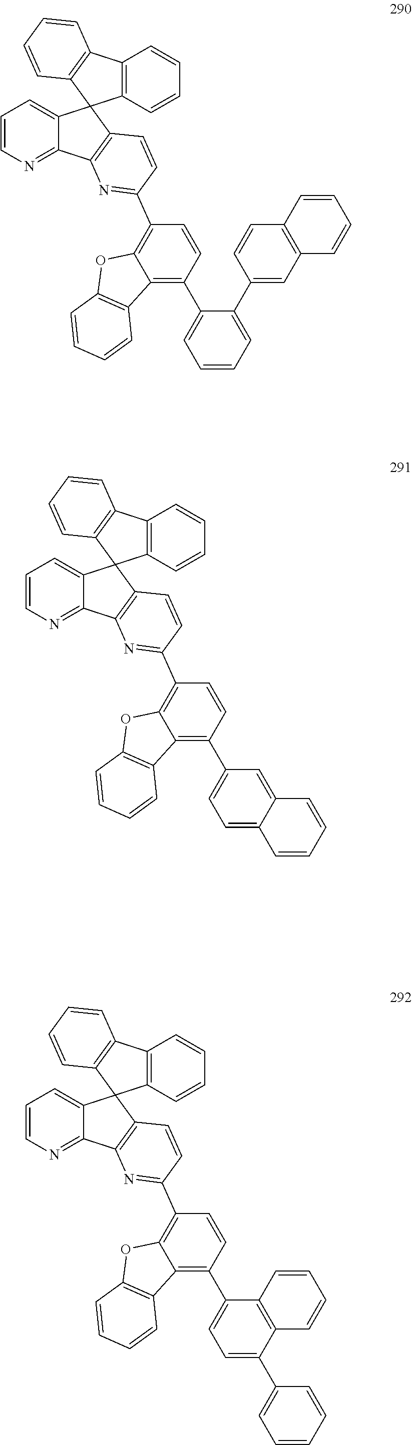 [SMILES: c1ccc(-c2ccc(-c3ccc4c(n3)-c3ncccc3C43c4ccccc4-c4ccccc43)c3oc4ccccc4c23)c(-c2ccc3ccccc3c2)c1 ; c1ccc2c(c1)-c1ccccc1C21c2cccnc2-c2nc(-c3ccc(-c4ccc5ccccc5c4)c4c3oc3ccccc34)ccc21 ; c1ccc(-c2ccc(-c3ccc(-c4ccc5c(n4)-c4ncccc4C54c5ccccc5-c5ccccc54)c4oc5ccccc5c34)c3ccccc23)cc1]

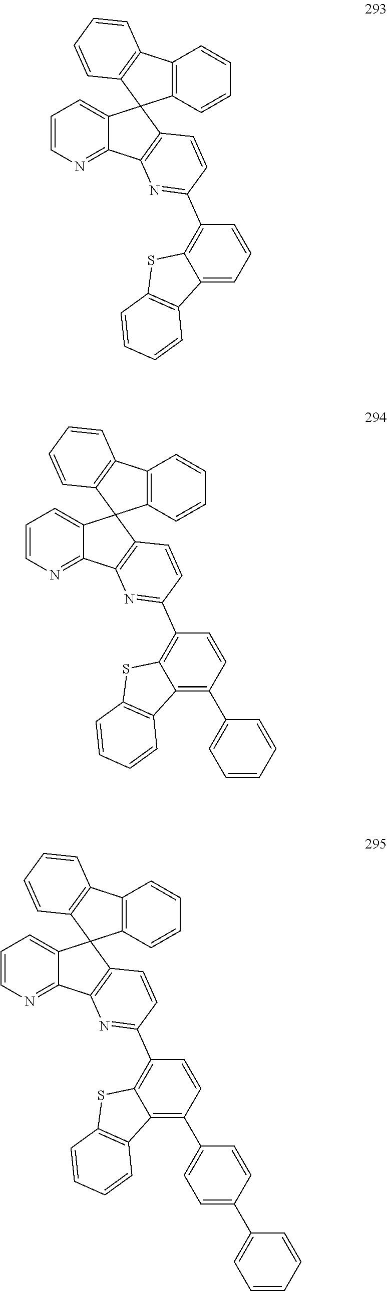 [SMILES: c1ccc2c(c1)-c1ccccc1C21c2cccnc2-c2nc(-c3cccc4c3sc3ccccc34)ccc21 ; c1ccc(-c2ccc(-c3ccc4c(n3)-c3ncccc3C43c4ccccc4-c4ccccc43)c3sc4ccccc4c23)cc1 ; c1ccc(-c2ccc(-c3ccc(-c4ccc5c(n4)-c4ncccc4C54c5ccccc5-c5ccccc54)c4sc5ccccc5c34)cc2)cc1]

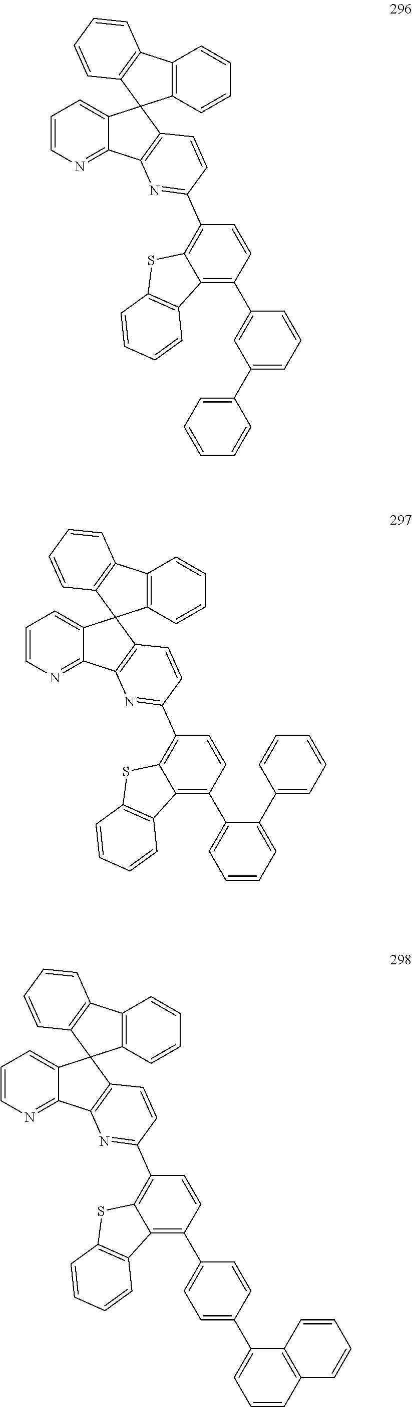 [SMILES: c1ccc(-c2cccc(-c3ccc(-c4ccc5c(n4)-c4ncccc4C54c5ccccc5-c5ccccc54)c4sc5ccccc5c34)c2)cc1 ; c1ccc(-c2ccccc2-c2ccc(-c3ccc4c(n3)-c3ncccc3C43c4ccccc4-c4ccccc43)c3sc4ccccc4c23)cc1 ; c1ccc2c(c1)-c1ccccc1C21c2cccnc2-c2nc(-c3ccc(-c4ccc(-c5cccc6ccccc56)cc4)c4c3sc3ccccc34)ccc21]

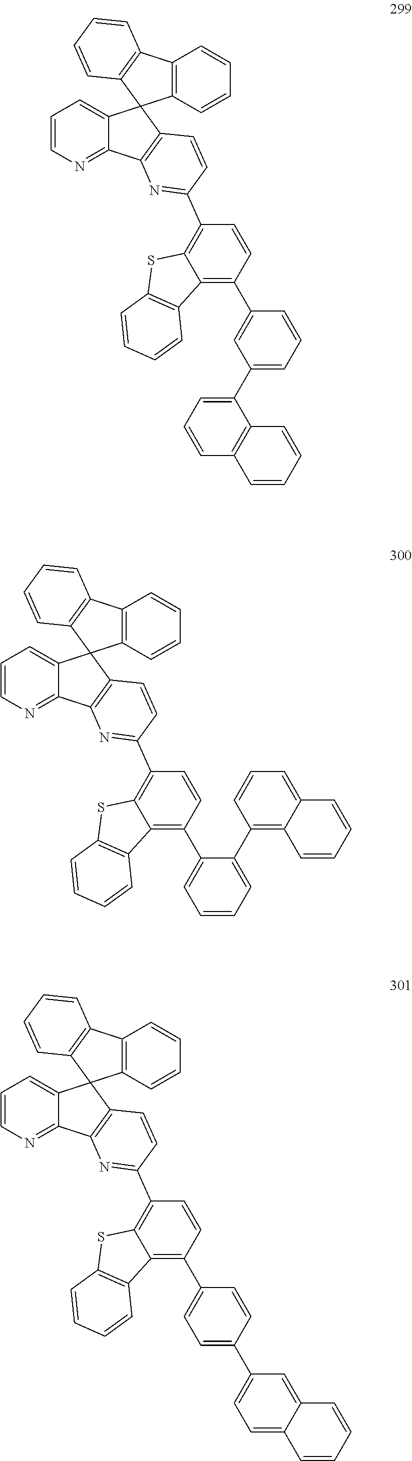 [SMILES: c1cc(-c2cccc3ccccc23)cc(-c2ccc(-c3ccc4c(n3)-c3ncccc3C43c4ccccc4-c4ccccc43)c3sc4ccccc4c23)c1 ; c1ccc(-c2ccc(-c3ccc4c(n3)-c3ncccc3C43c4ccccc4-c4ccccc43)c3sc4ccccc4c23)c(-c2cccc3ccccc23)c1 ; c1ccc2c(c1)-c1ccccc1C21c2cccnc2-c2nc(-c3ccc(-c4ccc(-c5ccc6ccccc6c5)cc4)c4c3sc3ccccc34)ccc21]

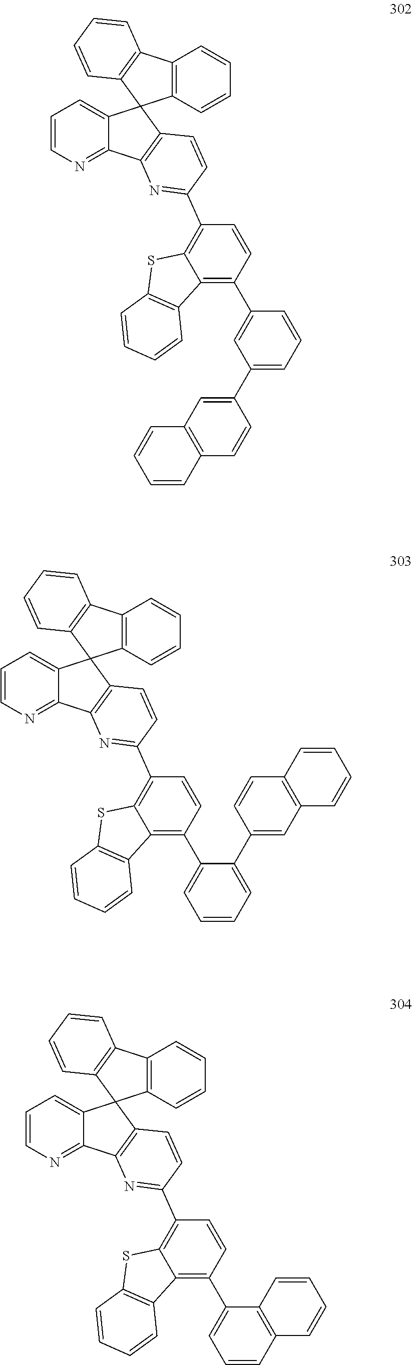 [SMILES: c1cc(-c2ccc3ccccc3c2)cc(-c2ccc(-c3ccc4c(n3)-c3ncccc3C43c4ccccc4-c4ccccc43)c3sc4ccccc4c23)c1 ; c1ccc(-c2ccc(-c3ccc4c(n3)-c3ncccc3C43c4ccccc4-c4ccccc43)c3sc4ccccc4c23)c(-c2ccc3ccccc3c2)c1 ; c1ccc2c(c1)-c1ccccc1C21c2cccnc2-c2nc(-c3ccc(-c4cccc5ccccc45)c4c3sc3ccccc34)ccc21]

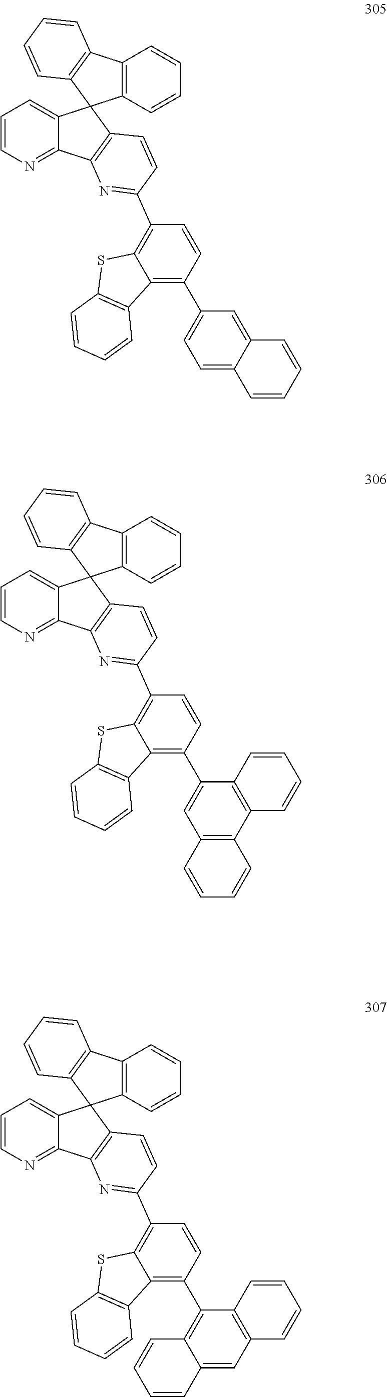 [SMILES: c1ccc2c(c1)-c1ccccc1C21c2cccnc2-c2nc(-c3ccc(-c4ccc5ccccc5c4)c4c3sc3ccccc34)ccc21 ; c1ccc2c(c1)-c1ccccc1C21c2cccnc2-c2nc(-c3ccc(-c4cc5ccccc5c5ccccc45)c4c3sc3ccccc34)ccc21 ; c1ccc2c(c1)-c1ccccc1C21c2cccnc2-c2nc(-c3ccc(-c4c5ccccc5cc5ccccc45)c4c3sc3ccccc34)ccc21]

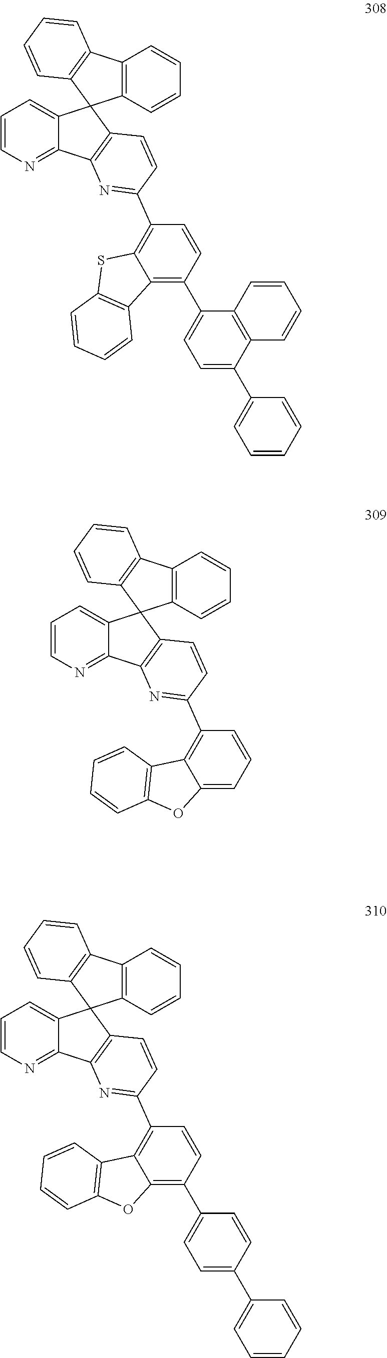 [SMILES: c1ccc(-c2ccc(-c3ccc(-c4ccc5c(n4)-c4ncccc4C54c5ccccc5-c5ccccc54)c4sc5ccccc5c34)c3ccccc23)cc1 ; c1ccc2c(c1)-c1ccccc1C21c2cccnc2-c2nc(-c3cccc4oc5ccccc5c34)ccc21 ; c1ccc(-c2ccc(-c3ccc(-c4ccc5c(n4)-c4ncccc4C54c5ccccc5-c5ccccc54)c4c3oc3ccccc34)cc2)cc1]

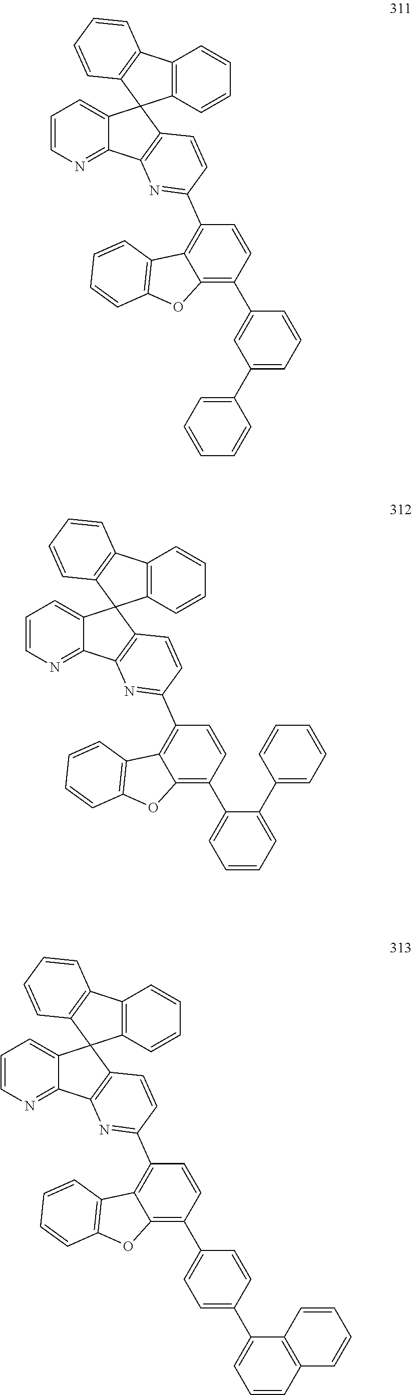 [SMILES: c1ccc(-c2cccc(-c3ccc(-c4ccc5c(n4)-c4ncccc4C54c5ccccc5-c5ccccc54)c4c3oc3ccccc34)c2)cc1 ; c1ccc(-c2ccccc2-c2ccc(-c3ccc4c(n3)-c3ncccc3C43c4ccccc4-c4ccccc43)c3c2oc2ccccc23)cc1 ; c1ccc2c(c1)-c1ccccc1C21c2cccnc2-c2nc(-c3ccc(-c4ccc(-c5cccc6ccccc56)cc4)c4oc5ccccc5c34)ccc21]

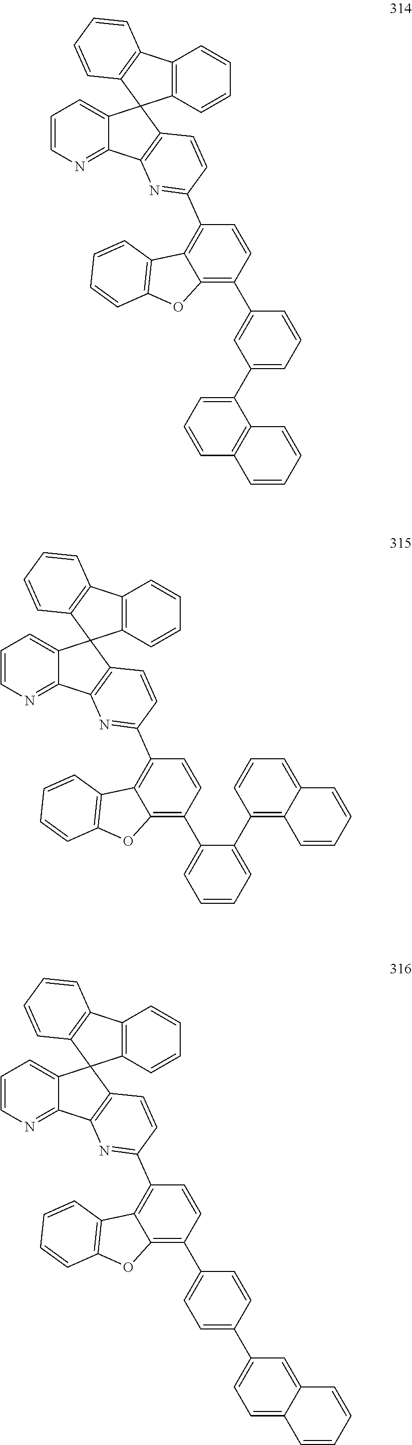 [SMILES: c1cc(-c2cccc3ccccc23)cc(-c2ccc(-c3ccc4c(n3)-c3ncccc3C43c4ccccc4-c4ccccc43)c3c2oc2ccccc23)c1 ; c1ccc(-c2ccc(-c3ccc4c(n3)-c3ncccc3C43c4ccccc4-c4ccccc43)c3c2oc2ccccc23)c(-c2cccc3ccccc23)c1 ; c1ccc2c(c1)-c1ccccc1C21c2cccnc2-c2nc(-c3ccc(-c4ccc(-c5ccc6ccccc6c5)cc4)c4oc5ccccc5c34)ccc21]

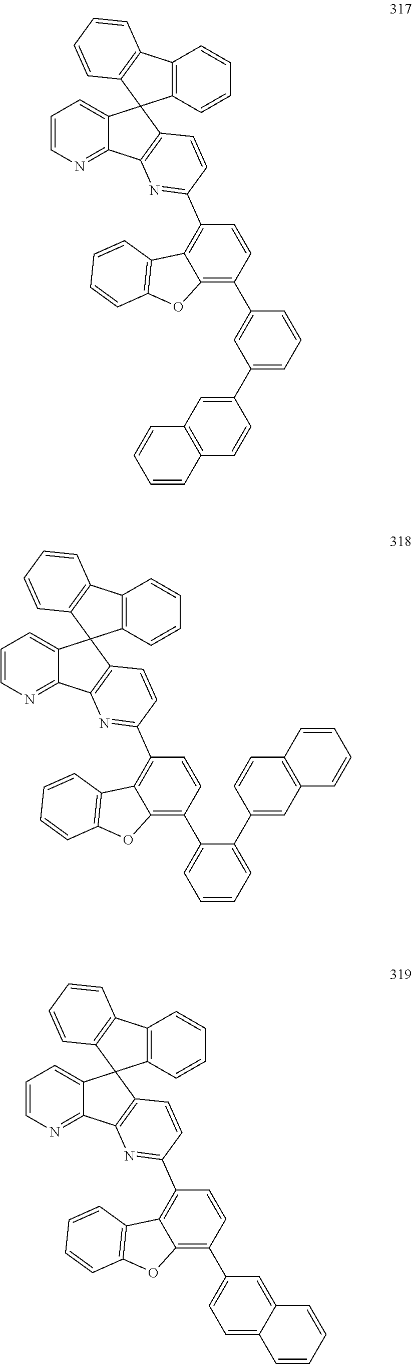 [SMILES: c1cc(-c2ccc3ccccc3c2)cc(-c2ccc(-c3ccc4c(n3)-c3ncccc3C43c4ccccc4-c4ccccc43)c3c2oc2ccccc23)c1 ; c1ccc(-c2ccc(-c3ccc4c(n3)-c3ncccc3C43c4ccccc4-c4ccccc43)c3c2oc2ccccc23)c(-c2ccc3ccccc3c2)c1 ; c1ccc2c(c1)-c1ccccc1C21c2cccnc2-c2nc(-c3ccc(-c4ccc5ccccc5c4)c4oc5ccccc5c34)ccc21]

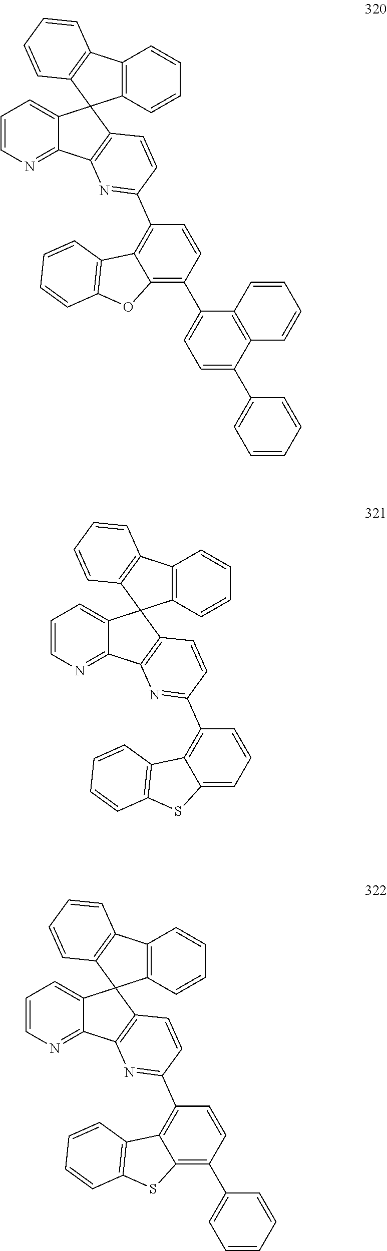 [SMILES: c1ccc(-c2ccc(-c3ccc(-c4ccc5c(n4)-c4ncccc4C54c5ccccc5-c5ccccc54)c4c3oc3ccccc34)c3ccccc23)cc1 ; c1ccc2c(c1)-c1ccccc1C21c2cccnc2-c2nc(-c3cccc4sc5ccccc5c34)ccc21 ; c1ccc(-c2ccc(-c3ccc4c(n3)-c3ncccc3C43c4ccccc4-c4ccccc43)c3c2sc2ccccc23)cc1]

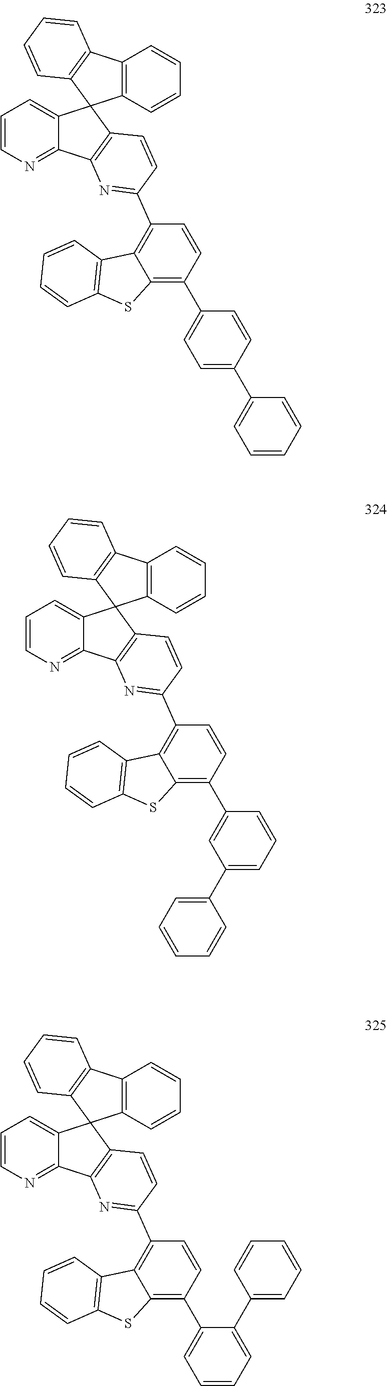 [SMILES: c1ccc(-c2ccc(-c3ccc(-c4ccc5c(n4)-c4ncccc4C54c5ccccc5-c5ccccc54)c4c3sc3ccccc34)cc2)cc1 ; c1ccc(-c2cccc(-c3ccc(-c4ccc5c(n4)-c4ncccc4C54c5ccccc5-c5ccccc54)c4c3sc3ccccc34)c2)cc1 ; c1ccc(-c2ccccc2-c2ccc(-c3ccc4c(n3)-c3ncccc3C43c4ccccc4-c4ccccc43)c3c2sc2ccccc23)cc1]

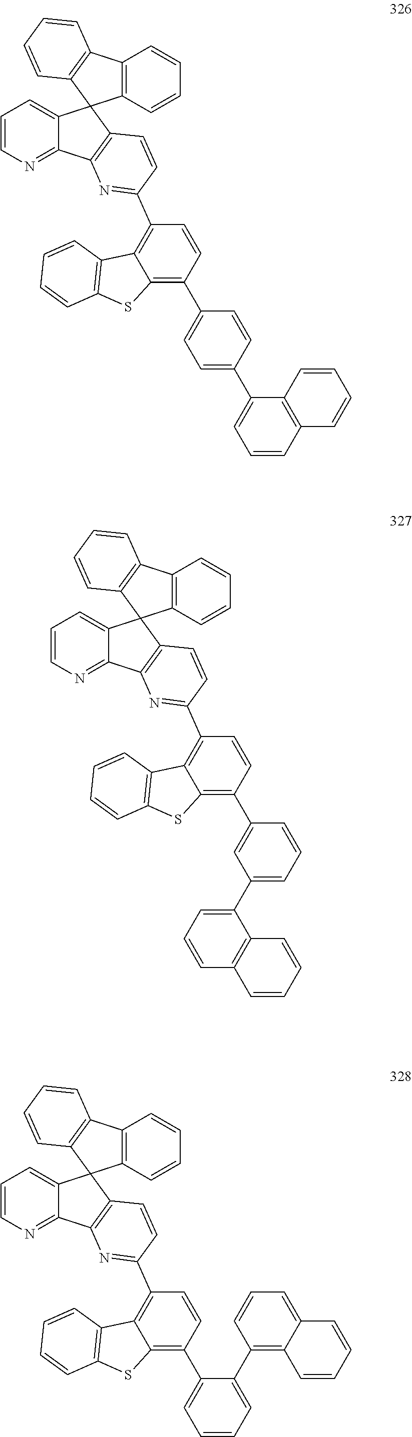 [SMILES: c1ccc2c(c1)-c1ccccc1C21c2cccnc2-c2nc(-c3ccc(-c4ccc(-c5cccc6ccccc56)cc4)c4sc5ccccc5c34)ccc21 ; c1cc(-c2cccc3ccccc23)cc(-c2ccc(-c3ccc4c(n3)-c3ncccc3C43c4ccccc4-c4ccccc43)c3c2sc2ccccc23)c1 ; c1ccc(-c2ccc(-c3ccc4c(n3)-c3ncccc3C43c4ccccc4-c4ccccc43)c3c2sc2ccccc23)c(-c2cccc3ccccc23)c1]

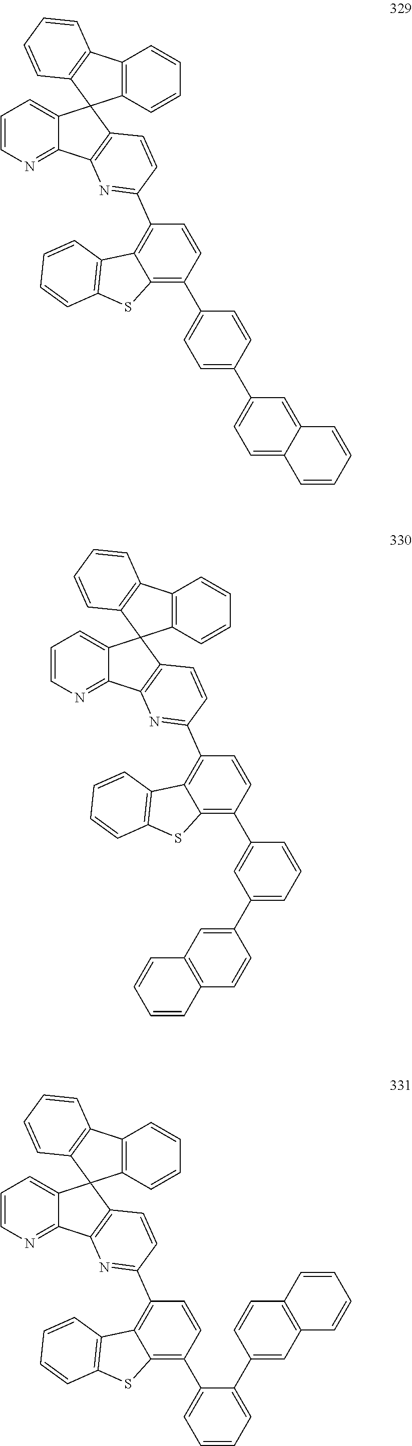 [SMILES: c1ccc2c(c1)-c1ccccc1C21c2cccnc2-c2nc(-c3ccc(-c4ccc(-c5ccc6ccccc6c5)cc4)c4sc5ccccc5c34)ccc21 ; c1cc(-c2ccc3ccccc3c2)cc(-c2ccc(-c3ccc4c(n3)-c3ncccc3C43c4ccccc4-c4ccccc43)c3c2sc2ccccc23)c1 ; c1ccc(-c2ccc(-c3ccc4c(n3)-c3ncccc3C43c4ccccc4-c4ccccc43)c3c2sc2ccccc23)c(-c2ccc3ccccc3c2)c1]

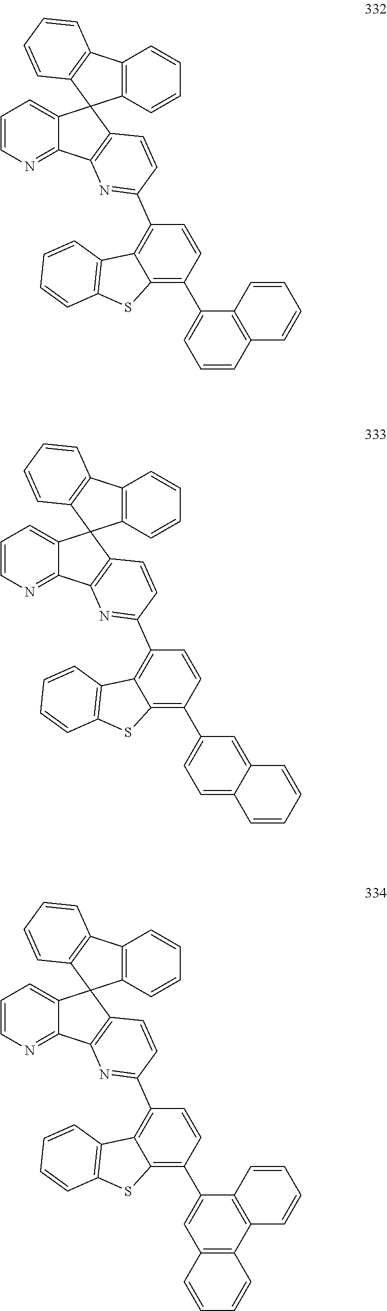 [SMILES: c1ccc2c(c1)-c1ccccc1C21c2cccnc2-c2nc(-c3ccc(-c4cccc5ccccc45)c4sc5ccccc5c34)ccc21 ; c1ccc2c(c1)-c1ccccc1C21c2cccnc2-c2nc(-c3ccc(-c4ccc5ccccc5c4)c4sc5ccccc5c34)ccc21 ; c1ccc2c(c1)-c1ccccc1C21c2cccnc2-c2nc(-c3ccc(-c4cc5ccccc5c5ccccc45)c4sc5ccccc5c34)ccc21]

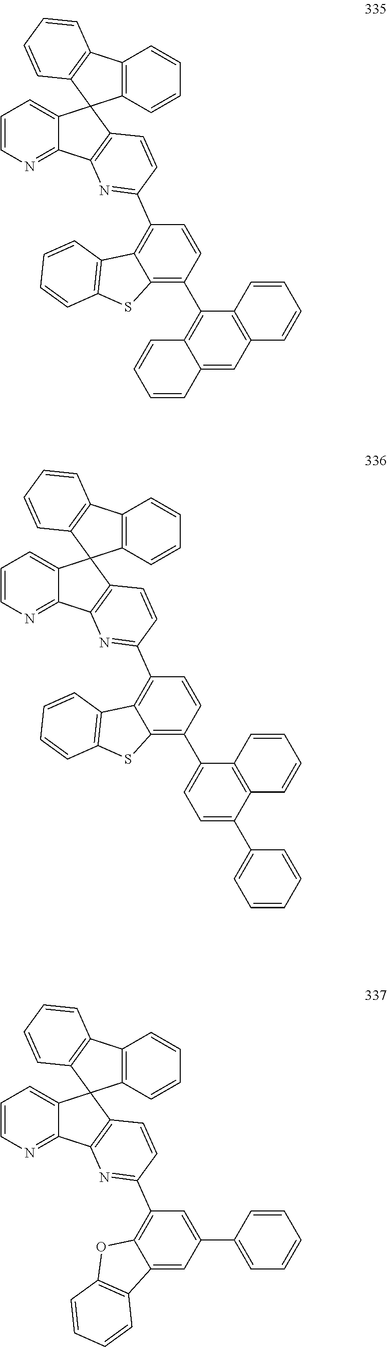 [SMILES: c1ccc2c(c1)-c1ccccc1C21c2cccnc2-c2nc(-c3ccc(-c4c5ccccc5cc5ccccc45)c4sc5ccccc5c34)ccc21 ; c1ccc(-c2ccc(-c3ccc(-c4ccc5c(n4)-c4ncccc4C54c5ccccc5-c5ccccc54)c4c3sc3ccccc34)c3ccccc23)cc1 ; c1ccc(-c2cc(-c3ccc4c(n3)-c3ncccc3C43c4ccccc4-c4ccccc43)c3oc4ccccc4c3c2)cc1]

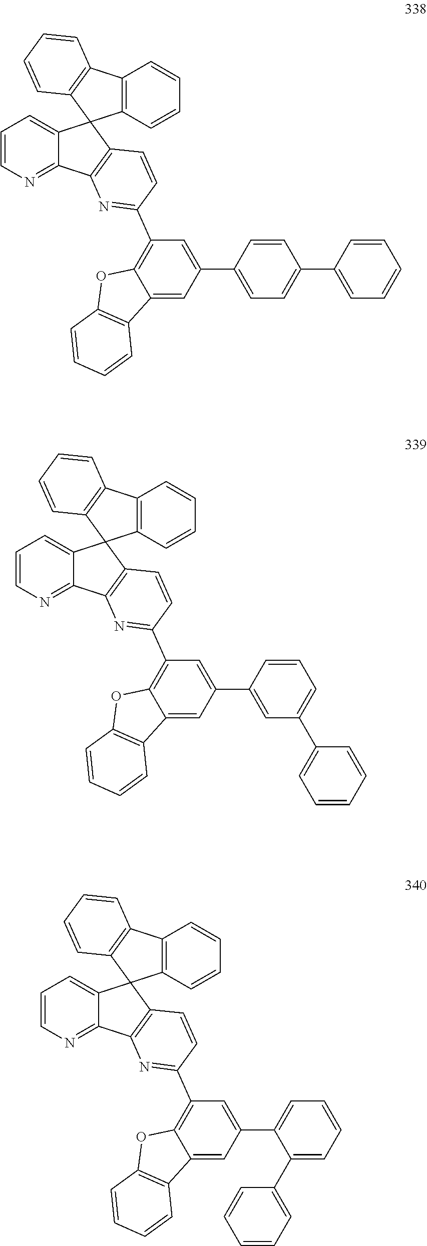 [SMILES: c1ccc(-c2ccc(-c3cc(-c4ccc5c(n4)-c4ncccc4C54c5ccccc5-c5ccccc54)c4oc5ccccc5c4c3)cc2)cc1 ; c1ccc(-c2cccc(-c3cc(-c4ccc5c(n4)-c4ncccc4C54c5ccccc5-c5ccccc54)c4oc5ccccc5c4c3)c2)cc1 ; c1ccc(-c2ccccc2-c2cc(-c3ccc4c(n3)-c3ncccc3C43c4ccccc4-c4ccccc43)c3oc4ccccc4c3c2)cc1]

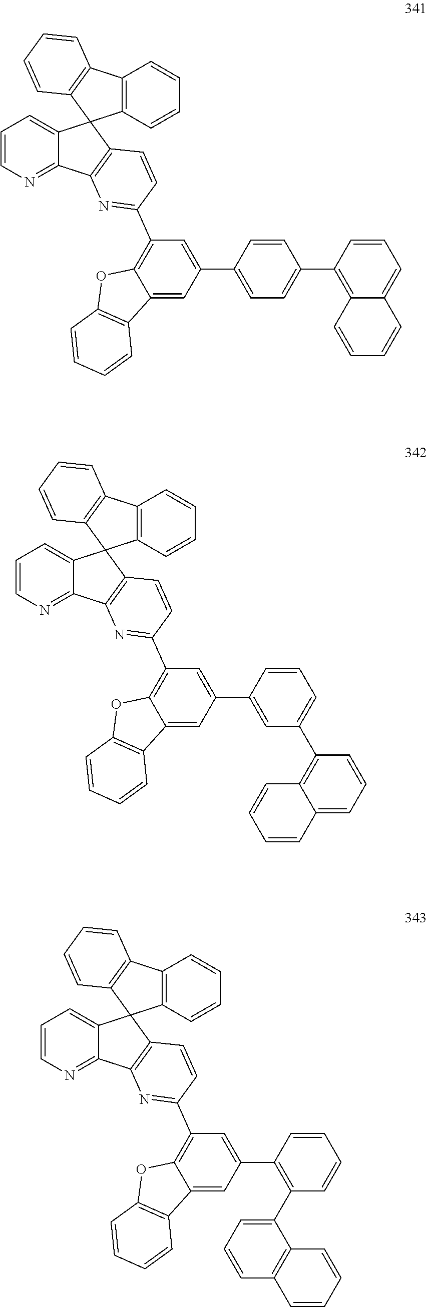 [SMILES: c1ccc2c(c1)-c1ccccc1C21c2cccnc2-c2nc(-c3cc(-c4ccc(-c5cccc6ccccc56)cc4)cc4c3oc3ccccc34)ccc21 ; c1cc(-c2cc(-c3ccc4c(n3)-c3ncccc3C43c4ccccc4-c4ccccc43)c3oc4ccccc4c3c2)cc(-c2cccc3ccccc23)c1 ; c1ccc(-c2cccc3ccccc23)c(-c2cc(-c3ccc4c(n3)-c3ncccc3C43c4ccccc4-c4ccccc43)c3oc4ccccc4c3c2)c1]

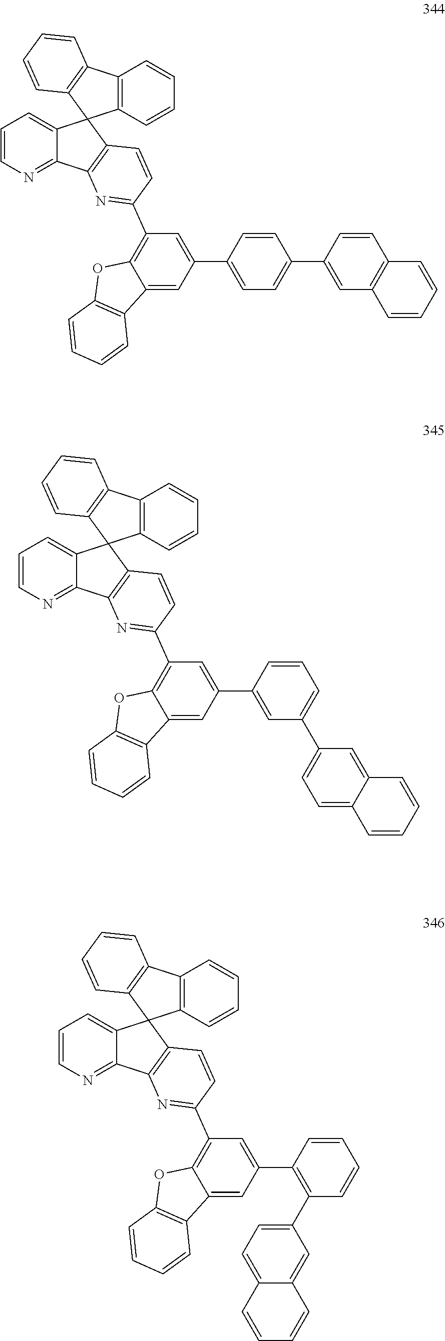 [SMILES: c1ccc2c(c1)-c1ccccc1C21c2cccnc2-c2nc(-c3cc(-c4ccc(-c5ccc6ccccc6c5)cc4)cc4c3oc3ccccc34)ccc21 ; c1cc(-c2ccc3ccccc3c2)cc(-c2cc(-c3ccc4c(n3)-c3ncccc3C43c4ccccc4-c4ccccc43)c3oc4ccccc4c3c2)c1 ; c1ccc(-c2cc(-c3ccc4c(n3)-c3ncccc3C43c4ccccc4-c4ccccc43)c3oc4ccccc4c3c2)c(-c2ccc3ccccc3c2)c1]

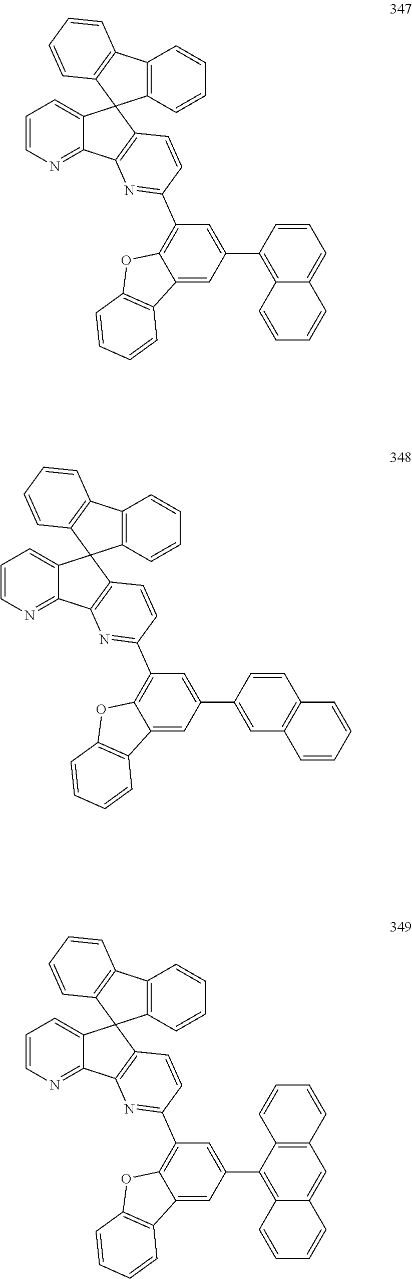 [SMILES: c1ccc2c(c1)-c1ccccc1C21c2cccnc2-c2nc(-c3cc(-c4cccc5ccccc45)cc4c3oc3ccccc34)ccc21 ; c1ccc2c(c1)-c1ccccc1C21c2cccnc2-c2nc(-c3cc(-c4ccc5ccccc5c4)cc4c3oc3ccccc34)ccc21 ; c1ccc2c(c1)-c1ccccc1C21c2cccnc2-c2nc(-c3cc(-c4c5ccccc5cc5ccccc45)cc4c3oc3ccccc34)ccc21]

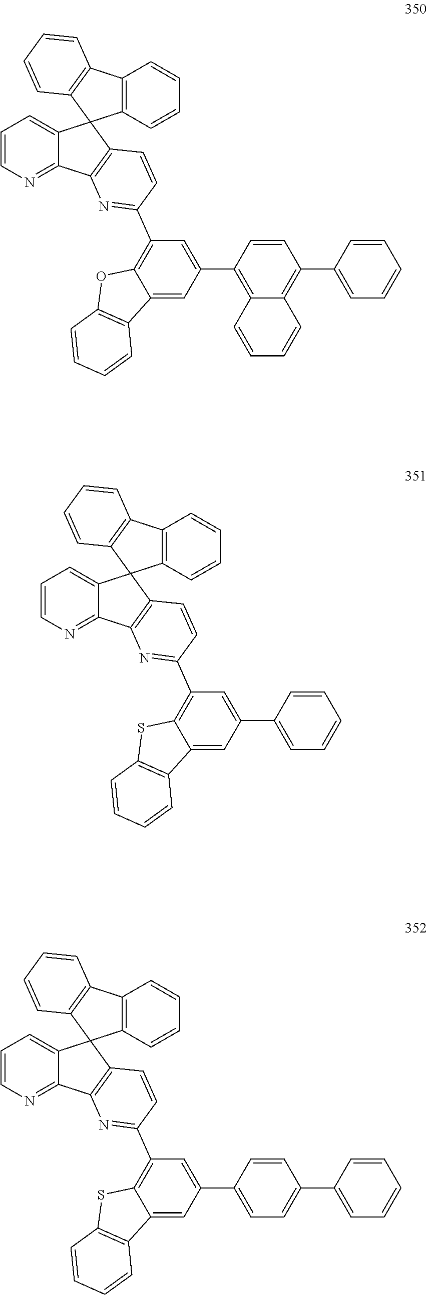 [SMILES: c1ccc(-c2ccc(-c3cc(-c4ccc5c(n4)-c4ncccc4C54c5ccccc5-c5ccccc54)c4oc5ccccc5c4c3)c3ccccc23)cc1 ; c1ccc(-c2cc(-c3ccc4c(n3)-c3ncccc3C43c4ccccc4-c4ccccc43)c3sc4ccccc4c3c2)cc1 ; c1ccc(-c2ccc(-c3cc(-c4ccc5c(n4)-c4ncccc4C54c5ccccc5-c5ccccc54)c4sc5ccccc5c4c3)cc2)cc1]

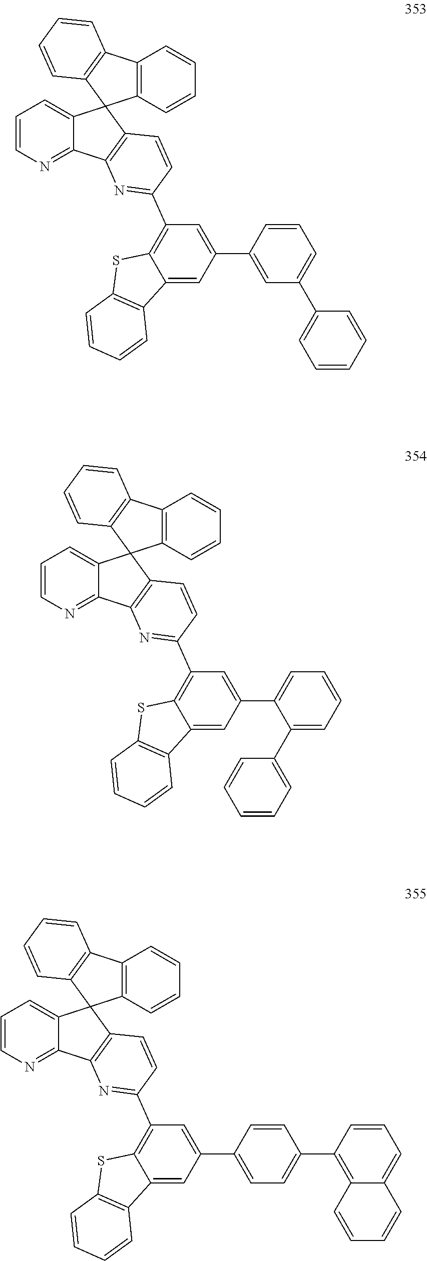 [SMILES: c1ccc(-c2cccc(-c3cc(-c4ccc5c(n4)-c4ncccc4C54c5ccccc5-c5ccccc54)c4sc5ccccc5c4c3)c2)cc1 ; c1ccc(-c2ccccc2-c2cc(-c3ccc4c(n3)-c3ncccc3C43c4ccccc4-c4ccccc43)c3sc4ccccc4c3c2)cc1 ; c1ccc2c(c1)-c1ccccc1C21c2cccnc2-c2nc(-c3cc(-c4ccc(-c5cccc6ccccc56)cc4)cc4c3sc3ccccc34)ccc21]

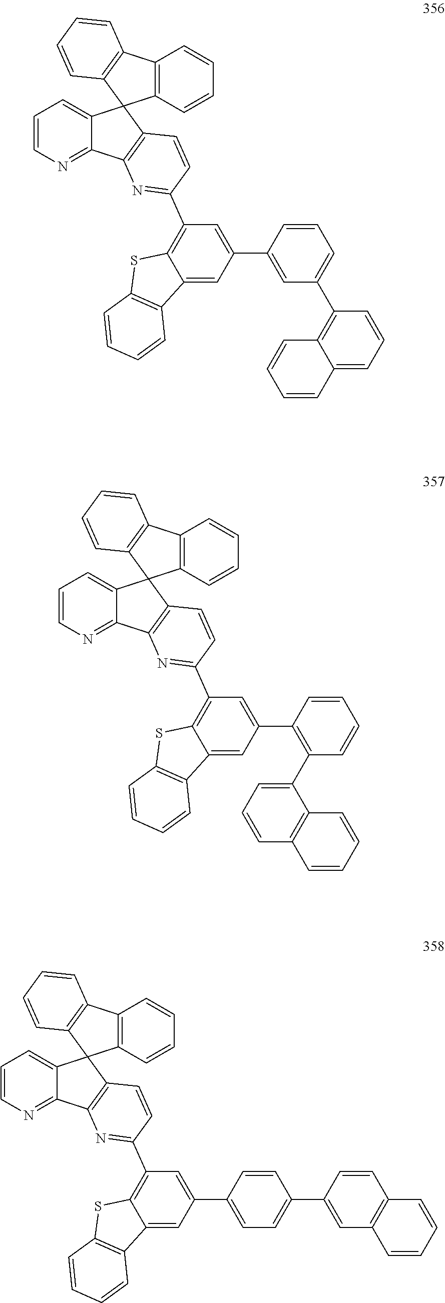 [SMILES: c1cc(-c2cc(-c3ccc4c(n3)-c3ncccc3C43c4ccccc4-c4ccccc43)c3sc4ccccc4c3c2)cc(-c2cccc3ccccc23)c1 ; c1ccc(-c2cccc3ccccc23)c(-c2cc(-c3ccc4c(n3)-c3ncccc3C43c4ccccc4-c4ccccc43)c3sc4ccccc4c3c2)c1 ; c1ccc2c(c1)-c1ccccc1C21c2cccnc2-c2nc(-c3cc(-c4ccc(-c5ccc6ccccc6c5)cc4)cc4c3sc3ccccc34)ccc21]

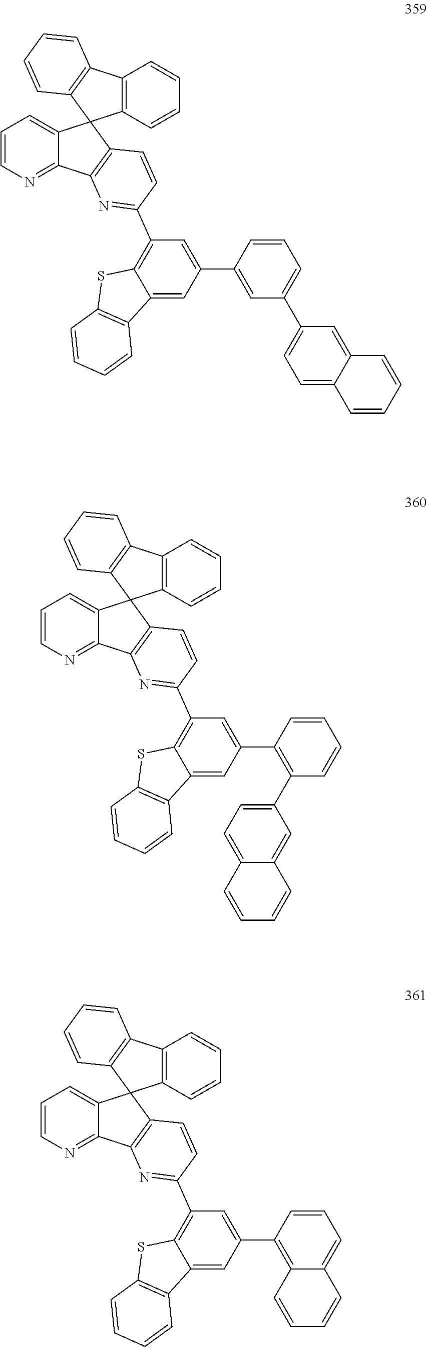 [SMILES: c1cc(-c2ccc3ccccc3c2)cc(-c2cc(-c3ccc4c(n3)-c3ncccc3C43c4ccccc4-c4ccccc43)c3sc4ccccc4c3c2)c1 ; c1ccc(-c2cc(-c3ccc4c(n3)-c3ncccc3C43c4ccccc4-c4ccccc43)c3sc4ccccc4c3c2)c(-c2ccc3ccccc3c2)c1 ; c1ccc2c(c1)-c1ccccc1C21c2cccnc2-c2nc(-c3cc(-c4cccc5ccccc45)cc4c3sc3ccccc34)ccc21]

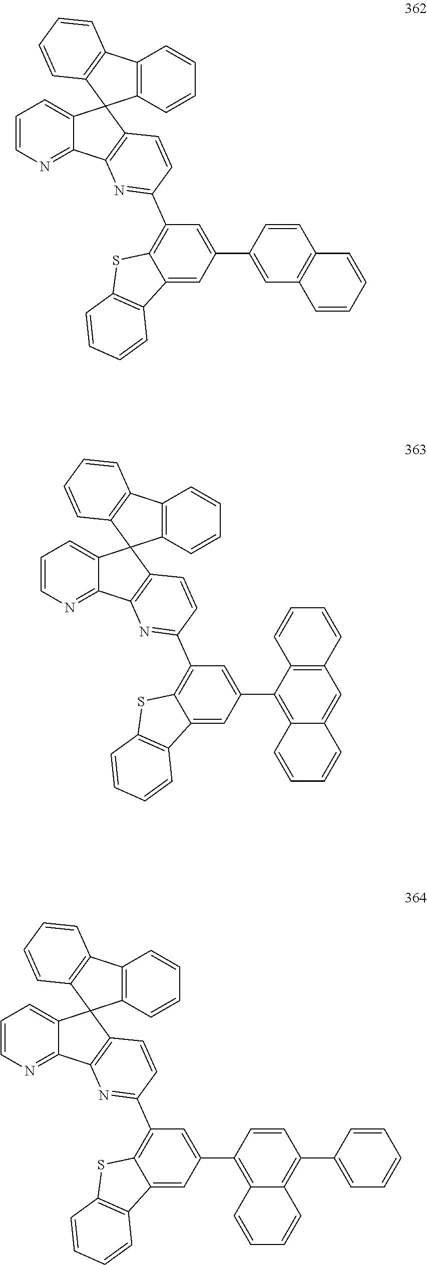 [SMILES: c1ccc2c(c1)-c1ccccc1C21c2cccnc2-c2nc(-c3cc(-c4ccc5ccccc5c4)cc4c3sc3ccccc34)ccc21 ; c1ccc2c(c1)-c1ccccc1C21c2cccnc2-c2nc(-c3cc(-c4c5ccccc5cc5ccccc45)cc4c3sc3ccccc34)ccc21 ; c1ccc(-c2ccc(-c3cc(-c4ccc5c(n4)-c4ncccc4C54c5ccccc5-c5ccccc54)c4sc5ccccc5c4c3)c3ccccc23)cc1]

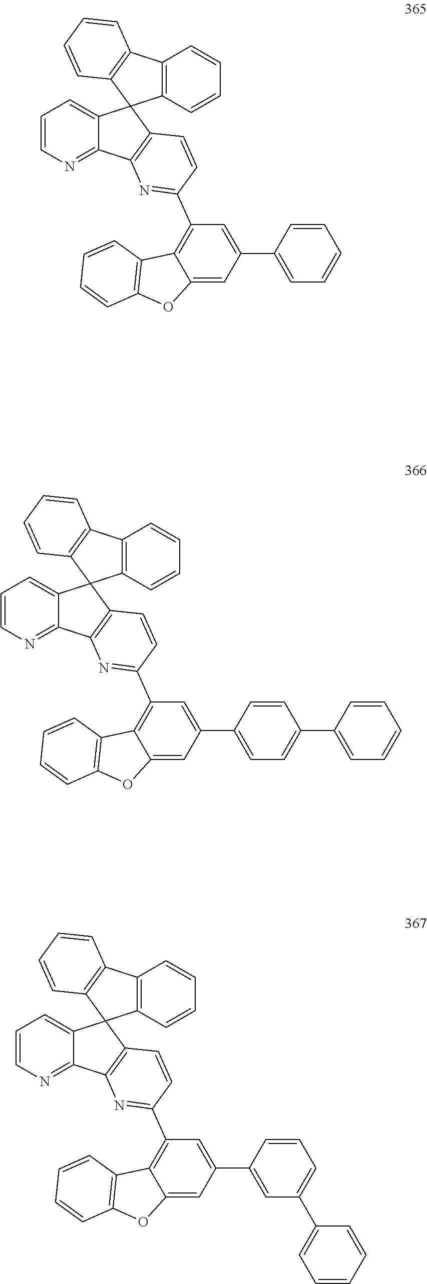 [SMILES: c1ccc(-c2cc(-c3ccc4c(n3)-c3ncccc3C43c4ccccc4-c4ccccc43)c3c(c2)oc2ccccc23)cc1 ; c1ccc(-c2ccc(-c3cc(-c4ccc5c(n4)-c4ncccc4C54c5ccccc5-c5ccccc54)c4c(c3)oc3ccccc34)cc2)cc1 ; c1ccc(-c2cccc(-c3cc(-c4ccc5c(n4)-c4ncccc4C54c5ccccc5-c5ccccc54)c4c(c3)oc3ccccc34)c2)cc1]

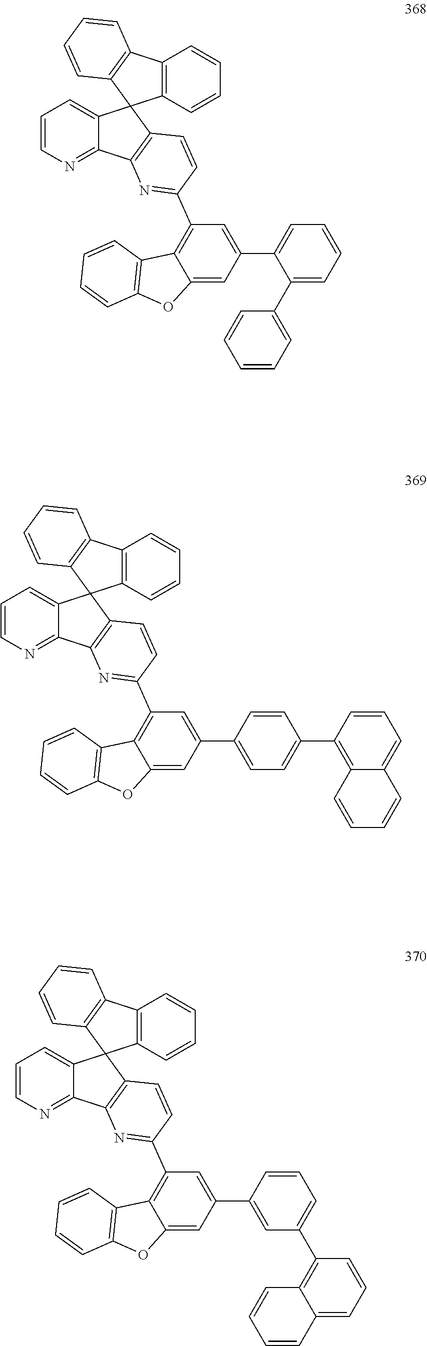 [SMILES: c1ccc(-c2ccccc2-c2cc(-c3ccc4c(n3)-c3ncccc3C43c4ccccc4-c4ccccc43)c3c(c2)oc2ccccc23)cc1 ; c1ccc2c(c1)-c1ccccc1C21c2cccnc2-c2nc(-c3cc(-c4ccc(-c5cccc6ccccc56)cc4)cc4oc5ccccc5c34)ccc21 ; c1cc(-c2cc(-c3ccc4c(n3)-c3ncccc3C43c4ccccc4-c4ccccc43)c3c(c2)oc2ccccc23)cc(-c2cccc3ccccc23)c1]

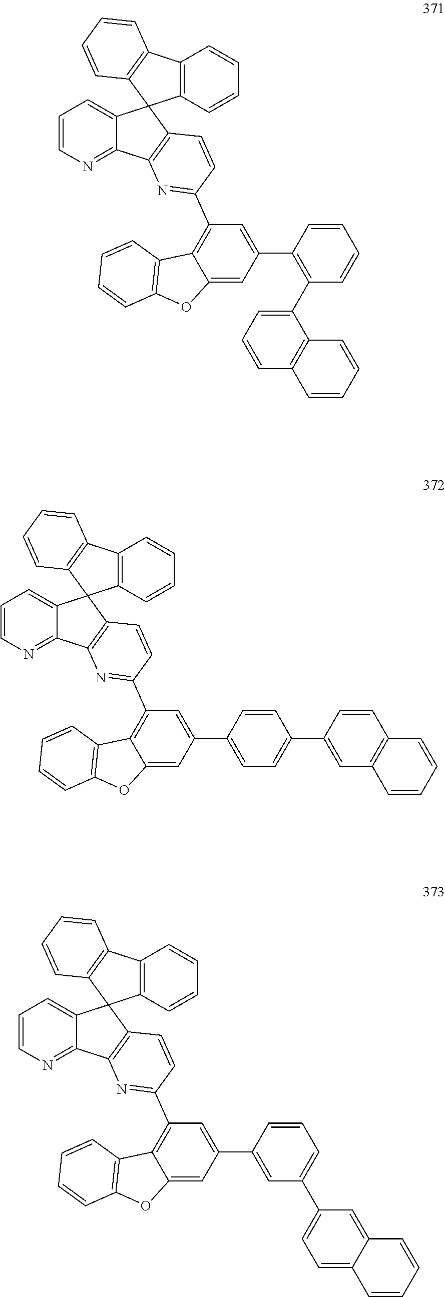 [SMILES: c1ccc(-c2cccc3ccccc23)c(-c2cc(-c3ccc4c(n3)-c3ncccc3C43c4ccccc4-c4ccccc43)c3c(c2)oc2ccccc23)c1 ; c1ccc2c(c1)-c1ccccc1C21c2cccnc2-c2nc(-c3cc(-c4ccc(-c5ccc6ccccc6c5)cc4)cc4oc5ccccc5c34)ccc21 ; c1cc(-c2ccc3ccccc3c2)cc(-c2cc(-c3ccc4c(n3)-c3ncccc3C43c4ccccc4-c4ccccc43)c3c(c2)oc2ccccc23)c1]

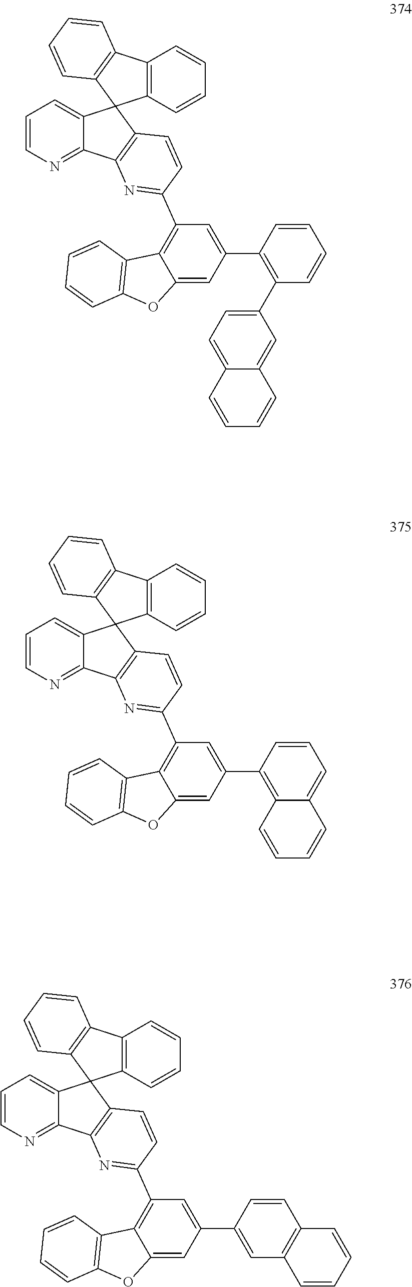 [SMILES: c1ccc(-c2cc(-c3ccc4c(n3)-c3ncccc3C43c4ccccc4-c4ccccc43)c3c(c2)oc2ccccc23)c(-c2ccc3ccccc3c2)c1 ; c1ccc2c(c1)-c1ccccc1C21c2cccnc2-c2nc(-c3cc(-c4cccc5ccccc45)cc4oc5ccccc5c34)ccc21 ; c1ccc2c(c1)-c1ccccc1C21c2cccnc2-c2nc(-c3cc(-c4ccc5ccccc5c4)cc4oc5ccccc5c34)ccc21]

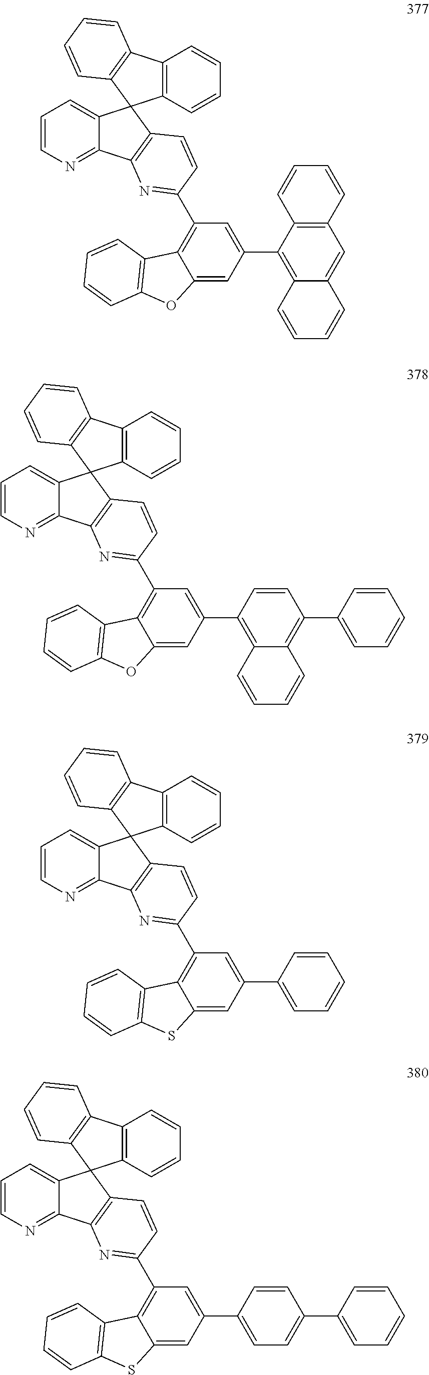 [SMILES: c1ccc2c(c1)-c1ccccc1C21c2cccnc2-c2nc(-c3cc(-c4c5ccccc5cc5ccccc45)cc4oc5ccccc5c34)ccc21 ; c1ccc(-c2ccc(-c3cc(-c4ccc5c(n4)-c4ncccc4C54c5ccccc5-c5ccccc54)c4c(c3)oc3ccccc34)c3ccccc23)cc1 ; c1ccc(-c2cc(-c3ccc4c(n3)-c3ncccc3C43c4ccccc4-c4ccccc43)c3c(c2)sc2ccccc23)cc1 ; c1ccc(-c2ccc(-c3cc(-c4ccc5c(n4)-c4ncccc4C54c5ccccc5-c5ccccc54)c4c(c3)sc3ccccc34)cc2)cc1]

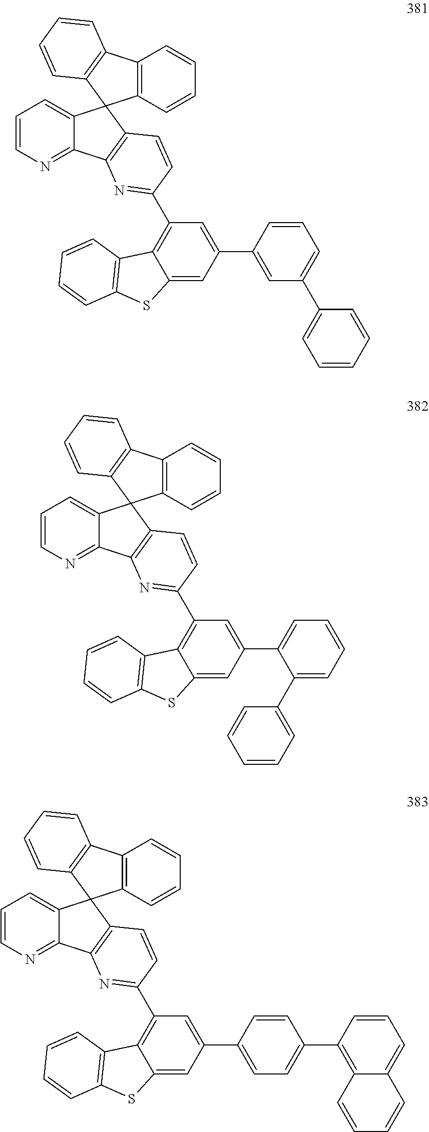 [SMILES: c1ccc(-c2cccc(-c3cc(-c4ccc5c(n4)-c4ncccc4C54c5ccccc5-c5ccccc54)c4c(c3)sc3ccccc34)c2)cc1 ; c1ccc(-c2ccccc2-c2cc(-c3ccc4c(n3)-c3ncccc3C43c4ccccc4-c4ccccc43)c3c(c2)sc2ccccc23)cc1 ; c1ccc2c(c1)-c1ccccc1C21c2cccnc2-c2nc(-c3cc(-c4ccc(-c5cccc6ccccc56)cc4)cc4sc5ccccc5c34)ccc21]

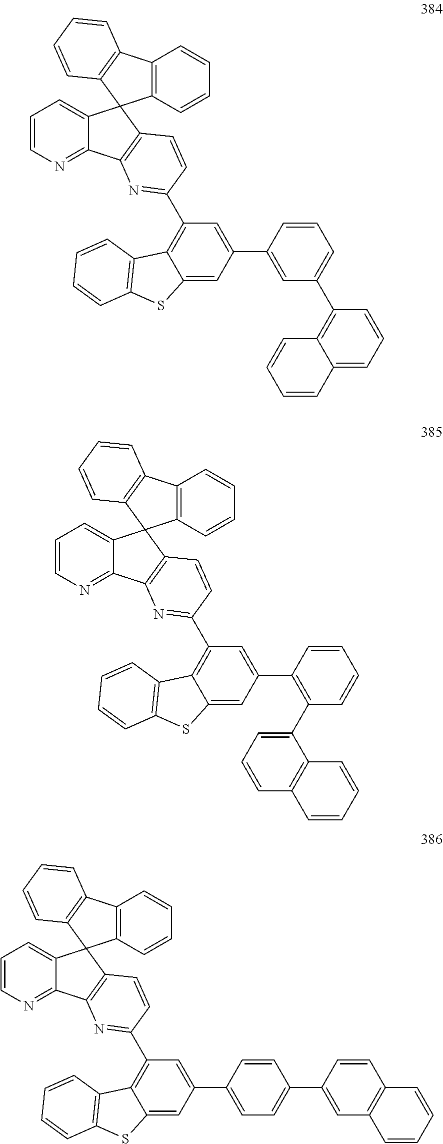 [SMILES: c1cc(-c2cc(-c3ccc4c(n3)-c3ncccc3C43c4ccccc4-c4ccccc43)c3c(c2)sc2ccccc23)cc(-c2cccc3ccccc23)c1 ; c1ccc(-c2cccc3ccccc23)c(-c2cc(-c3ccc4c(n3)-c3ncccc3C43c4ccccc4-c4ccccc43)c3c(c2)sc2ccccc23)c1 ; c1ccc2c(c1)-c1ccccc1C21c2cccnc2-c2nc(-c3cc(-c4ccc(-c5ccc6ccccc6c5)cc4)cc4sc5ccccc5c34)ccc21]

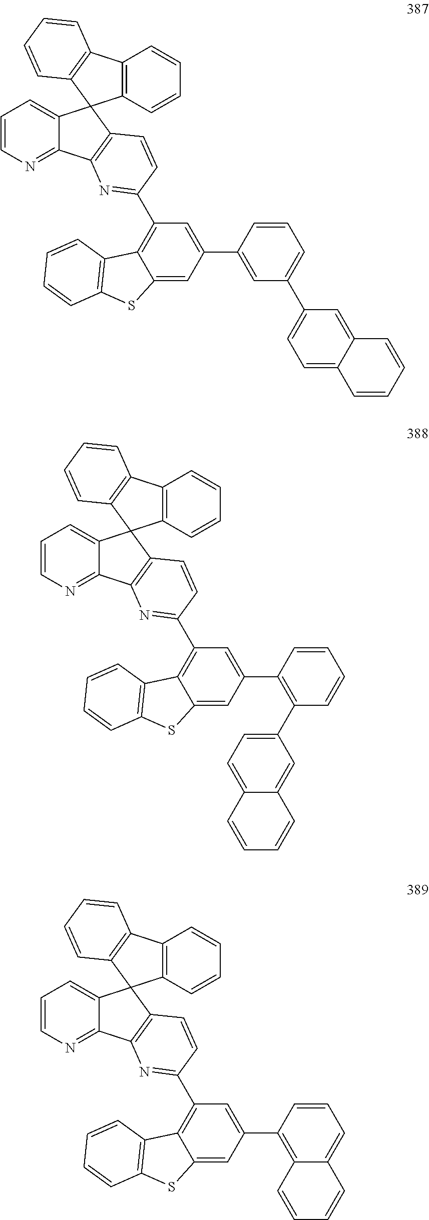 [SMILES: c1cc(-c2ccc3ccccc3c2)cc(-c2cc(-c3ccc4c(n3)-c3ncccc3C43c4ccccc4-c4ccccc43)c3c(c2)sc2ccccc23)c1 ; c1ccc(-c2cc(-c3ccc4c(n3)-c3ncccc3C43c4ccccc4-c4ccccc43)c3c(c2)sc2ccccc23)c(-c2ccc3ccccc3c2)c1 ; c1ccc2c(c1)-c1ccccc1C21c2cccnc2-c2nc(-c3cc(-c4cccc5ccccc45)cc4sc5ccccc5c34)ccc21]

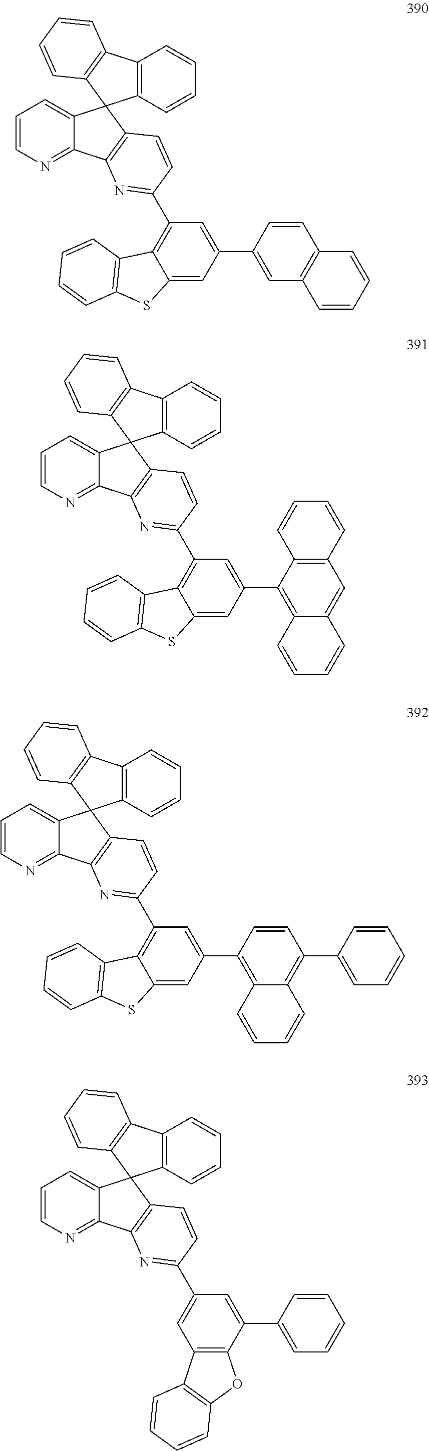 [SMILES: c1ccc2c(c1)-c1ccccc1C21c2cccnc2-c2nc(-c3cc(-c4ccc5ccccc5c4)cc4sc5ccccc5c34)ccc21 ; c1ccc2c(c1)-c1ccccc1C21c2cccnc2-c2nc(-c3cc(-c4c5ccccc5cc5ccccc45)cc4sc5ccccc5c34)ccc21 ; c1ccc(-c2ccc(-c3cc(-c4ccc5c(n4)-c4ncccc4C54c5ccccc5-c5ccccc54)c4c(c3)sc3ccccc34)c3ccccc23)cc1 ; c1ccc(-c2cc(-c3ccc4c(n3)-c3ncccc3C43c4ccccc4-c4ccccc43)cc3c2oc2ccccc23)cc1]

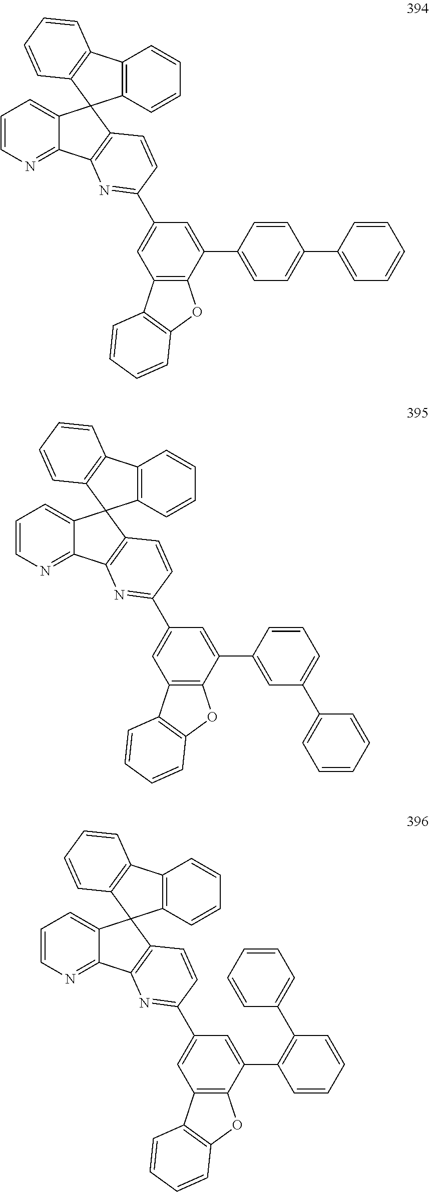 [SMILES: c1ccc(-c2ccc(-c3cc(-c4ccc5c(n4)-c4ncccc4C54c5ccccc5-c5ccccc54)cc4c3oc3ccccc34)cc2)cc1 ; c1ccc(-c2cccc(-c3cc(-c4ccc5c(n4)-c4ncccc4C54c5ccccc5-c5ccccc54)cc4c3oc3ccccc34)c2)cc1 ; c1ccc(-c2ccccc2-c2cc(-c3ccc4c(n3)-c3ncccc3C43c4ccccc4-c4ccccc43)cc3c2oc2ccccc23)cc1]

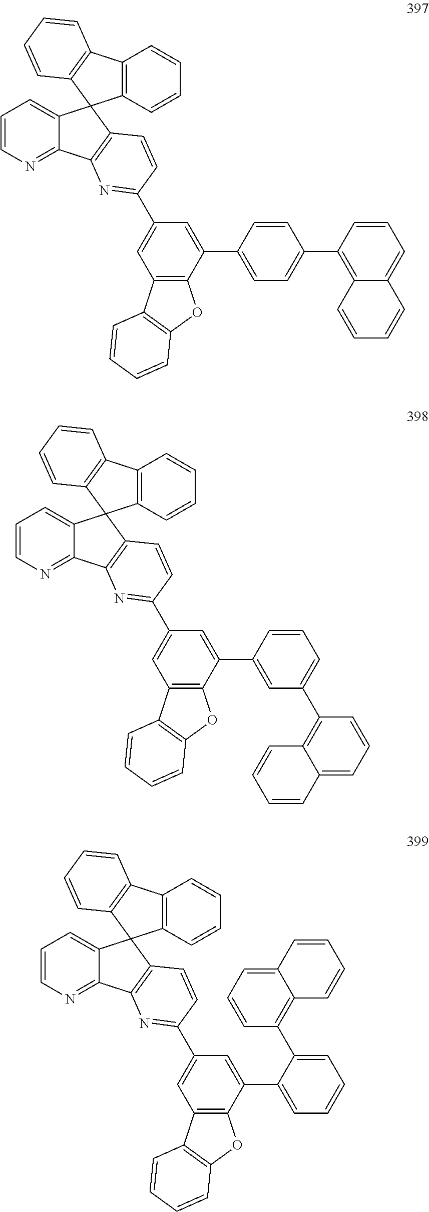 [SMILES: c1ccc2c(c1)-c1ccccc1C21c2cccnc2-c2nc(-c3cc(-c4ccc(-c5cccc6ccccc56)cc4)c4oc5ccccc5c4c3)ccc21 ; c1cc(-c2cccc3ccccc23)cc(-c2cc(-c3ccc4c(n3)-c3ncccc3C43c4ccccc4-c4ccccc43)cc3c2oc2ccccc23)c1 ; c1ccc(-c2cc(-c3ccc4c(n3)-c3ncccc3C43c4ccccc4-c4ccccc43)cc3c2oc2ccccc23)c(-c2cccc3ccccc23)c1]

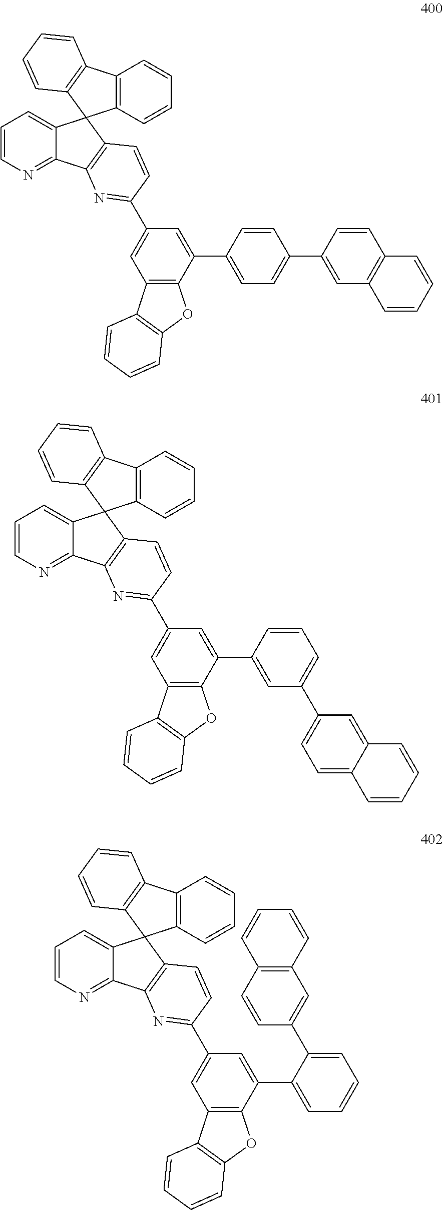 [SMILES: c1ccc2c(c1)-c1ccccc1C21c2cccnc2-c2nc(-c3cc(-c4ccc(-c5ccc6ccccc6c5)cc4)c4oc5ccccc5c4c3)ccc21 ; c1cc(-c2ccc3ccccc3c2)cc(-c2cc(-c3ccc4c(n3)-c3ncccc3C43c4ccccc4-c4ccccc43)cc3c2oc2ccccc23)c1 ; c1ccc(-c2cc(-c3ccc4c(n3)-c3ncccc3C43c4ccccc4-c4ccccc43)cc3c2oc2ccccc23)c(-c2ccc3ccccc3c2)c1]

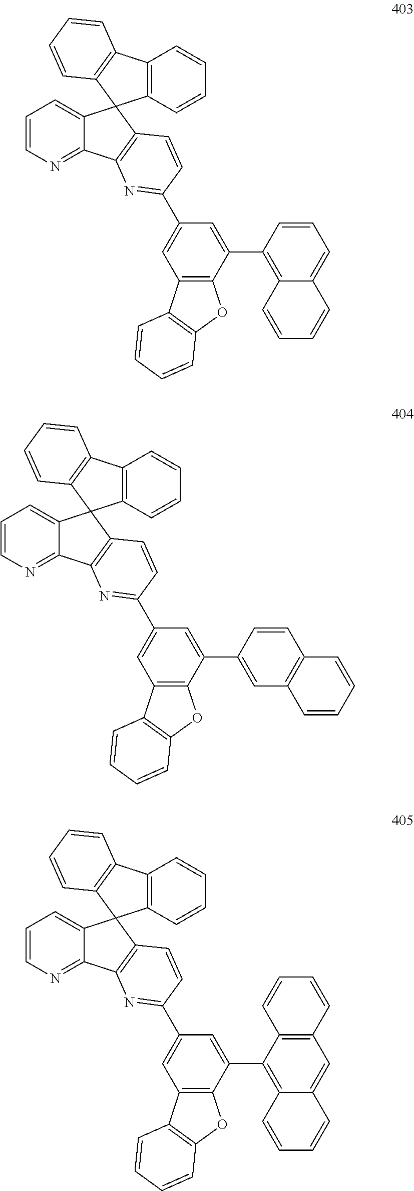 [SMILES: c1ccc2c(c1)-c1ccccc1C21c2cccnc2-c2nc(-c3cc(-c4cccc5ccccc45)c4oc5ccccc5c4c3)ccc21 ; c1ccc2c(c1)-c1ccccc1C21c2cccnc2-c2nc(-c3cc(-c4ccc5ccccc5c4)c4oc5ccccc5c4c3)ccc21 ; c1ccc2c(c1)-c1ccccc1C21c2cccnc2-c2nc(-c3cc(-c4c5ccccc5cc5ccccc45)c4oc5ccccc5c4c3)ccc21]

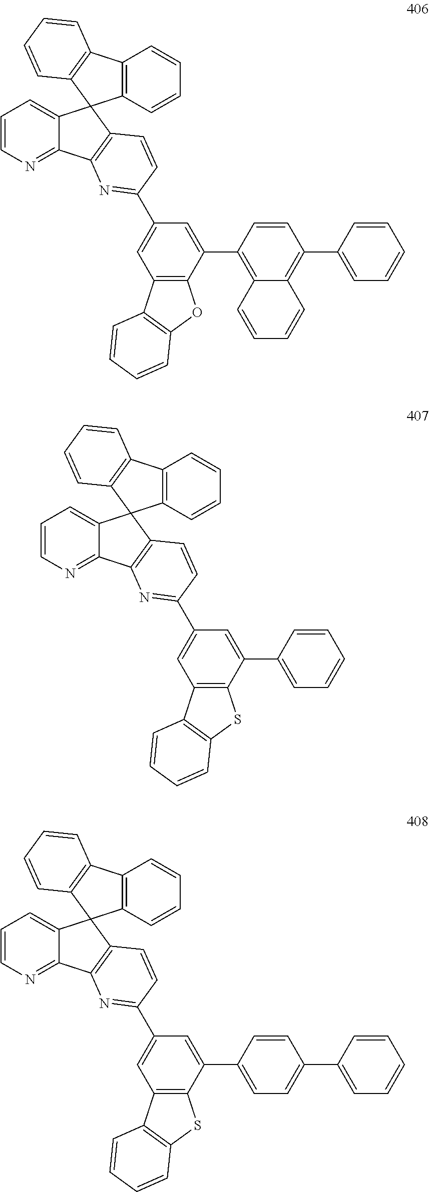 [SMILES: c1ccc(-c2ccc(-c3cc(-c4ccc5c(n4)-c4ncccc4C54c5ccccc5-c5ccccc54)cc4c3oc3ccccc34)c3ccccc23)cc1 ; c1ccc(-c2cc(-c3ccc4c(n3)-c3ncccc3C43c4ccccc4-c4ccccc43)cc3c2sc2ccccc23)cc1 ; c1ccc(-c2ccc(-c3cc(-c4ccc5c(n4)-c4ncccc4C54c5ccccc5-c5ccccc54)cc4c3sc3ccccc34)cc2)cc1]

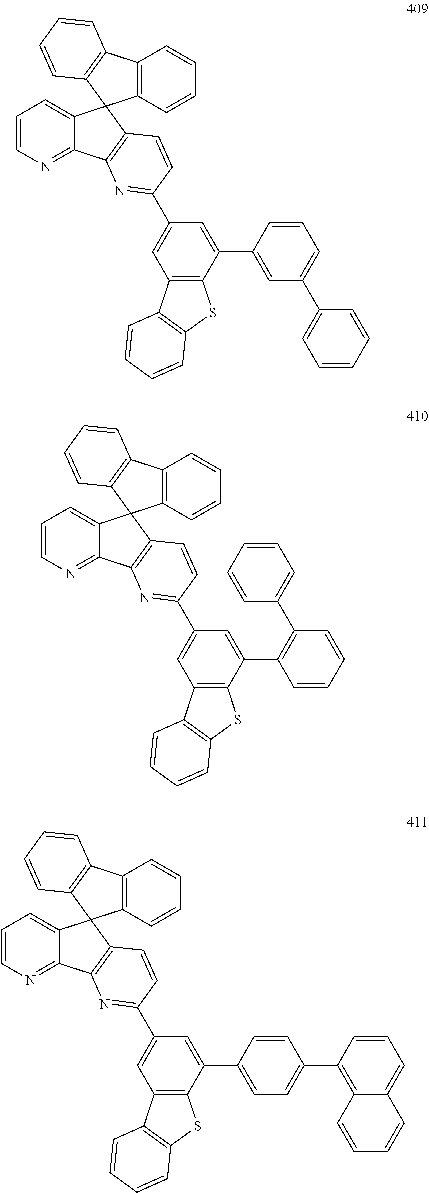 [SMILES: c1ccc(-c2cccc(-c3cc(-c4ccc5c(n4)-c4ncccc4C54c5ccccc5-c5ccccc54)cc4c3sc3ccccc34)c2)cc1 ; c1ccc(-c2ccccc2-c2cc(-c3ccc4c(n3)-c3ncccc3C43c4ccccc4-c4ccccc43)cc3c2sc2ccccc23)cc1 ; c1ccc2c(c1)-c1ccccc1C21c2cccnc2-c2nc(-c3cc(-c4ccc(-c5cccc6ccccc56)cc4)c4sc5ccccc5c4c3)ccc21]

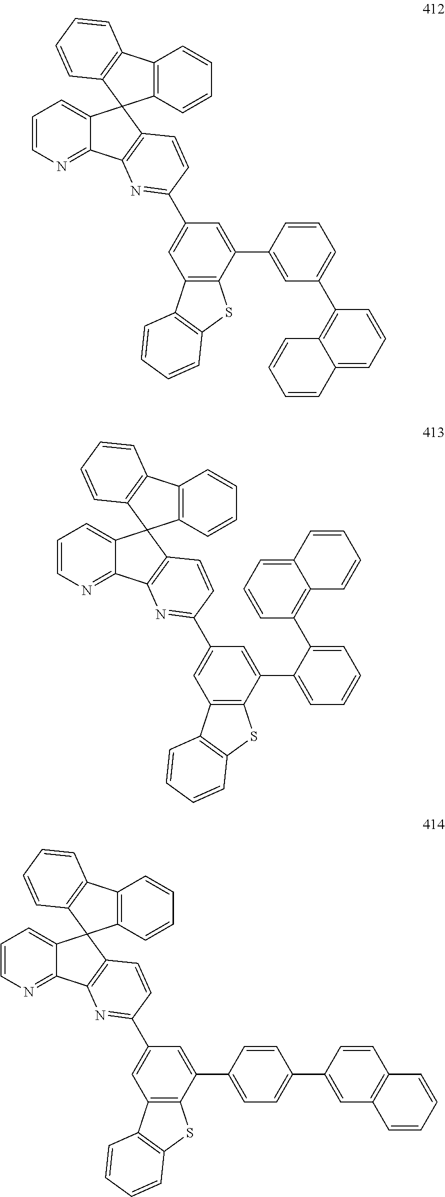 [SMILES: c1cc(-c2cccc3ccccc23)cc(-c2cc(-c3ccc4c(n3)-c3ncccc3C43c4ccccc4-c4ccccc43)cc3c2sc2ccccc23)c1 ; c1ccc(-c2cc(-c3ccc4c(n3)-c3ncccc3C43c4ccccc4-c4ccccc43)cc3c2sc2ccccc23)c(-c2cccc3ccccc23)c1 ; c1ccc2c(c1)-c1ccccc1C21c2cccnc2-c2nc(-c3cc(-c4ccc(-c5ccc6ccccc6c5)cc4)c4sc5ccccc5c4c3)ccc21]

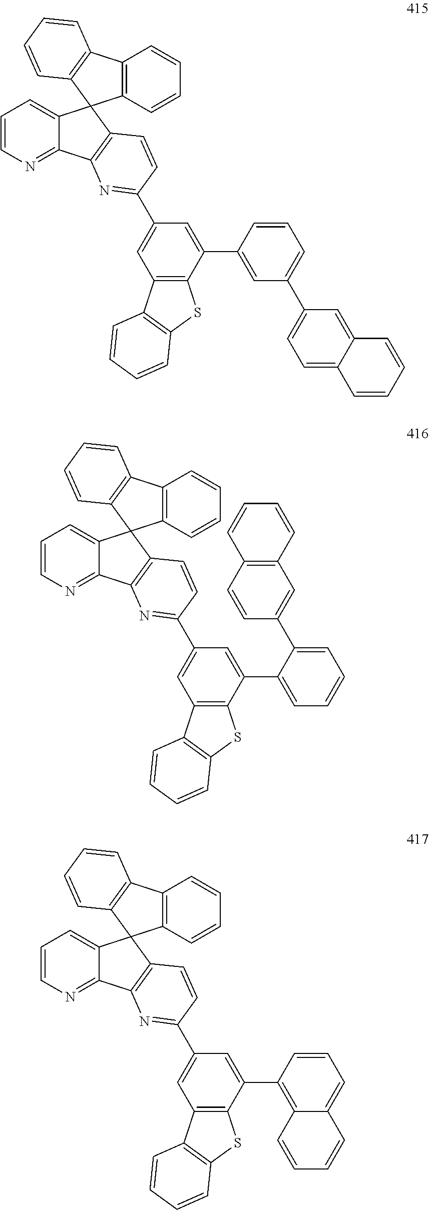 [SMILES: c1cc(-c2ccc3ccccc3c2)cc(-c2cc(-c3ccc4c(n3)-c3ncccc3C43c4ccccc4-c4ccccc43)cc3c2sc2ccccc23)c1 ; c1ccc(-c2cc(-c3ccc4c(n3)-c3ncccc3C43c4ccccc4-c4ccccc43)cc3c2sc2ccccc23)c(-c2ccc3ccccc3c2)c1 ; c1ccc2c(c1)-c1ccccc1C21c2cccnc2-c2nc(-c3cc(-c4cccc5ccccc45)c4sc5ccccc5c4c3)ccc21]

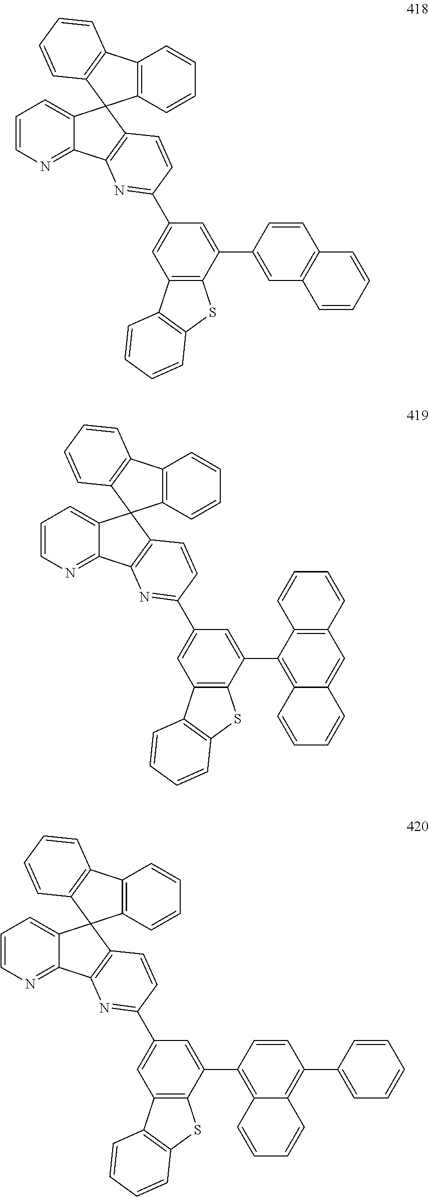 [SMILES: c1ccc2c(c1)-c1ccccc1C21c2cccnc2-c2nc(-c3cc(-c4ccc5ccccc5c4)c4sc5ccccc5c4c3)ccc21 ; c1ccc2c(c1)-c1ccccc1C21c2cccnc2-c2nc(-c3cc(-c4c5ccccc5cc5ccccc45)c4sc5ccccc5c4c3)ccc21 ; c1ccc(-c2ccc(-c3cc(-c4ccc5c(n4)-c4ncccc4C54c5ccccc5-c5ccccc54)cc4c3sc3ccccc34)c3ccccc23)cc1]

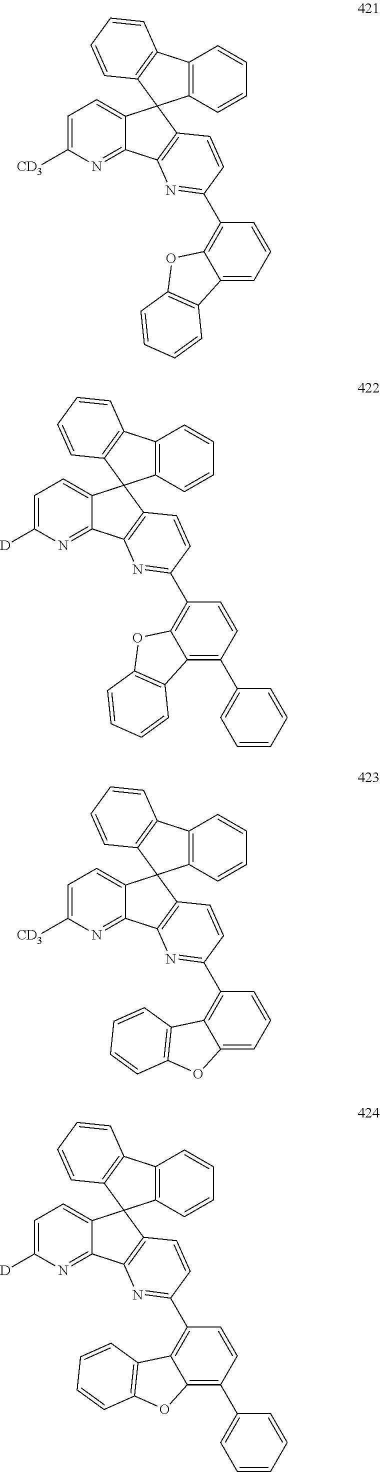 [SMILES: [2H]C([2H])([2H])c1ccc2c(n1)-c1nc(-c3cccc4c3oc3ccccc34)ccc1C21c2ccccc2-c2ccccc21 ; [2H]c1ccc2c(n1)-c1nc(-c3ccc(-c4ccccc4)c4c3oc3ccccc34)ccc1C21c2ccccc2-c2ccccc21 ; [2H]C([2H])([2H])c1ccc2c(n1)-c1nc(-c3cccc4oc5ccccc5c34)ccc1C21c2ccccc2-c2ccccc21 ; [2H]c1ccc2c(n1)-c1nc(-c3ccc(-c4ccccc4)c4oc5ccccc5c34)ccc1C21c2ccccc2-c2ccccc21]

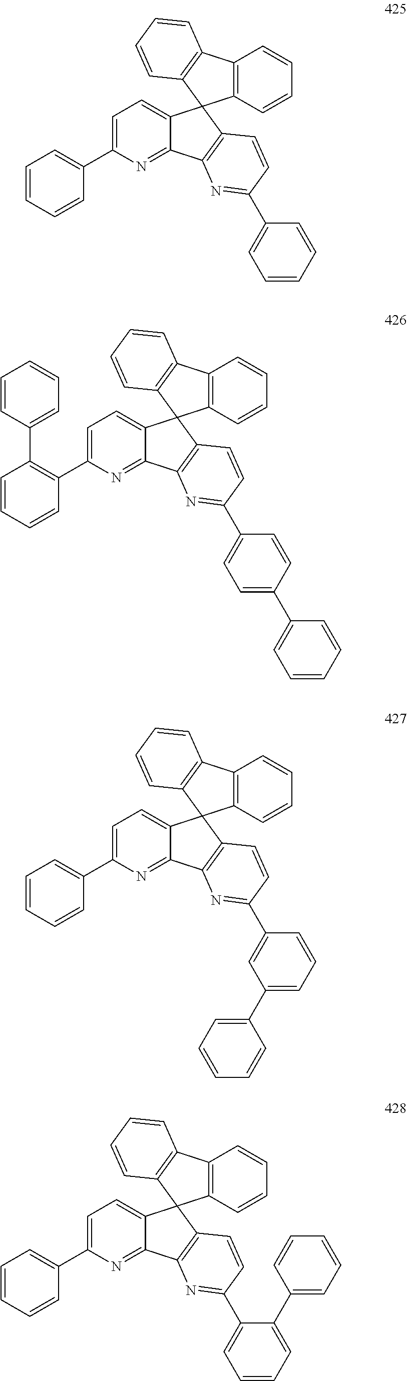 [SMILES: c1ccc(-c2ccc3c(n2)-c2nc(-c4ccccc4)ccc2C32c3ccccc3-c3ccccc32)cc1 ; c1ccc(-c2ccc(-c3ccc4c(n3)-c3nc(-c5ccccc5-c5ccccc5)ccc3C43c4ccccc4-c4ccccc43)cc2)cc1 ; c1ccc(-c2cccc(-c3ccc4c(n3)-c3nc(-c5ccccc5)ccc3C43c4ccccc4-c4ccccc43)c2)cc1 ; c1ccc(-c2ccc3c(n2)-c2nc(-c4ccccc4-c4ccccc4)ccc2C32c3ccccc3-c3ccccc32)cc1]

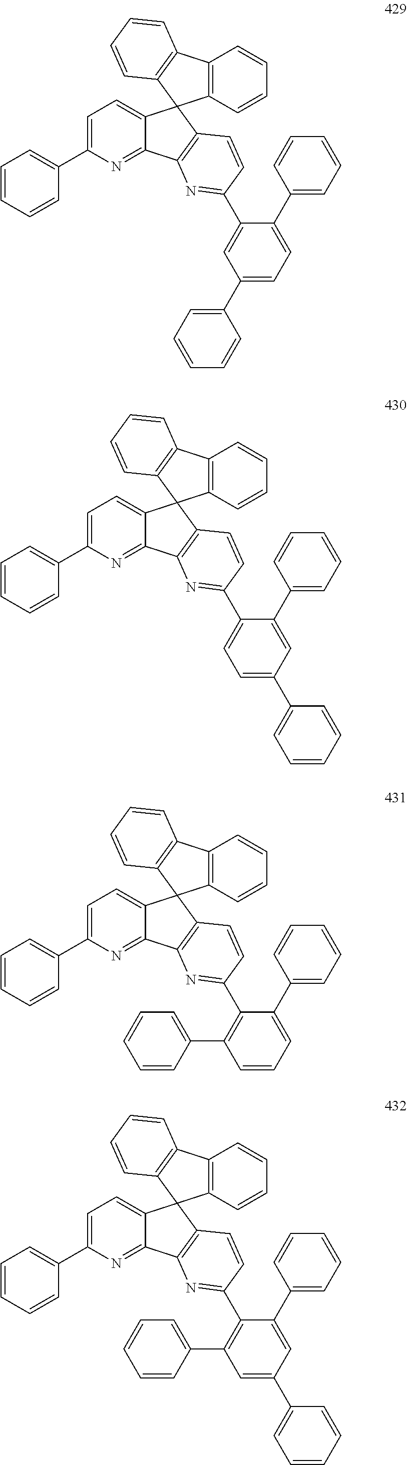

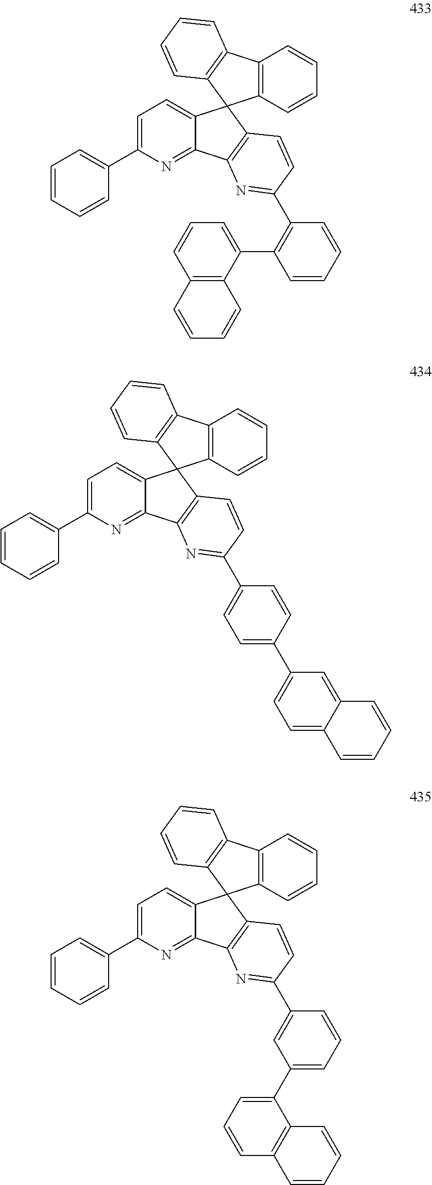 [SMILES: c1ccc(-c2ccc3c(n2)-c2nc(-c4ccccc4-c4cccc5ccccc45)ccc2C32c3ccccc3-c3ccccc32)cc1 ; c1ccc(-c2ccc3c(n2)-c2nc(-c4ccc(-c5ccc6ccccc6c5)cc4)ccc2C32c3ccccc3-c3ccccc32)cc1 ; c1ccc(-c2ccc3c(n2)-c2nc(-c4cccc(-c5cccc6ccccc56)c4)ccc2C32c3ccccc3-c3ccccc32)cc1]

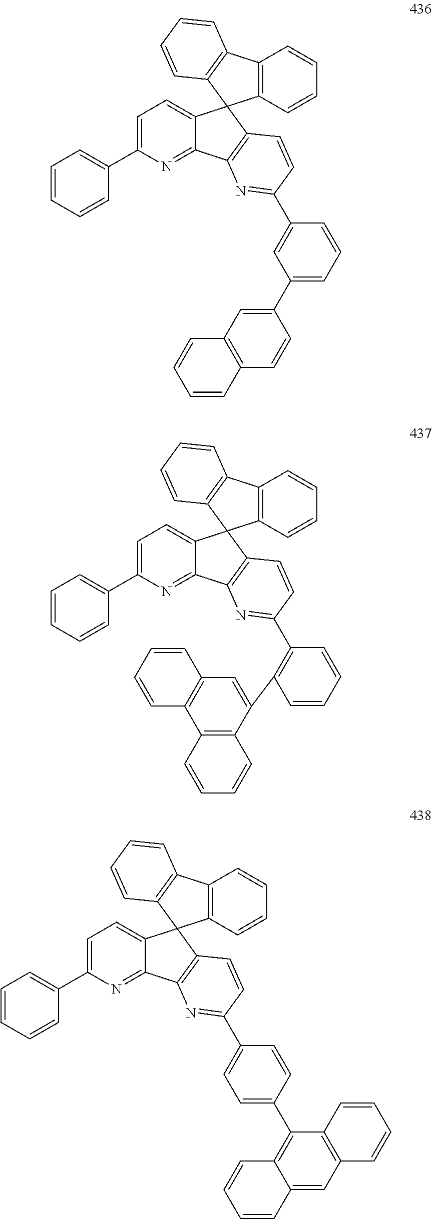 [SMILES: c1ccc(-c2ccc3c(n2)-c2nc(-c4cccc(-c5ccc6ccccc6c5)c4)ccc2C32c3ccccc3-c3ccccc32)cc1 ; c1ccc(-c2ccc3c(n2)-c2nc(-c4ccccc4-c4cc5ccccc5c5ccccc45)ccc2C32c3ccccc3-c3ccccc32)cc1 ; c1ccc(-c2ccc3c(n2)-c2nc(-c4ccc(-c5c6ccccc6cc6ccccc56)cc4)ccc2C32c3ccccc3-c3ccccc32)cc1]

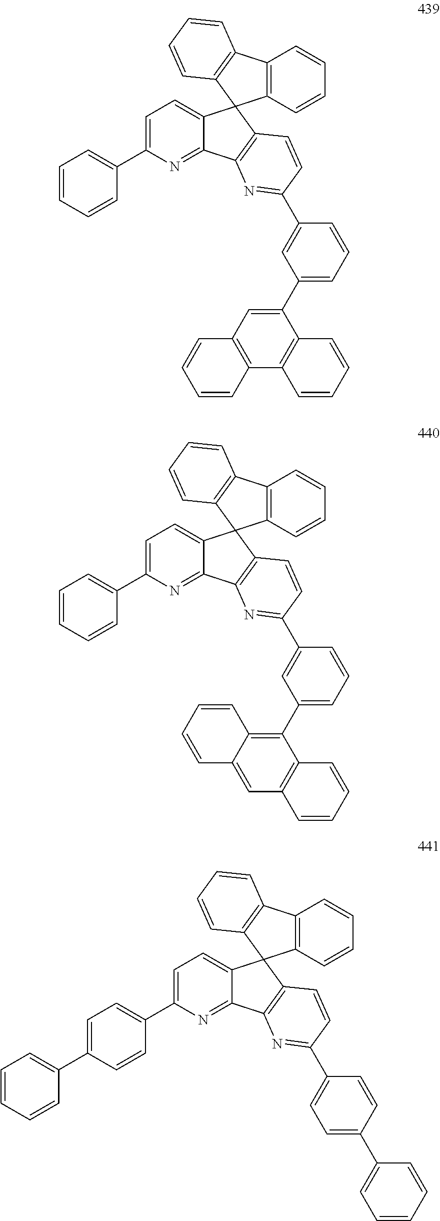 [SMILES: c1ccc(-c2ccc3c(n2)-c2nc(-c4cccc(-c5cc6ccccc6c6ccccc56)c4)ccc2C32c3ccccc3-c3ccccc32)cc1 ; c1ccc(-c2ccc3c(n2)-c2nc(-c4cccc(-c5c6ccccc6cc6ccccc56)c4)ccc2C32c3ccccc3-c3ccccc32)cc1 ; c1ccc(-c2ccc(-c3ccc4c(n3)-c3nc(-c5ccc(-c6ccccc6)cc5)ccc3C43c4ccccc4-c4ccccc43)cc2)cc1]

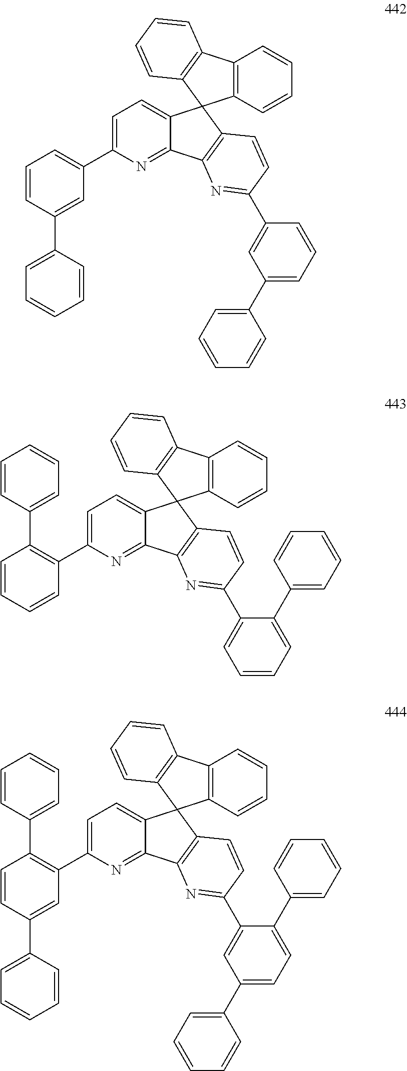 [SMILES: c1ccc(-c2cccc(-c3ccc4c(n3)-c3nc(-c5cccc(-c6ccccc6)c5)ccc3C43c4ccccc4-c4ccccc43)c2)cc1 ; c1ccc(-c2ccccc2-c2ccc3c(n2)-c2nc(-c4ccccc4-c4ccccc4)ccc2C32c3ccccc3-c3ccccc32)cc1 ; c1ccc(-c2ccc(-c3ccccc3)c(-c3ccc4c(n3)-c3nc(-c5cc(-c6ccccc6)ccc5-c5ccccc5)ccc3C43c4ccccc4-c4ccccc43)c2)cc1]

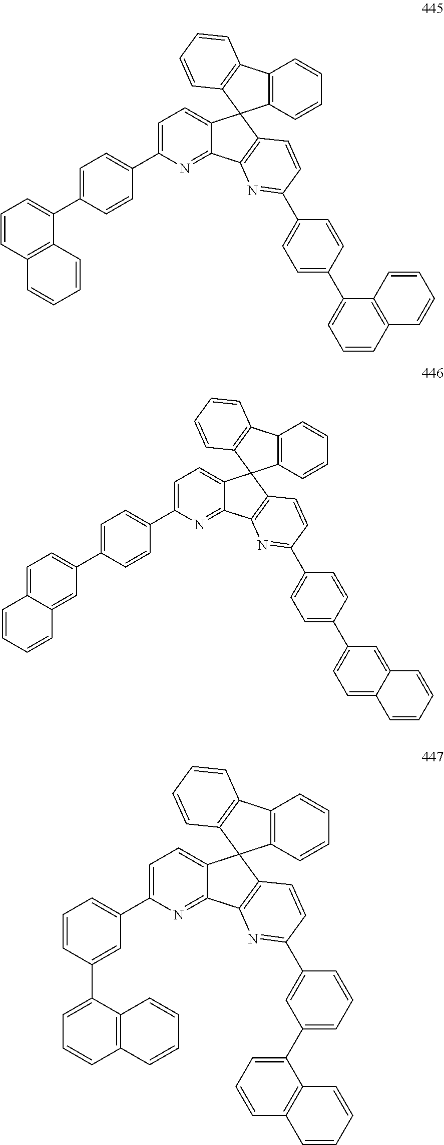 [SMILES: c1ccc2c(c1)-c1ccccc1C21c2ccc(-c3ccc(-c4cccc5ccccc45)cc3)nc2-c2nc(-c3ccc(-c4cccc5ccccc45)cc3)ccc21 ; c1ccc2c(c1)-c1ccccc1C21c2ccc(-c3ccc(-c4ccc5ccccc5c4)cc3)nc2-c2nc(-c3ccc(-c4ccc5ccccc5c4)cc3)ccc21 ; c1cc(-c2ccc3c(n2)-c2nc(-c4cccc(-c5cccc6ccccc56)c4)ccc2C32c3ccccc3-c3ccccc32)cc(-c2cccc3ccccc23)c1]

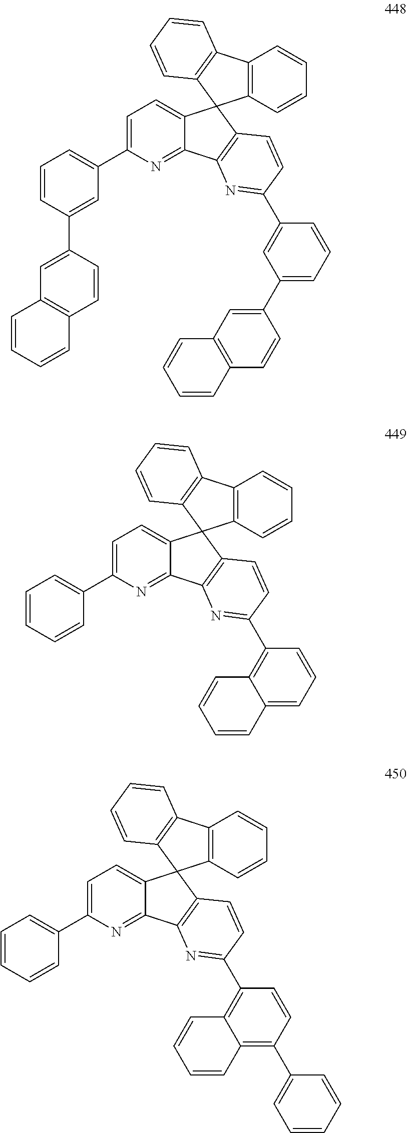 [SMILES: c1cc(-c2ccc3ccccc3c2)cc(-c2ccc3c(n2)-c2nc(-c4cccc(-c5ccc6ccccc6c5)c4)ccc2C32c3ccccc3-c3ccccc32)c1 ; c1ccc(-c2ccc3c(n2)-c2nc(-c4cccc5ccccc45)ccc2C32c3ccccc3-c3ccccc32)cc1 ; c1ccc(-c2ccc3c(n2)-c2nc(-c4ccc(-c5ccccc5)c5ccccc45)ccc2C32c3ccccc3-c3ccccc32)cc1]

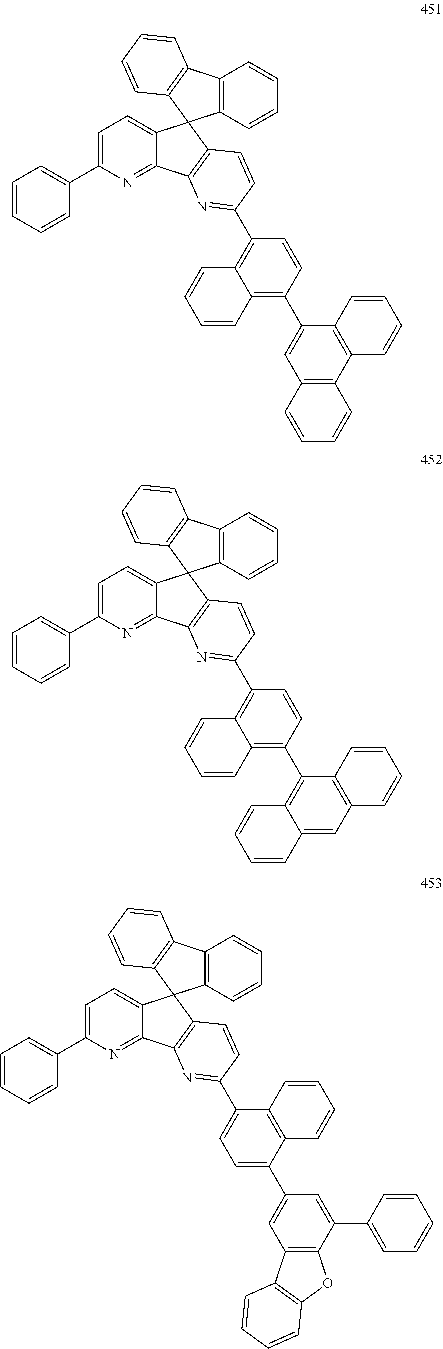 [SMILES: c1ccc(-c2ccc3c(n2)-c2nc(-c4ccc(-c5cc6ccccc6c6ccccc56)c5ccccc45)ccc2C32c3ccccc3-c3ccccc32)cc1 ; c1ccc(-c2ccc3c(n2)-c2nc(-c4ccc(-c5c6ccccc6cc6ccccc56)c5ccccc45)ccc2C32c3ccccc3-c3ccccc32)cc1 ; c1ccc(-c2ccc3c(n2)-c2nc(-c4ccc(-c5cc(-c6ccccc6)c6oc7ccccc7c6c5)c5ccccc45)ccc2C32c3ccccc3-c3ccccc32)cc1]

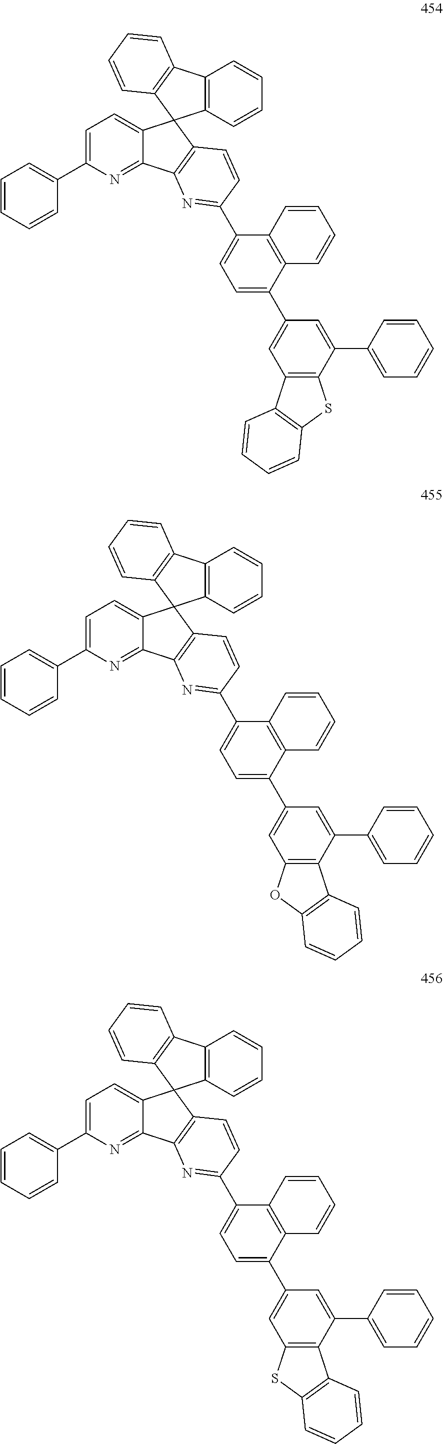 [SMILES: c1ccc(-c2ccc3c(n2)-c2nc(-c4ccc(-c5cc(-c6ccccc6)c6sc7ccccc7c6c5)c5ccccc45)ccc2C32c3ccccc3-c3ccccc32)cc1 ; c1ccc(-c2ccc3c(n2)-c2nc(-c4ccc(-c5cc(-c6ccccc6)c6c(c5)oc5ccccc56)c5ccccc45)ccc2C32c3ccccc3-c3ccccc32)cc1 ; c1ccc(-c2ccc3c(n2)-c2nc(-c4ccc(-c5cc(-c6ccccc6)c6c(c5)sc5ccccc56)c5ccccc45)ccc2C32c3ccccc3-c3ccccc32)cc1]

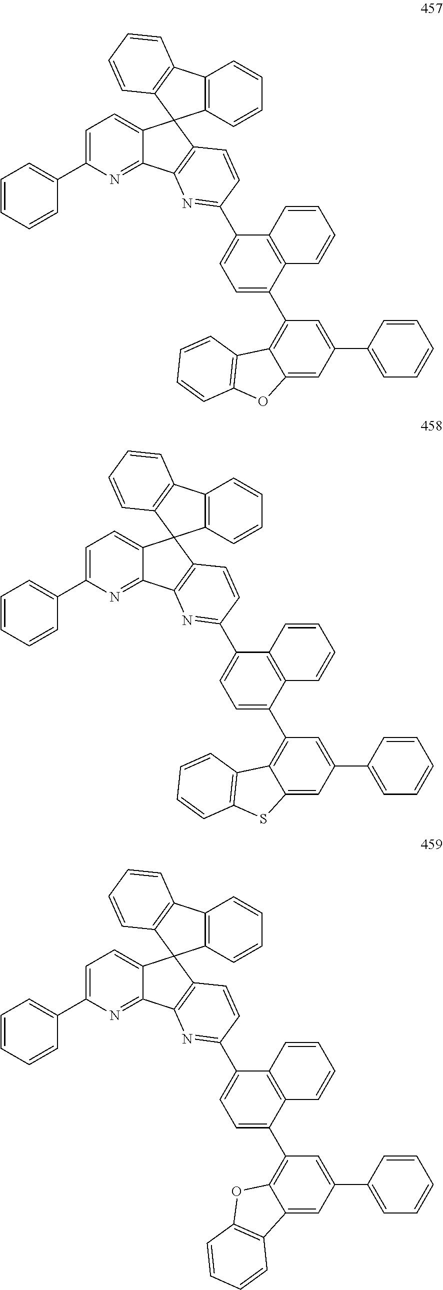 [SMILES: c1ccc(-c2cc(-c3ccc(-c4ccc5c(n4)-c4nc(-c6ccccc6)ccc4C54c5ccccc5-c5ccccc54)c4ccccc34)c3c(c2)oc2ccccc23)cc1 ; c1ccc(-c2cc(-c3ccc(-c4ccc5c(n4)-c4nc(-c6ccccc6)ccc4C54c5ccccc5-c5ccccc54)c4ccccc34)c3c(c2)sc2ccccc23)cc1 ; c1ccc(-c2cc(-c3ccc(-c4ccc5c(n4)-c4nc(-c6ccccc6)ccc4C54c5ccccc5-c5ccccc54)c4ccccc34)c3oc4ccccc4c3c2)cc1]

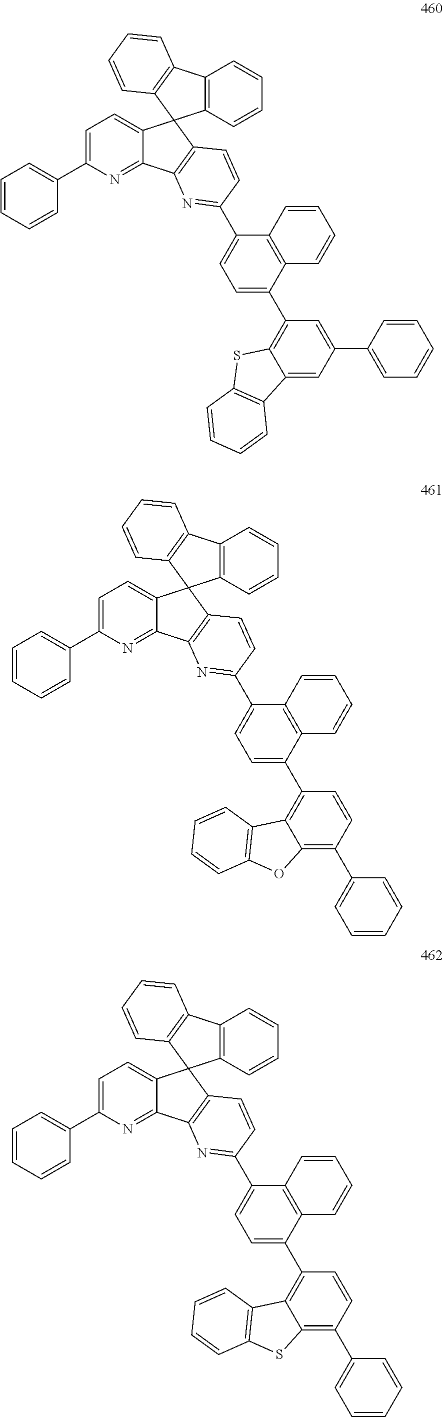 [SMILES: c1ccc(-c2cc(-c3ccc(-c4ccc5c(n4)-c4nc(-c6ccccc6)ccc4C54c5ccccc5-c5ccccc54)c4ccccc34)c3sc4ccccc4c3c2)cc1 ; c1ccc(-c2ccc3c(n2)-c2nc(-c4ccc(-c5ccc(-c6ccccc6)c6oc7ccccc7c56)c5ccccc45)ccc2C32c3ccccc3-c3ccccc32)cc1 ; c1ccc(-c2ccc3c(n2)-c2nc(-c4ccc(-c5ccc(-c6ccccc6)c6sc7ccccc7c56)c5ccccc45)ccc2C32c3ccccc3-c3ccccc32)cc1]

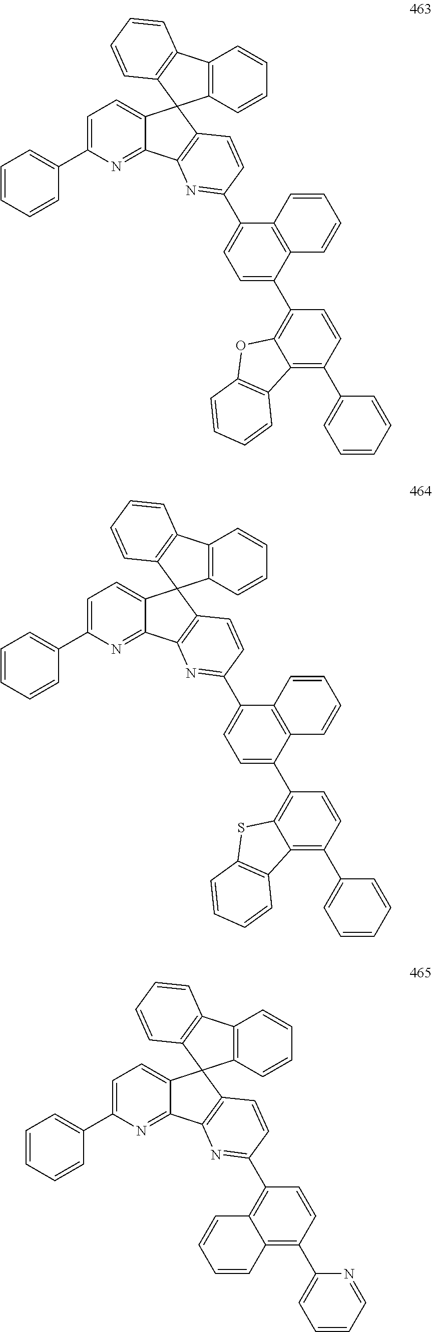 [SMILES: c1ccc(-c2ccc3c(n2)-c2nc(-c4ccc(-c5ccc(-c6ccccc6)c6c5oc5ccccc56)c5ccccc45)ccc2C32c3ccccc3-c3ccccc32)cc1 ; c1ccc(-c2ccc3c(n2)-c2nc(-c4ccc(-c5ccc(-c6ccccc6)c6c5sc5ccccc56)c5ccccc45)ccc2C32c3ccccc3-c3ccccc32)cc1 ; c1ccc(-c2ccc3c(n2)-c2nc(-c4ccc(-c5ccccn5)c5ccccc45)ccc2C32c3ccccc3-c3ccccc32)cc1]

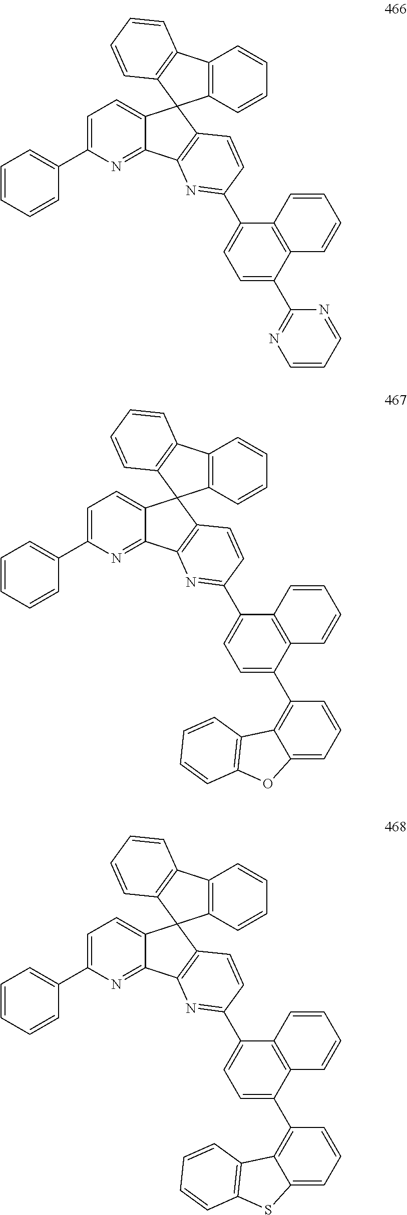 [SMILES: c1ccc(-c2ccc3c(n2)-c2nc(-c4ccc(-c5ncccn5)c5ccccc45)ccc2C32c3ccccc3-c3ccccc32)cc1 ; c1ccc(-c2ccc3c(n2)-c2nc(-c4ccc(-c5cccc6oc7ccccc7c56)c5ccccc45)ccc2C32c3ccccc3-c3ccccc32)cc1 ; c1ccc(-c2ccc3c(n2)-c2nc(-c4ccc(-c5cccc6sc7ccccc7c56)c5ccccc45)ccc2C32c3ccccc3-c3ccccc32)cc1]

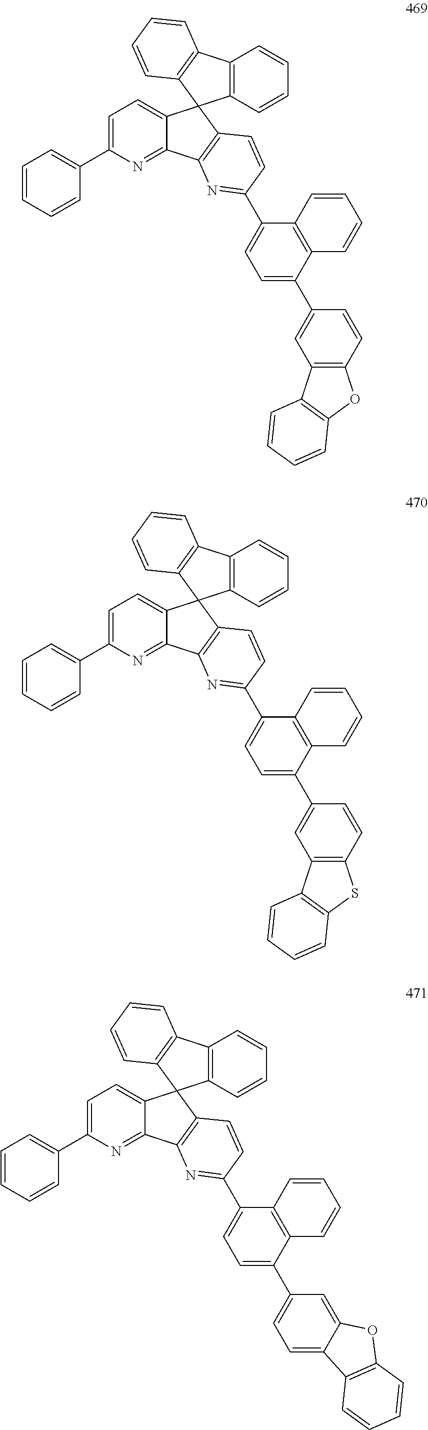 [SMILES: c1ccc(-c2ccc3c(n2)-c2nc(-c4ccc(-c5ccc6oc7ccccc7c6c5)c5ccccc45)ccc2C32c3ccccc3-c3ccccc32)cc1 ; c1ccc(-c2ccc3c(n2)-c2nc(-c4ccc(-c5ccc6sc7ccccc7c6c5)c5ccccc45)ccc2C32c3ccccc3-c3ccccc32)cc1 ; c1ccc(-c2ccc3c(n2)-c2nc(-c4ccc(-c5ccc6c(c5)oc5ccccc56)c5ccccc45)ccc2C32c3ccccc3-c3ccccc32)cc1]

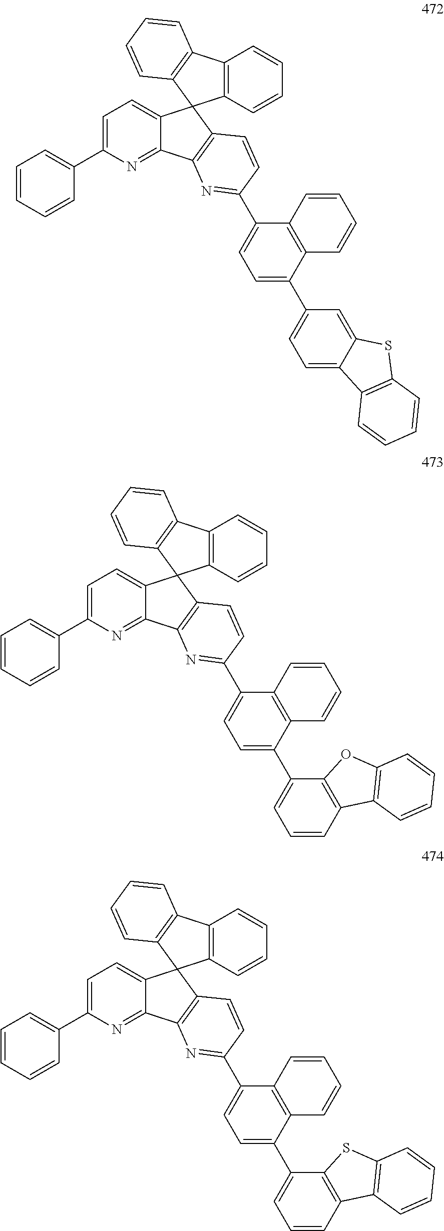 [SMILES: c1ccc(-c2ccc3c(n2)-c2nc(-c4ccc(-c5ccc6c(c5)sc5ccccc56)c5ccccc45)ccc2C32c3ccccc3-c3ccccc32)cc1 ; c1ccc(-c2ccc3c(n2)-c2nc(-c4ccc(-c5cccc6c5oc5ccccc56)c5ccccc45)ccc2C32c3ccccc3-c3ccccc32)cc1 ; c1ccc(-c2ccc3c(n2)-c2nc(-c4ccc(-c5cccc6c5sc5ccccc56)c5ccccc45)ccc2C32c3ccccc3-c3ccccc32)cc1]

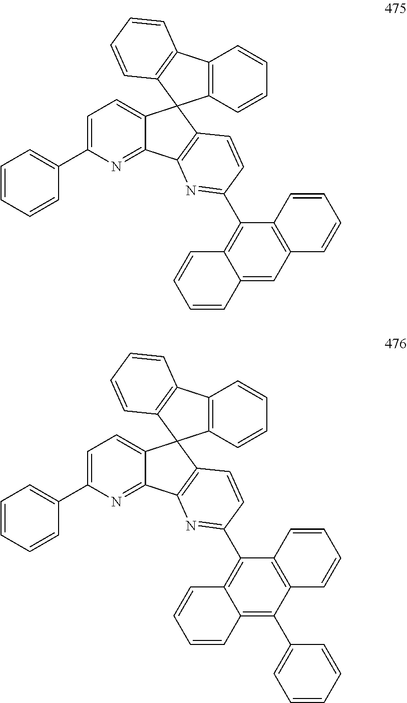 [SMILES: c1ccc(-c2ccc3c(n2)-c2nc(-c4c5ccccc5cc5ccccc45)ccc2C32c3ccccc3-c3ccccc32)cc1 ; c1ccc(-c2ccc3c(n2)-c2nc(-c4c5ccccc5c(-c5ccccc5)c5ccccc45)ccc2C32c3ccccc3-c3ccccc32)cc1]

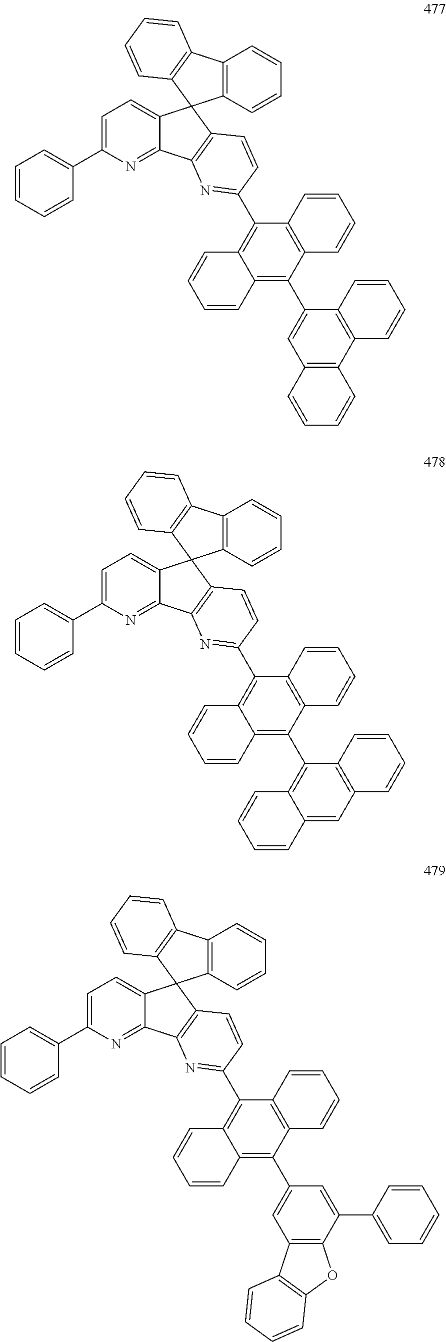 [SMILES: c1ccc(-c2ccc3c(n2)-c2nc(-c4c5ccccc5c(-c5cc6ccccc6c6ccccc56)c5ccccc45)ccc2C32c3ccccc3-c3ccccc32)cc1 ; c1ccc(-c2ccc3c(n2)-c2nc(-c4c5ccccc5c(-c5c6ccccc6cc6ccccc56)c5ccccc45)ccc2C32c3ccccc3-c3ccccc32)cc1 ; c1ccc(-c2ccc3c(n2)-c2nc(-c4c5ccccc5c(-c5cc(-c6ccccc6)c6oc7ccccc7c6c5)c5ccccc45)ccc2C32c3ccccc3-c3ccccc32)cc1]

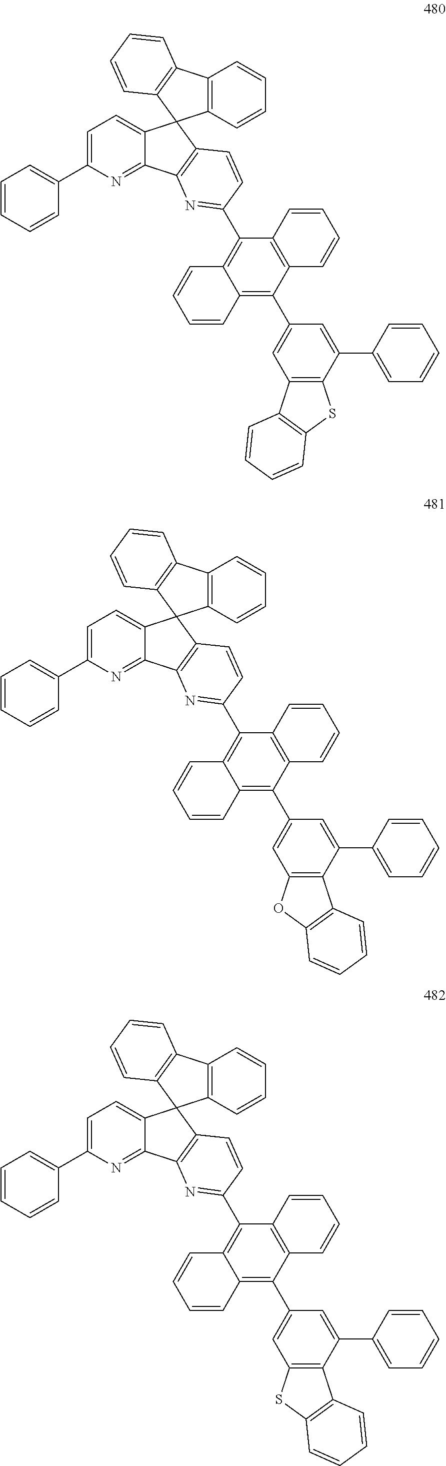 [SMILES: c1ccc(-c2ccc3c(n2)-c2nc(-c4c5ccccc5c(-c5cc(-c6ccccc6)c6sc7ccccc7c6c5)c5ccccc45)ccc2C32c3ccccc3-c3ccccc32)cc1 ; c1ccc(-c2ccc3c(n2)-c2nc(-c4c5ccccc5c(-c5cc(-c6ccccc6)c6c(c5)oc5ccccc56)c5ccccc45)ccc2C32c3ccccc3-c3ccccc32)cc1 ; c1ccc(-c2ccc3c(n2)-c2nc(-c4c5ccccc5c(-c5cc(-c6ccccc6)c6c(c5)sc5ccccc56)c5ccccc45)ccc2C32c3ccccc3-c3ccccc32)cc1]

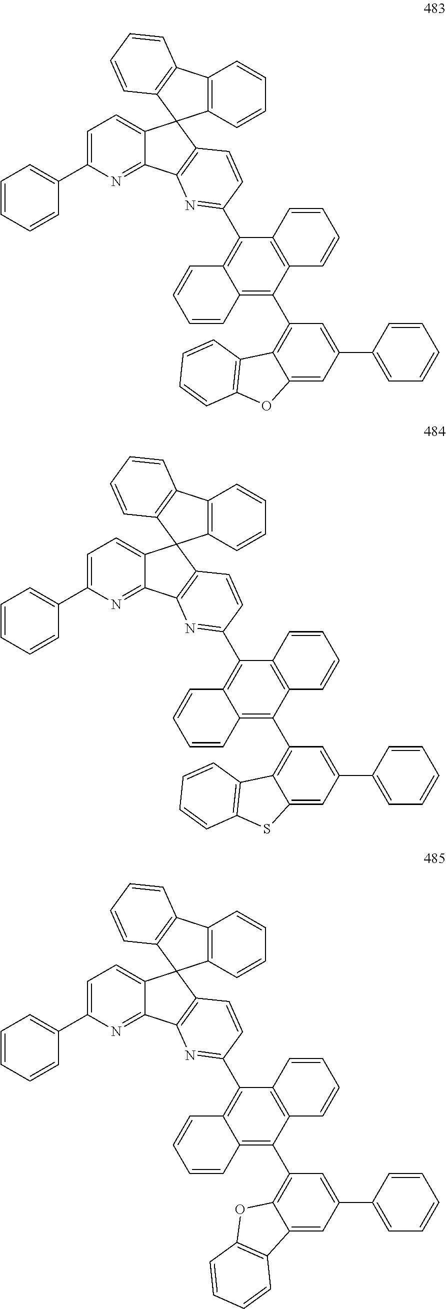 [SMILES: c1ccc(-c2cc(-c3c4ccccc4c(-c4ccc5c(n4)-c4nc(-c6ccccc6)ccc4C54c5ccccc5-c5ccccc54)c4ccccc34)c3c(c2)oc2ccccc23)cc1 ; c1ccc(-c2cc(-c3c4ccccc4c(-c4ccc5c(n4)-c4nc(-c6ccccc6)ccc4C54c5ccccc5-c5ccccc54)c4ccccc34)c3c(c2)sc2ccccc23)cc1 ; c1ccc(-c2cc(-c3c4ccccc4c(-c4ccc5c(n4)-c4nc(-c6ccccc6)ccc4C54c5ccccc5-c5ccccc54)c4ccccc34)c3oc4ccccc4c3c2)cc1]

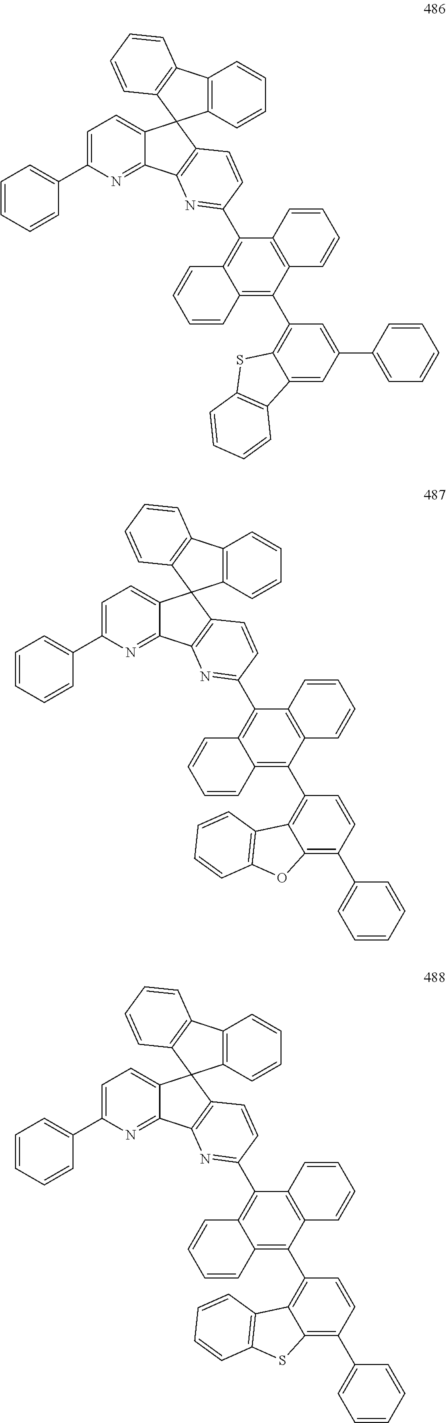 [SMILES: c1ccc(-c2cc(-c3c4ccccc4c(-c4ccc5c(n4)-c4nc(-c6ccccc6)ccc4C54c5ccccc5-c5ccccc54)c4ccccc34)c3sc4ccccc4c3c2)cc1 ; c1ccc(-c2ccc3c(n2)-c2nc(-c4c5ccccc5c(-c5ccc(-c6ccccc6)c6oc7ccccc7c56)c5ccccc45)ccc2C32c3ccccc3-c3ccccc32)cc1 ; c1ccc(-c2ccc3c(n2)-c2nc(-c4c5ccccc5c(-c5ccc(-c6ccccc6)c6sc7ccccc7c56)c5ccccc45)ccc2C32c3ccccc3-c3ccccc32)cc1]

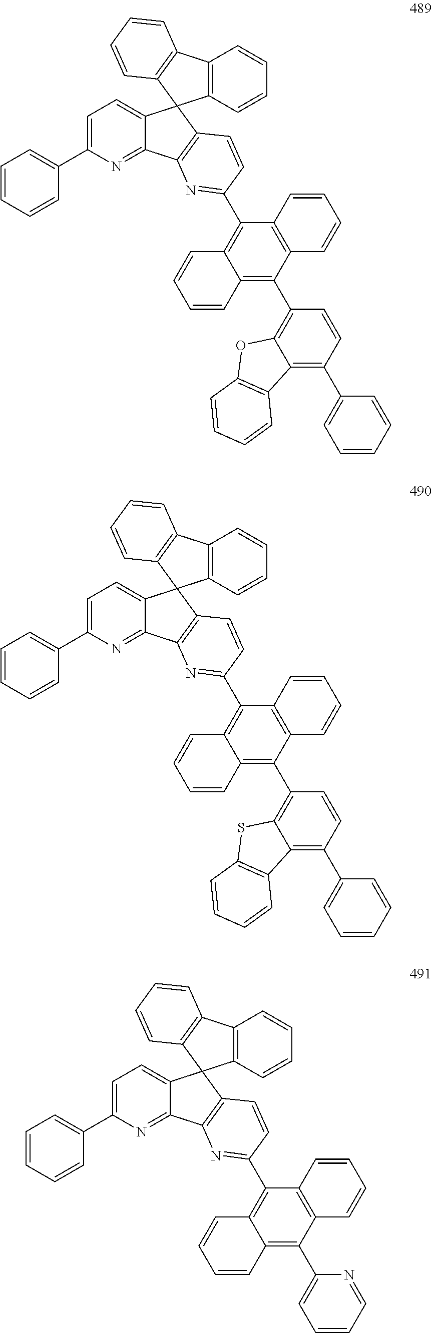 [SMILES: c1ccc(-c2ccc3c(n2)-c2nc(-c4c5ccccc5c(-c5ccc(-c6ccccc6)c6c5oc5ccccc56)c5ccccc45)ccc2C32c3ccccc3-c3ccccc32)cc1 ; c1ccc(-c2ccc3c(n2)-c2nc(-c4c5ccccc5c(-c5ccc(-c6ccccc6)c6c5sc5ccccc56)c5ccccc45)ccc2C32c3ccccc3-c3ccccc32)cc1 ; c1ccc(-c2ccc3c(n2)-c2nc(-c4c5ccccc5c(-c5ccccn5)c5ccccc45)ccc2C32c3ccccc3-c3ccccc32)cc1]

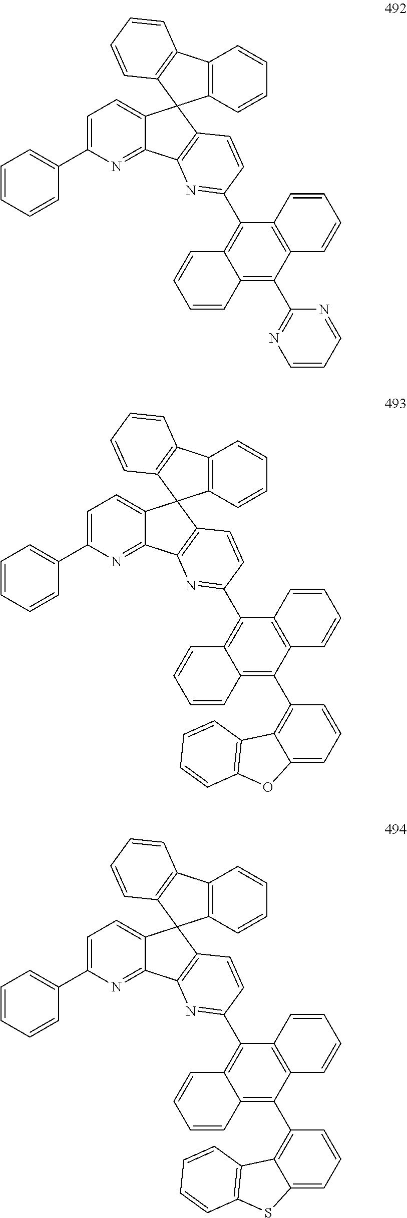 [SMILES: c1ccc(-c2ccc3c(n2)-c2nc(-c4c5ccccc5c(-c5ncccn5)c5ccccc45)ccc2C32c3ccccc3-c3ccccc32)cc1 ; c1ccc(-c2ccc3c(n2)-c2nc(-c4c5ccccc5c(-c5cccc6oc7ccccc7c56)c5ccccc45)ccc2C32c3ccccc3-c3ccccc32)cc1 ; c1ccc(-c2ccc3c(n2)-c2nc(-c4c5ccccc5c(-c5cccc6sc7ccccc7c56)c5ccccc45)ccc2C32c3ccccc3-c3ccccc32)cc1]

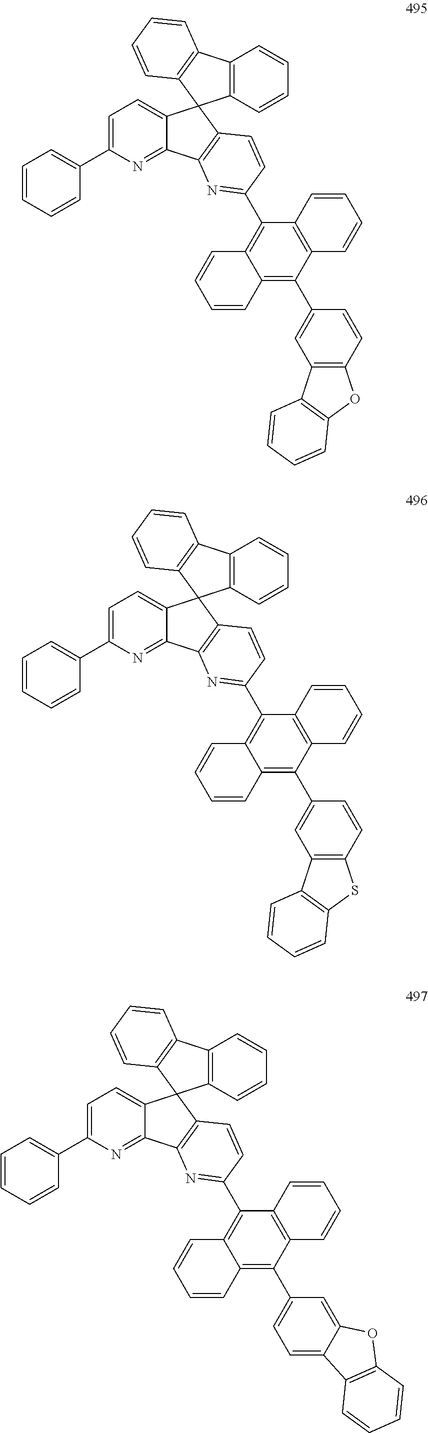 [SMILES: c1ccc(-c2ccc3c(n2)-c2nc(-c4c5ccccc5c(-c5ccc6oc7ccccc7c6c5)c5ccccc45)ccc2C32c3ccccc3-c3ccccc32)cc1 ; c1ccc(-c2ccc3c(n2)-c2nc(-c4c5ccccc5c(-c5ccc6sc7ccccc7c6c5)c5ccccc45)ccc2C32c3ccccc3-c3ccccc32)cc1 ; c1ccc(-c2ccc3c(n2)-c2nc(-c4c5ccccc5c(-c5ccc6c(c5)oc5ccccc56)c5ccccc45)ccc2C32c3ccccc3-c3ccccc32)cc1]

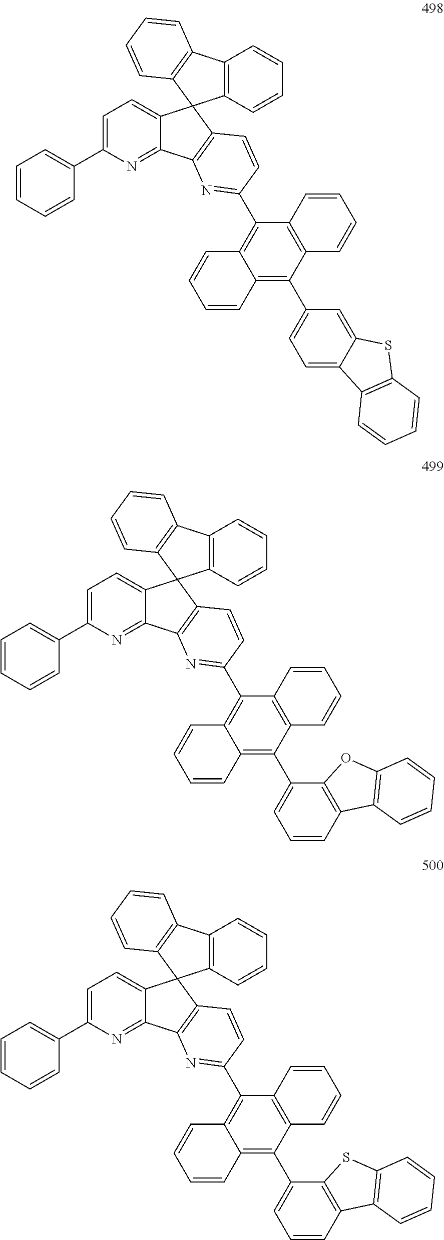 [SMILES: c1ccc(-c2ccc3c(n2)-c2nc(-c4c5ccccc5c(-c5ccc6c(c5)sc5ccccc56)c5ccccc45)ccc2C32c3ccccc3-c3ccccc32)cc1 ; c1ccc(-c2ccc3c(n2)-c2nc(-c4c5ccccc5c(-c5cccc6c5oc5ccccc56)c5ccccc45)ccc2C32c3ccccc3-c3ccccc32)cc1 ; c1ccc(-c2ccc3c(n2)-c2nc(-c4c5ccccc5c(-c5cccc6c5sc5ccccc56)c5ccccc45)ccc2C32c3ccccc3-c3ccccc32)cc1]

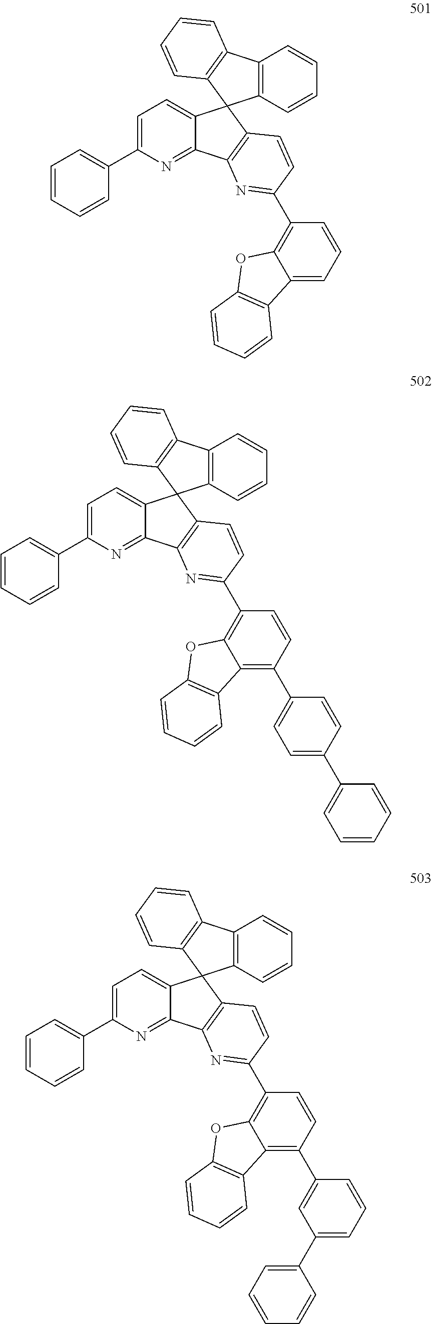 [SMILES: c1ccc(-c2ccc3c(n2)-c2nc(-c4cccc5c4oc4ccccc45)ccc2C32c3ccccc3-c3ccccc32)cc1 ; c1ccc(-c2ccc(-c3ccc(-c4ccc5c(n4)-c4nc(-c6ccccc6)ccc4C54c5ccccc5-c5ccccc54)c4oc5ccccc5c34)cc2)cc1 ; c1ccc(-c2cccc(-c3ccc(-c4ccc5c(n4)-c4nc(-c6ccccc6)ccc4C54c5ccccc5-c5ccccc54)c4oc5ccccc5c34)c2)cc1]

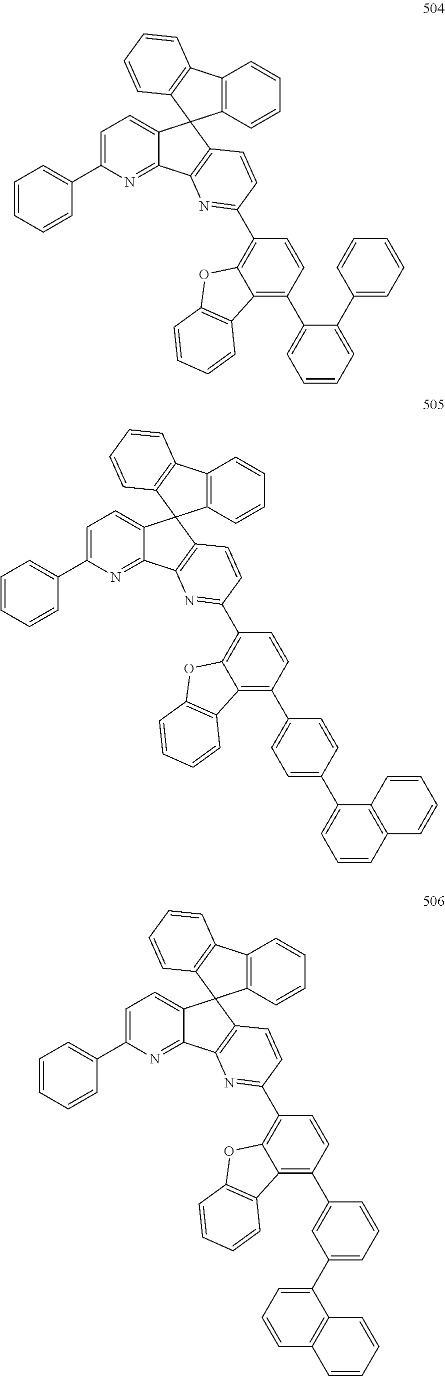 [SMILES: c1ccc(-c2ccc3c(n2)-c2nc(-c4ccc(-c5ccccc5-c5ccccc5)c5c4oc4ccccc45)ccc2C32c3ccccc3-c3ccccc32)cc1 ; c1ccc(-c2ccc3c(n2)-c2nc(-c4ccc(-c5ccc(-c6cccc7ccccc67)cc5)c5c4oc4ccccc45)ccc2C32c3ccccc3-c3ccccc32)cc1 ; c1ccc(-c2ccc3c(n2)-c2nc(-c4ccc(-c5cccc(-c6cccc7ccccc67)c5)c5c4oc4ccccc45)ccc2C32c3ccccc3-c3ccccc32)cc1]

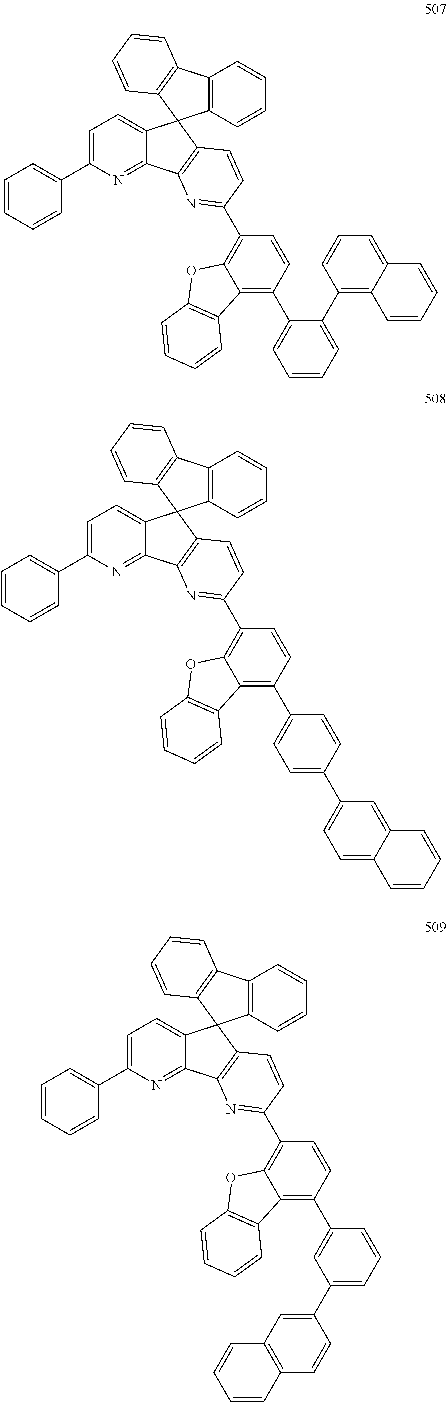 [SMILES: c1ccc(-c2ccc3c(n2)-c2nc(-c4ccc(-c5ccccc5-c5cccc6ccccc56)c5c4oc4ccccc45)ccc2C32c3ccccc3-c3ccccc32)cc1 ; c1ccc(-c2ccc3c(n2)-c2nc(-c4ccc(-c5ccc(-c6ccc7ccccc7c6)cc5)c5c4oc4ccccc45)ccc2C32c3ccccc3-c3ccccc32)cc1 ; c1ccc(-c2ccc3c(n2)-c2nc(-c4ccc(-c5cccc(-c6ccc7ccccc7c6)c5)c5c4oc4ccccc45)ccc2C32c3ccccc3-c3ccccc32)cc1]

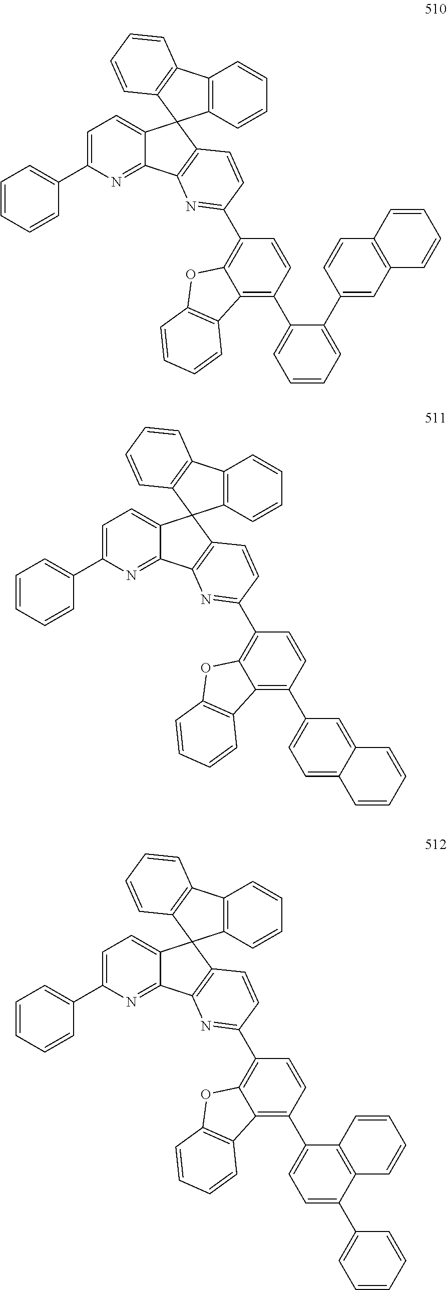 [SMILES: c1ccc(-c2ccc3c(n2)-c2nc(-c4ccc(-c5ccccc5-c5ccc6ccccc6c5)c5c4oc4ccccc45)ccc2C32c3ccccc3-c3ccccc32)cc1 ; c1ccc(-c2ccc3c(n2)-c2nc(-c4ccc(-c5ccc6ccccc6c5)c5c4oc4ccccc45)ccc2C32c3ccccc3-c3ccccc32)cc1 ; c1ccc(-c2ccc3c(n2)-c2nc(-c4ccc(-c5ccc(-c6ccccc6)c6ccccc56)c5c4oc4ccccc45)ccc2C32c3ccccc3-c3ccccc32)cc1]

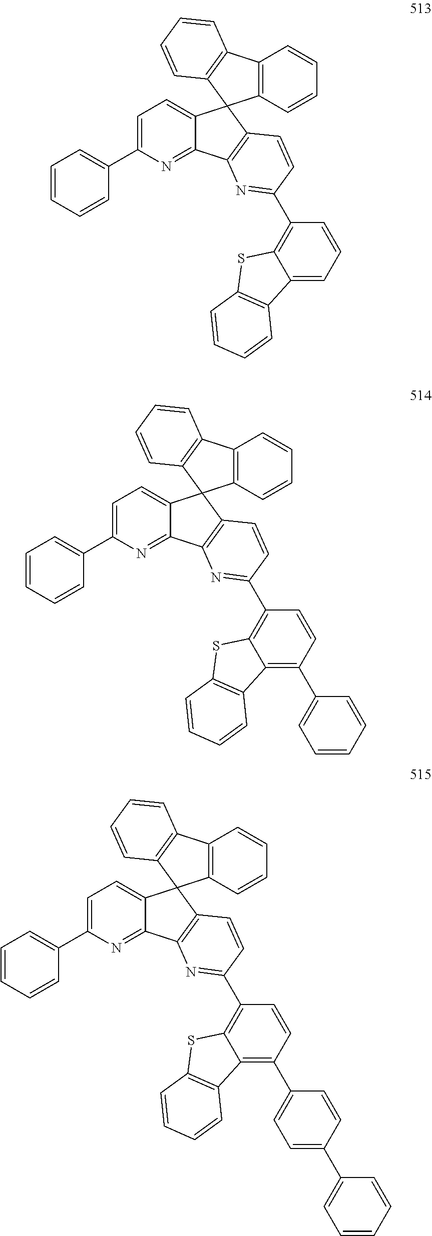 [SMILES: c1ccc(-c2ccc3c(n2)-c2nc(-c4cccc5c4sc4ccccc45)ccc2C32c3ccccc3-c3ccccc32)cc1 ; c1ccc(-c2ccc3c(n2)-c2nc(-c4ccc(-c5ccccc5)c5c4sc4ccccc45)ccc2C32c3ccccc3-c3ccccc32)cc1 ; c1ccc(-c2ccc(-c3ccc(-c4ccc5c(n4)-c4nc(-c6ccccc6)ccc4C54c5ccccc5-c5ccccc54)c4sc5ccccc5c34)cc2)cc1]

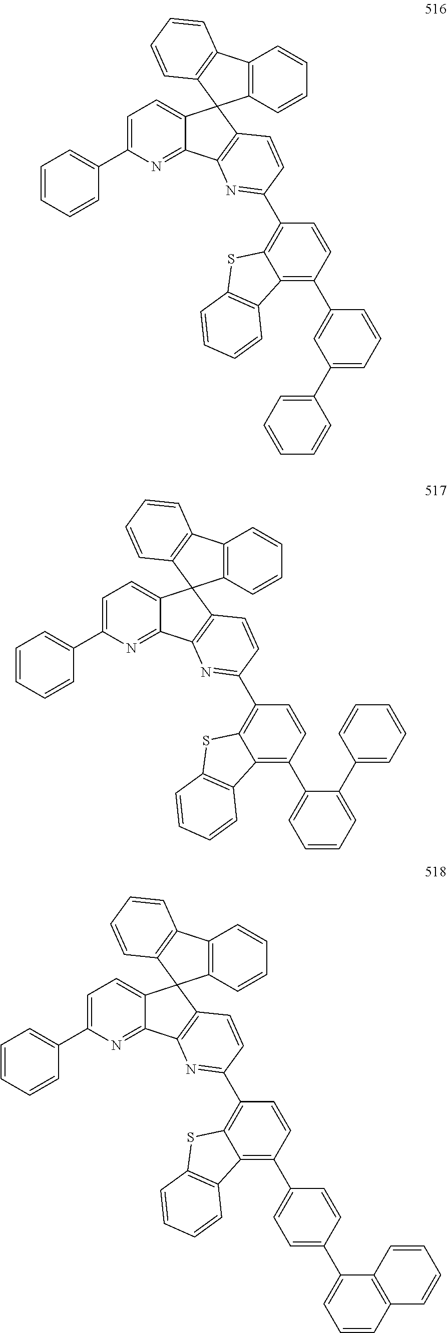 [SMILES: c1ccc(-c2cccc(-c3ccc(-c4ccc5c(n4)-c4nc(-c6ccccc6)ccc4C54c5ccccc5-c5ccccc54)c4sc5ccccc5c34)c2)cc1 ; c1ccc(-c2ccc3c(n2)-c2nc(-c4ccc(-c5ccccc5-c5ccccc5)c5c4sc4ccccc45)ccc2C32c3ccccc3-c3ccccc32)cc1 ; c1ccc(-c2ccc3c(n2)-c2nc(-c4ccc(-c5ccc(-c6cccc7ccccc67)cc5)c5c4sc4ccccc45)ccc2C32c3ccccc3-c3ccccc32)cc1]

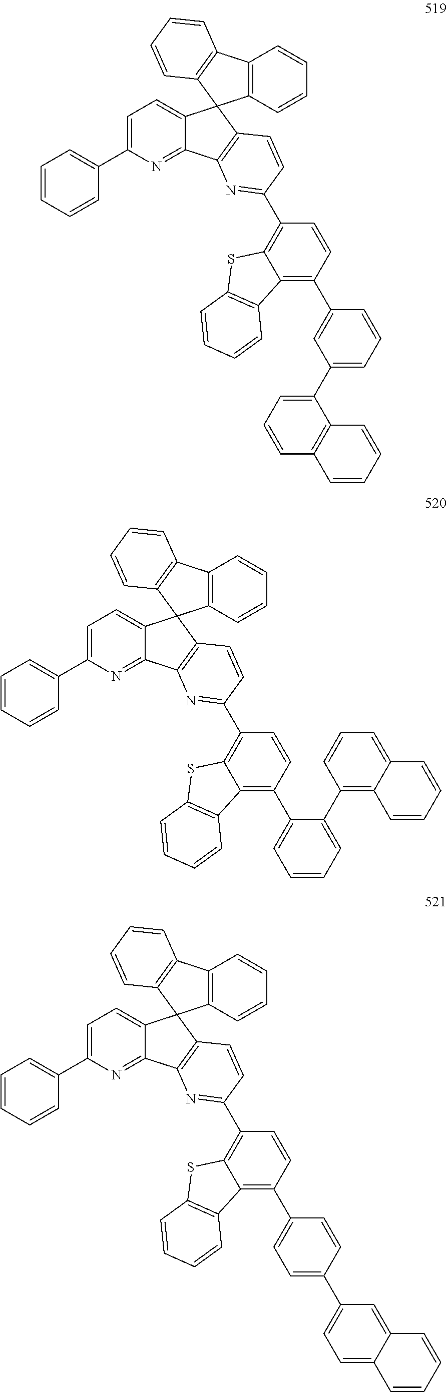 [SMILES: c1ccc(-c2ccc3c(n2)-c2nc(-c4ccc(-c5cccc(-c6cccc7ccccc67)c5)c5c4sc4ccccc45)ccc2C32c3ccccc3-c3ccccc32)cc1 ; c1ccc(-c2ccc3c(n2)-c2nc(-c4ccc(-c5ccccc5-c5cccc6ccccc56)c5c4sc4ccccc45)ccc2C32c3ccccc3-c3ccccc32)cc1 ; c1ccc(-c2ccc3c(n2)-c2nc(-c4ccc(-c5ccc(-c6ccc7ccccc7c6)cc5)c5c4sc4ccccc45)ccc2C32c3ccccc3-c3ccccc32)cc1]

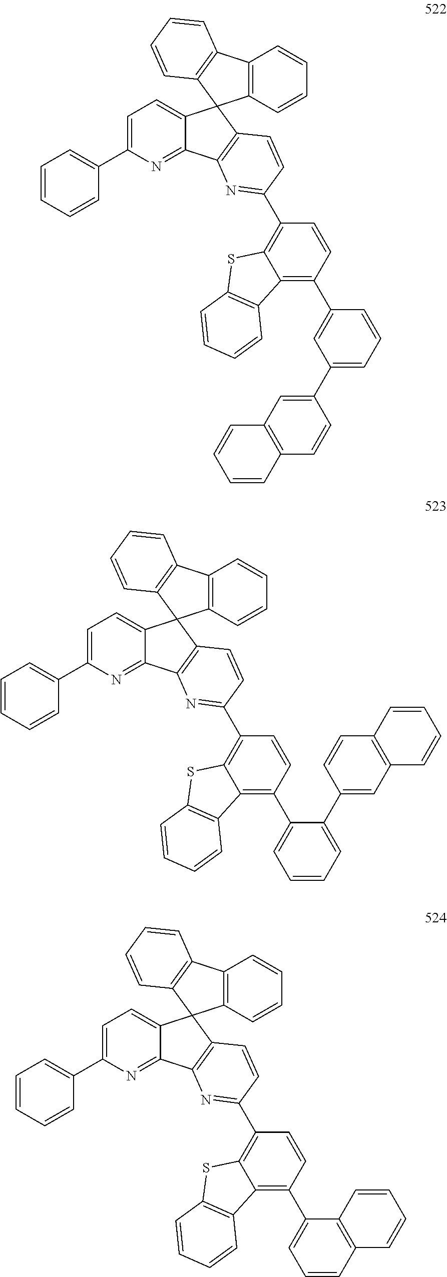 [SMILES: c1ccc(-c2ccc3c(n2)-c2nc(-c4ccc(-c5cccc(-c6ccc7ccccc7c6)c5)c5c4sc4ccccc45)ccc2C32c3ccccc3-c3ccccc32)cc1 ; c1ccc(-c2ccc3c(n2)-c2nc(-c4ccc(-c5ccccc5-c5ccc6ccccc6c5)c5c4sc4ccccc45)ccc2C32c3ccccc3-c3ccccc32)cc1 ; c1ccc(-c2ccc3c(n2)-c2nc(-c4ccc(-c5cccc6ccccc56)c5c4sc4ccccc45)ccc2C32c3ccccc3-c3ccccc32)cc1]

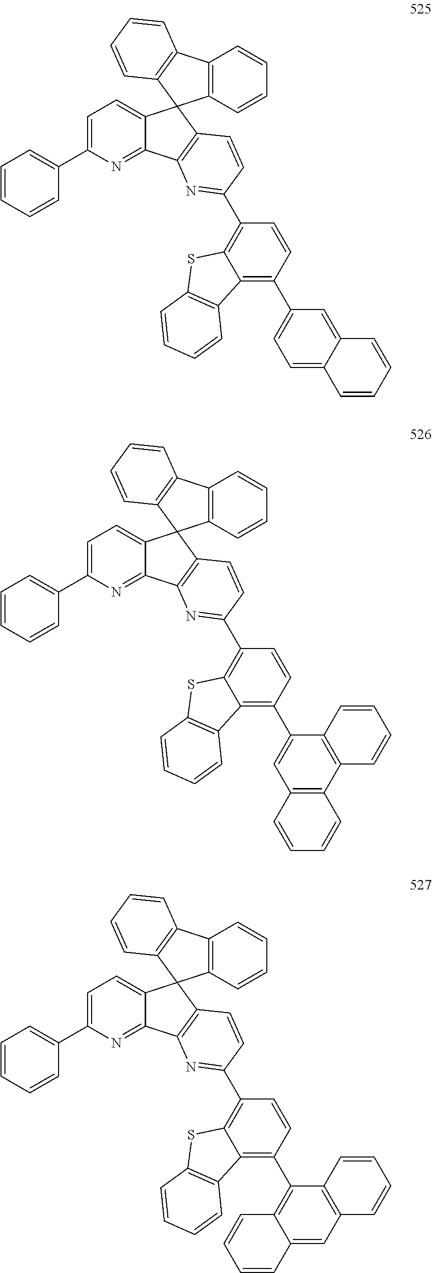 [SMILES: c1ccc(-c2ccc3c(n2)-c2nc(-c4ccc(-c5ccc6ccccc6c5)c5c4sc4ccccc45)ccc2C32c3ccccc3-c3ccccc32)cc1 ; c1ccc(-c2ccc3c(n2)-c2nc(-c4ccc(-c5cc6ccccc6c6ccccc56)c5c4sc4ccccc45)ccc2C32c3ccccc3-c3ccccc32)cc1 ; c1ccc(-c2ccc3c(n2)-c2nc(-c4ccc(-c5c6ccccc6cc6ccccc56)c5c4sc4ccccc45)ccc2C32c3ccccc3-c3ccccc32)cc1]

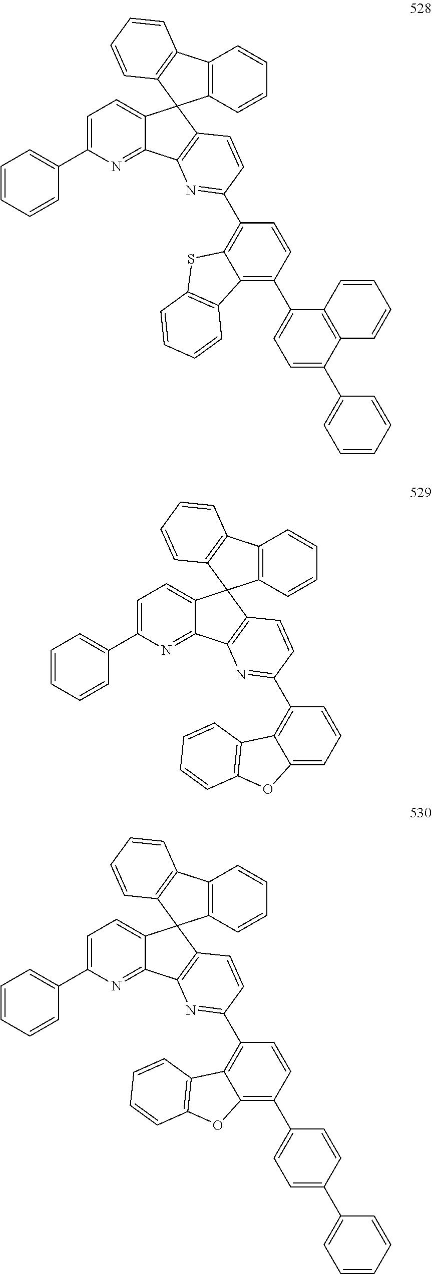 [SMILES: c1ccc(-c2ccc3c(n2)-c2nc(-c4ccc(-c5ccc(-c6ccccc6)c6ccccc56)c5c4sc4ccccc45)ccc2C32c3ccccc3-c3ccccc32)cc1 ; c1ccc(-c2ccc3c(n2)-c2nc(-c4cccc5oc6ccccc6c45)ccc2C32c3ccccc3-c3ccccc32)cc1 ; c1ccc(-c2ccc(-c3ccc(-c4ccc5c(n4)-c4nc(-c6ccccc6)ccc4C54c5ccccc5-c5ccccc54)c4c3oc3ccccc34)cc2)cc1]

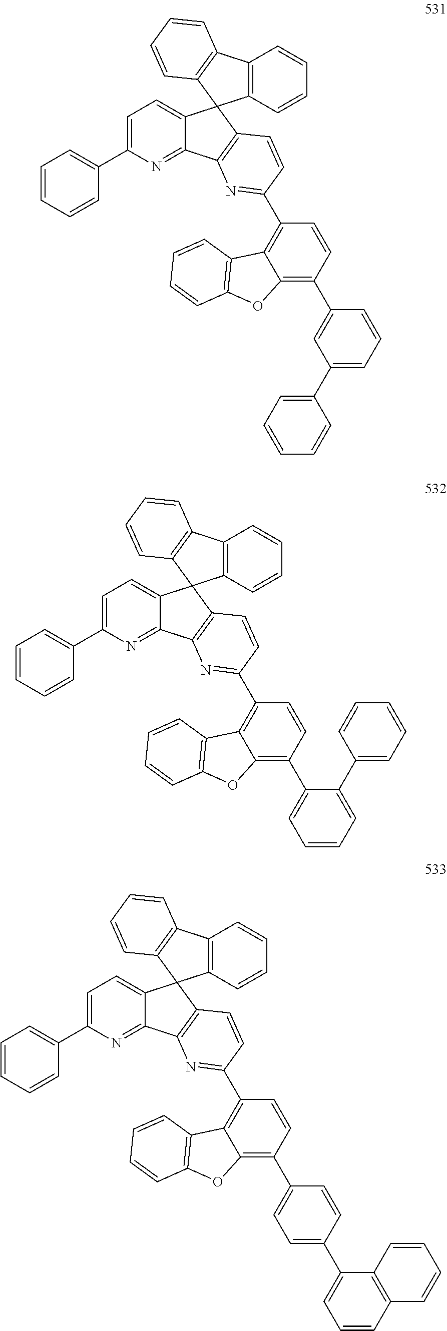 [SMILES: c1ccc(-c2cccc(-c3ccc(-c4ccc5c(n4)-c4nc(-c6ccccc6)ccc4C54c5ccccc5-c5ccccc54)c4c3oc3ccccc34)c2)cc1 ; c1ccc(-c2ccc3c(n2)-c2nc(-c4ccc(-c5ccccc5-c5ccccc5)c5oc6ccccc6c45)ccc2C32c3ccccc3-c3ccccc32)cc1 ; c1ccc(-c2ccc3c(n2)-c2nc(-c4ccc(-c5ccc(-c6cccc7ccccc67)cc5)c5oc6ccccc6c45)ccc2C32c3ccccc3-c3ccccc32)cc1]

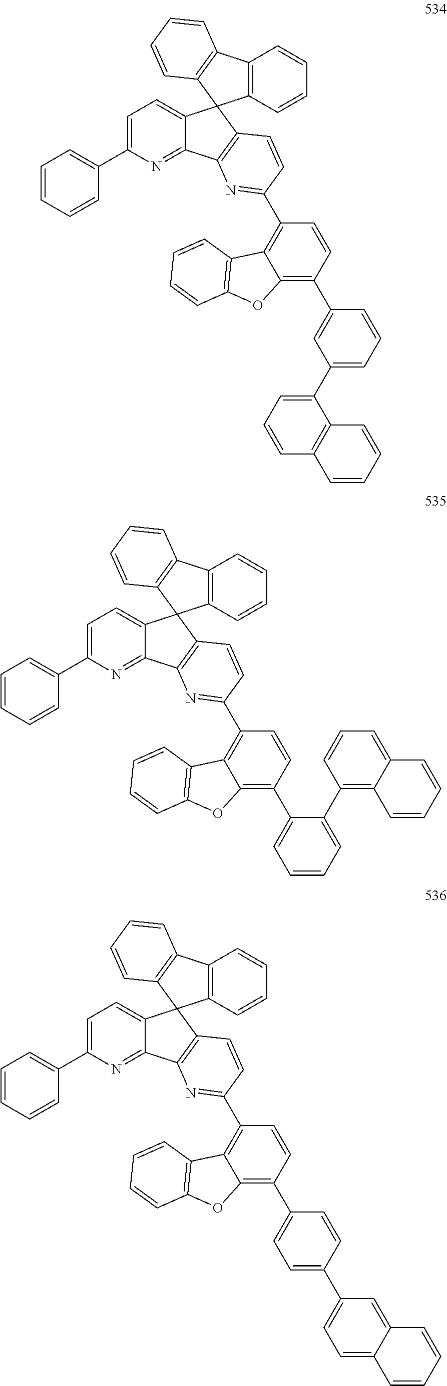 [SMILES: c1ccc(-c2ccc3c(n2)-c2nc(-c4ccc(-c5cccc(-c6cccc7ccccc67)c5)c5oc6ccccc6c45)ccc2C32c3ccccc3-c3ccccc32)cc1 ; c1ccc(-c2ccc3c(n2)-c2nc(-c4ccc(-c5ccccc5-c5cccc6ccccc56)c5oc6ccccc6c45)ccc2C32c3ccccc3-c3ccccc32)cc1 ; c1ccc(-c2ccc3c(n2)-c2nc(-c4ccc(-c5ccc(-c6ccc7ccccc7c6)cc5)c5oc6ccccc6c45)ccc2C32c3ccccc3-c3ccccc32)cc1]

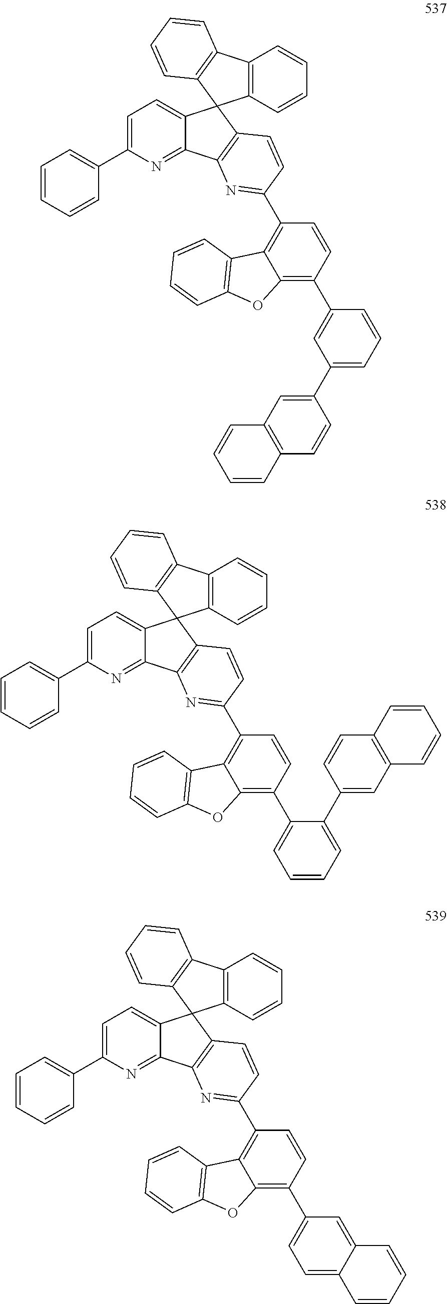 [SMILES: c1ccc(-c2ccc3c(n2)-c2nc(-c4ccc(-c5cccc(-c6ccc7ccccc7c6)c5)c5oc6ccccc6c45)ccc2C32c3ccccc3-c3ccccc32)cc1 ; c1ccc(-c2ccc3c(n2)-c2nc(-c4ccc(-c5ccccc5-c5ccc6ccccc6c5)c5oc6ccccc6c45)ccc2C32c3ccccc3-c3ccccc32)cc1 ; c1ccc(-c2ccc3c(n2)-c2nc(-c4ccc(-c5ccc6ccccc6c5)c5oc6ccccc6c45)ccc2C32c3ccccc3-c3ccccc32)cc1]

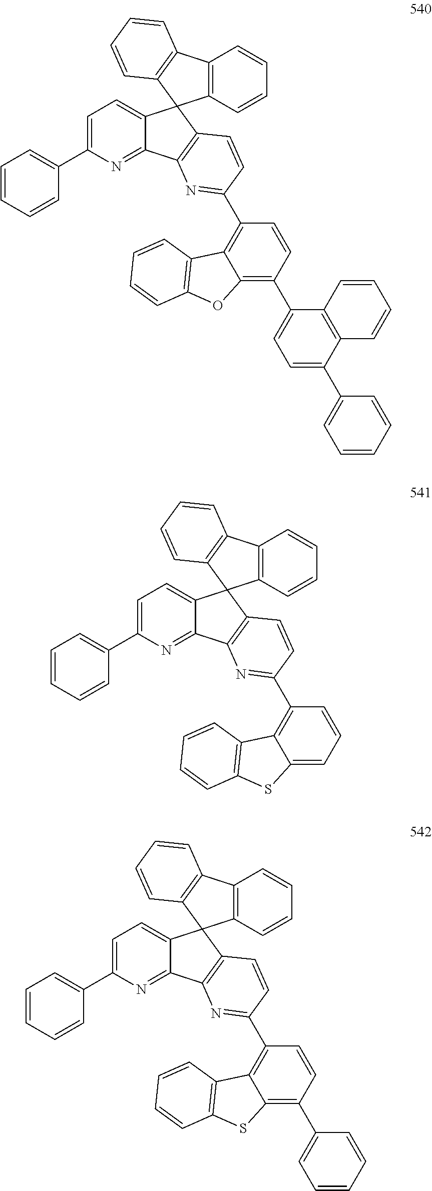 [SMILES: c1ccc(-c2ccc3c(n2)-c2nc(-c4ccc(-c5ccc(-c6ccccc6)c6ccccc56)c5oc6ccccc6c45)ccc2C32c3ccccc3-c3ccccc32)cc1 ; c1ccc(-c2ccc3c(n2)-c2nc(-c4cccc5sc6ccccc6c45)ccc2C32c3ccccc3-c3ccccc32)cc1 ; c1ccc(-c2ccc3c(n2)-c2nc(-c4ccc(-c5ccccc5)c5sc6ccccc6c45)ccc2C32c3ccccc3-c3ccccc32)cc1]

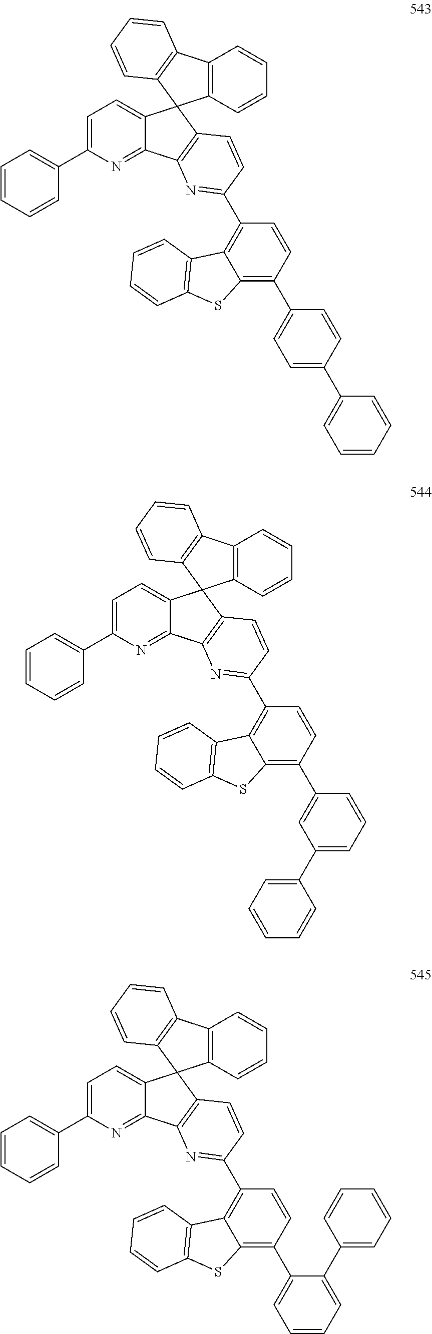 [SMILES: c1ccc(-c2ccc(-c3ccc(-c4ccc5c(n4)-c4nc(-c6ccccc6)ccc4C54c5ccccc5-c5ccccc54)c4c3sc3ccccc34)cc2)cc1 ; c1ccc(-c2cccc(-c3ccc(-c4ccc5c(n4)-c4nc(-c6ccccc6)ccc4C54c5ccccc5-c5ccccc54)c4c3sc3ccccc34)c2)cc1 ; c1ccc(-c2ccc3c(n2)-c2nc(-c4ccc(-c5ccccc5-c5ccccc5)c5sc6ccccc6c45)ccc2C32c3ccccc3-c3ccccc32)cc1]

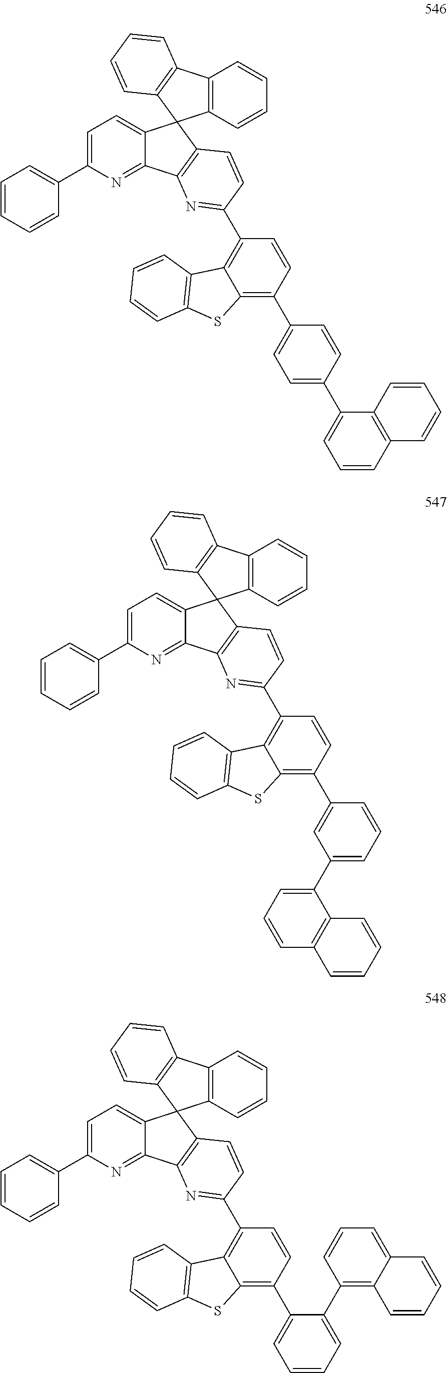 [SMILES: c1ccc(-c2ccc3c(n2)-c2nc(-c4ccc(-c5ccc(-c6cccc7ccccc67)cc5)c5sc6ccccc6c45)ccc2C32c3ccccc3-c3ccccc32)cc1 ; c1ccc(-c2ccc3c(n2)-c2nc(-c4ccc(-c5cccc(-c6cccc7ccccc67)c5)c5sc6ccccc6c45)ccc2C32c3ccccc3-c3ccccc32)cc1 ; c1ccc(-c2ccc3c(n2)-c2nc(-c4ccc(-c5ccccc5-c5cccc6ccccc56)c5sc6ccccc6c45)ccc2C32c3ccccc3-c3ccccc32)cc1]

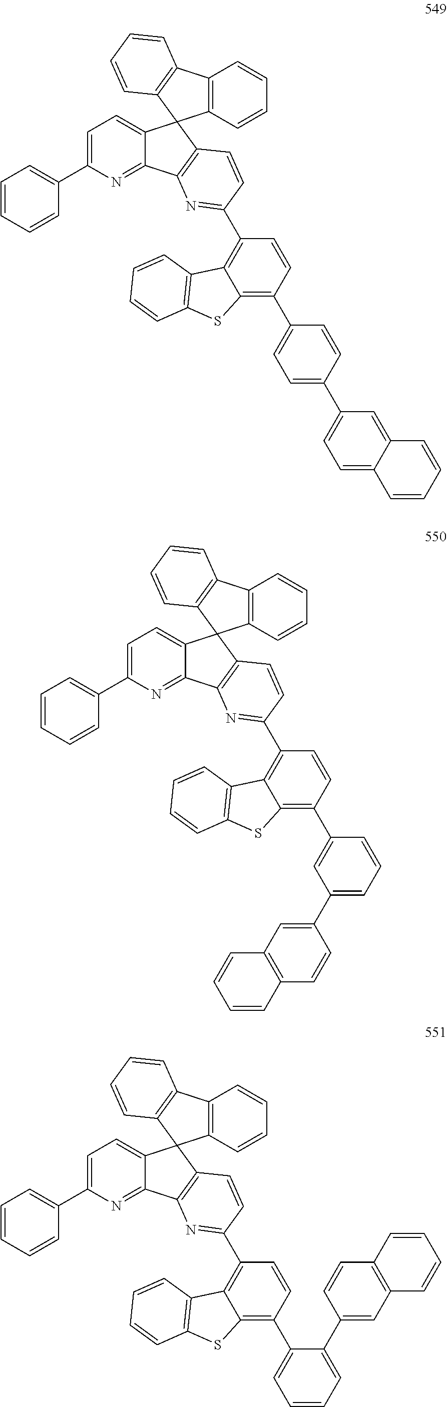 [SMILES: c1ccc(-c2ccc3c(n2)-c2nc(-c4ccc(-c5ccc(-c6ccc7ccccc7c6)cc5)c5sc6ccccc6c45)ccc2C32c3ccccc3-c3ccccc32)cc1 ; c1ccc(-c2ccc3c(n2)-c2nc(-c4ccc(-c5cccc(-c6ccc7ccccc7c6)c5)c5sc6ccccc6c45)ccc2C32c3ccccc3-c3ccccc32)cc1 ; c1ccc(-c2ccc3c(n2)-c2nc(-c4ccc(-c5ccccc5-c5ccc6ccccc6c5)c5sc6ccccc6c45)ccc2C32c3ccccc3-c3ccccc32)cc1]

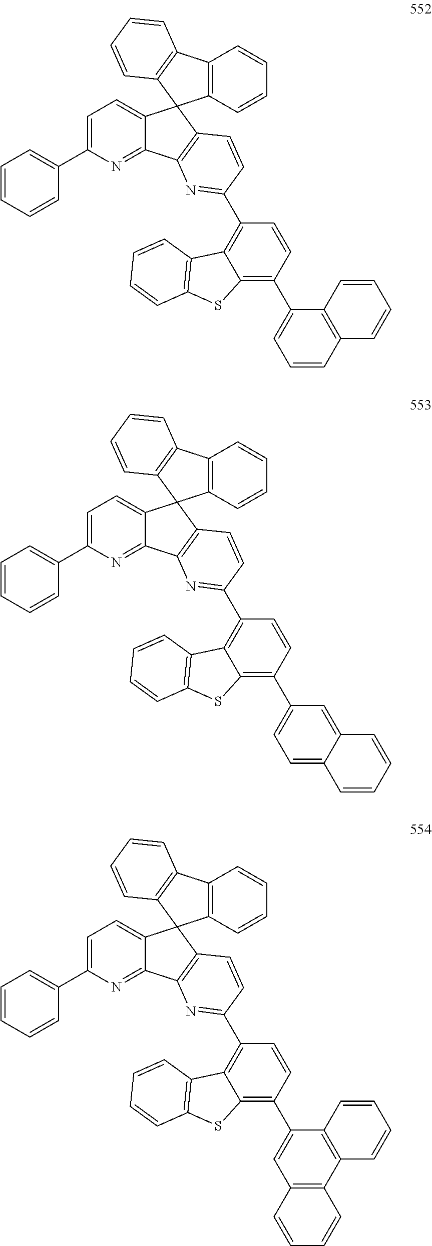 [SMILES: c1ccc(-c2ccc3c(n2)-c2nc(-c4ccc(-c5cccc6ccccc56)c5sc6ccccc6c45)ccc2C32c3ccccc3-c3ccccc32)cc1 ; c1ccc(-c2ccc3c(n2)-c2nc(-c4ccc(-c5ccc6ccccc6c5)c5sc6ccccc6c45)ccc2C32c3ccccc3-c3ccccc32)cc1 ; c1ccc(-c2ccc3c(n2)-c2nc(-c4ccc(-c5cc6ccccc6c6ccccc56)c5sc6ccccc6c45)ccc2C32c3ccccc3-c3ccccc32)cc1]

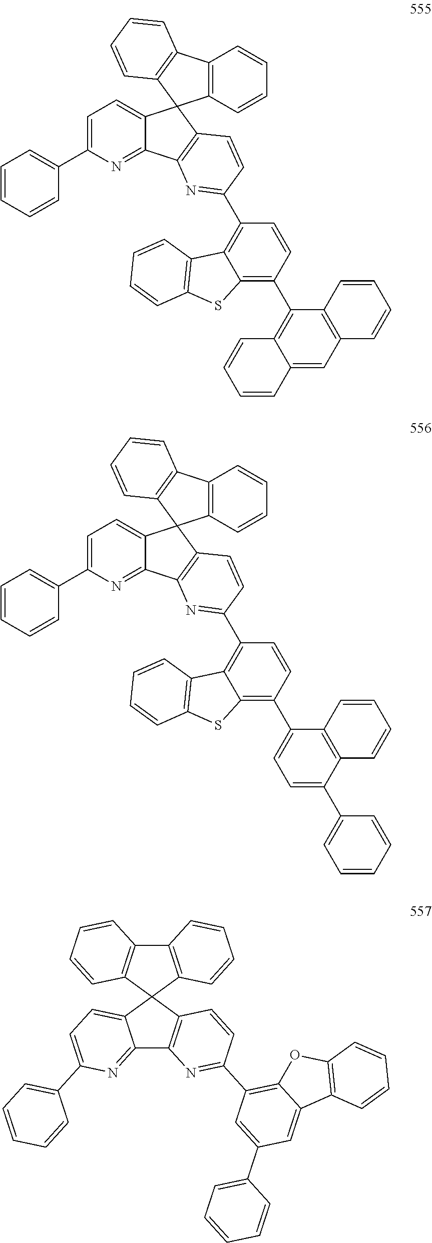 [SMILES: c1ccc(-c2ccc3c(n2)-c2nc(-c4ccc(-c5c6ccccc6cc6ccccc56)c5sc6ccccc6c45)ccc2C32c3ccccc3-c3ccccc32)cc1 ; c1ccc(-c2ccc3c(n2)-c2nc(-c4ccc(-c5ccc(-c6ccccc6)c6ccccc56)c5sc6ccccc6c45)ccc2C32c3ccccc3-c3ccccc32)cc1 ; c1ccc(-c2cc(-c3ccc4c(n3)-c3nc(-c5ccccc5)ccc3C43c4ccccc4-c4ccccc43)c3oc4ccccc4c3c2)cc1]

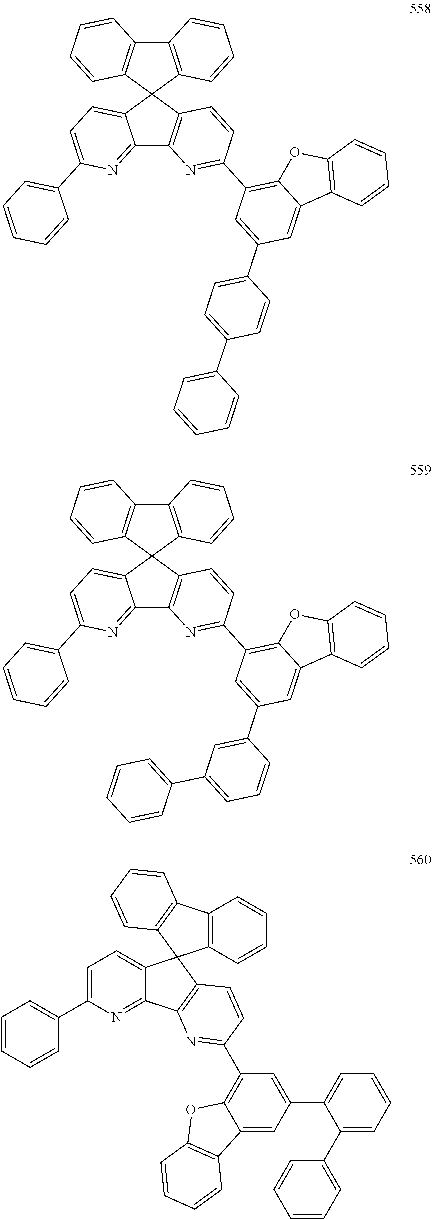

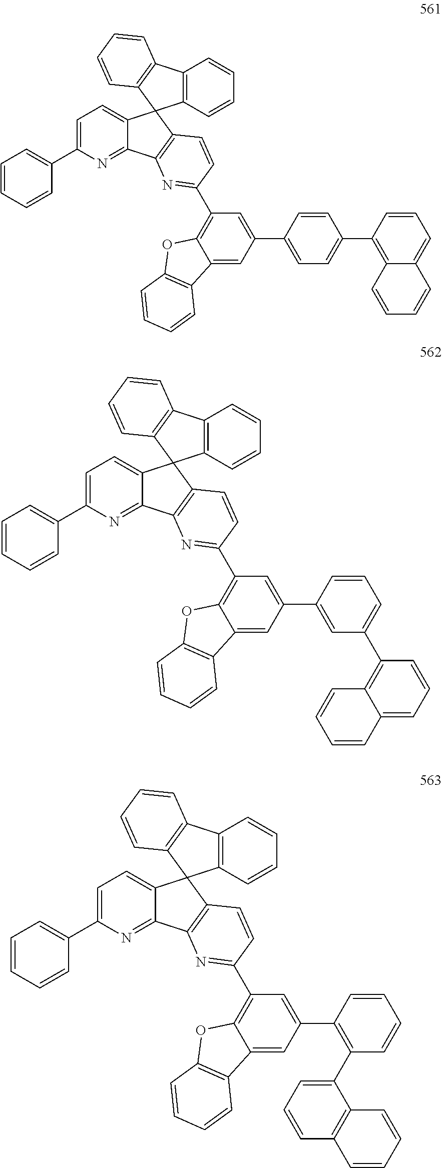 [SMILES: c1ccc(-c2ccc3c(n2)-c2nc(-c4cc(-c5ccc(-c6cccc7ccccc67)cc5)cc5c4oc4ccccc45)ccc2C32c3ccccc3-c3ccccc32)cc1 ; c1ccc(-c2ccc3c(n2)-c2nc(-c4cc(-c5cccc(-c6cccc7ccccc67)c5)cc5c4oc4ccccc45)ccc2C32c3ccccc3-c3ccccc32)cc1 ; c1ccc(-c2ccc3c(n2)-c2nc(-c4cc(-c5ccccc5-c5cccc6ccccc56)cc5c4oc4ccccc45)ccc2C32c3ccccc3-c3ccccc32)cc1]

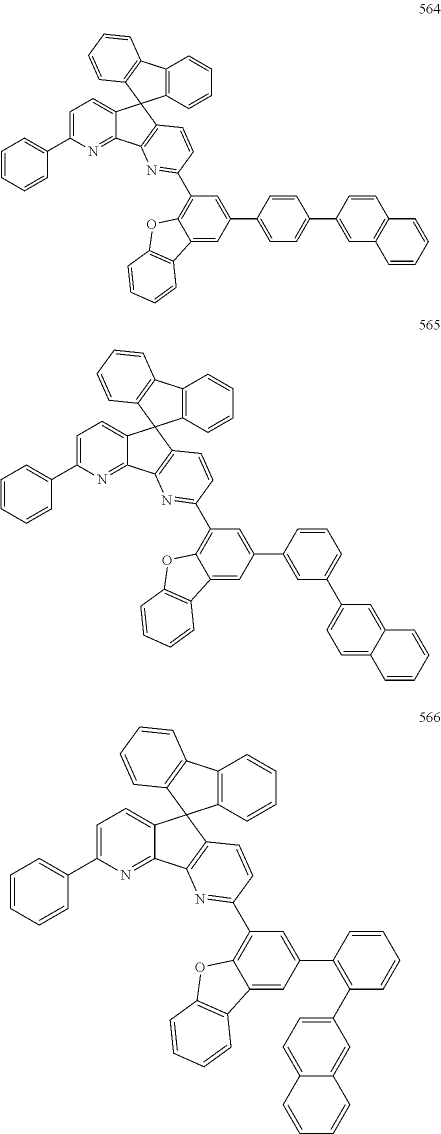 [SMILES: c1ccc(-c2ccc3c(n2)-c2nc(-c4cc(-c5ccc(-c6ccc7ccccc7c6)cc5)cc5c4oc4ccccc45)ccc2C32c3ccccc3-c3ccccc32)cc1 ; c1ccc(-c2ccc3c(n2)-c2nc(-c4cc(-c5cccc(-c6ccc7ccccc7c6)c5)cc5c4oc4ccccc45)ccc2C32c3ccccc3-c3ccccc32)cc1 ; c1ccc(-c2ccc3c(n2)-c2nc(-c4cc(-c5ccccc5-c5ccc6ccccc6c5)cc5c4oc4ccccc45)ccc2C32c3ccccc3-c3ccccc32)cc1]

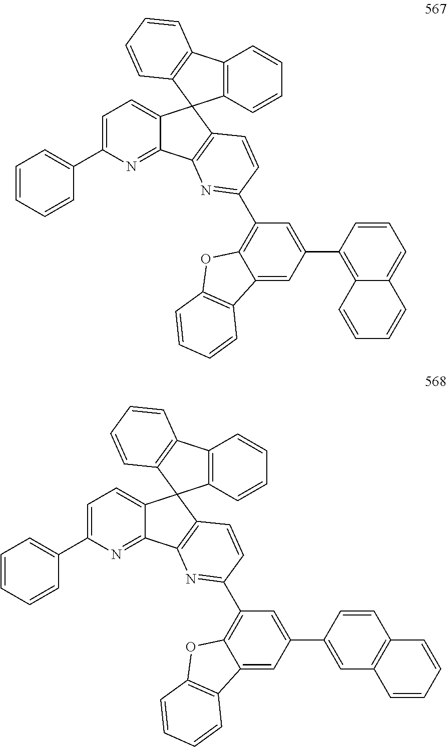 [SMILES: c1ccc(-c2ccc3c(n2)-c2nc(-c4cc(-c5cccc6ccccc56)cc5c4oc4ccccc45)ccc2C32c3ccccc3-c3ccccc32)cc1 ; c1ccc(-c2ccc3c(n2)-c2nc(-c4cc(-c5ccc6ccccc6c5)cc5c4oc4ccccc45)ccc2C32c3ccccc3-c3ccccc32)cc1]

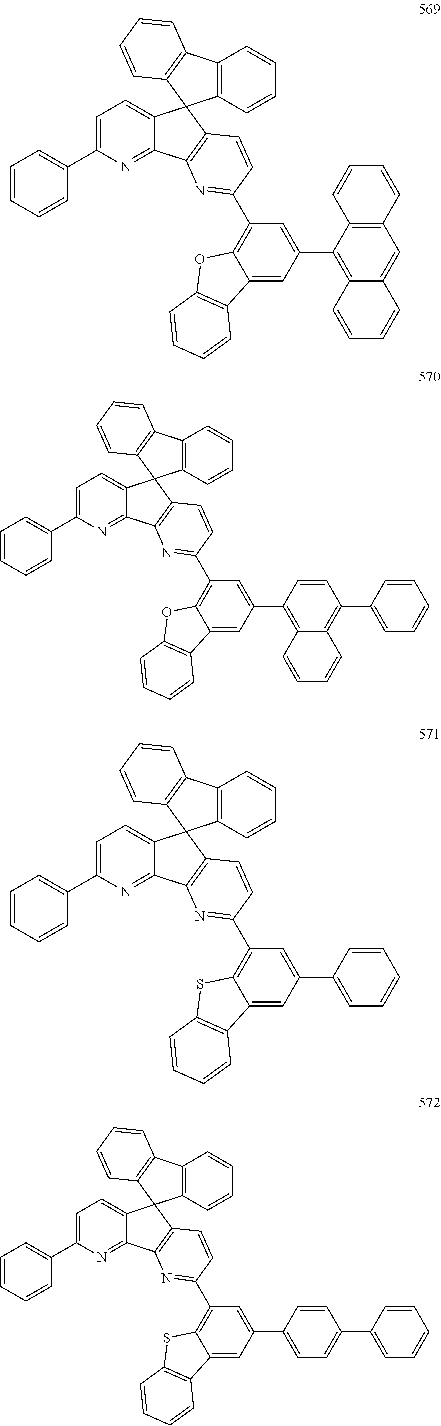 [SMILES: c1ccc(-c2ccc3c(n2)-c2nc(-c4cc(-c5c6ccccc6cc6ccccc56)cc5c4oc4ccccc45)ccc2C32c3ccccc3-c3ccccc32)cc1 ; c1ccc(-c2ccc3c(n2)-c2nc(-c4cc(-c5ccc(-c6ccccc6)c6ccccc56)cc5c4oc4ccccc45)ccc2C32c3ccccc3-c3ccccc32)cc1 ; c1ccc(-c2cc(-c3ccc4c(n3)-c3nc(-c5ccccc5)ccc3C43c4ccccc4-c4ccccc43)c3sc4ccccc4c3c2)cc1 ; c1ccc(-c2ccc(-c3cc(-c4ccc5c(n4)-c4nc(-c6ccccc6)ccc4C54c5ccccc5-c5ccccc54)c4sc5ccccc5c4c3)cc2)cc1]

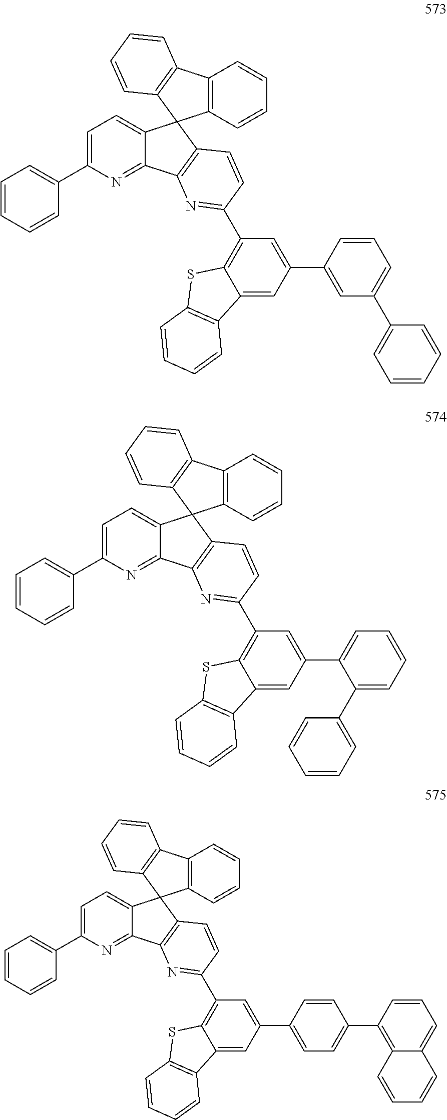 [SMILES: c1ccc(-c2cccc(-c3cc(-c4ccc5c(n4)-c4nc(-c6ccccc6)ccc4C54c5ccccc5-c5ccccc54)c4sc5ccccc5c4c3)c2)cc1 ; c1ccc(-c2ccc3c(n2)-c2nc(-c4cc(-c5ccccc5-c5ccccc5)cc5c4sc4ccccc45)ccc2C32c3ccccc3-c3ccccc32)cc1 ; c1ccc(-c2ccc3c(n2)-c2nc(-c4cc(-c5ccc(-c6cccc7ccccc67)cc5)cc5c4sc4ccccc45)ccc2C32c3ccccc3-c3ccccc32)cc1]

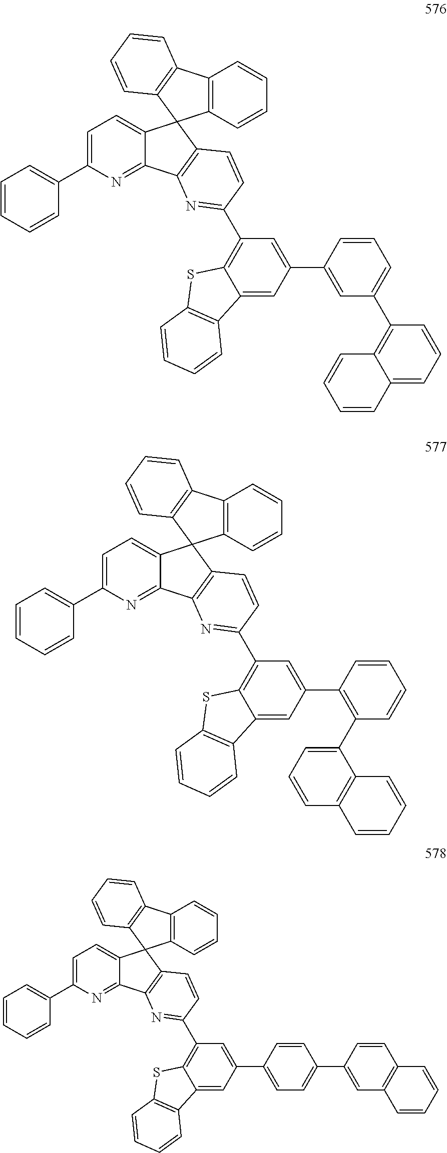 [SMILES: c1ccc(-c2ccc3c(n2)-c2nc(-c4cc(-c5cccc(-c6cccc7ccccc67)c5)cc5c4sc4ccccc45)ccc2C32c3ccccc3-c3ccccc32)cc1 ; c1ccc(-c2ccc3c(n2)-c2nc(-c4cc(-c5ccccc5-c5cccc6ccccc56)cc5c4sc4ccccc45)ccc2C32c3ccccc3-c3ccccc32)cc1 ; c1ccc(-c2ccc3c(n2)-c2nc(-c4cc(-c5ccc(-c6ccc7ccccc7c6)cc5)cc5c4sc4ccccc45)ccc2C32c3ccccc3-c3ccccc32)cc1]

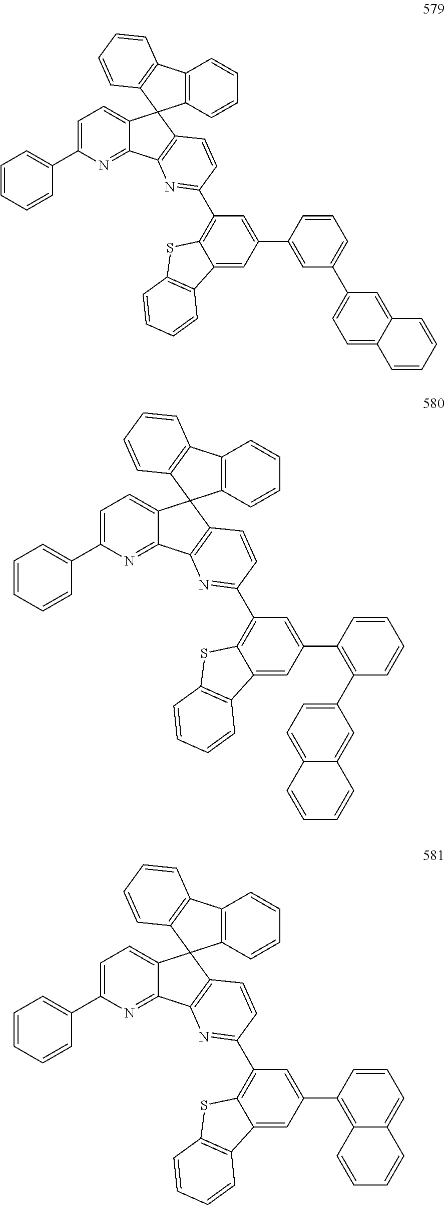 [SMILES: c1ccc(-c2ccc3c(n2)-c2nc(-c4cc(-c5cccc(-c6ccc7ccccc7c6)c5)cc5c4sc4ccccc45)ccc2C32c3ccccc3-c3ccccc32)cc1 ; c1ccc(-c2ccc3c(n2)-c2nc(-c4cc(-c5ccccc5-c5ccc6ccccc6c5)cc5c4sc4ccccc45)ccc2C32c3ccccc3-c3ccccc32)cc1 ; c1ccc(-c2ccc3c(n2)-c2nc(-c4cc(-c5cccc6ccccc56)cc5c4sc4ccccc45)ccc2C32c3ccccc3-c3ccccc32)cc1]

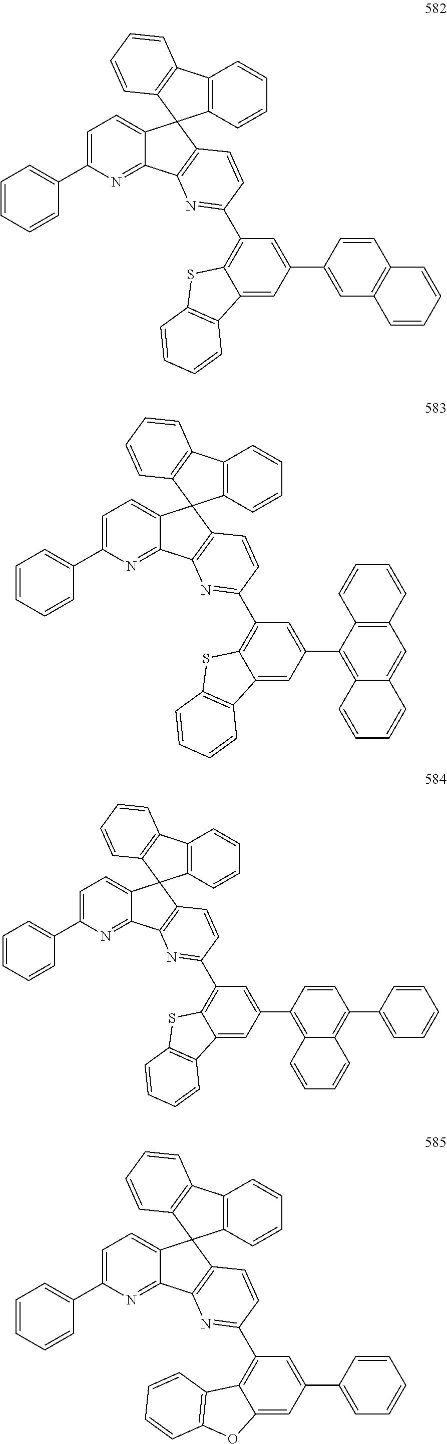 [SMILES: c1ccc(-c2ccc3c(n2)-c2nc(-c4cc(-c5ccc6ccccc6c5)cc5c4sc4ccccc45)ccc2C32c3ccccc3-c3ccccc32)cc1 ; c1ccc(-c2ccc3c(n2)-c2nc(-c4cc(-c5c6ccccc6cc6ccccc56)cc5c4sc4ccccc45)ccc2C32c3ccccc3-c3ccccc32)cc1 ; c1ccc(-c2ccc3c(n2)-c2nc(-c4cc(-c5ccc(-c6ccccc6)c6ccccc56)cc5c4sc4ccccc45)ccc2C32c3ccccc3-c3ccccc32)cc1 ; c1ccc(-c2cc(-c3ccc4c(n3)-c3nc(-c5ccccc5)ccc3C43c4ccccc4-c4ccccc43)c3c(c2)oc2ccccc23)cc1]

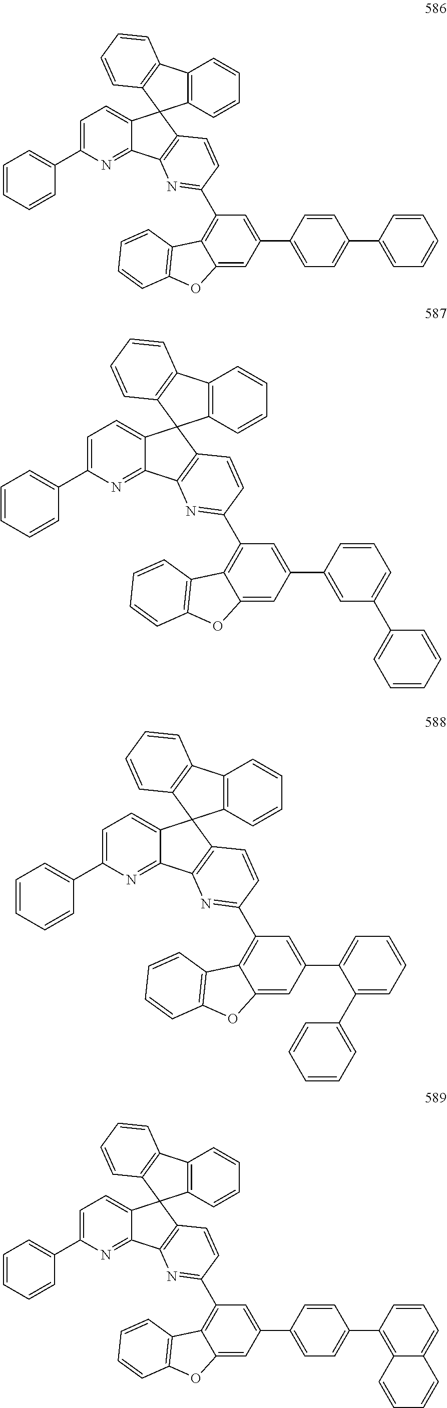 [SMILES: c1ccc(-c2ccc(-c3cc(-c4ccc5c(n4)-c4nc(-c6ccccc6)ccc4C54c5ccccc5-c5ccccc54)c4c(c3)oc3ccccc34)cc2)cc1 ; c1ccc(-c2cccc(-c3cc(-c4ccc5c(n4)-c4nc(-c6ccccc6)ccc4C54c5ccccc5-c5ccccc54)c4c(c3)oc3ccccc34)c2)cc1 ; c1ccc(-c2ccc3c(n2)-c2nc(-c4cc(-c5ccccc5-c5ccccc5)cc5oc6ccccc6c45)ccc2C32c3ccccc3-c3ccccc32)cc1 ; c1ccc(-c2ccc3c(n2)-c2nc(-c4cc(-c5ccc(-c6cccc7ccccc67)cc5)cc5oc6ccccc6c45)ccc2C32c3ccccc3-c3ccccc32)cc1]

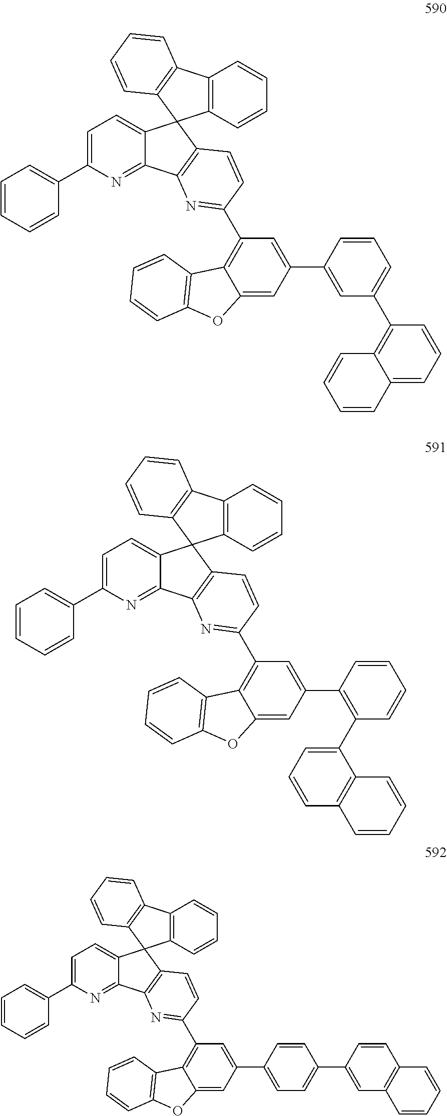 [SMILES: c1ccc(-c2ccc3c(n2)-c2nc(-c4cc(-c5cccc(-c6cccc7ccccc67)c5)cc5oc6ccccc6c45)ccc2C32c3ccccc3-c3ccccc32)cc1 ; c1ccc(-c2ccc3c(n2)-c2nc(-c4cc(-c5ccccc5-c5cccc6ccccc56)cc5oc6ccccc6c45)ccc2C32c3ccccc3-c3ccccc32)cc1 ; c1ccc(-c2ccc3c(n2)-c2nc(-c4cc(-c5ccc(-c6ccc7ccccc7c6)cc5)cc5oc6ccccc6c45)ccc2C32c3ccccc3-c3ccccc32)cc1]

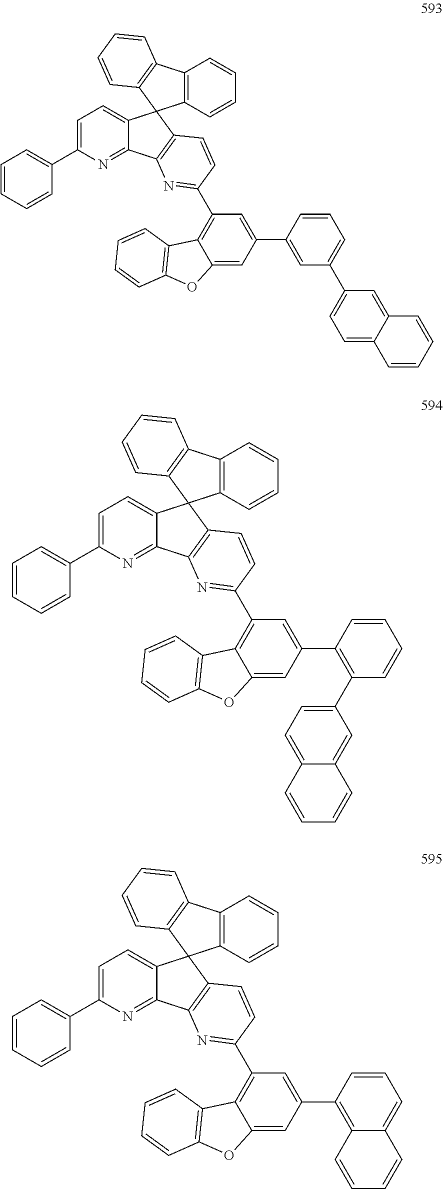 [SMILES: c1ccc(-c2ccc3c(n2)-c2nc(-c4cc(-c5cccc(-c6ccc7ccccc7c6)c5)cc5oc6ccccc6c45)ccc2C32c3ccccc3-c3ccccc32)cc1 ; c1ccc(-c2ccc3c(n2)-c2nc(-c4cc(-c5ccccc5-c5ccc6ccccc6c5)cc5oc6ccccc6c45)ccc2C32c3ccccc3-c3ccccc32)cc1 ; c1ccc(-c2ccc3c(n2)-c2nc(-c4cc(-c5cccc6ccccc56)cc5oc6ccccc6c45)ccc2C32c3ccccc3-c3ccccc32)cc1]

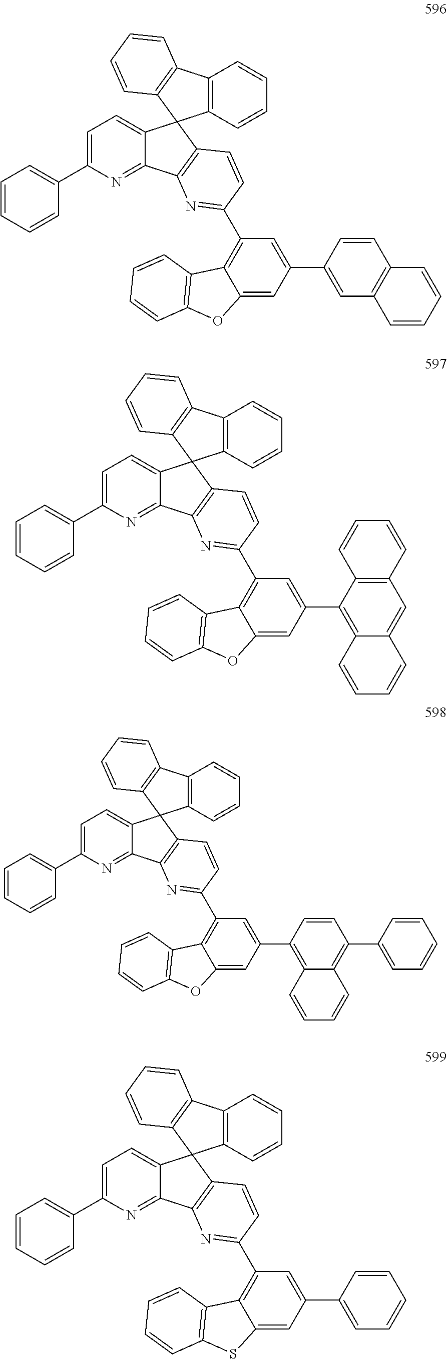 [SMILES: c1ccc(-c2ccc3c(n2)-c2nc(-c4cc(-c5ccc6ccccc6c5)cc5oc6ccccc6c45)ccc2C32c3ccccc3-c3ccccc32)cc1 ; c1ccc(-c2ccc3c(n2)-c2nc(-c4cc(-c5c6ccccc6cc6ccccc56)cc5oc6ccccc6c45)ccc2C32c3ccccc3-c3ccccc32)cc1 ; c1ccc(-c2ccc3c(n2)-c2nc(-c4cc(-c5ccc(-c6ccccc6)c6ccccc56)cc5oc6ccccc6c45)ccc2C32c3ccccc3-c3ccccc32)cc1 ; c1ccc(-c2cc(-c3ccc4c(n3)-c3nc(-c5ccccc5)ccc3C43c4ccccc4-c4ccccc43)c3c(c2)sc2ccccc23)cc1]

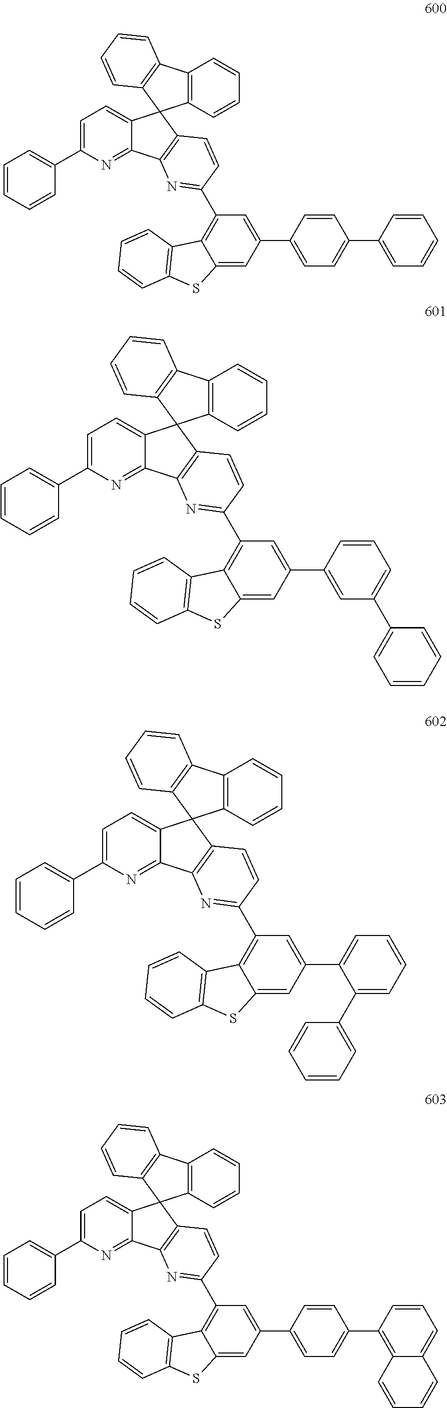 [SMILES: c1ccc(-c2ccc(-c3cc(-c4ccc5c(n4)-c4nc(-c6ccccc6)ccc4C54c5ccccc5-c5ccccc54)c4c(c3)sc3ccccc34)cc2)cc1 ; c1ccc(-c2cccc(-c3cc(-c4ccc5c(n4)-c4nc(-c6ccccc6)ccc4C54c5ccccc5-c5ccccc54)c4c(c3)sc3ccccc34)c2)cc1 ; c1ccc(-c2ccc3c(n2)-c2nc(-c4cc(-c5ccccc5-c5ccccc5)cc5sc6ccccc6c45)ccc2C32c3ccccc3-c3ccccc32)cc1 ; c1ccc(-c2ccc3c(n2)-c2nc(-c4cc(-c5ccc(-c6cccc7ccccc67)cc5)cc5sc6ccccc6c45)ccc2C32c3ccccc3-c3ccccc32)cc1]

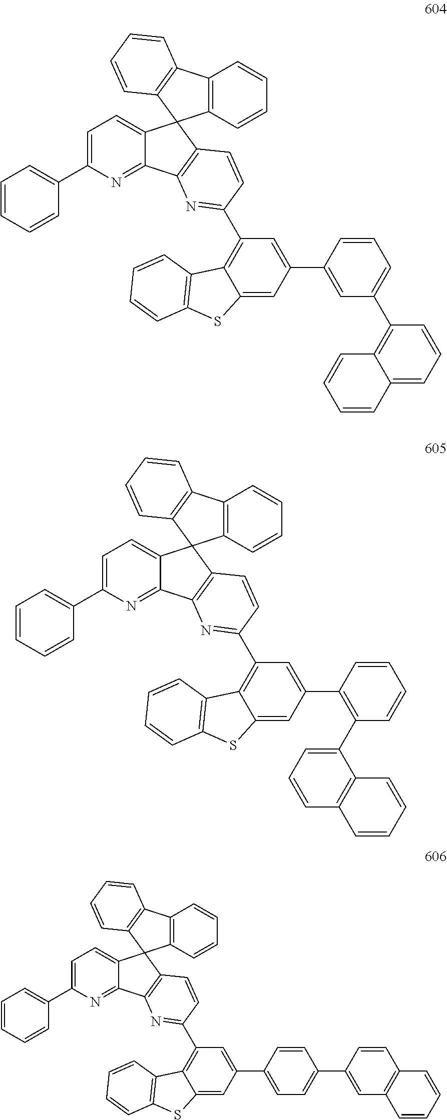 [SMILES: c1ccc(-c2ccc3c(n2)-c2nc(-c4cc(-c5cccc(-c6cccc7ccccc67)c5)cc5sc6ccccc6c45)ccc2C32c3ccccc3-c3ccccc32)cc1 ; c1ccc(-c2ccc3c(n2)-c2nc(-c4cc(-c5ccccc5-c5cccc6ccccc56)cc5sc6ccccc6c45)ccc2C32c3ccccc3-c3ccccc32)cc1 ; c1ccc(-c2ccc3c(n2)-c2nc(-c4cc(-c5ccc(-c6ccc7ccccc7c6)cc5)cc5sc6ccccc6c45)ccc2C32c3ccccc3-c3ccccc32)cc1]

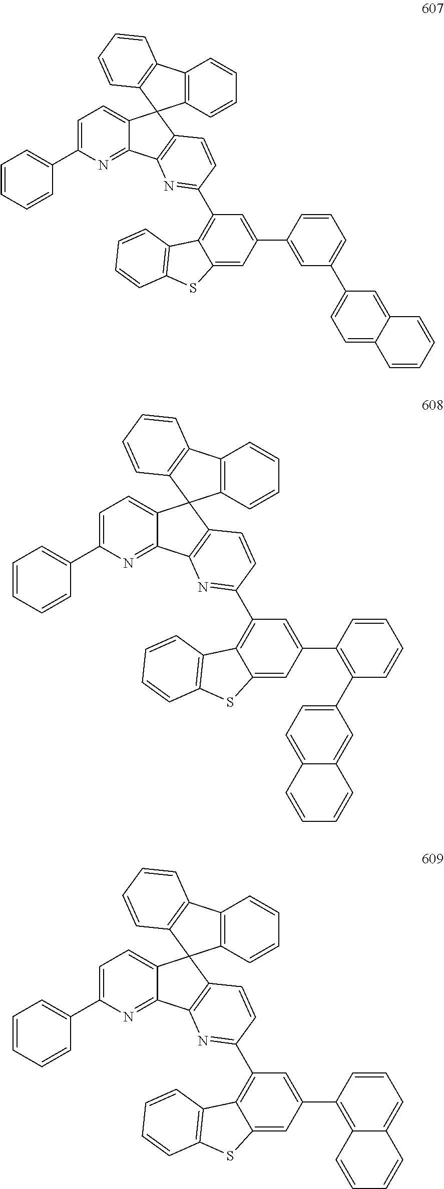 [SMILES: c1ccc(-c2ccc3c(n2)-c2nc(-c4cc(-c5cccc(-c6ccc7ccccc7c6)c5)cc5sc6ccccc6c45)ccc2C32c3ccccc3-c3ccccc32)cc1 ; c1ccc(-c2ccc3c(n2)-c2nc(-c4cc(-c5ccccc5-c5ccc6ccccc6c5)cc5sc6ccccc6c45)ccc2C32c3ccccc3-c3ccccc32)cc1 ; c1ccc(-c2ccc3c(n2)-c2nc(-c4cc(-c5cccc6ccccc56)cc5sc6ccccc6c45)ccc2C32c3ccccc3-c3ccccc32)cc1]

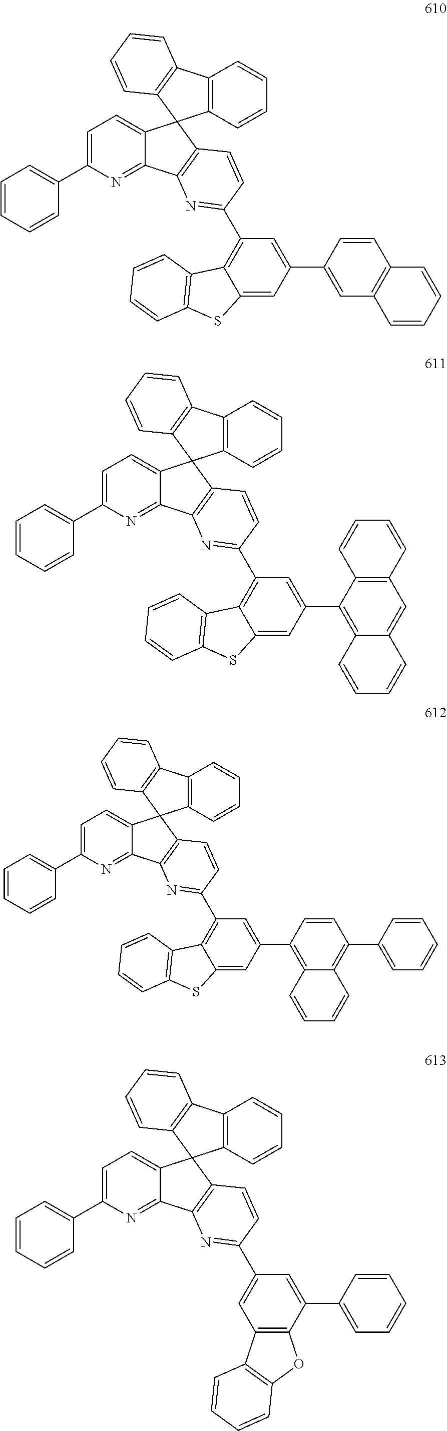 [SMILES: c1ccc(-c2ccc3c(n2)-c2nc(-c4cc(-c5ccc6ccccc6c5)cc5sc6ccccc6c45)ccc2C32c3ccccc3-c3ccccc32)cc1 ; c1ccc(-c2ccc3c(n2)-c2nc(-c4cc(-c5c6ccccc6cc6ccccc56)cc5sc6ccccc6c45)ccc2C32c3ccccc3-c3ccccc32)cc1 ; c1ccc(-c2ccc3c(n2)-c2nc(-c4cc(-c5ccc(-c6ccccc6)c6ccccc56)cc5sc6ccccc6c45)ccc2C32c3ccccc3-c3ccccc32)cc1 ; c1ccc(-c2ccc3c(n2)-c2nc(-c4cc(-c5ccccc5)c5oc6ccccc6c5c4)ccc2C32c3ccccc3-c3ccccc32)cc1]

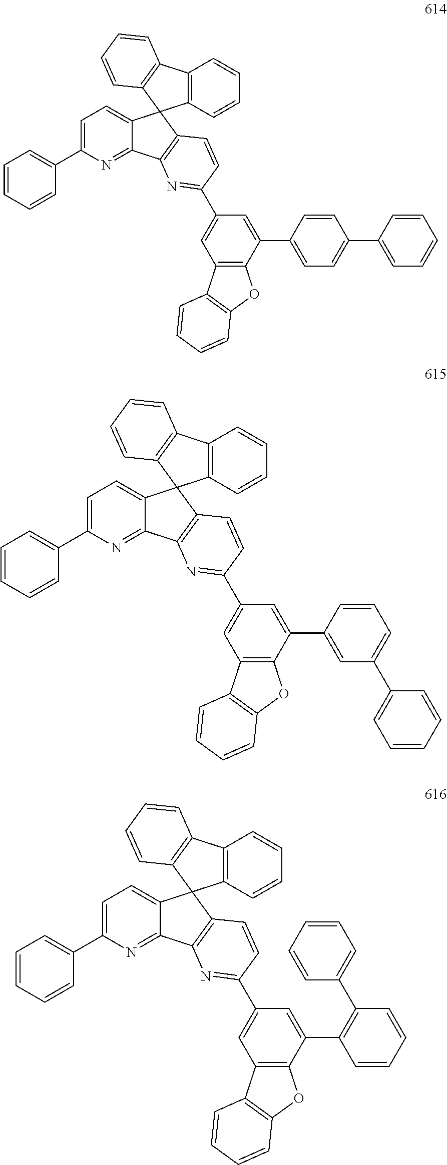 [SMILES: c1ccc(-c2ccc(-c3cc(-c4ccc5c(n4)-c4nc(-c6ccccc6)ccc4C54c5ccccc5-c5ccccc54)cc4c3oc3ccccc34)cc2)cc1 ; c1ccc(-c2cccc(-c3cc(-c4ccc5c(n4)-c4nc(-c6ccccc6)ccc4C54c5ccccc5-c5ccccc54)cc4c3oc3ccccc34)c2)cc1 ; c1ccc(-c2ccc3c(n2)-c2nc(-c4cc(-c5ccccc5-c5ccccc5)c5oc6ccccc6c5c4)ccc2C32c3ccccc3-c3ccccc32)cc1]

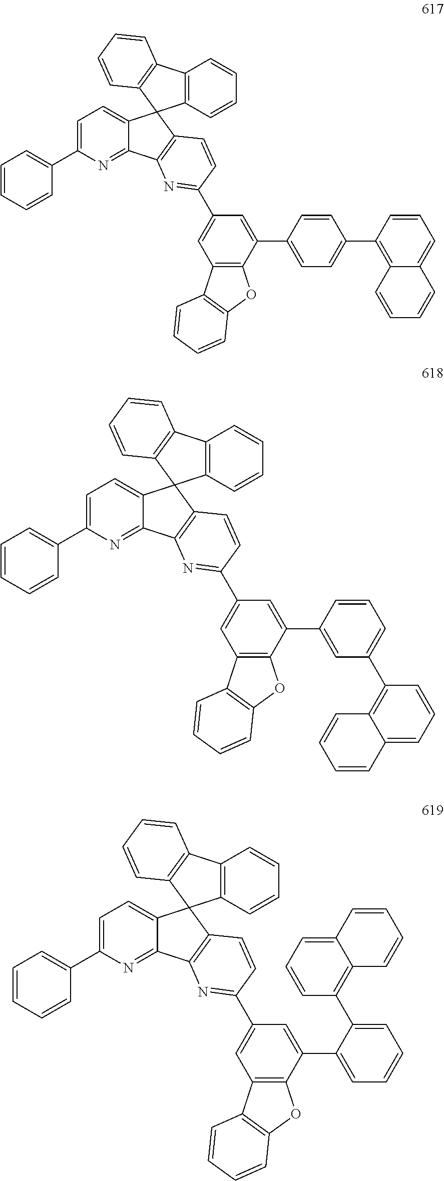 [SMILES: c1ccc(-c2ccc3c(n2)-c2nc(-c4cc(-c5ccc(-c6cccc7ccccc67)cc5)c5oc6ccccc6c5c4)ccc2C32c3ccccc3-c3ccccc32)cc1 ; c1ccc(-c2ccc3c(n2)-c2nc(-c4cc(-c5cccc(-c6cccc7ccccc67)c5)c5oc6ccccc6c5c4)ccc2C32c3ccccc3-c3ccccc32)cc1 ; c1ccc(-c2ccc3c(n2)-c2nc(-c4cc(-c5ccccc5-c5cccc6ccccc56)c5oc6ccccc6c5c4)ccc2C32c3ccccc3-c3ccccc32)cc1]

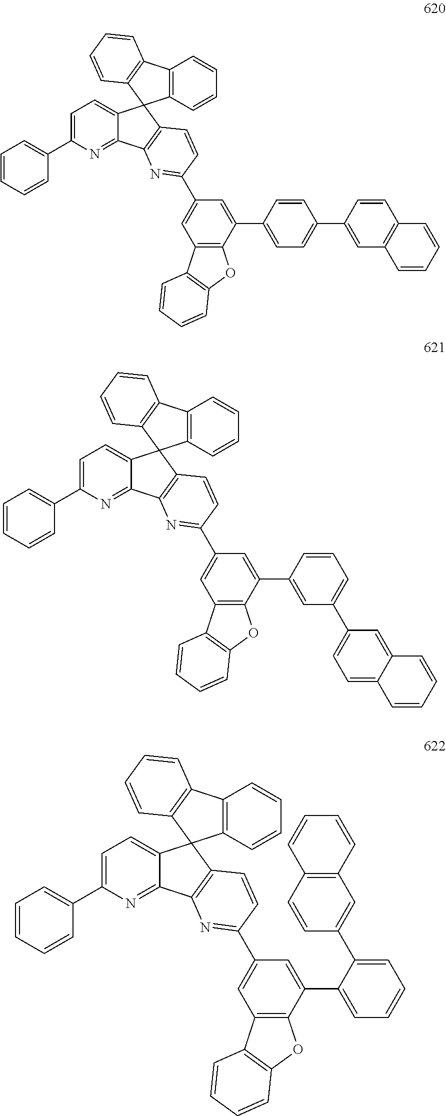 [SMILES: c1ccc(-c2ccc3c(n2)-c2nc(-c4cc(-c5ccc(-c6ccc7ccccc7c6)cc5)c5oc6ccccc6c5c4)ccc2C32c3ccccc3-c3ccccc32)cc1 ; c1ccc(-c2ccc3c(n2)-c2nc(-c4cc(-c5cccc(-c6ccc7ccccc7c6)c5)c5oc6ccccc6c5c4)ccc2C32c3ccccc3-c3ccccc32)cc1 ; c1ccc(-c2ccc3c(n2)-c2nc(-c4cc(-c5ccccc5-c5ccc6ccccc6c5)c5oc6ccccc6c5c4)ccc2C32c3ccccc3-c3ccccc32)cc1]

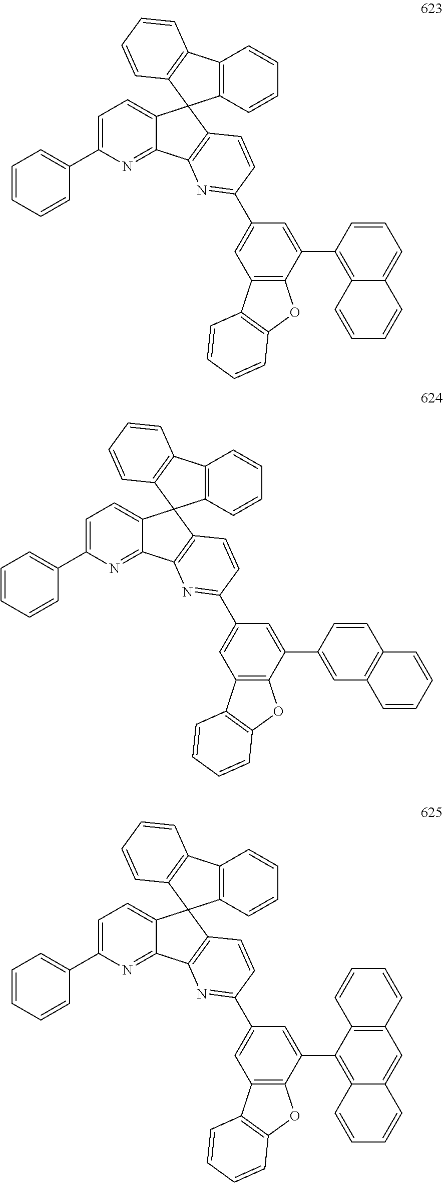 [SMILES: c1ccc(-c2ccc3c(n2)-c2nc(-c4cc(-c5cccc6ccccc56)c5oc6ccccc6c5c4)ccc2C32c3ccccc3-c3ccccc32)cc1 ; c1ccc(-c2ccc3c(n2)-c2nc(-c4cc(-c5ccc6ccccc6c5)c5oc6ccccc6c5c4)ccc2C32c3ccccc3-c3ccccc32)cc1 ; c1ccc(-c2ccc3c(n2)-c2nc(-c4cc(-c5c6ccccc6cc6ccccc56)c5oc6ccccc6c5c4)ccc2C32c3ccccc3-c3ccccc32)cc1]

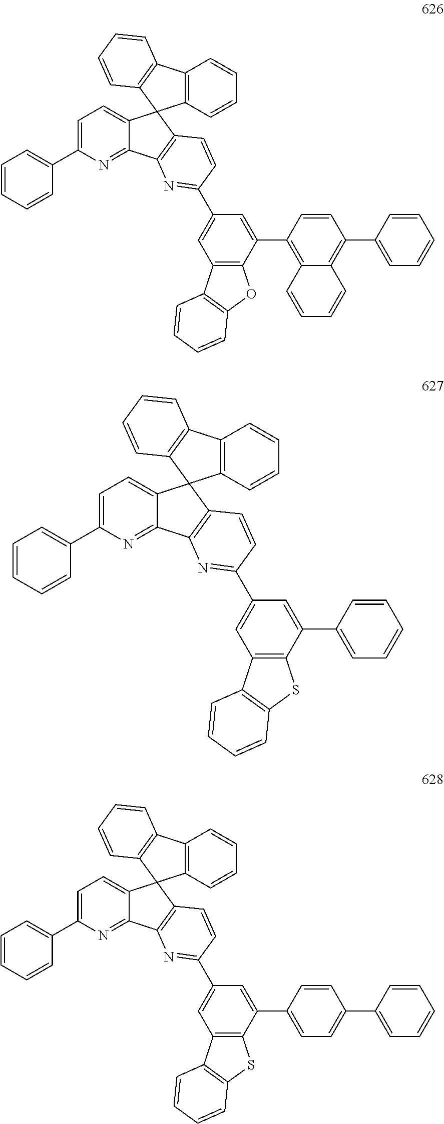 [SMILES: c1ccc(-c2ccc3c(n2)-c2nc(-c4cc(-c5ccc(-c6ccccc6)c6ccccc56)c5oc6ccccc6c5c4)ccc2C32c3ccccc3-c3ccccc32)cc1 ; c1ccc(-c2ccc3c(n2)-c2nc(-c4cc(-c5ccccc5)c5sc6ccccc6c5c4)ccc2C32c3ccccc3-c3ccccc32)cc1 ; c1ccc(-c2ccc(-c3cc(-c4ccc5c(n4)-c4nc(-c6ccccc6)ccc4C54c5ccccc5-c5ccccc54)cc4c3sc3ccccc34)cc2)cc1]

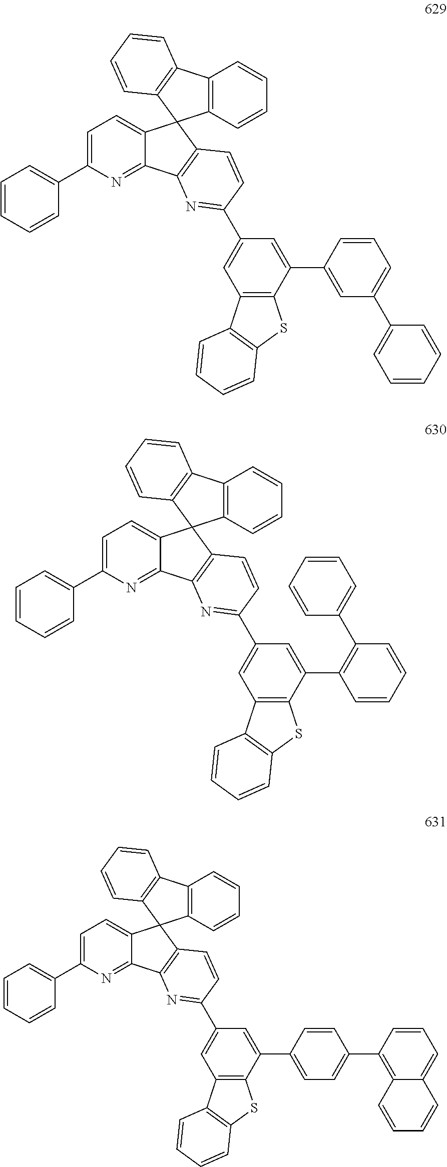 [SMILES: c1ccc(-c2cccc(-c3cc(-c4ccc5c(n4)-c4nc(-c6ccccc6)ccc4C54c5ccccc5-c5ccccc54)cc4c3sc3ccccc34)c2)cc1 ; c1ccc(-c2ccc3c(n2)-c2nc(-c4cc(-c5ccccc5-c5ccccc5)c5sc6ccccc6c5c4)ccc2C32c3ccccc3-c3ccccc32)cc1 ; c1ccc(-c2ccc3c(n2)-c2nc(-c4cc(-c5ccc(-c6cccc7ccccc67)cc5)c5sc6ccccc6c5c4)ccc2C32c3ccccc3-c3ccccc32)cc1]

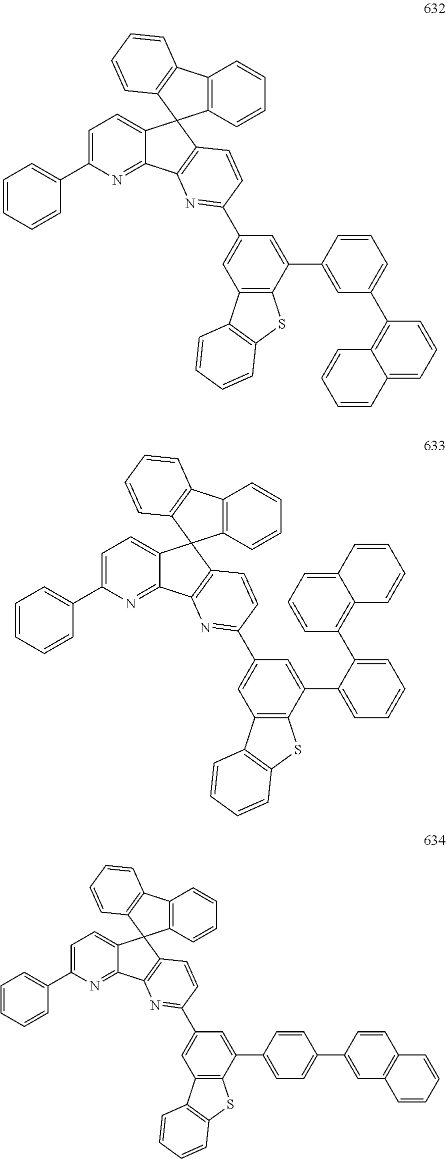 [SMILES: c1ccc(-c2ccc3c(n2)-c2nc(-c4cc(-c5cccc(-c6cccc7ccccc67)c5)c5sc6ccccc6c5c4)ccc2C32c3ccccc3-c3ccccc32)cc1 ; c1ccc(-c2ccc3c(n2)-c2nc(-c4cc(-c5ccccc5-c5cccc6ccccc56)c5sc6ccccc6c5c4)ccc2C32c3ccccc3-c3ccccc32)cc1 ; c1ccc(-c2ccc3c(n2)-c2nc(-c4cc(-c5ccc(-c6ccc7ccccc7c6)cc5)c5sc6ccccc6c5c4)ccc2C32c3ccccc3-c3ccccc32)cc1]

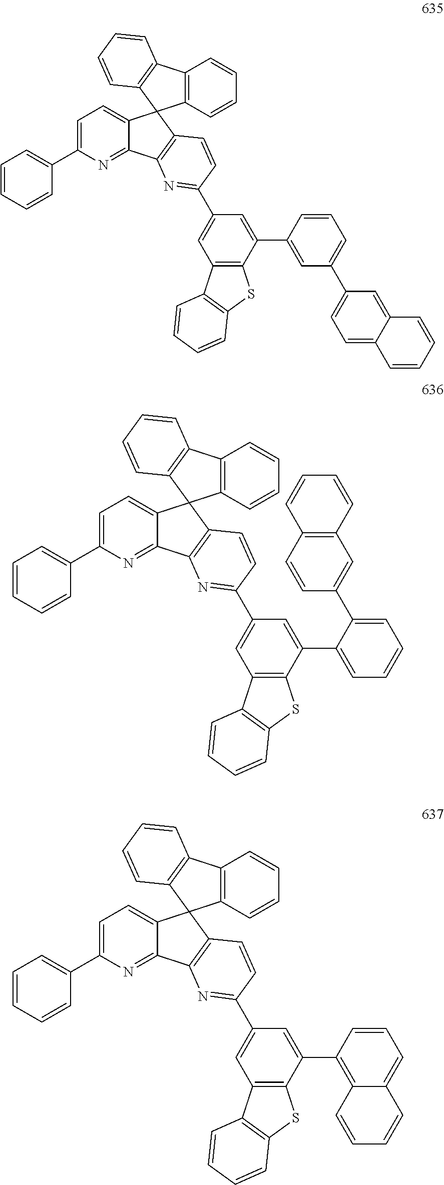 [SMILES: c1ccc(-c2ccc3c(n2)-c2nc(-c4cc(-c5cccc(-c6ccc7ccccc7c6)c5)c5sc6ccccc6c5c4)ccc2C32c3ccccc3-c3ccccc32)cc1 ; c1ccc(-c2ccc3c(n2)-c2nc(-c4cc(-c5ccccc5-c5ccc6ccccc6c5)c5sc6ccccc6c5c4)ccc2C32c3ccccc3-c3ccccc32)cc1 ; c1ccc(-c2ccc3c(n2)-c2nc(-c4cc(-c5cccc6ccccc56)c5sc6ccccc6c5c4)ccc2C32c3ccccc3-c3ccccc32)cc1]

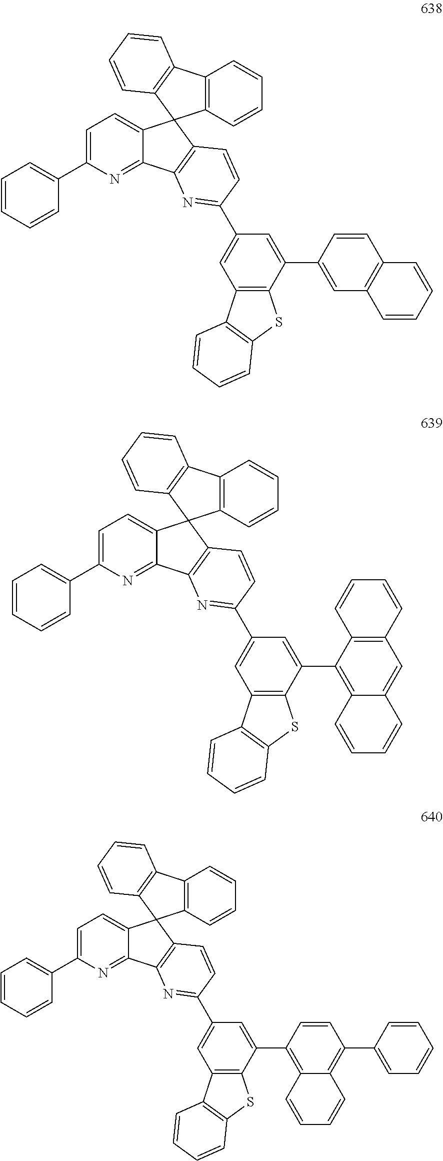 [SMILES: c1ccc(-c2ccc3c(n2)-c2nc(-c4cc(-c5ccc6ccccc6c5)c5sc6ccccc6c5c4)ccc2C32c3ccccc3-c3ccccc32)cc1 ; c1ccc(-c2ccc3c(n2)-c2nc(-c4cc(-c5c6ccccc6cc6ccccc56)c5sc6ccccc6c5c4)ccc2C32c3ccccc3-c3ccccc32)cc1 ; c1ccc(-c2ccc3c(n2)-c2nc(-c4cc(-c5ccc(-c6ccccc6)c6ccccc56)c5sc6ccccc6c5c4)ccc2C32c3ccccc3-c3ccccc32)cc1]

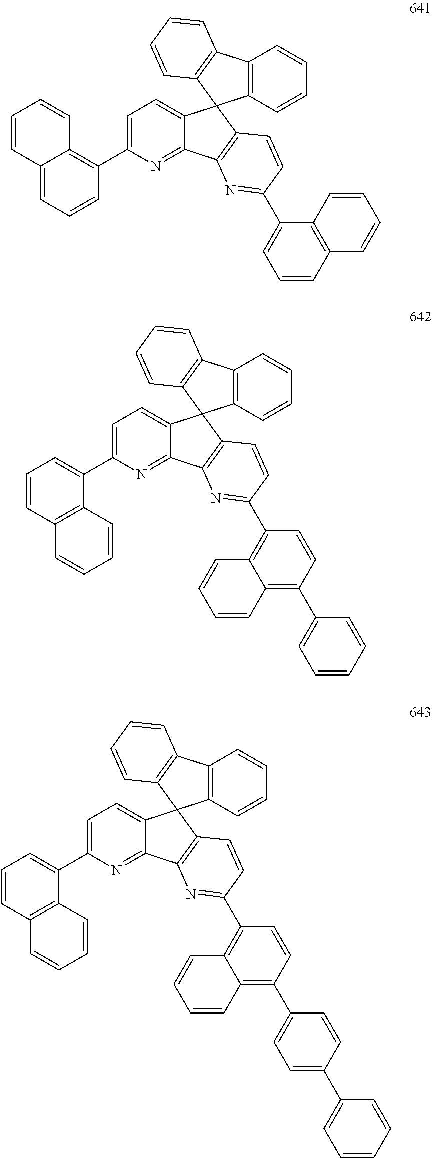 [SMILES: c1ccc2c(c1)-c1ccccc1C21c2ccc(-c3cccc4ccccc34)nc2-c2nc(-c3cccc4ccccc34)ccc21 ; c1ccc(-c2ccc(-c3ccc4c(n3)-c3nc(-c5cccc6ccccc56)ccc3C43c4ccccc4-c4ccccc43)c3ccccc23)cc1 ; c1ccc(-c2ccc(-c3ccc(-c4ccc5c(n4)-c4nc(-c6cccc7ccccc67)ccc4C54c5ccccc5-c5ccccc54)c4ccccc34)cc2)cc1]

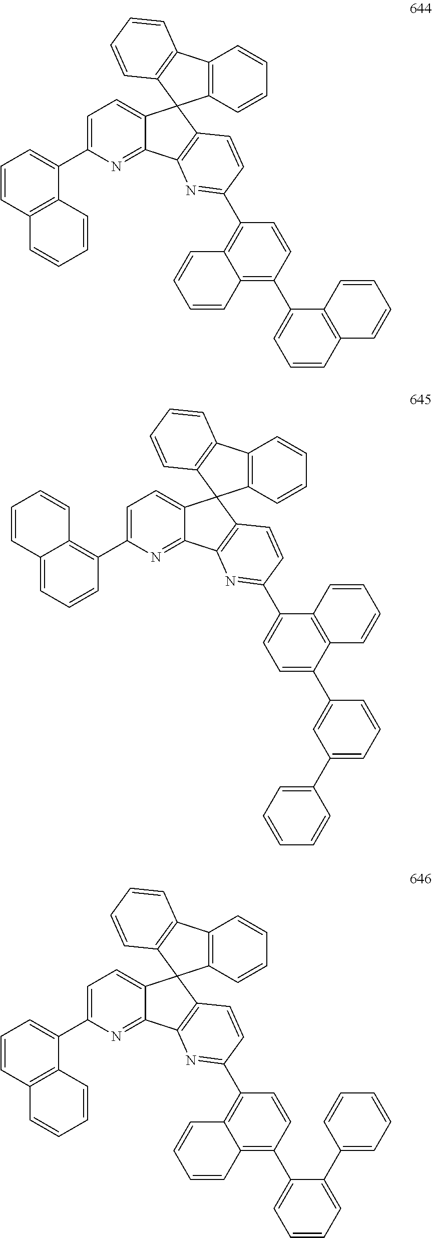 [SMILES: c1ccc2c(c1)-c1ccccc1C21c2ccc(-c3cccc4ccccc34)nc2-c2nc(-c3ccc(-c4cccc5ccccc45)c4ccccc34)ccc21 ; c1ccc(-c2cccc(-c3ccc(-c4ccc5c(n4)-c4nc(-c6cccc7ccccc67)ccc4C54c5ccccc5-c5ccccc54)c4ccccc34)c2)cc1 ; c1ccc(-c2ccccc2-c2ccc(-c3ccc4c(n3)-c3nc(-c5cccc6ccccc56)ccc3C43c4ccccc4-c4ccccc43)c3ccccc23)cc1]

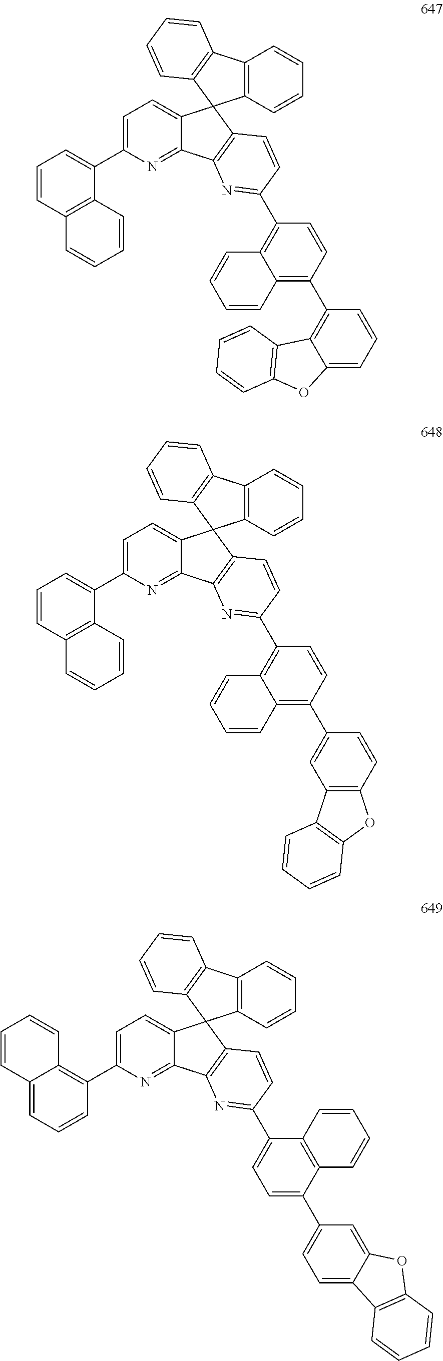 [SMILES: c1ccc2c(c1)-c1ccccc1C21c2ccc(-c3cccc4ccccc34)nc2-c2nc(-c3ccc(-c4cccc5oc6ccccc6c45)c4ccccc34)ccc21 ; c1ccc2c(c1)-c1ccccc1C21c2ccc(-c3cccc4ccccc34)nc2-c2nc(-c3ccc(-c4ccc5oc6ccccc6c5c4)c4ccccc34)ccc21 ; c1ccc2c(c1)-c1ccccc1C21c2ccc(-c3cccc4ccccc34)nc2-c2nc(-c3ccc(-c4ccc5c(c4)oc4ccccc45)c4ccccc34)ccc21]

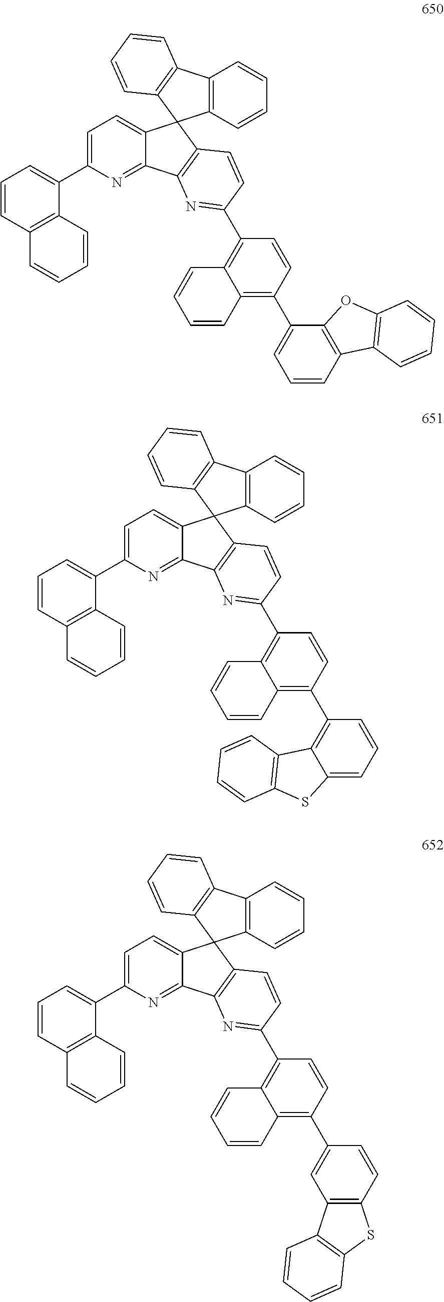 [SMILES: c1ccc2c(c1)-c1ccccc1C21c2ccc(-c3cccc4ccccc34)nc2-c2nc(-c3ccc(-c4cccc5c4oc4ccccc45)c4ccccc34)ccc21 ; c1ccc2c(c1)-c1ccccc1C21c2ccc(-c3cccc4ccccc34)nc2-c2nc(-c3ccc(-c4cccc5sc6ccccc6c45)c4ccccc34)ccc21 ; c1ccc2c(c1)-c1ccccc1C21c2ccc(-c3cccc4ccccc34)nc2-c2nc(-c3ccc(-c4ccc5sc6ccccc6c5c4)c4ccccc34)ccc21]

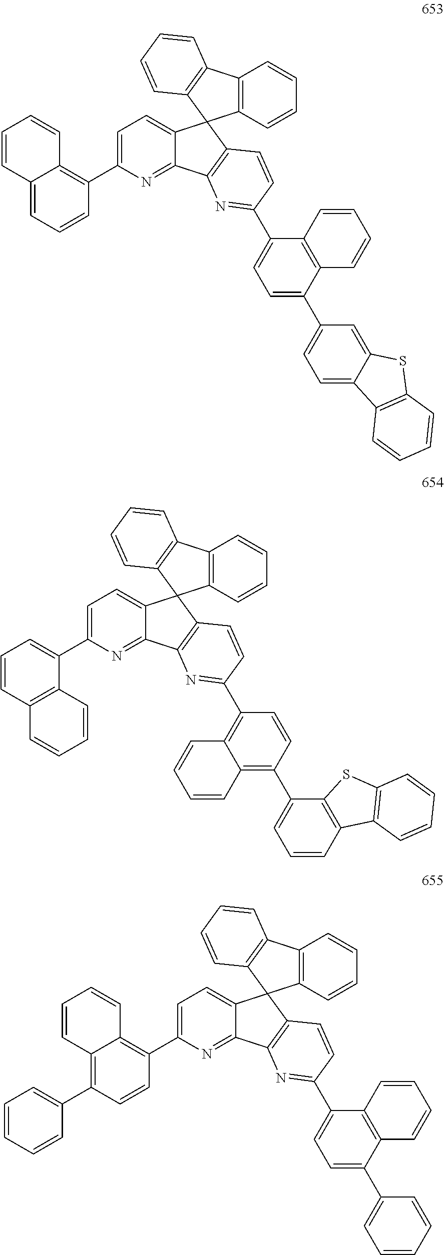 [SMILES: c1ccc2c(c1)-c1ccccc1C21c2ccc(-c3cccc4ccccc34)nc2-c2nc(-c3ccc(-c4ccc5c(c4)sc4ccccc45)c4ccccc34)ccc21 ; c1ccc2c(c1)-c1ccccc1C21c2ccc(-c3cccc4ccccc34)nc2-c2nc(-c3ccc(-c4cccc5c4sc4ccccc45)c4ccccc34)ccc21 ; c1ccc(-c2ccc(-c3ccc4c(n3)-c3nc(-c5ccc(-c6ccccc6)c6ccccc56)ccc3C43c4ccccc4-c4ccccc43)c3ccccc23)cc1]

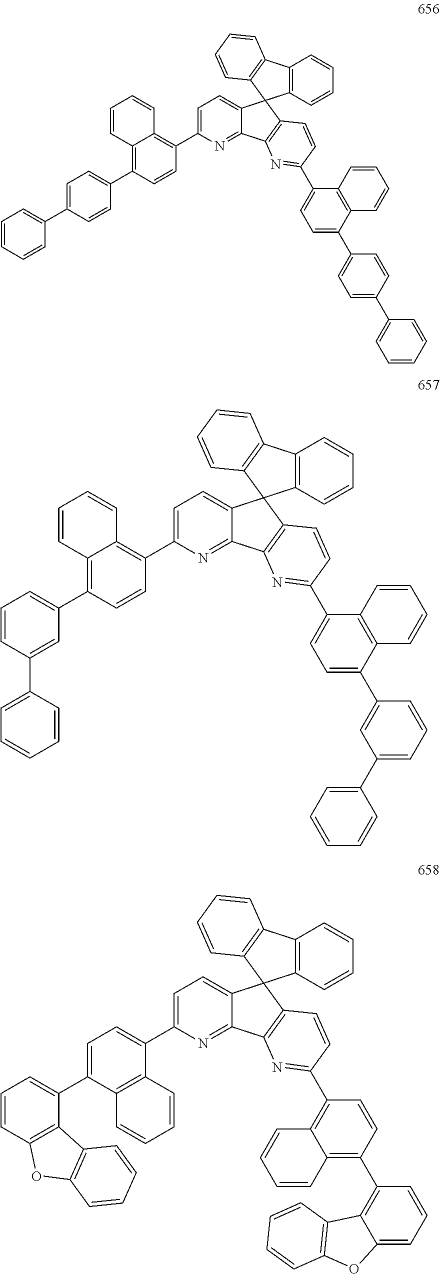 [SMILES: c1ccc(-c2ccc(-c3ccc(-c4ccc5c(n4)-c4nc(-c6ccc(-c7ccc(-c8ccccc8)cc7)c7ccccc67)ccc4C54c5ccccc5-c5ccccc54)c4ccccc34)cc2)cc1 ; c1ccc(-c2cccc(-c3ccc(-c4ccc5c(n4)-c4nc(-c6ccc(-c7cccc(-c8ccccc8)c7)c7ccccc67)ccc4C54c5ccccc5-c5ccccc54)c4ccccc34)c2)cc1 ; c1ccc2c(c1)-c1ccccc1C21c2ccc(-c3ccc(-c4cccc5oc6ccccc6c45)c4ccccc34)nc2-c2nc(-c3ccc(-c4cccc5oc6ccccc6c45)c4ccccc34)ccc21]

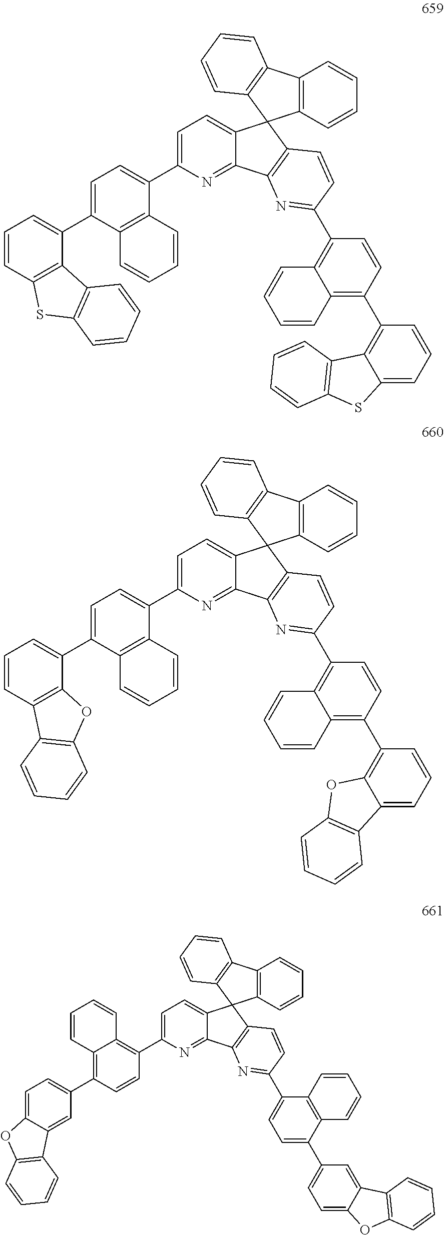 [SMILES: c1ccc2c(c1)-c1ccccc1C21c2ccc(-c3ccc(-c4cccc5sc6ccccc6c45)c4ccccc34)nc2-c2nc(-c3ccc(-c4cccc5sc6ccccc6c45)c4ccccc34)ccc21 ; c1ccc2c(c1)-c1ccccc1C21c2ccc(-c3ccc(-c4cccc5c4oc4ccccc45)c4ccccc34)nc2-c2nc(-c3ccc(-c4cccc5c4oc4ccccc45)c4ccccc34)ccc21 ; c1ccc2c(c1)-c1ccccc1C21c2ccc(-c3ccc(-c4ccc5oc6ccccc6c5c4)c4ccccc34)nc2-c2nc(-c3ccc(-c4ccc5oc6ccccc6c5c4)c4ccccc34)ccc21]

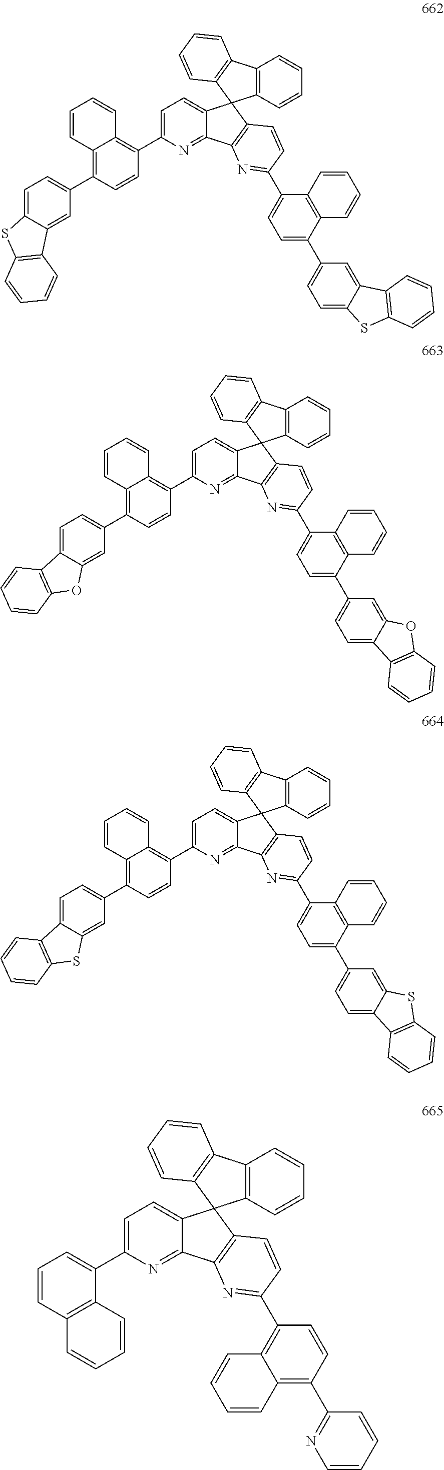 [SMILES: c1ccc2c(c1)-c1ccccc1C21c2ccc(-c3ccc(-c4ccc5sc6ccccc6c5c4)c4ccccc34)nc2-c2nc(-c3ccc(-c4ccc5sc6ccccc6c5c4)c4ccccc34)ccc21 ; c1ccc2c(c1)-c1ccccc1C21c2ccc(-c3ccc(-c4ccc5c(c4)oc4ccccc45)c4ccccc34)nc2-c2nc(-c3ccc(-c4ccc5c(c4)oc4ccccc45)c4ccccc34)ccc21 ; c1ccc2c(c1)-c1ccccc1C21c2ccc(-c3ccc(-c4ccc5c(c4)sc4ccccc45)c4ccccc34)nc2-c2nc(-c3ccc(-c4ccc5c(c4)sc4ccccc45)c4ccccc34)ccc21 ; c1ccc(-c2ccc(-c3ccc4c(n3)-c3nc(-c5cccc6ccccc56)ccc3C43c4ccccc4-c4ccccc43)c3ccccc23)nc1]

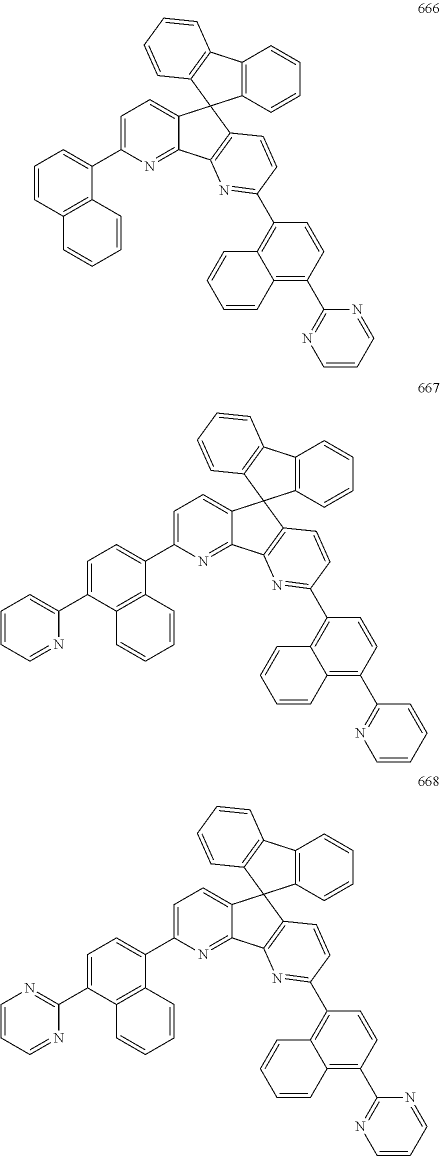 [SMILES: c1cnc(-c2ccc(-c3ccc4c(n3)-c3nc(-c5cccc6ccccc56)ccc3C43c4ccccc4-c4ccccc43)c3ccccc23)nc1 ; c1ccc(-c2ccc(-c3ccc4c(n3)-c3nc(-c5ccc(-c6ccccn6)c6ccccc56)ccc3C43c4ccccc4-c4ccccc43)c3ccccc23)nc1 ; c1cnc(-c2ccc(-c3ccc4c(n3)-c3nc(-c5ccc(-c6ncccn6)c6ccccc56)ccc3C43c4ccccc4-c4ccccc43)c3ccccc23)nc1]

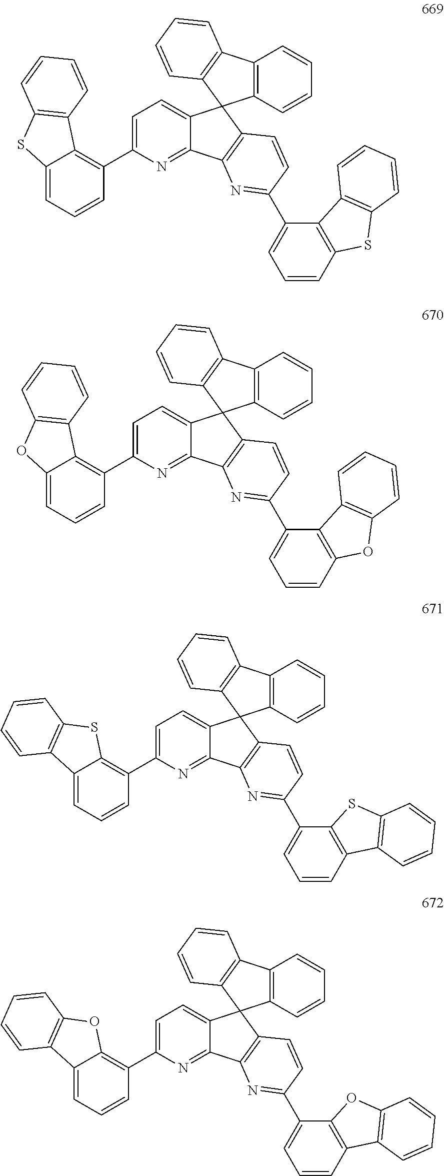 [SMILES: c1ccc2c(c1)-c1ccccc1C21c2ccc(-c3cccc4sc5ccccc5c34)nc2-c2nc(-c3cccc4sc5ccccc5c34)ccc21 ; c1ccc2c(c1)-c1ccccc1C21c2ccc(-c3cccc4oc5ccccc5c34)nc2-c2nc(-c3cccc4oc5ccccc5c34)ccc21 ; c1ccc2c(c1)-c1ccccc1C21c2ccc(-c3cccc4c3sc3ccccc34)nc2-c2nc(-c3cccc4c3sc3ccccc34)ccc21 ; c1ccc2c(c1)-c1ccccc1C21c2ccc(-c3cccc4c3oc3ccccc34)nc2-c2nc(-c3cccc4c3oc3ccccc34)ccc21]

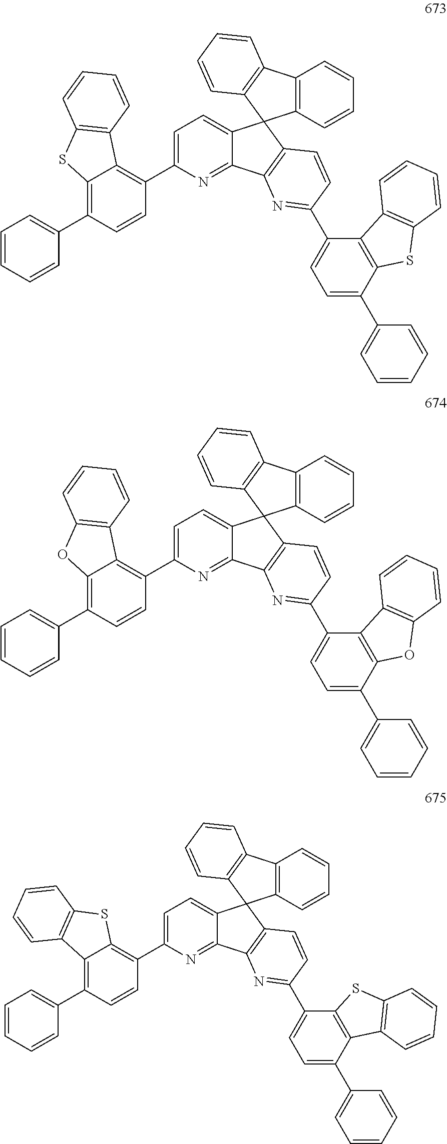 [SMILES: c1ccc(-c2ccc(-c3ccc4c(n3)-c3nc(-c5ccc(-c6ccccc6)c6sc7ccccc7c56)ccc3C43c4ccccc4-c4ccccc43)c3c2sc2ccccc23)cc1 ; c1ccc(-c2ccc(-c3ccc4c(n3)-c3nc(-c5ccc(-c6ccccc6)c6oc7ccccc7c56)ccc3C43c4ccccc4-c4ccccc43)c3c2oc2ccccc23)cc1 ; c1ccc(-c2ccc(-c3ccc4c(n3)-c3nc(-c5ccc(-c6ccccc6)c6c5sc5ccccc56)ccc3C43c4ccccc4-c4ccccc43)c3sc4ccccc4c23)cc1]

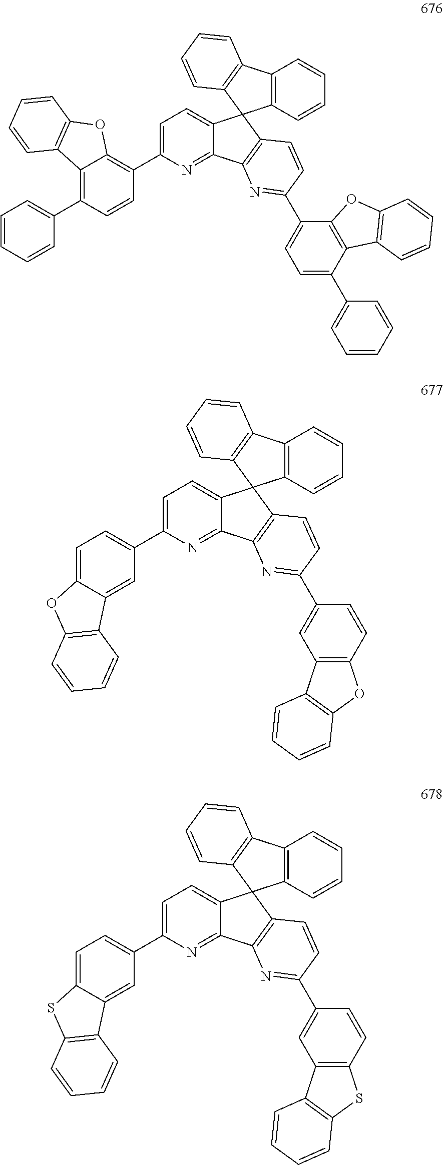 [SMILES: c1ccc(-c2ccc(-c3ccc4c(n3)-c3nc(-c5ccc(-c6ccccc6)c6c5oc5ccccc56)ccc3C43c4ccccc4-c4ccccc43)c3oc4ccccc4c23)cc1 ; c1ccc2c(c1)-c1ccccc1C21c2ccc(-c3ccc4oc5ccccc5c4c3)nc2-c2nc(-c3ccc4oc5ccccc5c4c3)ccc21 ; c1ccc2c(c1)-c1ccccc1C21c2ccc(-c3ccc4sc5ccccc5c4c3)nc2-c2nc(-c3ccc4sc5ccccc5c4c3)ccc21]

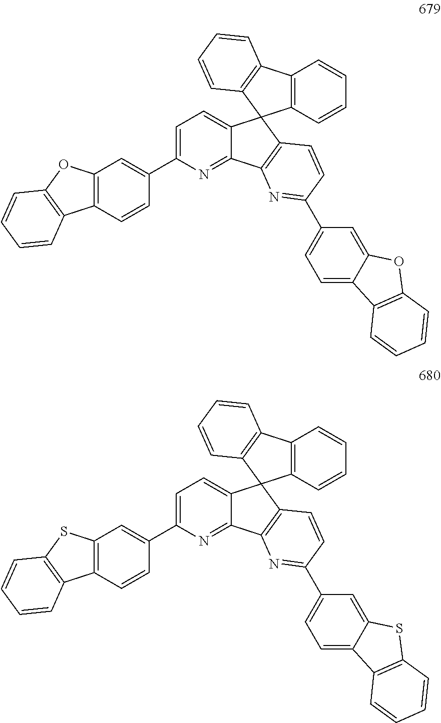 [SMILES: c1ccc2c(c1)-c1ccccc1C21c2ccc(-c3ccc4c(c3)oc3ccccc34)nc2-c2nc(-c3ccc4c(c3)oc3ccccc34)ccc21 ; c1ccc2c(c1)-c1ccccc1C21c2ccc(-c3ccc4c(c3)sc3ccccc34)nc2-c2nc(-c3ccc4c(c3)sc3ccccc34)ccc21]

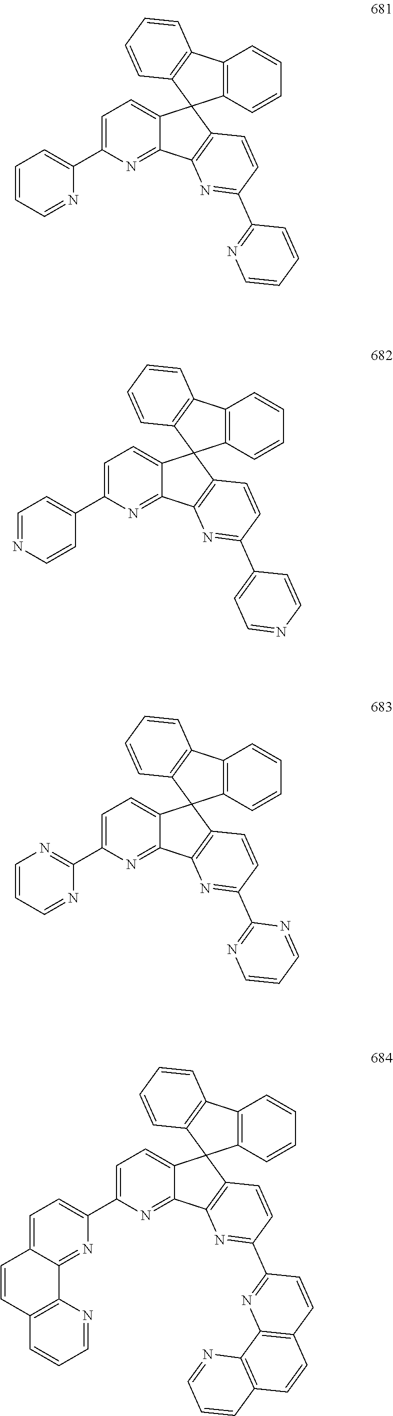 [SMILES: c1ccc(-c2ccc3c(n2)-c2nc(-c4ccccn4)ccc2C32c3ccccc3-c3ccccc32)nc1 ; c1ccc2c(c1)-c1ccccc1C21c2ccc(-c3ccncc3)nc2-c2nc(-c3ccncc3)ccc21 ; c1cnc(-c2ccc3c(n2)-c2nc(-c4ncccn4)ccc2C32c3ccccc3-c3ccccc32)nc1 ; c1ccc2c(c1)-c1ccccc1C21c2ccc(-c3ccc4ccc5cccnc5c4n3)nc2-c2nc(-c3ccc4ccc5cccnc5c4n3)ccc21]

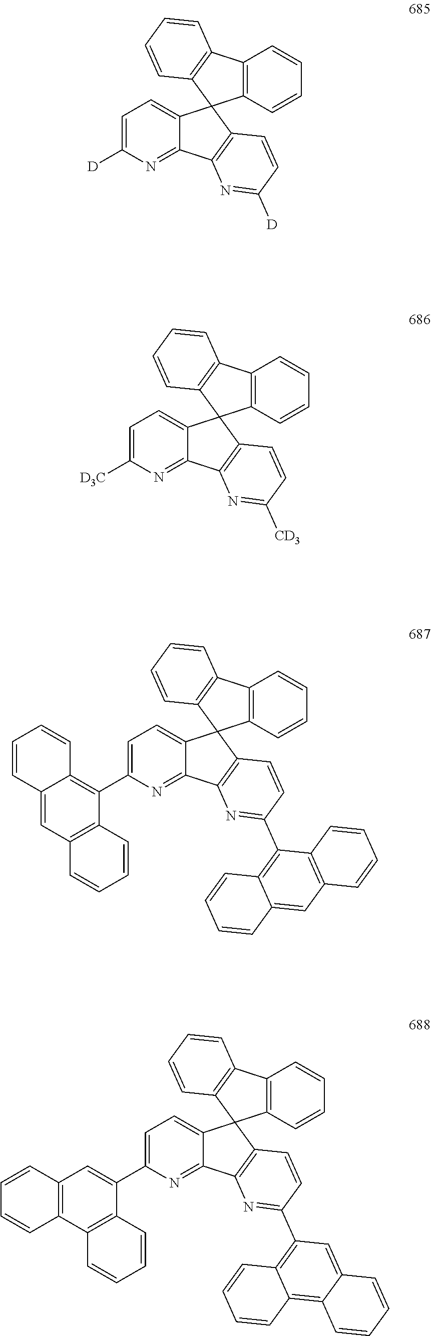 [SMILES: [2H]c1ccc2c(n1)-c1nc([2H])ccc1C21c2ccccc2-c2ccccc21 ; [2H]C([2H])([2H])c1ccc2c(n1)-c1nc(C([2H])([2H])[2H])ccc1C21c2ccccc2-c2ccccc21 ; c1ccc2c(c1)-c1ccccc1C21c2ccc(-c3c4ccccc4cc4ccccc34)nc2-c2nc(-c3c4ccccc4cc4ccccc34)ccc21 ; c1ccc2c(c1)-c1ccccc1C21c2ccc(-c3cc4ccccc4c4ccccc34)nc2-c2nc(-c3cc4ccccc4c4ccccc34)ccc21]

An organic light-emitting diode of the present disclosure may be an organic light-emitting diode having a tandem structure.

The organic light-emitting diode having the tandem structure of the present disclosure may include positive and negative electrodes facing each other and disposed on a substrate, and a plurality of light emitting stacks (or light emitting units) stacked between the positive and negative electrodes, wherein each light emitting stack includes a light emitting layer to emit light of a specific wavelength band.

The plurality of light emitting stacks may be configured to emit light of the same color or emit light of different colors. In addition, one light emitting stack may include one or more light emitting layers. In this case, when one light emitting stack includes two or more light emitting layers, they may emit light of the same or different colors.

The tandem organic light-emitting diode according to an embodiment of the present disclosure may include a positive electrode; a negative electrode facing the positive electrode; N light emitting stacks disposed between the positive electrode and the negative electrode include, wherein N is an integer of 2 or larger; and N−1 n-type charge generation layers and N−1 p-type charge generation layers, wherein each of the N−1 n-type charge generation layers and each of the N−1 p-type charge generation layers are disposed on top of each other and contact each other and are disposed between different and adjacent light emitting stacks of the N light emitting stacks, wherein at least one of the N−1 n-type charge generation layers includes the compound represented by the Chemical Formula 1-1.

In one embodiment of the organic light-emitting diode, N is one of 2, 3, 4, and 5. For example, if N is 2, it may include 1 n-type charge generation layers and 1 p-type charge generation layers. And if N is 3, it may include 2 n-type charge generation layers and 2 p-type charge generation layers.

In the present disclosure, the light emitting stack refers to a unit structure including an organic layer including an electron transport layer (ETL) and a hole transport layer (HTL), and an light emitting layer (EML) disposed between the electron transport layer and the hole transport layer. The organic layer may further include an electron injection layer (EIL), a hole injection layer (HIL), an electron transport auxiliary layer, a hole transport auxiliary layer, and the like. The organic light-emitting diode may further include other organic layers depending on the structure or design of the organic light-emitting diode.

Hereinafter, various embodiments of the disclosure will be described with reference to the accompanying drawings.

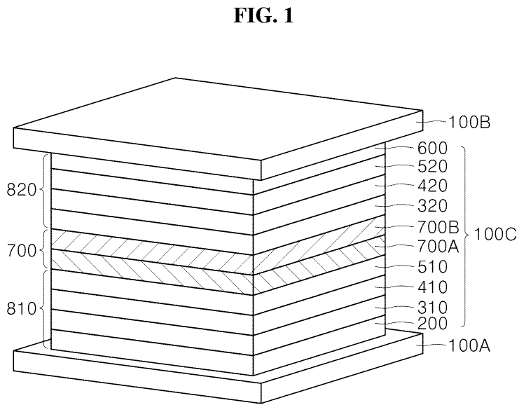

FIG. 1 is a view illustrating a structure of an organic light-emitting diode according to an embodiment.

As shown in FIG. 1, an organic light-emitting diode 100 of the present disclosure includes a positive electrode 100A and a negative electrode 100B facing each other, and an intermediate layer 100C positioned between the positive electrode 100A and the negative electrode 100B. The intermediate layer 100C includes a first light emitting stack 810 positioned between the positive electrode 100A and the negative electrode 100B and including a first light emitting layer 410; a second light emitting stack 820 positioned between the first light emitting stack 810 and the negative electrode 100B and including a second light emitting layer 420; and a charge generation layer 700 positioned between the first and second light emitting stacks 810 and 820. The first light emitting stack 810 includes a hole injection layer 200, a first hole transport layer 310, the first light emitting layer 410, and a first electron transport layer 510, while the second light emitting stack 820 includes a second hole transport layer 320, the second light emitting layer 420, a second electron transport layer 520, and a hole injection layer 600.

The positive electrode 100A may include a material such as indium tin oxide (ITO), indium zinc oxide (IZO), tin oxide (SnO₂), zinc oxide (ZnO), or the like, which is transparent, has a relatively large work function value, and has excellent conductivity. However, the present disclosure is not limited thereto.

The negative electrode 100B may include a material such as lithium (Li), aluminum (Al), aluminum-lithium (Al—Li), calcium (Ca), magnesium (Mg), magnesium-indium (Mg—In), or magnesium-silver (Mg—Ag). In addition, in the case of the top emission organic light-emitting diode, a transparent negative electrode through which light can transmit may be formed using indium tin oxide (ITO) or indium zinc oxide (IZO). However, the present disclosure is not limited thereto. In order to prevent external moisture or the like from penetrating into the organic light-emitting diode, a capping layer (CPL) and/or a protective film (a protecting layer or an encapsulation layer or a seal cap) formed on top of the negative electrode of the organic light-emitting diode may be further included in the diode.

The hole transport layers 310 and 320 may be made of a material having excellent hole transport characteristics. For example, the hole transport layers 310 and 320 may be made of phthalocyanine derivatives, porphyrin derivatives, triarylamine derivatives, and indolocarbazole derivatives. For example, the hole transport layers 310 and 320 may be made of 1,4,5,8,9,11-hexaazatriphenylene-hexacarbonitrile (HAT-CN), copper phthalocyanine (CuPc), 4,4′,4″-tris(3-methylphenylamino)triphenylamine (m-MTDATA), 4,4′,4″-tris(3-methylphenylamino) phenoxybenzene (m-MTDAPB), 4,4′,4″-tri(N-carbazolyl)triphenylamine (TCTA), 4,4′,4″-tris(N-(2-naphthyl)-N-phenylamino)-triphenylamine (2-TNATA), N4,N4,N4′1-tetra′ Phenyl]-4-yl)-[1,1′-biphenyl]-4,4′-diamine (N4,N4,N4′,N4′-Tetra([1,1′-biphenyl]-4-yl)-[1,1′-N4]-4,4′-biphenyl, bis(diamine-(1-naphthyl-N-phenyl))benzidine (α-NPD), n,N′-di(naphthalen-1-yl)-N,N′-biphenyl-benzidine (NPB), N,N′-biphenyl-N,N N′-bis(3-methylphenyl)-1,1′-biphenyl-4,4′-diamine (TPD), and the like. However, the present disclosure is not limited thereto.

Although not shown in FIG. 1, an electron transport auxiliary layer or a hole transport auxiliary layer in contact with the light emitting layer may be further included in the diode to increase efficiency of the light emitting layer.

The light emitting layers 410 and 420 may be formed by doping a host with dopants to improve luminous efficiency of the diode, and each of the light emitting layers 410 and 420 may emit light of blue, green, or red. However, the present disclosure is not limited thereto, and each of the light emitting layers 410 and 420 may emit light of combinations of various colors and color coordinates used in the organic light-emitting diode. For example, the CIEx coordinate of a color of light emitted from a blue light emitting layer may have a range of 0.01 to 0.15, the CIEy coordinate of a color of light emitted from the blue light emitting layer may have a range of 0.03 to 0.07, the CIEx coordinate of a color of light emitted from the green light emitting layer may have a range of 0.19 to 0.32, and the CIEy coordinate of a color of light emitted from the green light emitting layer may have a range of 0.65 to 0.76.

The light emitting layer dopant material and the light emitting layer host material may be selected according to a color of light emitted from each of the light emitting layers 410 and 420 included in the organic light-emitting diode of the present disclosure. For example, when a total weight of the light emitting layer host is 100 wt %, the doping concentration of the light emitting layer dopant may be adjusted to be within a range of 1 to 20 wt %. However, the present disclosure is not limited thereto. The doping concentration of the light emitting layer dopant may be adjusted to be within a range of, for example, 3 to 15 wt %, for example, 5 to 10 wt %, for example, 3 to 8 wt %, for example, 2 to 7 wt %. However, the present disclosure is not limited thereto.

For example, the dopants of each of the light emitting layers 410 and 420 may include N1,N1,N6,N6-tetrakis(4-(1-silyl)phenyl)pyrene-1,6-diamine, an iridium metal complex compound (e.g., Ir(ppy)₃), t-DABNA-dtB (2,12-Di-tert-butyl-5,9-bis(4-(tert-butyl)phenyl)-7-(3,5-di-tert-butylphenyl)-5,9-dihydro5,9-diaza-13b-boranaphtho[3,2,1-de]anthracene) and the like, as general materials used in the art. However, the present disclosure is not limited thereto.

The electron transport layers 510 and 520 and the electron injection layer 600 may stably supply electrons to the light emitting layer under smooth electron transport. The materials of the electron transport layers 510 and 520 and the electron injection layer 600 require high electron mobility. For example, the materials of the electron transport layers 510 and 520 and the electron injection layer 600 independently include a pyridine derivative, a naphthalene derivative, an anthracene derivative, a phenanthroline derivative, a perinone derivative, a coumarin derivative, a naphthalimide derivative, an anthraquinone derivative, a diphenoquinone derivative, a diphenylquinone derivative, a perylene derivative, an oxadiazole derivative, a thiophene derivative, a triazole derivative, a thiadiazole derivative, a metal complex of an oxin derivative, a quinolinol-based metal complex, a quinoxaline derivative, a polymer of a quinoxaline derivative, a benzazole compound, a gallium complex, a pyrazole derivative, a perfluorinated phenylene derivative, a triazine derivative, a pyrazine derivative, a benzoquinoline derivative, an imidazopyridine derivative, a benzothioline derivative, a benzothioline derivative, a pyridine derivative, a pyridine derivative, a pyridine derivative, etc. Examples thereof include derivatives, terpyridine derivatives, naphthyridine derivatives, aldazine derivatives, carbazole derivatives, indole derivatives, phosphine oxide derivatives, bisstyryl derivatives, quinolinol-based metal complexes, hydroxyazole-based metal complexes, azo methine-based metal complexes, tropolone-based metal complexes, flavonol-based metal complexes, benzoquinoline-based metal complexes, metal salts, and the like. These compounds may be used alone or in combination with other materials. For example, a material such as 2-(4-(9,10-di(naphthalene-2-yl)anthracene-2-yl)phenyl)-1-phenyl-1H-benzo[d]imidazole, tris(8-hydroxyquinolinato)aluminum (Alq₃), LiF, Liq, Li₂O, BaO, NaCl, CsF, or the like may be used as the materials of the electron transport layers 510 and 520 and the electron injection layer 600. However, the present disclosure is not limited thereto.

The charge generation layer 700 is disposed between the first light emitting stack 810 and the second light emitting stack 820 to supply charges to the first light emitting stack and the second light emitting stack. In addition, the charge generation layer may serve to control a charge balance in the first light emitting stack and the second light emitting stack, and may be composed of a plurality of layers including the n-type charge generation layer (n-CGL) 700A and the p-type charge generation layer (p-CGL) 700B. However, the present disclosure is not limited thereto, and the charge generation layer may be composed of a single layer.

The n-type charge generation layer 700A injects electrons into the first light emitting stack 810. According to an embodiment of the present disclosure, the n-type charge generation layer may be made of a single n-type host material component. According to another embodiment of the present disclosure, the n-type charge generation layer may be formed by adding a n-type dopant material to a n-type host material. The n-type host material may include the compound represented by the Chemical Formula 1 according to the present disclosure, for example, the compound represented by the Chemical Formula 1-1, and thus the organic light-emitting diode according to the present disclosure may secure low operation voltage, high efficiency, and long lifespan characteristics.

When the n-type charge generation layer 700A of the present disclosure includes the n-type dopant material, the n-type dopant material may be an alkali metal or alkaline earth metal compound for electron injection or electron transport. Examples of the alkali metal or alkaline earth metal compound may include, but are not limited to, alkali metals such as lithium (Li), sodium (Na), potassium (K), cesium (Cs), and/or alkaline earth metals such as magnesium (Mg), strontium (Sr), barium (Ba), and radium (Ra).

The n-type charge generation layer 700A including the compound represented by the Chemical Formula 1 of the present disclosure may have excellent controlled charge balance, so that even if a small amount of the dopant is added thereto, the n-type charge generation layer 700A may sufficiently perform an unique function of the n-type charge generation layer. Accordingly, the amount of dopants used may be reduced, and thus the diode may be economically manufactured. In addition, the dopant has sufficient electrons. Thus, the amount of electrons is maintained at the same level even when a content of the added dopants is 5 wt % or greater. Accordingly, even when the dopant is added in an amount of 5 wt % or greater, a better effect may not be expected. Further, when the dopant is added in an amount of 10 wt % or greater, a leakage current and a short-circuit of the organic light-emitting diode may occur. Accordingly, when the weight of the compound represented by the Chemical Formula 1 according to the present disclosure used as the n-type host material is 100 wt %, the n-type dopant may be added at a content of about 0.1 to 10 wt %, for example, about 0.5 to 5 wt %. When the content of the n-type dopant is in the above defined range, the organic light-emitting diode of the present disclosure may exhibit the best characteristics.

The p-type charge generation layer 700B injects holes into the second light emitting stack 820. The p-type charge generation layer may include a p-type dopant material and a p-type host material. The p-type charge generation layer is disposed on the n-type charge generation layer and binds to the n-type charge generation layer. The p-type dopant material may include an organic material such as a metal oxide, an organic material such as tetrafluoro-tetracyanodimethane (F4-TCNQ), hexaazatriphenylene-hexaazatriphenylene (HAT-CN), hexaazatriphenylene derivative, or a metal material such as V₂O₅, MoOₓ, WO₃. However, the present disclosure is not limited thereto. The p-type host material may include a material capable of transferring holes, for example, a material including one or more of NPD (N,N′-bis(naphthalene-1-yl)-N,N′-bis(phenyl)-2,2′dimethylbenzidine), TPD (N,N′-bis-(3-methylphenyl)-N,N′-bis-(phenyl)-benzidine), and MTDATA (4,4′,4tris(N-3-methylphenyl-amino)-triphenylamine). However, the present disclosure is not limited thereto.

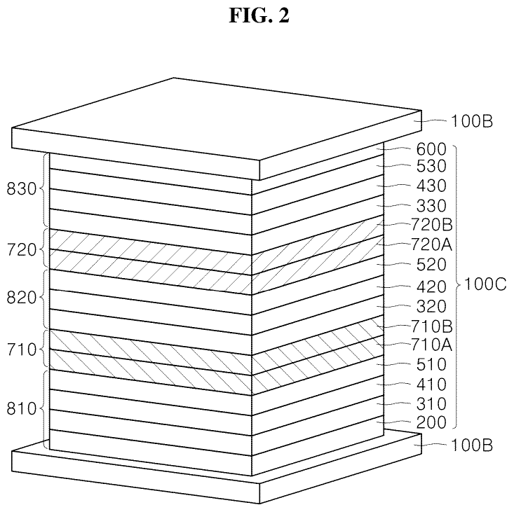

FIG. 2 is a diagram illustrating a structure of an organic light-emitting diode according to an embodiment.

As shown in FIG. 2, the organic light-emitting diode of the present disclosure includes an positive electrode 100A and a negative electrode 100B facing each other, and an interlayer 100C positioned between the positive electrode 100A and the negative electrode 100B. The intermediate layer 100C includes a first light emitting stack 810 positioned between the positive electrode 100A and the negative electrode 100B and including a first light emitting layer 410; a second light emitting stack 820 positioned between the positive electrode 100A and the negative electrode 100B and including a second light emitting layer 420; a third light emitting stack 830 positioned between the positive electrode 100A and the negative electrode 100B and including a third light emitting layer 430; a first charge generation layer 710 positioned between the first light emitting stack 810 and second light emitting stack 820; and a second charge generation layer 720 positioned between the second light emitting stack 820 and third light emitting stack 830. The first charge generation layer 710 and second charge generation layer 720 may include first n-type charge generation layer 710A and second n-type charge generation layer and 720A and first p-type charge generation layer 710B and second p-type charge generation layer 720B, respectively. The first n-type charge generation layer 710A and the second n-type charge generation layer 720A may be identical with or different from each other, and at least one thereof may include the compound represented by the Chemical Formula 1 according to the present disclosure. According to an embodiment of the present disclosure, both the first n-type charge generation layer and the second n-type charge generation layer may include the compound represented by the Chemical Formula 1 according to the present disclosure.

According to an embodiment of the present disclosure, one of the first or second n-type charge generation layers may include the compound represented by the Chemical Formula 1 according to the present disclosure, and the other thereof may use the compound disclosed in JP 4595346B2.

The descriptions about the n-type charge generation layer 700A and the p-type charge generation layer 700B described with reference to FIG. 1 may be applied to the first n-type charge generation layer 710A and second n-type charge generation layer 720A and the first p-type charge generation layer 710B and second p-type charge generation layer 720B of FIG. 2. For example, at least one of the first n-type charge generation layer 710A and second n-type charge generation layer 720A may include the compound represented by the Chemical Formula 1 according to the present disclosure, and thus, the organic light-emitting diode may secure low operation voltage, high efficiency, and long lifespan characteristics.

For example, when one of the first n-type charge generation layer 710A and second n-type charge generation layer 720A includes the compound represented by the Chemical Formula 1 according to the present disclosure, one of the first n-type charge generation layer 710A and second n-type charge generation layer 720A may be made of a n-type host material single component, or may be formed by adding the n-type dopant material to the n-type host material. The descriptions of the material types, the n-type host and the n-type dopant of each of the first n-type charge generation layer 710A and the second n-type charge generation layer 720A may be the same as the descriptions of the n-type charge generation layer 700A.

The type of the compound used for forming each of the first p-type charge generation layer 710B and second p-type charge generation layer 720B of FIG. 2 may be the same as that as described with reference to the p-type charge generation layer 700B in FIG. 1. The first p-type charge generation layer 710B and the second p-type charge generation layer 720B may be identical with or different from each other.

The descriptions of the positive electrode 100A, the negative electrode 100B, the hole injection layer 200, and the electron injection layer 600 of FIG. 2 may refer to the descriptions set forth above with reference to FIG. 1. The first HTL 310, the second HTL 320, and the third HTL 330 of FIG. 2 may refer to the descriptions set forth above with reference to FIG. 1. The compounds respectively constituting the first HTL 310, the second HTL 320, and the third HTL 330 may be identical with or different from each other. Each of the first light emitting layer 410, the second light emitting layer 420, and the third light emitting layer 430 of FIG. 2 may be subjected to descriptions as set forth above with reference to the light emitting layers 410 and 420 of FIG. 1. The compounds respectively constituting the first light emitting layer 410, the second light emitting layer 420, and the third light emitting layer 430 may be identical with or different from each other. The first ETL 510, the second ETL 520, and the third ETL 530 of FIG. 2 may be subjected to the descriptions as set forth above with respect to the ETLs 510 and 520 of FIG. 1. The compounds respectively constituting the first ETL 510, the second ETL 520, and the third ETL 530 may be identical with or different from each other.

Further, although not shown in the drawings, the organic light-emitting diode according to an embodiment of the present disclosure may include a tandem structure in which four or more light emitting stacks and three or more charge generation layers are disposed between the positive electrode and the negative electrode. In this case, at least one of the n-type charge generation layers may include the compound represented by the Chemical Formula 1 according to the present disclosure.

Hereinafter, Examples and Experimental Examples of the compounds will be described based on representative examples below. However, a synthesis method of the compounds of the present disclosure is limited to the method as set forth below, or the practice of the present disclosure is not limited to the following Examples and Experimental Examples.

The compound of the present disclosure may be synthesized in a manner shown in Reaction Formulas 1 to 3 of the following Synthesis Examples. However, the present disclosure is not limited thereto. The following Reaction Formulas 1 to 3 representatively describe Synthesis Examples of products P1, P7, and P11. The compounds represented by the Chemical Formula 1 of the present disclosure may be synthesized in a similar manner to the reaction of products P1, P7, or P11.

### Synthesis Example

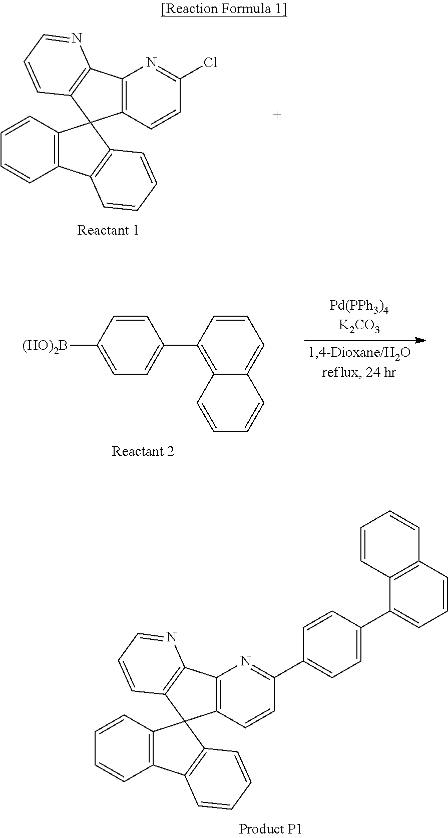 [SMILES: Clc1ccc2c(n1)-c1ncccc1C21c2ccccc2-c2ccccc21 ; OB(O)c1ccc(-c2cccc3ccccc23)cc1 ; c1ccc2c(c1)-c1ccccc1C21c2cccnc2-c2nc(-c3ccc(-c4cccc5ccccc45)cc3)ccc21]

As shown in Reaction Formula 1, Reactant 1 (20 mmol, 7.06 g), reactant 2 (20 mmol, 4.96 g), K₂CO₃ (60 mmol, 8.3 g), Pd(PPh₃)₄ (0.1 mmol, 1.2 g), 1,4-dioxane (200 mL), and water (40 mL) were added to a 500 mL flask under a nitrogen gas flow, and the mixed solution was stirred for 24 hours under reflux condition. After completion of the reaction, an organic layer was extracted using CH₂Cl₂ and water. The extracted solution was treated with MgSO₄ to remove residual moisture therefrom, concentrated under reduced pressure, purified using column chromatography, and recrystallized to obtain the product P1. (88%, 9.17 g)

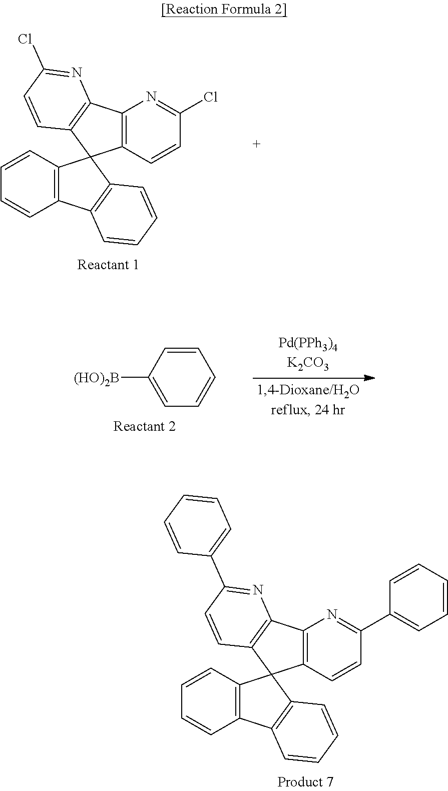 [SMILES: Clc1ccc2c(n1)-c1nc(Cl)ccc1C21c2ccccc2-c2ccccc21 ; OB(O)c1ccccc1 ; c1ccc(-c2ccc3c(n2)-c2nc(-c4ccccc4)ccc2C32c3ccccc3-c3ccccc32)cc1]

As shown in Reaction Formula 2, Reactant 1 (20 mmol, 7.75 g), reactant 2 (40 mmol, 4.48 g), K₂CO₃ (120 mmol, 16.6 g), Pd(PPh₃)₄ (0.2 mmol, 2.4 g), 1,4-dioxane (200 mL), water (40 mL) were added to a 500 mL flask under a nitrogen gas flow, and the mixed solution was stirred for 24 hours under reflux condition. After completion of the reaction, an organic layer was extracted using CH₂Cl₂ and water. The extracted solution was treated with MgSO₄ to remove residual moisture therefrom, concentrated under reduced pressure, purified using column chromatography, and recrystallized to obtain the product P7. (83%, 7.80 g)

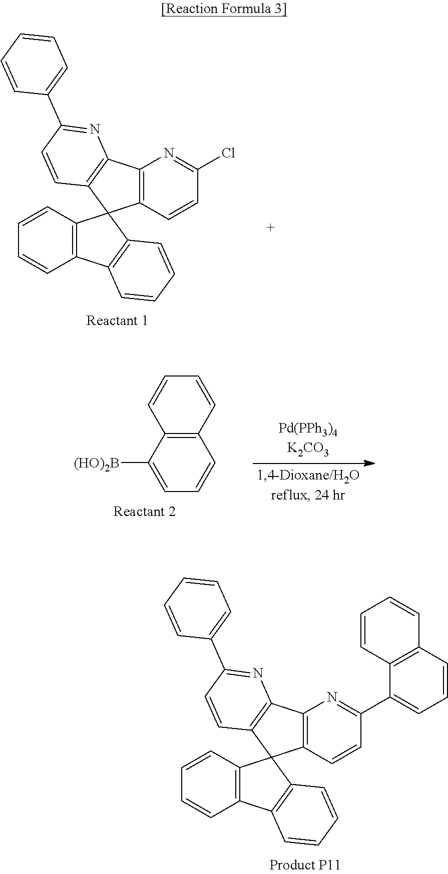 [SMILES: Clc1ccc2c(n1)-c1nc(-c3ccccc3)ccc1C21c2ccccc2-c2ccccc21 ; OB(O)c1cccc2ccccc12 ; c1ccc(-c2ccc3c(n2)-c2nc(-c4cccc5ccccc45)ccc2C32c3ccccc3-c3ccccc32)cc1]

As shown in Reaction Formula 3, Reactant 1 (20 mmol, 8.58 g), reactant 2 (20 mmol, 3.44 g), K₂CO₃ (60 mmol, 8.3 g), Pd(PPh₃)₄ (0.1 mmol, 1.2 g), 1,4-dioxane (200 mL), water (40 mL) were added to a 500 mL flask under a nitrogen gas flow, and the mixed solution was stirred for 24 hours under reflux condition. After completion of the reaction, an organic layer was extracted using CH₂Cl₂ and water. The extracted solution was treated with MgSO₄ to remove residual moisture therefrom, concentrated under reduced pressure, purified using column chromatography, and recrystallized to obtain the product P11. (88%, 9.15 g)

TABLE 1

| | Reactant 1 | Reactant 2 | Product | Yield | [M + H]⁺ |
|---|---|---|---|---|---|
| P1 | 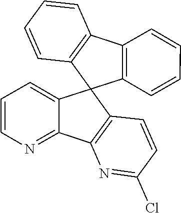 [SMILES: Clc1ccc2c(n1)-c1ncccc1C21c2ccccc2-c2ccccc21] | 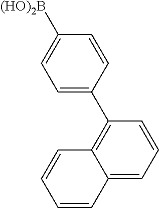 [SMILES: OB(O)c1ccc(-c2cccc3ccccc23)cc1] | 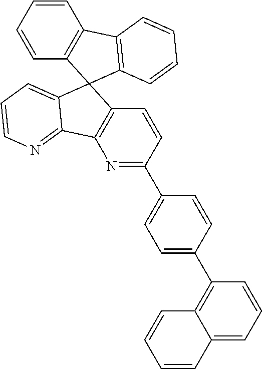 [SMILES: c1ccc2c(c1)-c1ccccc1C21c2cccnc2-c2nc(-c3ccc(-c4cccc5ccccc45)cc3)ccc21] | 9.17 g (88%) | 521.2 |
| | | | Compound 2 | | |
| P2 | 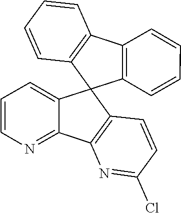 [SMILES: Clc1ccc2c(n1)-c1ncccc1C21c2ccccc2-c2ccccc21] | 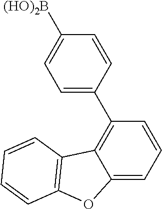 [SMILES: OB(O)c1ccc(-c2cccc3oc4ccccc4c23)cc1] | 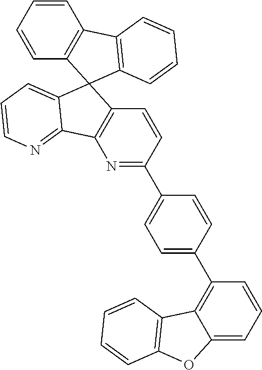 [SMILES: c1ccc2c(c1)-c1ccccc1C21c2cccnc2-c2nc(-c3ccc(-c4cccc5oc6ccccc6c45)cc3)ccc21] | 9.41 g (84%) | 561.2 |
| | | | Compound 107 | | |
| P3 | 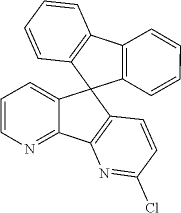 [SMILES: Clc1ccc2c(n1)-c1ncccc1C21c2ccccc2-c2ccccc21] | 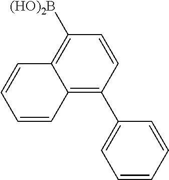 [SMILES: OB(O)c1ccc(-c2ccccc2)c2ccccc12] | 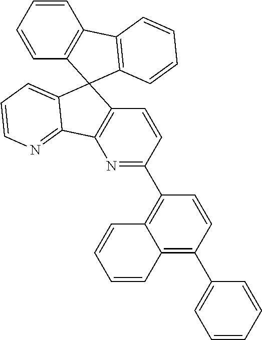 [SMILES: c1ccc(-c2ccc(-c3ccc4c(n3)-c3ncccc3C43c4ccccc4-c4ccccc43)c3ccccc23)cc1] | 9.05 g (87%) | 521.2 |
| | | | Compound 9 | | |
| P4 | 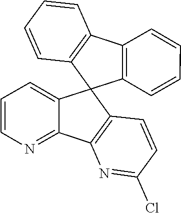 [SMILES: Clc1ccc2c(n1)-c1ncccc1C21c2ccccc2-c2ccccc21] | 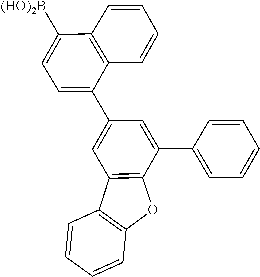 [SMILES: OB(O)c1ccc(-c2cc(-c3ccccc3)c3oc4ccccc4c3c2)c2ccccc12] | 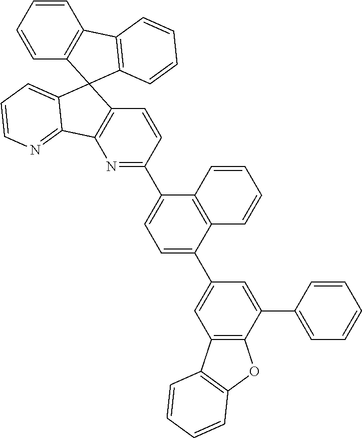 [SMILES: c1ccc(-c2cc(-c3ccc(-c4ccc5c(n4)-c4ncccc4C54c5ccccc5-c5ccccc54)c4ccccc34)cc3c2oc2ccccc23)cc1] | 11.11 g (81%) | 687.2 |
| | | | Compound 230 | | |
| P5 | 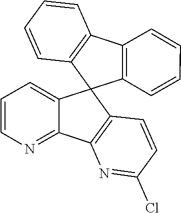 [SMILES: Clc1ccc2c(n1)-c1ncccc1C21c2ccccc2-c2ccccc21] | 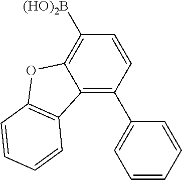 [SMILES: OB(O)c1ccc(-c2ccccc2)c2c1oc1ccccc12] | 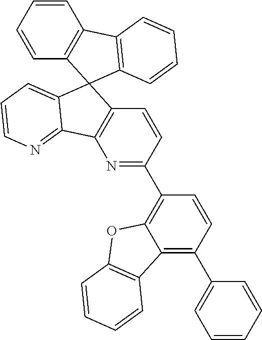 [SMILES: c1ccc(-c2ccc(-c3ccc4c(n3)-c3ncccc3C43c4ccccc4-c4ccccc43)c3oc4ccccc4c23)cc1] | 9.63 g (86%) | 561.2 |
| | | | Compound 20 | | |
| P6 | 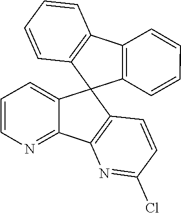 [SMILES: Clc1ccc2c(n1)-c1ncccc1C21c2ccccc2-c2ccccc21] | 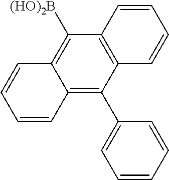 [SMILES: OB(O)c1c2ccccc2c(-c2ccccc2)c2ccccc12] | 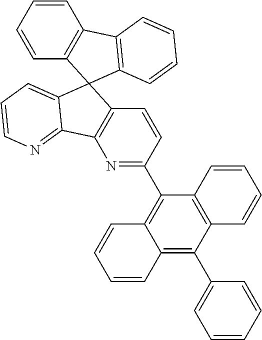 [SMILES: c1ccc(-c2c3ccccc3c(-c3ccc4c(n3)-c3ncccc3C43c4ccccc4-c4ccccc43)c3ccccc23)cc1] | 9.13 g (80%) | 571.2 |
| | | | Compound 16 | | |
| P7 | 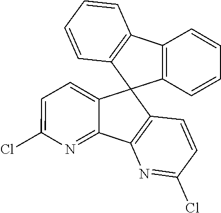 [SMILES: Clc1ccc2c(n1)-c1nc(Cl)ccc1C21c2ccccc2-c2ccccc21] | 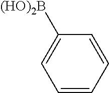 | 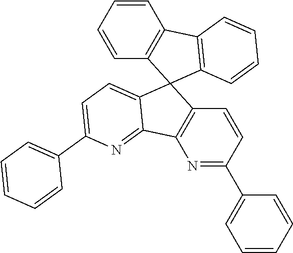 [SMILES: c1ccc(-c2ccc3c(n2)-c2nc(-c4ccccc4)ccc2C32c3ccccc3-c3ccccc32)cc1] | 7.80 g (83%) | 471.2 |
| | | | Compound 425 | | |
| P8 | 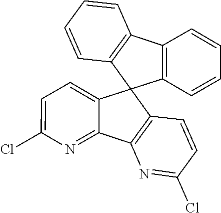 [SMILES: Clc1ccc2c(n1)-c1nc(Cl)ccc1C21c2ccccc2-c2ccccc21] | 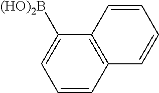 [SMILES: OB(O)c1cccc2ccccc12] | 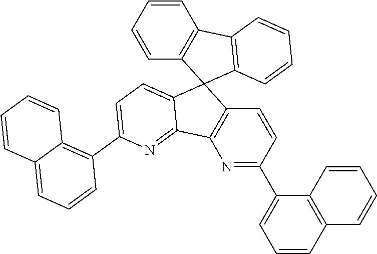 [SMILES: c1ccc2c(c1)-c1ccccc1C21c2ccc(-c3cccc4ccccc34)nc2-c2nc(-c3cccc4ccccc34)ccc21] | 9.35 g (82%) | 571.2 |
| | | | Compound 641 | | |
| P9 | 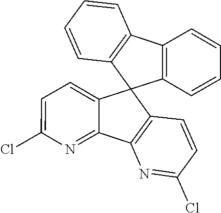 [SMILES: Clc1ccc2c(n1)-c1nc(Cl)ccc1C21c2ccccc2-c2ccccc21] | 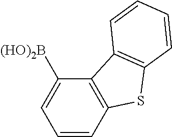 [SMILES: OB(O)c1cccc2sc3ccccc3c12] | 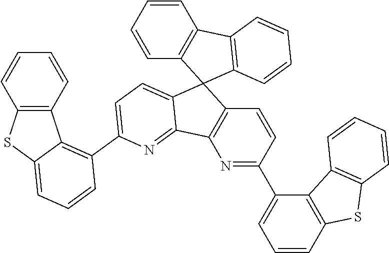 [SMILES: c1ccc2c(c1)-c1ccccc1C21c2ccc(-c3cccc4sc5ccccc5c34)nc2-c2nc(-c3cccc4sc5ccccc5c34)ccc21] | 10.25 g (75%) | 683.2 |
| | | | Compound 669 | | |
| P10 | 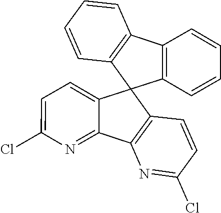 [SMILES: Clc1ccc2c(n1)-c1nc(Cl)ccc1C21c2ccccc2-c2ccccc21] | 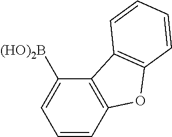 [SMILES: OB(O)c1cccc2oc3ccccc3c12] | 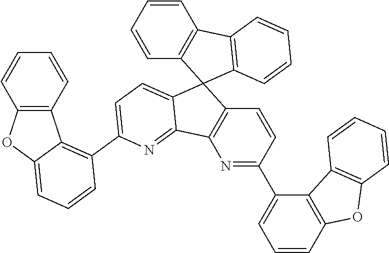 [SMILES: c1ccc2c(c1)-c1ccccc1C21c2ccc(-c3cccc4oc5ccccc5c34)nc2-c2nc(-c3cccc4oc5ccccc5c34)ccc21] | 10.01 g (77%) | 651.2 |
| | | | Compound 670 | | |
| P11 | 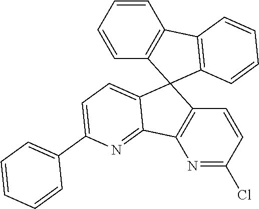 [SMILES: Clc1ccc2c(n1)-c1nc(-c3ccccc3)ccc1C21c2ccccc2-c2ccccc21] | 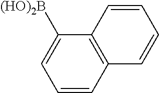 [SMILES: OB(O)c1cccc2ccccc12] | 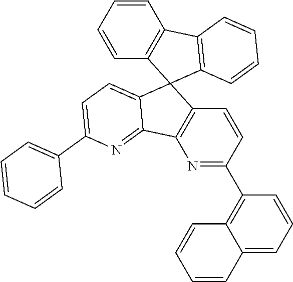 [SMILES: c1ccc(-c2ccc3c(n2)-c2nc(-c4cccc5ccccc45)ccc2C32c3ccccc3-c3ccccc32)cc1] | 9.15 g (88%) | 521.2 |
| | | | Compound 449 | | |
| P12 | 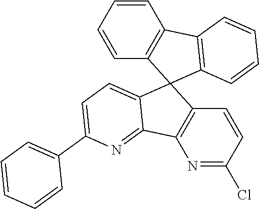 [SMILES: Clc1ccc2c(n1)-c1nc(-c3ccccc3)ccc1C21c2ccccc2-c2ccccc21] | 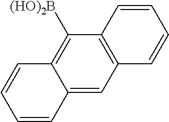 [SMILES: OB(O)c1c2ccccc2cc2ccccc12] | 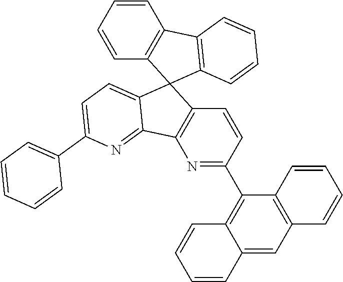 [SMILES: c1ccc(-c2ccc3c(n2)-c2nc(-c4c5ccccc5cc5ccccc45)ccc2C32c3ccccc3-c3ccccc32)cc1] | 8.56 g (75%) | 571.2 |
| | | | Compound 475 | | |
| P13 | 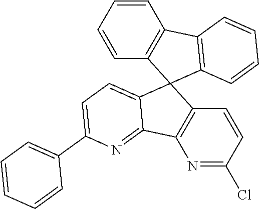 [SMILES: Clc1ccc2c(n1)-c1nc(-c3ccccc3)ccc1C21c2ccccc2-c2ccccc21] | 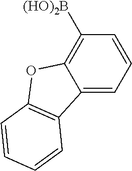 [SMILES: OB(O)c1cccc2c1oc1ccccc12] | 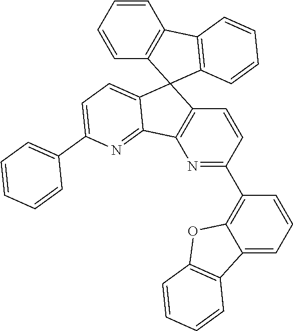 [SMILES: c1ccc(-c2ccc3c(n2)-c2nc(-c4cccc5c4oc4ccccc45)ccc2C32c3ccccc3-c3ccccc32)cc1] | 8.96 g (80%) | 561.2 |
| | | | Compound 501 | | |

### [Experimental Example 1] Identification of HOMO and LUMO Levels of n-Type Charge Generation Layer

The charge generation layer serves as a negative electrode and an positive electrode in each connection unit of the diode in the plurality of light emitting stacks or a tandem structure, generates holes and electrons, and injects the holes and electrons into the light emitting layer having the tandem structure. The n-type charge generation layer serves to transfer electrons at the interface to the electron transport layer via adjusting of a HOMO-LUMO level between the hole transport layer (or the p-type charge generation layer) and the electron transport layer. To this end, it is preferable that the HOMO and LUMO levels of the n-type charge generation layer are similar to the LUMO of the electron transport layer and the HOMO level of the hole transport layer (or the p-type charge generation layer).

In order to identify whether the compound represented by the Chemical Formula 1 of the present disclosure is suitable as the n-type charge generation layer material, HOMO and LUMO thereof were calculated using Gaussian software (B3LYP DFT 6-31G(d) by Gaussian′16.0), and are shown in Table 2 as set forth below.

TABLE 2

| Compound | HOMO (calculation) | LUMO (calculation) |
|---|---|---|
| 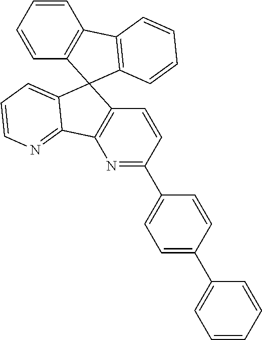 [SMILES: c1ccc(-c2ccc(-c3ccc4c(n3)-c3ncccc3C43c4ccccc4-c4ccccc43)cc2)cc1] | 5.52 | 1.62 |
| Compound 1 | | |
| 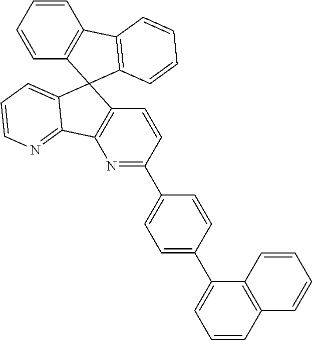 [SMILES: c1ccc2c(c1)-c1ccccc1C21c2cccnc2-c2nc(-c3ccc(-c4cccc5ccccc45)cc3)ccc21] | 5.55 | 1.65 |
| Compound 2 | | |
| 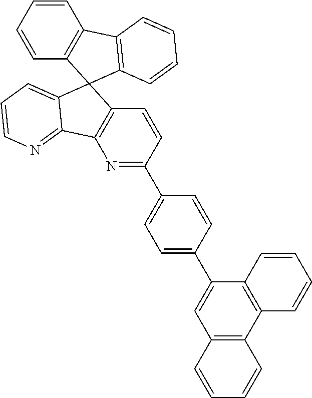 [SMILES: c1ccc2c(c1)-c1ccccc1C21c2cccnc2-c2nc(-c3ccc(-c4cc5ccccc5c5ccccc45)cc3)ccc21] | 5.54 | 1.63 |
| Compound 3 | | |

### Present Example 1: Manufacture of Organic Light-Emitting Diode

A substrate on which ITO (100 nm) as a positive electrode of an organic light-emitting diode was deposited was patterned in a distinguishing manner of a positive electrode area, a negative electrode area, and an insulating layer area from each other in an exposure (Photo-Lithography) process. Then, for the purpose of increasing a work-function of the positive electrode, that is, ITO and cleaning, a surface-treatment was performed thereon using UV-ozone and O₂:N₂ plasma.

Next, 1,4,5,8,9,11-hexaazatriphenylene-hexacarbonitrile (HAT-CN) was formed on the positive electrode so as to have a thickness of 10 nm to form a hole injection layer (HIL). On the hole injection layer, N4,N4,N4′,N4′-tetra([1,1′-biphenyl]-4-yl)-[1,1′-biphenyl]-4,4′-diamine was vacuum deposited to form a first hole transport layer (HTL 1) having a thickness of 90 nm. N-phenyl-N-(4-(spiro[benzo[d, e]anthracene-7,9′-fluorene]-2′-yl)phenyl)dibenzo[b,d]furan-4-amine was formed on the first hole transport layer (HTL 1) so as to have a thickness of 15 nm to form an electron blocking layer (EBL).

On the electron blocking layer (EBL), 9,10-bis(2-naphthyl)anthracene (ADN) as a host was deposited, and 2,12-di-tert-butyl-5,9-bis(4-(tert-butyl)phenyl)-7-(3,5-di-tert-butylphenyl)-5,9-dihydro5,9-diaza-13b-boranaphtho[3,2,1-de]anthracene (t-DABNA-dtB) as a dopant was doped into the host in a content of 3 wt % (host:dopant=97:3, a weight ratio). Thus, a first light emission layer (EML 1) having a thickness of 25 nm was formed.

On the first light emission layer (EML 1), 2-(4-(9,10-di(naphthalene-2-yl)anthracene-2-yl)phenyl)-1-phenyl-1H-benzo[d]imidazole was deposited so as to have a thickness of 25 nm to form a first electron transport layer (ETL 1).

A mixture of Compound 641 as a host compound (n-type host) of the n-type charge generation layer and Li as a dopant compound (n-type dopant) of the n-type charge generation layer was deposited on the first electron transport layer (ETL 1) to form the n-type charge generation layer (n-CGL) of a thickness of 185 Å. In this regard, when the weight of Compound 641 which is the n-type host was 100 wt %, a content of Li as the n-type dopant was 2 wt %.

HAT-CN as the p-type dopant was co-deposited on the n-type charge generation layer (n-CGL) to form the p-type charge generation layer (p-CGL) having a thickness of 80 Å.

N4,N4,N4′,N4′-tetra([1,1′-biphenyl]-4-yl)-[1,1′-biphenyl]-4,4′-diamine was deposited on the p-type charge generation layer (p-CGL) so as to have a thickness of 30 nm to form a second hole transport layer (HTL 2).

On the second hole transport layer, 4,4′-N,N′-dicarbazole-biphenyl (CBP) as a host was deposited and was doped with Ir(ppy)₃[tris(2-phenylpyridine)-iridium] as a dopant in an amount of 5 wt % (host:dopant=95:5, weight ratio). Thus, a second light emitting layer (EML 2) of a thickness of 35 nm was deposited on the second hole transport layer.

N-phenyl-N-(4-(10-phenylanthracen-9-yl)phenyl)pyridin-4-amine and Liq were mixed with each other in a ratio of 1:1, and the mixture was deposited on the second light emitting layer (EML 2). Thus, a second electron transport layer (ETL 2) was deposited on the second EML 2 so as to have a thickness of 30 nm. An electron injection layer (EIL) made of Liq and having 1 nm was deposited on the second electron transport layer (ETL 2). A negative electrode made of aluminum (Al) was deposited on the electron injection layer (EIL) so as to have a thickness of 16 nm. N4,N4′-bis[4-[bis(3-Methylphenyl)amino]phenyl]-N4,N4′-diphenyl-[1,1′-biphenyl]-4,4′-diamine (DNTPD) as a capping layer was deposited above the negative electrode in a thickness of 60 nm. A seal cap containing a moisture absorbent is bonded to the capping layer (CPL) via a UV curable adhesive to protect the organic light-emitting device from oxygen or moisture in the atmosphere. In this way, the organic light-emitting device was manufactured.

### Present Examples 2 to 56

Organic light-emitting diodes of Present Examples 2 to 56 were manufactured in the same manner as in Present Example 1, except that the compound 641 used as the n-type host of the n-type charge generation layer of Present Example 1 was changed to what is shown in Table 3 as set forth below.

### Comparative Examples 1 to 4

Organic light-emitting diodes of Comparative Examples 1 to 4 were manufactured in the same manner as in Present Example 1, except that the Compound 641 used as the n-type host of the n-type charge generation layer of Present Example 1 was changed to Comparative Compounds A to D as set forth below.

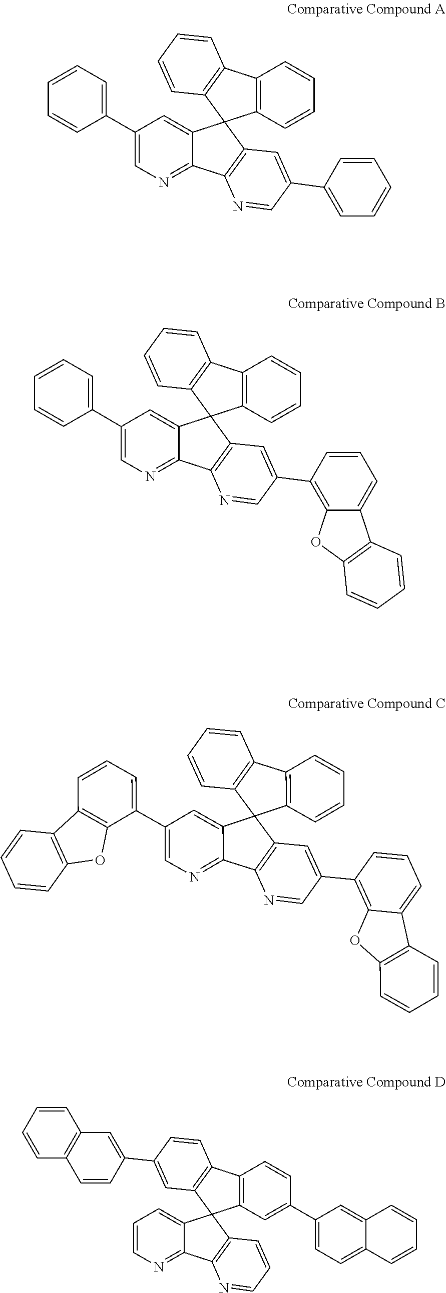 [SMILES: c1ccc(-c2cnc3c(c2)C2(c4ccccc4-c4ccccc42)c2cc(-c4ccccc4)cnc2-3)cc1 ; c1ccc(-c2cnc3c(c2)C2(c4ccccc4-c4ccccc42)c2cc(-c4cccc5c4oc4ccccc45)cnc2-3)cc1 ; c1ccc2c(c1)-c1ccccc1C21c2cc(-c3cccc4c3oc3ccccc34)cnc2-c2ncc(-c3cccc4c3oc3ccccc34)cc21 ; c1cnc2c(c1)C1(c3cc(-c4ccc5ccccc5c4)ccc3-c3ccc(-c4ccc5ccccc5c4)cc31)c1cccnc1-2]

### [Experimental Example 2] Performance Evaluation of Organic Light-Emitting Diode

The efficiency (Cd/A) of the organic light-emitting diode of each of Present Examples 1 to 56 and Comparative Examples 1 to 4 was measured by applying a 10 mA/cm² current thereto using KONICA MINOLTA CS-2000, and the lifetime (LT95) thereof was measured based on a time for which from the initial luminance was reduced to 95% of the initial luminance under operation at a constant current at 10 mA/cm² using McScience M6000.

The measurement results are shown in Table 3 as set forth below.

TABLE 3

| | N-type charge | Operation | | |
|---|---|---|---|---|
| | generation | voltage | Efficiency | Lifetime |
| Examples | layer material | (V) | (Cd/A) | (hr) |
| Present Example 1 | Compound 641 | 7.45 | 8.8 | 139 |
| Present Example 2 | Compound 3 | 7.55 | 8.4 | 110 |
| Present Example 3 | Compound 2 | 7.54 | 8.5 | 112 |
| Present Example 4 | Compound 107 | 7.55 | 8.4 | 112 |
| Present Example 5 | Compound 115 | 7.52 | 8.7 | 119 |
| Present Example 6 | Compound 124 | 7.52 | 8.8 | 120 |
| Present Example 7 | Compound 125 | 7.55 | 8.4 | 111 |
| Present Example 8 | Compound 148 | 7.52 | 8.9 | 120 |
| Present Example 9 | Compound 7 | 7.52 | 8.9 | 130 |
| Present Example 10 | Compound 175 | 7.54 | 8.5 | 113 |
| Present Example 11 | Compound 181 | 7.51 | 8.7 | 111 |
| Present Example 12 | Compound 9 | 7.50 | 8.9 | 128 |
| Present Example 13 | Compound 11 | 7.50 | 8.9 | 126 |
| Present Example 14 | Compound 207 | 7.51 | 8.8 | 122 |
| Present Example 15 | Compound 230 | 7.49 | 8.8 | 128 |
| Present Example 16 | Compound 16 | 7.58 | 8.0 | 100 |
| Present Example 17 | Compound 17 | 7.58 | 8.0 | 103 |
| Present Example 18 | Compound 18 | 7.58 | 8.0 | 103 |
| Present Example 19 | Compound 257 | 7.57 | 8.2 | 109 |
| Present Example 20 | Compound 279 | 7.57 | 8.2 | 110 |
| Present Example 21 | Compound 20 | 7.51 | 8.6 | 121 |
| Present Example 22 | Compound 21 | 7.51 | 8.6 | 122 |
| Present Example 23 | Compound 22 | 7.51 | 8.7 | 126 |
| Present Example 24 | Compound 24 | 7.50 | 8.8 | 129 |
| Present Example 25 | Compound 25 | 7.50 | 8.7 | 128 |
| Present Example 26 | Compound 26 | 7.50 | 8.8 | 129 |
| Present Example 27 | Compound 425 | 7.53 | 8.5 | 125 |
| Present Example 28 | Compound 428 | 7.52 | 8.7 | 129 |
| Present Example 29 | Compound 29 | 7.53 | 8.6 | 125 |
| Present Example 30 | Compound 30 | 7.53 | 8.4 | 120 |
| Present Example 31 | Compound 449 | 7.48 | 8.9 | 131 |
| Present Example 32 | Compound 450 | 7.49 | 8.8 | 130 |
| Present Example 33 | Compound 451 | 7.47 | 8.9 | 133 |
| Present Example 34 | Compound 475 | 7.57 | 8.3 | 113 |
| Present Example 35 | Compound 476 | 7.56 | 8.3 | 115 |
| Present Example 36 | Compound 477 | 7.57 | 8.3 | 113 |
| Present Example 37 | Compound 493 | 7.56 | 8.3 | 117 |
| Present Example 38 | Compound 501 | 7.49 | 8.7 | 130 |
| Present Example 39 | Compound 32 | 7.49 | 8.8 | 131 |
| Present Example 40 | Compound 514 | 7.51 | 8.6 | 120 |
| Present Example 41 | Compound 36 | 7.47 | 8.8 | 133 |
| Present Example 42 | Compound 1 | 7.55 | 8.4 | 111 |
| Present Example 43 | Compound 642 | 7.48 | 8.8 | 130 |
| Present Example 44 | Compound 647 | 7.47 | 8.9 | 133 |
| Present Example 45 | Compound 669 | 7.50 | 8.7 | 129 |
| Present Example 46 | Compound 670 | 7.45 | 8.9 | 138 |
| Present Example 47 | Compound 655 | 7.45 | 8.8 | 138 |
| Present Example 48 | Compound 656 | 7.46 | 8.8 | 135 |
| Present Example 49 | Compound 657 | 7.46 | 8.8 | 135 |
| Present Example 50 | Compound 658 | 7.46 | 8.9 | 135 |
| Present Example 51 | Compound 659 | 7.48 | 8.7 | 132 |
| Present Example 52 | Compound 660 | 7.47 | 8.8 | 133 |
| Present Example 53 | Compound 677 | 7.46 | 8.8 | 135 |
| Present Example 54 | Compound 678 | 7.51 | 8.6 | 122 |
| Present Example 55 | Compound 681 | 7.53 | 8.8 | 131 |
| Present Example 56 | Compound 683 | 7.51 | 8.7 | 130 |
| Comparative | Comparative | 8.12 | 7.6 | 86 |
| Example 1 | Compound A | | | |
| Comparative | Comparative | 8.16 | 7.5 | 92 |
| Example 2 | Compound B | | | |
| Comparative | Comparative | 8.19 | 7.3 | 95 |
| Example 3 | Compound C | | | |
| Comparative | Comparative | 8.20 | 7.9 | 90 |
| Example 4 | Compound D | | | |

As shown in Table 3, it was identified that the organic light-emitting diode including the compound of the present disclosure as the n-type charge generation layer exhibited a lower operation voltage and significantly better lifespan than those in Comparative Example. In addition, it was identified that when R₃ or Ar is a naphthyl group or a dibenzofuranyl group, overall life span of the diode is improved.

### Present Examples 57 to 59, and Comparative Examples 5 and 6

The n-type charge generation layer (n-CGL) of Present Example 1 includes a n-type charge generation layer compound and an alkali metal. An organic light-emitting diode including the n-type charge generation layer including Compound 641 as the n-type charge generation layer compound and including Li as the alkali metal was manufactured while the weight of the alkali metal based on 100 wt % of the n-type charge generation layer compound was changed. In this regard, a mixing ratio therebetween is as shown in Table 4 as set forth below. An organic light-emitting diode was manufactured under the same conditions as in Present Example 1 except for the varying mixing ratio therebetween.

### Experimental Example 3: Diode Performance Evaluation Based on Content of n-Type Dopant

The organic light-emitting diodes of Present Examples 57 to 59 and Comparative Examples 5 and 6 were manufactured in the same manner as in Present Example 1, except that the amount of Li as the n-type dopant added to Compound 641 used as the n-type host of the n-type charge generation layer of Present Example 1 was changed as shown in Table 4 as set forth below. The amount of the n-type dopant in Table 4 as set forth below is based on a total weight 100% of Compound 641.

In the same manner as in Experimental Example 2, the operation voltage, the efficiency (EQE), and the lifetime (LT95) were measured on each of the organic light-emitting diodes of Present Examples 57 to 59, Comparative Example 5, and Comparative Example 6, and the measurement results are shown in Table 4 as set forth below.

TABLE 4

| | | n-type dopant | Operation | Efficiency | Lifetime |
|---|---|---|---|---|---|
| Examples | n-type host | (Amount Added) | voltage (V) | (EQE, %) | (hr) |
| Present | Compound 641 | Li (0.5 wt %) | 7.79 | 8.3 | 125 |
| Example 57 | | | | | |
| Present | Compound 641 | Li (2 wt %) | 7.56 | 8.8 | 139 |
| Example 58 | | | | | |
| Present | Compound 641 | Li (5 wt %) | 7.55 | 8.1 | 121 |
| Example 59 | | | | | |
| Comparative | Compound 641 | Li (10 wt %) | 7.95 | 7.5 | 98 |
| Example 5 | | | | | |
| Comparative | Compound 641 | Li (0.1 wt %) | 8.03 | 8.1 | 119 |
| Example 6 | | | | | |

As shown in Table 4, it may be identified that when Li as the n-type dopant is added in an amount of 0.5 to 5 wt % based on 100 wt % of the n-type host (compound 641), the organic light-emitting diode exhibits excellent characteristics. Li as the n-type dopant has sufficient electrons. Thus, even when Li is added to the n-type host compound in excess of 5 wt %, the total amount of electrons is maintained at the same level, and thus no further improvement effect could be expected. In addition, when an excessive amount of the n-type dopants is added, leakage current and short-circuit of the organic light-emitting diode may occur, and rather, the operation voltage of the organic light-emitting diode may be increased and efficiency and lifespan of the organic light-emitting diode may be reduced.

Although embodiments of the present disclosure have been described with reference to the accompanying drawings, the present disclosure is not limited to the above embodiments, but may be implemented in various different forms. A person skilled in the art may appreciate that the present disclosure may be practiced in other concrete forms without changing the technical spirit or essential characteristics of the present disclosure. Therefore, it should be appreciated that the embodiments as described above is not restrictive but illustrative in all respects.

What is claimed is:

1. An organic compound represented by a following Chemical Formula 1-1: 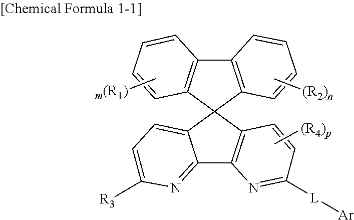
wherein in the Chemical Formula 1-1,
m and n are identical with or different from each other, and each of m and n is independently an integer of 1 to 4,
p is independently an integer of 1 to 2,
L is selected from a single bond, a substituted or unsubstituted arylene group having 6 to 30 carbon atoms, and a substituted or unsubstituted heteroarylene group having 5 to 60 carbon atoms,
Ar is selected from hydrogen, deuterium, a substituted or unsubstituted aryl group having 6 to 30 carbon atoms, a substituted or unsubstituted arylalkyl group having 7 to 30 carbon atoms, a substituted or unsubstituted heteroaryl group having 5 to 60 carbon atoms, a substituted or unsubstituted heteroarylalkyl group having 6 to 60 carbon atoms, a substituted or unsubstituted alkyl group having 1 to 30 carbon atoms, a substituted or unsubstituted arylamino group having 6 to 30 carbon atoms, a substituted or unsubstituted arylalkylamino group having 6 to 30 carbon atoms, a substituted or unsubstituted heteroarylamino group having 5 to 60 carbon atoms, a substituted or unsubstituted arylsilyl group having 6 to 30 carbon atoms, and a substituted or unsubstituted aryloxy having 6 to 30 carbon atoms,
R₁ to R₄ are identical with or different from each other, and each of R₁ to R₄ is independently selected from hydrogen, deuterium, a substituted or unsubstituted aryl group having 6 to 30 carbon atoms, a substituted or unsubstituted arylalkyl group having 7 to 30 carbon atoms, a substituted or unsubstituted heteroaryl group having 5 to 60 carbon atoms, a substituted or unsubstituted heteroarylalkyl group having 6 to 60 carbon atoms, a substituted or unsubstituted alkyl group having 1 to 30 carbon atoms, a substituted or unsubstituted arylamino group having 6 to 30 carbon atoms, a substituted or unsubstituted arylalkylamino group having 6 to 30 carbon atoms, a substituted or unsubstituted heteroarylamino group having 5 to 60 carbon atoms, a substituted or unsubstituted arylsilyl group having 6 to 30 carbon atoms, and a substituted or unsubstituted aryloxy group having 6 to 30 carbon atoms,
wherein when L, Ar, R₁, R₂, R₃, and R₄ are respectively substituted with substituents, the substituents are identical with or different from each other, wherein each of the substituents independently includes at least one selected from deuterium, a cyano group, a trifluoromethyl group, a nitro group, a halogen group, a hydroxy group, a trimethylsilyl group (TMS), an alkyl group having 1 to 30 carbon atoms, a cycloalkyl group having 3 to 20 carbon atoms, an alkenyl group having 2 to 30 carbon atoms, a cycloalkenyl group having 3 to 20 carbon atoms, an alkynyl group having 2 to 30 carbon atoms, a cycloalkynyl group having 3 to 20 carbon atoms, an aryl group having 6 to 30 carbon atoms, an arylalkyl group having 7 to 30 carbon atoms, a heteroaryl group having 5 to 60 carbon atoms, a heteroarylalkyl group having 6 to 60 carbon atoms, an amine group, an alkylamino group having 1 to 30 carbon atoms, an arylalkylamino group having 7 to 30 carbon atoms, an arylamino group having 6 to 30 carbon atoms, a heteroarylamino group having 5 to 60 carbon atoms, a silyl group, an alkylsilyl group having 1 to 30 carbon atoms, an arylsilyl group having 6 to 30 carbon atoms, an alkoxy group having 1 to 30 carbon atoms, an aryloxy group having 6 to 30 carbon atoms, an alkylthio group having 1 to 30 carbon atoms, and an arylthio group having 6 to 30 carbon atoms, when a plurality of substituents are present, each substituent is the same as or different from each other.

2. The organic compound of claim 1, wherein the organic compound represented by the Chemical Formula 1-1 is represented by one of following Chemical Formulas 10 to 21: 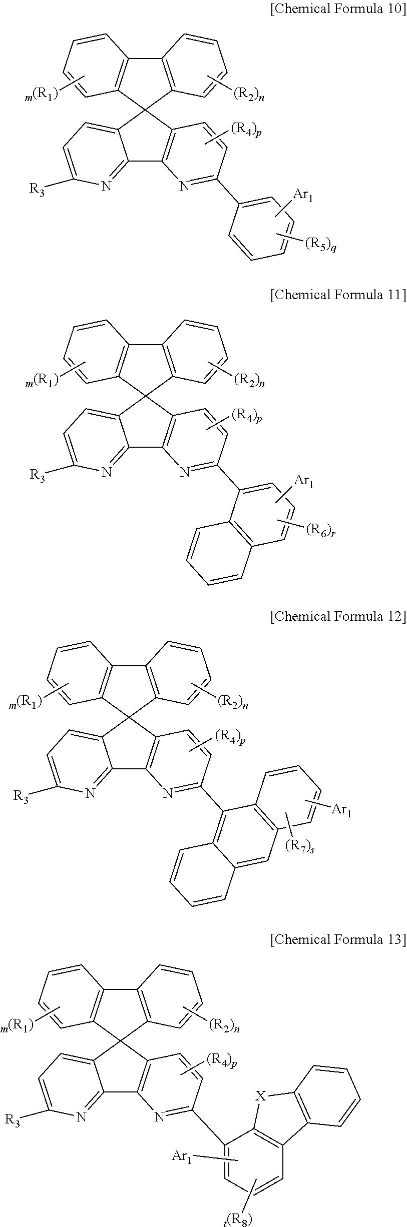 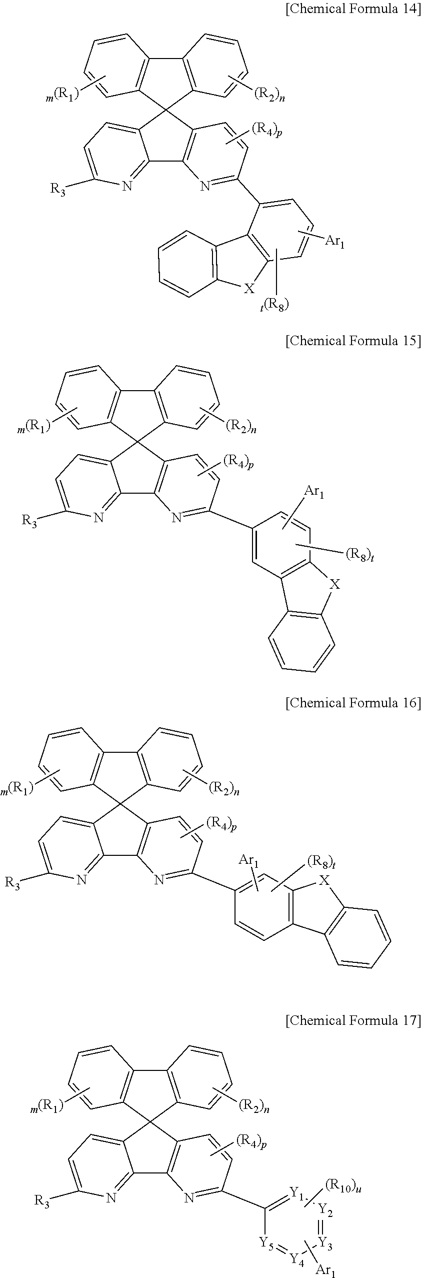 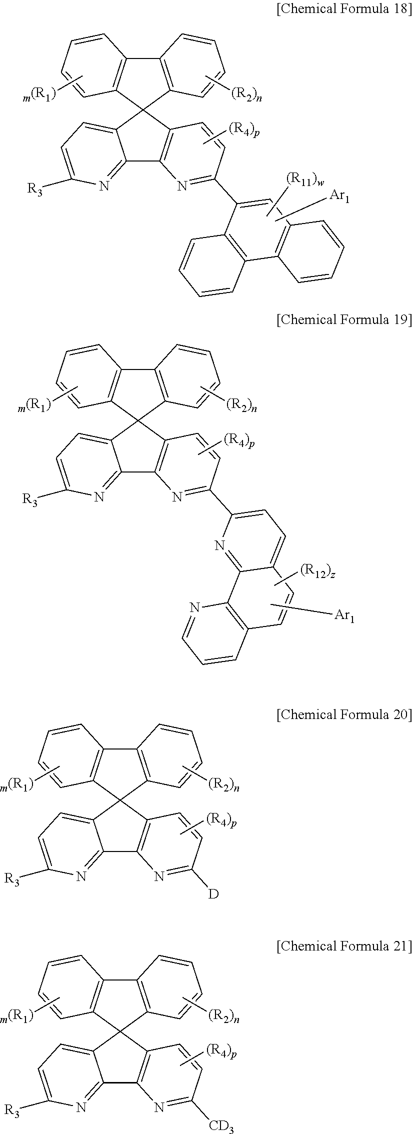
wherein in the Chemical Formulas 10 to 21,
Ar₁ is the same as the definition of Ar described in the Chemical Formula 1-1,
each of R₁ to R₄, n, m, and p is the same as defined in the Chemical Formula 1-1,
q is an integer of 1 to 4, r is an integer of 1 to 6, s is an integer of 1 to 8, t is an integer of 1 to 6, u is an integer of 1 to 4, w is an integer of 0 to 9, and z is an integer of 0 to 7,
X is O or S,
R₅ to R₈ and R₁₀ to R₁₂ are identical with or different from each other, and each of R₅ to R₈ and R₁₀ to R₁₂ is independently selected from hydrogen, deuterium, a substituted or unsubstituted aryl group having 6 to 30 carbon atoms, a substituted or unsubstituted arylalkyl group having 7 to 30 carbon atoms, a substituted or unsubstituted heteroaryl group having 5 to 60 carbon atoms, a substituted or unsubstituted heteroarylalkyl group having 6 to 60 carbon atoms, a substituted or unsubstituted arylamino group having 6 to 30 carbon atoms, a substituted or unsubstituted arylalkylamino group having 6 to 30 carbon atoms, a substituted or unsubstituted heteroarylamino group having 5 to 60 carbon atoms, a substituted or unsubstituted arylsilyl group having 6 to 30 carbon atoms, and a substituted or unsubstituted aryloxy group having 6 to 30 carbon atoms,
wherein when Ar₁, R₁ to R₈, and R₁₀ to R₁₂ are respectively substituted with substituents, the substituents are identical with or different from each other, wherein each of the substituents independently includes at least one selected from deuterium, a cyano group, a trifluoromethyl group, a nitro group, a halogen group, a hydroxy group, a trimethylsilyl group (TMS), an alkyl group having 1 to 30 carbon atoms, a cycloalkyl group having 3 to 20 carbon atoms, an alkenyl group having 2 to 30 carbon atoms, a cycloalkenyl group having 3 to 20 carbon atoms, an alkynyl group having 2 to 30 carbon atoms, a cycloalkynyl group having 3 to 20 carbon atoms, an aryl group having 6 to 30 carbon atoms, an arylalkyl group having 7 to 30 carbon atoms, a heteroaryl group having 5 to 60 carbon atoms, a heteroarylalkyl group having 6 to 60 carbon atoms, an amine group, an alkylamino group having 1 to 30 carbon atoms, an arylalkylamino group having 7 to 30 carbon atoms, an arylamino group having 6 to 30 carbon atoms, a heteroarylamino group having 5 to 60 carbon atoms, a silyl group, an alkylsilyl group having 1 to 30 carbon atoms, an arylsilyl group having 1 to 30 carbon atoms, an alkoxy group having 1 to 30 carbon atoms, an aryloxy group having 6 to 30 carbon atoms, an alkylthio group having 1 to 30 carbon atoms, and an arylthio group having 6 to 30 carbon atoms, when a plurality of substituents are present, each substituent is the same as or different from each other.

3. An organic light-emitting diode comprising:
a positive electrode;
a negative electrode facing the positive electrode;
N light emitting stacks disposed between the positive electrode and the negative electrode include, wherein N is an integer of 2 or larger; and
N−1 n-type charge generation layers and N−1 p-type charge generation layers, wherein each of the N−1 n-type charge generation layers and each of the N−1 p-type charge generation layers are disposed on top of each other and contact each other and are disposed between different and adjacent light emitting stacks of the N light emitting stacks,
wherein at least one of the N−1 n-type charge generation layers includes the compound represented by the Chemical Formula 1-1 of claim 1.

4. The organic light-emitting diode of claim 3, wherein the compound represented by the Chemical Formula 1-1 included in the n-type charge generation layer is a n-type host material,
wherein the n-type charge generation layer includes the n-type host material and a n-type dopant material doped thereto.

5. The organic light-emitting diode of claim 4, wherein the n-type dopant material is an alkali metal or alkaline earth metal compound.

6. The organic light-emitting diode of claim 4, wherein a content of the n-type dopant material is in a range of about 0.1 wt % to about 10 wt %, based on 100 wt % of a total weight of the n-type host material.

7. The organic light-emitting diode of claim 3, wherein N is one of 2, 3, 4, and 5.

8. The organic light-emitting diode of claim 3, wherein each of the N light emitting stacks includes an electron transport layer, a hole transport layer, and a light emitting layer disposed between the electron transport layer and the hole transport layer.
